SEQUENCE LISTING (210 sequences)

SEQ ID NO: 1 - protein, 7 aa, synthetic construct
GYTFTIY

SEQ ID NO: 2 - protein, 6 aa, synthetic construct
NPSGGS

SEQ ID NO: 3 - protein, 16 aa, synthetic construct
NDYVDYVTTDYYGMDV

SEQ ID NO: 4 - protein, 11 aa, synthetic construct
RASQGISNYLA

SEQ ID NO: 5 - protein, 7 aa, synthetic construct
AASSLQS

SEQ ID NO: 6 - protein, 9 aa, synthetic construct
QQYNSSPYT

SEQ ID NO: 7 - protein, 125 aa, synthetic construct
QVHLVQSGAEVKKPGASVKVSCKASGYTFTIYYIHWVRQAPGQGLEWMGIINPSGGSTSYAQKFQGRVTMTRDTSTSTVYMELSSLRSEDTAVYYCSGNDYVDYVTTDYYGMDVWGQGTTVTVSS

SEQ ID NO: 8 - protein, 107 aa, synthetic construct
DIQMTQSPSSLSASVGDRVTITCRASQGISNYLAWFQQKPGKAPKSLIYAASSLQSGVPSKFSGSGSGTDFTLTISSLQPEDFATYYCQQYNSSPYTFGQGTKLEIK

SEQ ID NO: 9 - DNA, 375 bp, synthetic construct
  source 1..375: mol_type = other DNA
caggtgcatctggtgcagtctggggctgaggtgaagaagcctggggcctcagtgaaggtttcttgcaaggcatctggatacaccttcaccatctactatattcactgggtgcgacaggcccctggacaggggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtatattactgttcgggtaatgactacgttgactacgtaactactgactactacggtatggacgtctggggccaagggaccacggtcaccgtctcctca

SEQ ID NO: 10 - DNA, 321 bp, synthetic construct
  source 1..321: mol_type = other DNA
gacatccagatgacccagtctccatcctcactgtctgcatctgtaggagatagagtcaccatcacttgtcgggcgagtcagggcattagcaattatttagcctggtttcagcagaaaccagggaaagcccctaagtccctgatctatgctgcatccagtttgcaaagtggggtcccatcaaagttcagcggcagtggatctgggacagatttcacactcaccatcagcagcctgcagcctgaagattttgcaacttattactgccaacagtataatagttccccgtacacttttggccaggggaccaagctggagatcaaa

SEQ ID NO: 11 - protein, 7 aa, synthetic construct
GFTFSSY

SEQ ID NO: 12 - protein, 6 aa, synthetic construct
SGRGGD

SEQ ID NO: 13 - protein, 8 aa, synthetic construct
ERGLGFDY

SEQ ID NO: 14 - protein, 17 aa, synthetic construct
RSSQSLLDSDDGNTYLD

SEQ ID NO: 15 - protein, 7 aa, synthetic construct
TLSYRAS

SEQ ID NO: 16 - protein, 9 aa, synthetic construct
MQHLEFPLT

SEQ ID NO: 17 - protein, 117 aa, synthetic construct
EVQLLESGGGLVQPGGSLRLSCAASGFTFSSYAMGWVRQTPGKGLEWVSTISGRGGDTYYADSVKGRFTISRDNSKNTLYLQMNSLRAEDTAIYYCAKERGLGFDYWGQGTLVTVSS

SEQ ID NO: 18 - protein, 113 aa, synthetic construct
DIVMTQTPLSLPVTPGEPASISCRSSQSLLDSDDGNTYLDWYLQKPGQSPQLLIYTLSYRASGVPDRFSGSGSGTDFTLKISRVEAEDVGVYYCMQHLEFPLTFGGGTKVEIK

SEQ ID NO: 19 - DNA, 351 bp, synthetic construct
  source 1..351: mol_type = other DNA
gaggtgcagctgttggagtctgggggaggcttggtacagcctggggggtccctgagactctcctgtgcagcctctggattcacctttagcagctatgccatgggctgggtccgccagactccagggaaggggctggagtgggtctcaactattagtggtcgtggtggtgacacatactacgcagactccgtgaagggccggttcaccatctccagagacaattccaagaacacgctgtatctgcaaatgaacagcctgagagccgaggacacggccatatattactgtgcgaaagagaggggactggggtttgactactggggccagggaaccctggtcaccgtctcctca

SEQ ID NO: 20 - DNA, 339 bp, synthetic construct
  source 1..339: mol_type = other DNA
gatattgtgatgacccagactccactctccctgcccgtcacccctggagagccggcctccatctcctgcaggtctagtcagagcctcttggatagtgatgatggaaacacctatttggactggtacctgcagaagccagggcagtctccacagctcctgatctatacgctttcctatcgggcctctggagtcccagacaggttcagtggcagtgggtcaggcactgatttcacactgaaaatcagcagggtggaggctgaggatgttggagtttattactgcatgcaacatctagagtttccgctcactttcggcggagggaccaaggtggagatcaaa

SEQ ID NO: 21 - protein, 7 aa, synthetic construct
GYTFTSY

SEQ ID NO: 22 - protein, 15 aa, synthetic construct
GDYDILTDYYYNMDV

SEQ ID NO: 23 - protein, 9 aa, synthetic construct
LQHNSYPYT

SEQ ID NO: 24 - protein, 124 aa, synthetic construct
QVQLVQSGAEVKKPGASVKVSCKASGYTFTSYYMHWVRQAPGQGLEWMGIINPSGGSTSYAQKFRGRVTMTRDTSTSTVYMELSSLRSEDTAVYYCARGDYDILTDYYYNMDVWGQGTTVTVSS

SEQ ID NO: 25 - protein, 107 aa, synthetic construct
DIQMTQSPSAMSASVGDRVTITCRASQGISNYLAWFQQKPGKVPKRLIYAASSLQSGVPSRFSGSGSGTEFTLTISSLQPEDFATYYCLQHNSYPYTFGQGTKLEIK

SEQ ID NO: 26 - DNA, 372 bp, synthetic construct
  source 1..372: mol_type = other DNA
caggtgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacagaagttccggggcagagtcaccatgaccagggacacgtccacgagcacagtctacatggagctgagcagcctgagatctgaggacacggccgtttattactgtgcgagaggggattacgatattttgacggactactactacaatatggacgtctggggccaagggaccacggtcaccgtctcctca

SEQ ID NO: 27 - DNA, 321 bp, synthetic construct
  source 1..321: mol_type = other DNA
gacatccagatgacccagtctccatctgccatgtctgcatctgtaggagacagagtcaccatcacttgtcgggcgagtcagggcattagcaattatttagcctggtttcagcagaaaccagggaaagtccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttacccgtacactttcggccaggggaccaagctggagatcaaa

SEQ ID NO: 28 - protein, 9 aa, synthetic construct
GFSLSTSGM

SEQ ID NO: 29 - protein, 5 aa, synthetic construct
DWDDN

SEQ ID NO: 30 - protein, 13 aa, synthetic construct
IRGYSGSYDAFDI

SEQ ID NO: 31 - protein, 16 aa, synthetic construct
RSSQSLLHSNGYNYLD

SEQ ID NO: 32 - protein, 7 aa, synthetic construct
LGSNRAP

SEQ ID NO: 33 - protein, 9 aa, synthetic construct
MQALQTPFT

SEQ ID NO: 34 - protein, 123 aa, synthetic construct
QVTLRESGPALVKSTQTLTLTCTFSGFSLSTSGMCVSWIRQTPGKALEWLALIDWDDNKYYSTSLKTRLIISKDTSKNQVVLTMTNMEPVDTATYYCARIRGYSGSYDAFDIWGQGTMVTVSS

SEQ ID NO: 35 - protein, 112 aa, synthetic construct
DIVMTQSPLSLPVTPGEPASISCRSSQSLLHSNGYNYLDWYLQKPGQSPHILLYLGSNRAPGVPDRFSGSGSGTDFTLKISRVEAEDVGVYYCMQALQTPFTFGPGTKVDIK

SEQ ID NO: 36 - DNA, 369 bp, synthetic construct
  source 1..369: mol_type = other DNA
caggtcaccttgagggagtctggtcctgcgctggtgaaatccacacagaccctcacactgacctgcaccttctctgggttctcactcagtactagtggaatgtgtgtgagctggatccgtcagaccccagggaaggccctggagtggcttgcactcattgattgggatgataataaatactacagcacatctctgaagaccaggctcatcatctccaaggacacctccaaaaaccaggtggtccttacaatgaccaatatggaacctgtggacacagccacgtattattgtgcacggatacgggggtatagtgggagctacgatgcttttgatatctggggccaagggacaatggtcaccgtctcttca

SEQ ID NO: 37 - DNA, 336 bp, synthetic construct
  source 1..336: mol_type = other DNA
gatattgtgatgactcagtctccactctccctgcccgtcacccctggagagccggcctccatctcctgcaggtctagtcagagcctcctgcatagtaatggatacaactatttggattggtacctgcagaagccagggcagtctccacacatcctgctctatttgggttctaatcgggcccccggggtccctgacaggttcagtggcagtggatcaggcacagattttacactgaaaatcagcagagtggaggctgaggatgttggggtttattactgcatgcaagctctacaaactccattcactttcggccctgggaccaaagtggatatcaaa

SEQ ID NO: 38 - protein, 6 aa, synthetic construct
NSSGGS

SEQ ID NO: 39 - protein, 15 aa, synthetic construct
GYCTNGVYYDEDFDY

SEQ ID NO: 40 - protein, 11 aa, synthetic construct
RASQGISHYLA

SEQ ID NO: 41 - protein, 9 aa, synthetic construct
QQYNSYPLT

SEQ ID NO: 42 - protein, 124 aa, synthetic construct
QVQLVQSGAEVKKPGASVKVSCKASGYTFTSYYMHWVRQAPGQGLEWMGVINSSGGSTSYAQKFQGRVTMTRDTSTSTVYMDLSSLRSEDTAVYYCARGYCTNGVYYDEDFDYWGQGTLVTVSS

SEQ ID NO: 43 - protein, 107 aa, synthetic construct
DIQMTQSPSSLSASVGDRVTITCRASQGISHYLAWFQQKPGKAPTSLIYAASSLQSGVPSKFSGSGSGTDFTLTISSLQPEDFATYYCQQYNSYPLTFGGGTKVEIK

SEQ ID NO: 44 - DNA, 372 bp, synthetic construct
  source 1..372: mol_type = other DNA
caggtgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggagtaatcaattctagtggtggtagcacaagctacgcacagaagttccagggcagagtcaccatgaccagggacacgtccacgagcacagtctacatggacctgagcagcctgagatctgaggacacggccgtgtattactgtgcgagagggtattgtactaatggtgtttactatgatgaggactttgactactggggccagggaaccctggtcaccgtctcctca

SEQ ID NO: 45 - DNA, 321 bp, synthetic construct
  source 1..321: mol_type = other DNA
gacatccagatgacccagtctccatcctcactgtctgcatctgtaggagacagagtcaccatcacttgtcgggcgagtcagggcattagccattatttagcctggtttcagcagaaaccagggaaagcccctacgtccctgatctatgctgcatccagtttgcaaagtggggtcccatcaaagttcagcggcagtggatctgggacagatttcactctcaccatcagcagcctgcagcctgaagattttgcaacttattactgccaacagtataatagttaccctctcactttcggcggagggaccaaggtggagatcaaa

SEQ ID NO: 46 - protein, 7 aa, synthetic construct
GGSISSY

SEQ ID NO: 47 - protein, 5 aa, synthetic construct
YTSGS

SEQ ID NO: 48 - protein, 10 aa, synthetic construct
EDWESWTFDI

SEQ ID NO: 49 - protein, 16 aa, synthetic construct
RSGQSLVYSDGDTYLN

SEQ ID NO: 50 - protein, 7 aa, synthetic construct
KVSNRDS

SEQ ID NO: 51 - protein, 9 aa, synthetic construct
MQGTHWPLS

SEQ ID NO: 52 - protein, 118 aa, synthetic construct
QVQLQESGPGLVKPSETLFLTCTVSGGSISSYYWTWIRQPAGKGLEWIGRIYTSGSTNYNPSLKSRVTMSVDTSKNQFSLKLSSVTAADTAVYYCAREDWESWTFDIWGQGTMVTVSS

SEQ ID NO: 53 - protein, 112 aa, synthetic construct
DVVMTQSPLSLPVTLGQPASISCRSGQSLVYSDGDTYLNWFQQRPGQSPRRLIYKVSNRDSGVPDRFSGSGSGTDFTLKISRVEAEDVGVYYCMQGTHWPLSFGGGTKVEIK

SEQ ID NO: 54 - DNA, 354 bp, synthetic construct
  source 1..354: mol_type = other DNA
caggtgcagctgcaggagtcgggcccaggactggtgaagccttcggagaccctgttcctcacctgcactgtctctggtggctccatcagtagttactactggacctggatccggcagcccgccgggaagggactggagtggattgggcgtatctataccagtgggagcaccaactacaacccctccctcaagagtcgagtcaccatgtcagtagacacgtccaagaaccagttctccctgaagctgagctctgtgaccgccgcggacacggccgtgtattactgtgcgagagaggactgggaatcgtggacttttgatatctggggccaagggacaatggtcaccgtctcttca

SEQ ID NO: 55 - DNA, 336 bp, synthetic construct
  source 1..336: mol_type = other DNA
gatgttgtgatgactcagtctccactctccctgcccgtcacccttggacagccggcctccatctcctgcaggtctggtcaaagcctcgtttacagtgatggagacacctacttgaattggtttcagcagaggccaggccaatctccaaggcgcctaatttataaggtttctaaccgggactctggggtcccagacagattcagcggcagtgggtcaggcactgatttcacactgaaaatcagcagggtggaggctgaggatgttggggtttattactgcatgcaaggtacacactggccgctctctttcggcggagggaccaaggtggagatcaaa

SEQ ID NO: 56 - protein, 11 aa, synthetic construct
QLELLGNAFDI

SEQ ID NO: 57 - protein, 12 aa, synthetic construct
RASQSVSSSYLA

SEQ ID NO: 58 - protein, 7 aa, synthetic construct
GASSRAT

SEQ ID NO: 59 - protein, 9 aa, synthetic construct
QQYGSSPYT

SEQ ID NO: 60 - protein, 120 aa, synthetic construct
QVQLVQSGAEVKKPGASVKVSCKASGYTFTSYYIHWVRQAPGQGLEWMGIINPSGGSKSYAQKFQGRVTMTRDTSTGTVYMELSSLRSEDTAVYYCAGQLELLGNAFDIWGQGTMVTVSS

SEQ ID NO: 61 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPYTFGQGTKLEIK

SEQ ID NO: 62 - DNA, 360 bp, synthetic construct
  source 1..360: mol_type = other DNA
caggtgcagctggtgcagtctggggctgaggtgaagaagcctggggcctcagtgaaggtttcctgcaaggcatctggatacaccttcaccagctactatatacactgggtgcgacaggcccctggacaagggcttgagtggatgggaataatcaaccctagtggtggtagtaaaagctacgcacagaagttccagggcagagtcaccatgaccagggacacgtccacgggaacagtctatatggagctgagcagcctgagatctgaggacacggccgtgtattactgtgcgggacaactggaacttttggggaatgcttttgatatctggggccaagggacaatggtcaccgtctcttca

SEQ ID NO: 63 - DNA, 324 bp, synthetic construct
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccgtacacttttggccaggggaccaagctggagatcaaa

SEQ ID NO: 64 - protein, 14 aa, synthetic construct
ERQQLDYYYFAMDV

SEQ ID NO: 65 - protein, 9 aa, synthetic construct
QQYNSYPPT

SEQ ID NO: 66 - protein, 123 aa, synthetic construct
QVQLVQSGAEVKKPGASVKVSCKASGYTFTSYYLHWVRQAPGHGLEWMAIINPSGGSTSYAQKFLGRVTMTRDTSTNTVYMELSSLRSEDTAVYYCARERQQLDYYYFAMDVWGQGTTVTVSS

SEQ ID NO: 67 - protein, 107 aa, synthetic construct
DIQMTQSPSSLSASVGDRVTITCRASQGISNYLAWFQQKPGKAPKSLIYAASSLQSGVPSKFSGSGSGTDFTLTISSLQPEDFATYYCQQYNSYPPTFGQGTKVEIK

SEQ ID NO: 68 - DNA, 369 bp, synthetic construct
  source 1..369: mol_type = other DNA
caggtgcagctggtgcagtctggggctgaggtgaagaagcctggggcctcagtgaaggtttcctgcaaggcatctggatacaccttcaccagctactatttgcactgggtgcgacaggcccctggacacgggcttgagtggatggcaataatcaaccctagtggtggtagtacaagctacgcacagaagttcctgggcagagtcaccatgaccagggacacgtccacgaacacagtctacatggagctgagcagcctgagatctgaggacacggccgtgtattactgtgcgagagaacggcagcagctggactactactacttcgctatggacgtctggggccaagggaccacggtcaccgtctcctca

SEQ ID NO: 69 - DNA, 321 bp, synthetic construct
  source 1..321: mol_type = other DNA
gacatccagatgacccagtctccatcctcactgtctgcatctgtaggagacagagtcaccatcacttgtcgggcgagtcagggcattagcaattatttagcctggtttcagcagaaaccagggaaagcccctaagtccctgatctatgctgcatccagtttgcaaagtggggtcccatcaaagttcagcggcagtggatctgggacagatttcactctcaccatcagcagcctgcagcctgaagattttgcaacttattactgccaacagtataatagttaccctccgacgttcggccaagggaccaaggtggaaatcaaa

SEQ ID NO: 70 - protein, 7 aa, synthetic construct
GFTFSFY

SEQ ID NO: 71 - protein, 6 aa, synthetic construct
SSSSSY

SEQ ID NO: 72 - protein, 11 aa, synthetic construct
NYDFWRGAFDI

SEQ ID NO: 73 - protein, 11 aa, synthetic construct
RASQSVSSNLA

SEQ ID NO: 74 - protein, 7 aa, synthetic construct
GASTRAT

SEQ ID NO: 75 - protein, 10 aa, synthetic construct
QQYNNWPTWT

SEQ ID NO: 76 - protein, 120 aa, synthetic construct
EVQLVESGGGLVKPGGSLRLSCAASGFTFSFYSMNWVRQAPGKGLEWVSSISSSSSYIYYADSVKGRFTISRDNAKNSLYLQMNSLRAEDTAVYYCARNYDFWRGAFDIWGQGTMVTVSS

SEQ ID NO: 77 - protein, 108 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPTWTFGQGTKVEIK

SEQ ID NO: 78 - DNA, 360 bp, synthetic construct
  source 1..360: mol_type = other DNA
gaggtgcagctggtggagtctggggggggcctggtcaagcctggagggtccctgagactctcctgtgcagcctctggattcaccttcagtttctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagaaattacgatttttggagaggtgcttttgatatctggggccaagggacaatggtcaccgtctcttca

SEQ ID NO: 79 - DNA, 324 bp, synthetic construct
  source 1..324: mol_type = other DNA
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcccacgtggacgttcggccaagggaccaaggtggaaatcaaa

SEQ ID NO: 80 - protein, 6 aa, synthetic construct
NYSGGS

SEQ ID NO: 81 - protein, 11 aa, synthetic construct
QGRYSGSYLDY

SEQ ID NO: 82 - protein, 7 aa, synthetic construct
QQYGSSP

SEQ ID NO: 83 - protein, 120 aa, synthetic construct
QVQLVQSGAEVKKPGASVKVSCKASGYTFTSYYMHWVRQAPGQGLEWMGIINYSGGSKSYAQKFQGRVTMTRDTSTSTVYMELSSLRSEDTGVYYCARQGRYSGSYLDYWGQGTLVTVSS

SEQ ID NO: 84 - protein, 106 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPFGQGTKLEIK

SEQ ID NO: 85 - DNA, 360 bp, synthetic construct
  source 1..360: mol_type = other DNA
caggtgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggaataatcaactatagtggtggtagcaaaagctacgcacagaagttccagggcagagtcaccatgacccgggacacgtccacgagcacagtctacatggagctgagcagcctgagatctgaggacacgggcgtgtattactgtgcgagacaggggcggtatagtgggagctaccttgactactggggccagggaaccctggtcaccgtctcctca

SEQ ID NO: 86 - DNA, 318 bp, synthetic construct
  source 1..318: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccttttggccaggggaccaagctggagatcaaa

SEQ ID NO: 87 - protein, 7 aa, synthetic construct
GYTFNSY

SEQ ID NO: 88 - protein, 6 aa, synthetic construct
NPSGGT

SEQ ID NO: 89 - protein, 10 aa, synthetic construct
DWGEQYGMDV

SEQ ID NO: 90 - protein, 16 aa, synthetic construct
RSSQSLLHSNGYNYLD

SEQ ID NO: 91 - protein, 9 aa, synthetic construct
MQALQTPLT

SEQ ID NO: 92 - protein, 119 aa, synthetic construct
QVQLVQSGAEVKKPGASLKVSCKTSGYTFNSYYMHWVRQAPGQGLEWMGINNPSGGTTSYAQKFQGRVTMTRDTSTSTVYMELSSLRSEDTAVYYCARDWGEQYGMDVWGQGTTVTVSS

SEQ ID NO: 93 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEIK

SEQ ID NO: 94 - DNA, 357 bp, synthetic construct
  source 1..357: mol_type = other DNA
caggtgcagctggtgcagtctggggctgaggtgaagaagcctggggcctcactgaaggtttcctgcaagacatctggatacaccttcaacagctactatatgcactgggtgcgacaggcccctggacaagggcttgagtggatgggaataaacaaccctagtggtggtaccacaagctacgcacagaagttccagggcagagtcaccatgaccagggacacgtccacgagcacagtctacatggagctgagcagcctgagatctgaggacacggccgtgtattactgtgcgagagattggggggagcagtacggtatggacgtctggggccaagggaccacggtcaccgtctcctca

SEQ ID NO: 95 - DNA, 336 bp, synthetic construct
  source 1..336: mol_type = other DNA
gatattgtgatgactcagtctccactctccctgcccgtcacccctggagagccggcctccatctcctgcaggtctagtcagagcctcctgcatagtaatggatacaactatttggattggtacctgcagaagccaggacagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactccgctcactttcggcggagggaccaaggtggagatcaaa

SEQ ID NO: 96 - protein, 7 aa, synthetic construct
GYTFTGY

SEQ ID NO: 97 - protein, 6 aa, synthetic construct
NPHSGA

SEQ ID NO: 98 - protein, 12 aa, synthetic construct
DSSGWYGDAFDI

SEQ ID NO: 99 - protein, 16 aa, synthetic construct
RSSQSLVHSDGNTYLS

SEQ ID NO: 100 - protein, 7 aa, synthetic construct
KISNRFS

SEQ ID NO: 101 - protein, 9 aa, synthetic construct
MQATQFPLT

SEQ ID NO: 102 - protein, 121 aa, synthetic construct
QVQLVQSGAEVKKPGASVKVSCKASGYTFTGYYMHWVRQAPGQGLEWMGWINPHSGAPNSAQKFQVRVTMTRDTSISTAYMELSRLRSDDTAVYYCARDSSGWYGDAFDIWGQGTMVTVSS

SEQ ID NO: 103 - protein, 112 aa, synthetic construct
DIVMTQTPLSSPVTLGQPASISCRSSQSLVHSDGNTYLSWLQQRPGQPPRLLIYKISNRFSGVPDRFSGSGAGTDFTLKISRVEAEDVGVYYCMQATQFPLTFGGGTKVEIK

SEQ ID NO: 104 - DNA, 363 bp, synthetic construct
  source 1..363: mol_type = other DNA
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctcacagtggtgccccaaactctgcacagaagtttcaggtcagggtcaccatgaccagggacacgtccatcagcacagcctacatggagctgagcaggctgagatctgacgacacggccgtgtattactgtgcgagggatagcagcggctggtacggtgatgcttttgatatctggggccaagggacaatggtcaccgtctcttca

SEQ ID NO: 105 - DNA, 336 bp, synthetic construct
  source 1..336: mol_type = other DNA
gatattgtgatgacccagactccactctcctcacctgtcacccttggacagccggcctccatctcctgcaggtctagtcaaagcctcgtacacagtgatggcaacacctacttgagttggcttcagcagaggccaggccagcctccaagactcctaatttataagatttctaaccggttctctggggtcccagacagattcagtggcagtggggcagggacagatttcacactgaaaatcagcagggtggaagctgaggatgtcggggtttattactgcatgcaagctacacaatttccgctcactttcggcggagggaccaaggtggagatcaaa

SEQ ID NO: 106 - protein, 6 aa, synthetic construct
DPSGGS

SEQ ID NO: 107 - protein, 11 aa, synthetic construct
QGRYVGSYLDY

SEQ ID NO: 108 - protein, 120 aa, synthetic construct
QVQLVQSGAEVKKPGASVKVSCKASGYTFTSYYMHWVRQAPGQGLEWMGIIDPSGGSTSYAQKFQGRVTMTRDTSTNTVYMDLSSLRSEDTAVYYCARQGRYVGSYLDYWGQGTLVTVSS

SEQ ID NO: 109 - protein, 106 aa, synthetic construct; identical to SEQ ID NO: 84

SEQ ID NO: 110 - DNA, 360 bp, synthetic construct
  source 1..360: mol_type = other DNA
caggtgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggaataatcgaccctagtggtggtagcacaagctacgcacagaagttccagggcagagtcaccatgaccagggacacgtccacgaacacagtctacatggacctgagcagcctgagatctgaggacacggccgtgtattactgtgcgagacaggggcggtatgttgggagctaccttgactactggggccagggaaccctggtcaccgtctcctca

SEQ ID NO: 111 - DNA, 318 bp, synthetic construct; identical to SEQ ID NO: 86
  source 1..318: mol_type = other DNA

SEQ ID NO: 112 - protein, 9 aa, synthetic construct
QQYNSYPYT

SEQ ID NO: 113 - protein, 107 aa, synthetic construct
DIQMTQSPSSLSASVGDRVTITCRASQGISNYLAWFQQKPGKAPKSVIYAASSLQSGVPSKFSGSGSGTDFTLTISSMQPEDFATYYCQQYNSYPYTFGQGTKLEIK

SEQ ID NO: 114 - DNA, 321 bp, synthetic construct
  source 1..321: mol_type = other DNA
gacatccagatgacccagtctccatcctcactgtctgcatctgtaggagacagagtcaccatcacttgtcgggcgagtcagggcattagcaattatttagcctggtttcagcagaaaccagggaaagcccctaagtccgtgatctatgctgcatccagtttgcaaagtggggtcccatcaaagttcagcggcagtggatctgggacagatttcactctcaccatcagcagcatgcagcctgaagattttgcaacttattactgccaacagtataatagttacccgtacacttttggccaggggaccaagctggagatcaaa

SEQ ID NO: 115 - protein, 6 aa, synthetic construct
NSYSGG

SEQ ID NO: 116 - protein, 12 aa, synthetic construct
DGSGWYGSYFDF

SEQ ID NO: 117 - protein, 16 aa, synthetic construct
RSSQSLVHSDGNTYLS

SEQ ID NO: 118 - protein, 9 aa, synthetic construct
IQTTQFPLT

SEQ ID NO: 119 - protein, 121 aa, synthetic construct
QVQLVQSGAEVRKPGASVKVSCKASGYTFTGYYMHWVRQAPGQGLEWMGWINSYSGGANYAQKFQDRVTMTRDTSVSTASMELSRLRSDDTAVYYCARDGSGWYGSYFDFWGQGTLVTVSS

SEQ ID NO: 120 - protein, 112 aa, synthetic construct
DIVMTQTPLSSPVTLGQPASISCRSSQSLVHSDGNTYLSWLQQRPGQPPRLLIYKISNRFSGVPDRFSGSGAGTDFTLKISRVEAEDVGFYYCIQTTQFPLTFGGGTKVEIK

SEQ ID NO: 121 - DNA, 363 bp, synthetic construct
  source 1..363: mol_type = other DNA
caggtgcagctggtgcagtctggggctgaggtgaggaagcctggggcctcagtgaaggtctcctgcaaggcttctggatacaccttcaccggctactatatgcactgggtgcgacaggcccctggacaagggcttgagtggatgggatggatcaactcttacagtggtggcgcaaactatgcacagaagtttcaggacagggtcaccatgaccagggacacgtccgtcagcacagcctccatggagctgagcaggctgagatctgacgacacggccgtgtattactgtgcgagagatggcagtggctggtacgggtcctactttgacttctggggccagggaaccctggtcaccgtctcctca

SEQ ID NO: 122 - DNA, 336 bp, synthetic construct
  source 1..336: mol_type = other DNA
gatattgtgatgacccagactccactctcctcacctgtcacccttggacagccggcctccatctcctgcaggtctagtcaaagcctcgtacacagtgatggaaacacctacttgagttggcttcagcagaggccaggccagcctccaagactcctaatttataagatttctaaccggttctctggggtcccagacagattcagtggcagtggggcagggacagatttcacactgaaaatcagcagggtggaagctgaggatgtcgggttttattactgcatacaaactacacaatttccgctcactttcggcggagggaccaaggtggagatcaaa

SEQ ID NO: 123 - protein, 13 aa, synthetic construct
QGGAQWLVLAFDI

SEQ ID NO: 124 - protein, 11 aa, synthetic construct
RASQSVSSYLA

SEQ ID NO: 125 - protein, 9 aa, synthetic construct
QQYDSSPYT

SEQ ID NO: 126 - protein, 122 aa, synthetic construct
QVQLVQSGAEVKKPGASVQVSCKASGYTFTSYYMHWVRQAPGQGLEWMGIINPSGGSTSYAQKFQGRVTMTRDTSTSTVYMELSSLRSEDTAVYYCARQGGAQWLVLAFDIWGQGTMVTVSS

SEQ ID NO: 127 - protein, 107 aa, synthetic construct
EIVLTQSPGTLSLSPGERATLSCRASQSVSSYLAWYQQKPGQPPRLLIYGASSRATGIPDRFSGGGSGTDFTLTISRLEPEDFAVYYCQQYDSSPYTFGQGTKLEIK

SEQ ID NO: 128 - DNA, 366 bp, synthetic construct
  source 1..366: mol_type = other DNA
caggtgcagctggtgcagtctggggctgaggtgaagaagcctggggcctcagtgcaggtttcctgcaaggcatctggatacaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagacaggggggagcccagtggctggtacttgcttttgatatctggggccaagggacaatggtcaccgtctcttca

SEQ ID NO: 129 - DNA, 321 bp, synthetic construct
  source 1..321: mol_type = other DNA
gaaattgtgttgacgcagtctccaggcaccctgtctttgtctccaggggaaagagccaccctctcctgcagggccagtcagagtgttagcagctacttagcctggtaccagcagaaacctggccagcctcccaggctcctcatctatggtgcatccagcagggccactggcatcccagacaggttcagtggcggtgggtctgggacagacttcactctcaccatcagcagactggagcctgaagattttgcagtgtattactgtcagcagtatgatagctcaccgtacacttttggccaggggaccaagctggagatcaaa

SEQ ID NO: 130 - protein, 9 aa, synthetic construct
QQYGNSPYT

SEQ ID NO: 131 - protein, 120 aa, synthetic construct
QVQLVQSGAEVKKPGASVKVSCKASGYTFTSYYMHWVRQAPGQGLEWMGIINPSGGSRSYAQKFQGRVTMTRDTSTSTVYMELSSLRSEDTAVYYCARQLELLGNAFDIWGQGTMVTVSS

SEQ ID NO: 132 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGNSPYTFGQGTKLEIK

SEQ ID NO: 133 - DNA, 360 bp, synthetic construct
  source 1..360: mol_type = other DNA
caggtgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagtagaagctacgcacagaagttccagggcagagtcactatgaccagggacacgtccacgagcacagtctacatggagctgagcagcctgagatctgaggacacggccgtgtattactgtgcgagacaactggaacttttggggaatgcttttgatatctggggccaagggacaatggtcaccgtctcttca

SEQ ID NO: 134 - DNA, 324 bp, synthetic construct
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtaactcgccgtacacttttggccaggggaccaagctggagatcaaa

SEQ ID NO: 135 - protein, 7 aa, synthetic construct
GFTFNNF

SEQ ID NO: 136 - protein, 6 aa, synthetic construct
KQDGSE

SEQ ID NO: 137 - protein, 6 aa, synthetic construct
ESYFDL

SEQ ID NO: 138 - protein, 14 aa, synthetic construct
RSSTGAVTTSNYAN

SEQ ID NO: 139 - protein, 7 aa, synthetic construct
GTNNRAP

SEQ ID NO: 140 - protein, 9 aa, synthetic construct
ALWYSNRWV

SEQ ID NO: 141 - protein, 115 aa, synthetic construct
EVQLVESGGGLVQPGGSLRLSCAASGFTFNNFWMSWVRQAPGKGLEWVANIKQDGSEKYYVDSVKGRFTISRDNAKNSLYLQMNSLRAEDTAVYYCARESYFDLWGRGTLVTVSS

SEQ ID NO: 142 - protein, 109 aa, synthetic construct
QAVVTQESALTTSPGETVTLTCRSSTGAVTTSNYANWVQEKPDHLFTGLIGGTNNRAPGVPARFSGSLIGDKAALTITGAQTEDEAIYFCALWYSNRWVFGGGTKLTVL

SEQ ID NO: 143 - DNA, 345 bp, synthetic construct
  source 1..345: mol_type = other DNA
gaggtgcagctggtggagtctgggggaggcttggtccagcctggggggtccctgagactctcctgtgcagcctctggattcacctttaataacttttggatgagctgggtccgccaggctccagggaaggggctggagtgggtggccaacataaagcaagatggaagtgagaaatactatgtggactctgtgaagggccgattcaccatctccagagacaacgccaagaactcactgtatctgcaaatgaacagcctgagagccgaggacacggctgtgtattactgtgcgagagagagttacttcgatctctggggccgtggcaccctggtcactgtctcctca

SEQ ID NO: 144 - DNA, 327 bp, synthetic construct
  source 1..327: mol_type = other DNA
caggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccgctgggtgttcggtggaggaaccaaactgactgtccta

SEQ ID NO: 145 - protein, 11 aa, synthetic construct
VADIVGATFDY

SEQ ID NO: 146 - protein, 7 aa, synthetic construct
GASSWAT

SEQ ID NO: 147 - protein, 120 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARVADIVGATFDYWGQGTLVTVSS

SEQ ID NO: 148 - protein, 108 aa, synthetic construct
EIVLTQSPGTLSLSPGERATLSCRASQSVSSSYLAWYQQKPGQAPRLLIYGASSWATGIPDRFSGSGSGTDFTLTISRLEPEDFAVYYCQQYDSSPYTFGQGTKLEIK

SEQ ID NO: 149 - DNA, 360 bp, synthetic construct
  source 1..360: mol_type = other DNA
caggtgcagctggtgcagtctggggctgaggtgaagaagcctggggcctcagtgaaagtttcctgcaaggcatctggatacaccttcaccagctactatatgcactgggtgcgacaggcccctggacaagggcttgagtggatgggaataatcaaccctagtggtggtagcactagctacgcacagaagttccagggcagagtcaccatgaccagggacacgtccacgagcacagtctacatggagctgagcagcctgagatctgaggacacggccgtgtattactgtgcgagagtggcggacatagtgggagctacttttgactactggggccagggaaccctggtcaccgtctcctca

SEQ ID NO: 150 - DNA, 324 bp, synthetic construct
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagctgggccactggcatcccagacaggttcagtggcagtgggtctgggacagacttcactctcaccatcagcagactggaacctgaagattttgcagtgtattactgtcagcagtatgatagctcaccctacacttttggccaggggaccaagctggagatcaaa

SEQ ID NO: 151 - protein, 7 aa, synthetic construct
GYTFTTY

SEQ ID NO: 152 - protein, 11 aa, synthetic construct
QGHYIGNYLDY

SEQ ID NO: 153 - protein, 120 aa, synthetic construct
QVQLVQSGAEVKKPGASVKVSCKASGYTFTTYYMHWVRQAPGQGLEWMGIINPSGGSTSYAQKFQGRVTMTRDTSTSTVYMELSSLRSEDTAVYYCARQGHYIGNYLDYWGQGTLVTVSS

SEQ ID NO: 154 - protein, 106 aa, synthetic construct; identical to SEQ ID NO: 84

SEQ ID NO: 155 - DNA, 360 bp, synthetic construct
  source 1..360: mol_type = other DNA
caggtgcagctggtgcagtctggggctgaggtgaagaagcctggggcctcagtgaaggtttcctgcaaggcatctggatacaccttcaccacctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagacagggccattatattgggaactaccttgactactggggccagggaaccctggtcaccgtctcctca

SEQ ID NO: 156 - DNA, 318 bp, synthetic construct; identical to SEQ ID NO: 86
  source 1..318: mol_type = other DNA

SEQ ID NO: 157 - protein, 122 aa, synthetic construct; identical to SEQ ID NO: 126

SEQ ID NO: 158 - protein, 107 aa, synthetic construct
EIVLTQSPGTLSLSPGERATLSCRASQSVSSYLAWYQQKPGQAPRLLIYGASSRATGIPDRFSGGGSGTDFTLTISRLEPEDFAVYYCQQYGSSPYTFGQGTKLEIK

SEQ ID NO: 159 - DNA, 366 bp, synthetic construct; identical to SEQ ID NO: 128
  source 1..366: mol_type = other DNA

SEQ ID NO: 160 - DNA, 321 bp, synthetic construct
  source 1..321: mol_type = other DNA
gaaattgtgttgacgcagtctccaggcaccctgtctttgtctccaggggaaagagccaccctctcctgcagggccagtcagagtgttagcagctacttagcctggtaccagcagaaacctggccaggctcccaggctcctcatctatggtgcatccagcagggccactggcatcccagacaggttcagtggcggtgggtctgggacagacttcactctcaccatcagcagactggagcctgaagattttgcagtgtattactgtcagcagtatggtagctcaccgtacacttttggccaggggaccaagctggagatcaaa

SEQ ID NO: 161 - protein, 10 aa, synthetic construct
EWLRFGGLVY

SEQ ID NO: 162 - protein, 9 aa, synthetic construct
QQYNNWPLT

SEQ ID NO: 163 - protein, 119 aa, synthetic construct
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCAREWLRFGGLVYWGQGTLVTVSS

SEQ ID NO: 164 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPLTFGGGTKVEIK

SEQ ID NO: 165 - DNA, 357 bp, synthetic construct
  source 1..357: mol_type = other DNA
gaggtgcagctggtggagtctgggggaggcttggtccagcctggggggtccctgagactctcctgtgcagcctctggattcacctttagtagctattggatgagctgggtccgccaggctccagggaaggggctggagtgggtggccaacataaagcaagatggaagtgagaaatactatgtggactctgtgaagggccgattcaccatctccagagacaacgccaagaactcactgtatctgcaaatgaacagcctgagagccgaggacacggctgtgtattactgtgcgagagagtggctacgatttgggggcttagtgtactggggccagggaaccctggtcaccgtctcctca

SEQ ID NO: 166 - DNA, 321 bp, synthetic construct
  source 1..321: mol_type = other DNA
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggccgctcactttcggcggagggaccaaggtggagatcaaa

SEQ ID NO: 167 - protein, 6 aa, synthetic construct
NPNSGG

SEQ ID NO: 168 - protein, 9 aa, synthetic construct
DGVITSFDY

SEQ ID NO: 169 - protein, 17 aa, synthetic construct
KSSQSVLYSSNIKTYLA

SEQ ID NO: 170 - protein, 7 aa, synthetic construct
WASTRES

SEQ ID NO: 171 - protein, 9 aa, synthetic construct
QQYYSAPYT

SEQ ID NO: 172 - protein, 118 aa, synthetic construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDGVITSFDYWGQGTLVTVSS

SEQ ID NO: 173 - protein, 113 aa, synthetic construct
DIVMTQSPDSLAVSLGERATINCKSSQSVLYSSNIKTYLAWYQQKPGQPPKLLIYWASTRESGVPDRFSGGGSGTDFTLTITSLQAEDVAVYYCQQYYSAPYTFGQGTKLEIK

SEQ ID NO: 174 - DNA, 354 bp, synthetic construct
  source 1..354: mol_type = other DNA
caggtgcagctggtgcagtctggggctgaggtgaagaagcctggggcctcagtgaaggtctcctgcaaggcttctggatacactttcaccggctactatatgcactgggtgcgacaggcccctggacaagggcttgagtggatgggatggatcaaccctaacagtggtggcacaaactatgcacagaagtttcagggcagggtcaccatgaccagggacacgtccatcagcacagcctacatggagctgagcaggctgagatctgacgacacggccgtgtattactgtgcgagagacggggtgattacgtcttttgactactggggccagggaaccctggtcaccgtctcctca

SEQ ID NO: 175 - DNA, 339 bp, synthetic construct
  source 1..339: mol_type = other DNA
gacatcgtgatgacccagtctccagactccctggctgtgtctctgggcgagagggccaccatcaactgcaagtccagccagagtgttttatacagctccaacattaagacctacttagcttggtaccagcagaaaccaggacagcctcctaagctgctcatttactgggcatctacccgggaatccggagtccctgaccgattcagtggcggcgggtctgggacagatttcactctcaccatcaccagcctgcaggctgaagatgtggcagtttattactgtcagcagtattatagtgctccgtacacttttggccaggggaccaagctggagatcaaa

SEQ ID NO: 176 - protein, 6 aa, synthetic construct
DPSDGS

SEQ ID NO: 177 - protein, 11 aa, synthetic construct
DREYNYYGLDV

SEQ ID NO: 178 - protein, 16 aa, synthetic construct
RSSQSLVYRDGNTYLN

SEQ ID NO: 179 - protein, 9 aa, synthetic construct
MQGTHWPPT

SEQ ID NO: 180 - protein, 120 aa, synthetic construct
QVQLVQSGAEVKKPGASVKVSCKASGYTFTSYYIHWVRQAPGQGLEWMGIIDPSDGSTNYAQKFQGRVTMTRDTSTSTVYMELSSLRSEDTAVYYCARDREYNYYGLDVWGQGTTVTVSS

SEQ ID NO: 181 - protein, 112 aa, synthetic construct
DVVMTQSPLSLPVTLGQPASISCRSSQSLVYRDGNTYLNWFQQRPGQSPRRLIYKVSNRDSGVPDRFRGSGSGTDFTLKISRVEAEDVGVYYCMQGTHWPPTFGQGTKVEIK

SEQ ID NO: 182 - DNA, 360 bp, synthetic construct
  source 1..360: mol_type = other DNA
caggtgcagctggtgcagtctggggctgaggtgaagaagcctggggcctcagtgaaggtttcctgcaaggcatctggatacaccttcaccagctactatatacactgggtgcgacaggcccctggacaagggcttgagtggatgggaataatcgacccaagtgatggtagcacaaactacgcacagaagttccagggcagagtcaccatgaccagggacacgtccacgagcacagtctacatggagctgagcagcctgagatctgaggacacggccgtgtattactgtgcgagagatcgggaatataactactacggtttggacgtctggggccaagggaccacggtcaccgtctcctca

SEQ ID NO: 183 - DNA, 336 bp, synthetic construct
  source 1..336: mol_type = other DNA
gatgttgtgatgactcagtctccactctccctgcccgtcacccttggacagccggcctccatctcctgcaggtctagtcaaagcctcgtataccgtgatggaaacacctacttgaattggtttcagcagaggccaggccaatctccaaggcgcctaatttataaggtttctaaccgggactctggggtcccagacagattccgcggcagtgggtcaggcactgatttcacactgaaaatcagccgggtggaggctgaggatgttggggtttattactgcatgcaaggtacacactggcctccgacgttcggccaagggaccaaggtggaaatcaaa

SEQ ID NO: 184 - protein, 8 aa, synthetic construct
GFTFSNTW

SEQ ID NO: 185 - protein, 10 aa, synthetic construct
IKSKSDGGTT

SEQ ID NO: 186 - protein, 10 aa, synthetic construct
TQYYWNSFDY

SEQ ID NO: 187 - protein, 11 aa, synthetic construct
QASQDISNYLN

SEQ ID NO: 188 - protein, 7 aa, synthetic construct
DASNLET

SEQ ID NO: 189 - protein, 9 aa, synthetic construct
QQYDNLPLT

SEQ ID NO: 190 - protein, 119 aa, synthetic construct
EVQLVESGGGLVKPGGSLRLSCAASGFTFSNTWMSWVRQAPGKGLEWVGRIKSKSDGGTTDYAAPVKGRFTISRDDSKNTLYLQMNSLKTEDTAVYYCTQYYWNSFDYWGQGTLVTVSS

SEQ ID NO: 191 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYDNLPLTFGGGTKVEIK

SEQ ID NO: 192 - DNA, 357 bp, synthetic construct
  source 1..357: mol_type = other DNA
gaggtgcagctggtggagtctggggggggcttggtaaagcctggggggtcccttagactctcctgtgcagcctctggattcactttcagtaacacctggatgagctgggtccgccaggctccagggaaggggctggagtgggttggccgtattaaaagcaaatctgatggtgggacaacagactacgctgcacccgtgaaaggcagattcaccatctcaagagatgattcaaaaaacacgctgtatctgcaaatgaacagcctgaaaaccgaggacacagccgtgtattactgtacccagtattattggaactcctttgactactggggccagggaaccctggtcaccgtctcctca

SEQ ID NO: 193 - DNA, 321 bp, synthetic construct
  source 1..321: mol_type = other DNA
gacatccagatgacccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattactgtcaacagtatgataatctcccgctcactttcggcggagggaccaaggtggagatcaaa

SEQ ID NO: 194 - protein, 6 aa, synthetic construct
KEDGSE

SEQ ID NO: 195 - protein, 9 aa, synthetic construct
DPGWAPFDY

SEQ ID NO: 196 - protein, 9 aa, synthetic construct
QQYNSFPYT

SEQ ID NO: 197 - protein, 118 aa, synthetic construct
EVQLVESGGGLVQPGGSQRLSCAASGFTFSSYWMNWVRQAPGKGLEWVANIKEDGSEKYYVDSVKGRFTISRDNAKNSLYLQMNSLRAEDTAVYYCARDPGWAPFDYWGQGTLVTVSS

SEQ ID NO: 198 - protein, 107 aa, synthetic construct
DIQMSQSPSSLSASVGDRVTITCRASQGISNYLAWFQQKPGKAPKSLIYAASSLQSGVPSKFSGSGSGTDFTLAISSLQPEDFATYYCQQYNSFPYTFGQGTTLEIK

SEQ ID NO: 199 - DNA, 354 bp, synthetic construct
  source 1..354: mol_type = other DNA
gaggtgcagctggtggagtctgggggaggcttggtccagcctggggggtcccagagactctcctgtgcagcctctggattcacctttagtagctattggatgaactgggtccgccaggctccagggaaggggctggagtgggtggccaacataaaggaagatggaagtgagaaatactatgtggactctgtgaagggccgattcaccatctccagagacaacgccaagaactcactgtatctgcaaatgaacagcctgagagccgaggacacggctgtgtattactgtgcgagagatccgggctgggctccctttgactactggggccagggaaccctggtcaccgtctcctca

SEQ ID NO: 200 - DNA, 321 bp, synthetic construct
  source 1..321: mol_type = other DNA
gacatccagatgtcccagtctccatcctcactgtctgcatctgtaggagacagagtcaccatcacttgtcgggcgagtcagggcattagcaattatttagcctggtttcagcagaaaccagggaaagcccctaagtccctgatctatgctgcatccagtttgcaaagtggggtcccatcaaagttcagcggcagtggatctgggacagatttcactctcgccatcagcagcctgcagcctgaagattttgcaacttattactgccaacagtataatagtttcccgtacacttttggccaggggaccacgctggagatcaaa

SEQ ID NO: 201 - protein, 7 aa, synthetic construct
GGSINSY

SEQ ID NO: 202 - protein, 5 aa, synthetic construct
FYSGI

SEQ ID NO: 203 - protein, 10 aa, synthetic construct
IGVAGFYFDY

SEQ ID NO: 204 - protein, 9 aa, synthetic construct
QQYGTSPLT

SEQ ID NO: 205 - protein, 118 aa, synthetic construct
QVQLQESGPGLVKPSETLSLTCTVSGGSINSYYWSWIRQPPGKGLEWIGYIFYSGITNYNPSLKSRVTISLDTSKNQFSLKLSSVTAADTAVYYCARIGVAGFYFDYWGQGTLVTVSS

SEQ ID NO: 206 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGTSPLTFGGGTKVEIK

SEQ ID NO: 207 - DNA, 354 bp, synthetic construct
  source 1..354: mol_type = other DNA
caggtgcagctgcaggagtcgggcccaggactggtgaagccttcggagaccctgtccctcacctgcactgtctctggtggctccatcaatagttactactggagctggatccggcagcccccagggaagggactggagtggattgggtatatcttttacagtgggatcaccaactacaacccctccctcaagagtcgagtcaccatatcattagacacgtccaagaaccagttctccctgaagctgagctctgtgaccgctgcggacacggccgtgtattactgtgcgagaatcggcgtggctggtttttactttgactactggggccagggaaccctggtcaccgtctcctca

SEQ ID NO: 208 - DNA, 324 bp, synthetic construct
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtacctcaccgctcactttcggcggagggaccaaggtggagatcaaa

SEQ ID NO: 209 - protein, 618 aa, Homo sapiens
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREVATPLFPPLHTGRAGQRQHLLFPYPSSILSVK

SEQ ID NO: 210 - protein, 7 aa, synthetic construct
LGSNRAS

(19) United States
(12) Patent Application Publication
(10) Pub. No.: US 2024/0216557 A1
(43) Pub. Date: Jul. 4, 2024

(54) RADIOLIGAND BINDING TO ANTI-DLL3 ANTIBODIES FOR PRETARGETED PET IMAGING AND THERAPEUTIC USES THEREOF

(71) Applicants: Memorial Sloan-Kettering Cancer Center, New York, NY (US); Sloan-Kettering Institute for Cancer Research, New York, NY (US); Memorial Hospital for Cancer and Allied Diseases, New York, NY (US); Tri-Institutional Therapeutics Discovery Institute, Inc., New York, NY (US)

(72) Inventors: John T. POIRIER, New York, NY (US); Charles Rudin, New York, NY (US); Jason Lewis, New York, NY (US); Abdul Khan, Cedar Park, TX (US); David Andrew, Sandyhook, CT (US); Xinlei Chen, New York, NY (US); Ivo C. Lorenz, New York, NY (US); Kathryn M. Tully, New York, NY (US); Salomon Tendler, New York, NY (US)

(73) Assignees: Memorial Sloan-Kettering Cancer Center, New York, NY (US); Sloan-Kettering Institute for Cancer Research, New York, NY (US); Memorial Hospital for Cancer and Allied Diseases, New York, NY (US); Tri-Institutional Therapeutics Discovery Institute, Inc., New York, NY (US)

(21) Appl. No.: 18/589,649

(22) Filed: Feb. 28, 2024

Related U.S. Application Data
(63) Continuation of application No. PCT/US22/42436, filed on Sep. 2, 2022.
(60) Provisional application No. 63/240,237, filed on Sep. 2, 2021.

(51) Int. Cl. A61K 51/10

(52) U.S. Cl. CPC A61K 51/1096; A61K 51/1018

(57) ABSTRACT

The presently disclosed subject matter provides compositions and methods for in vivo diagnosis and treatment of diseases or disorders associated with DLL3. In particular, the presently disclosed subject matter provides novel anti-DLL3 antibodies-TCO conjugates, which can form a radioimmunoconjugate with a radioligand comprising a radioactive isotope.

### CROSS REFERENCE TO RELATED APPLICATIONS

This application is a continuation of International Application No. PCT/US2022/042436, filed on Sep. 2, 2022, which claims priority to U.S. Provisional Patent Application No. 63/240,237, filed on Sep. 2, 2021, the contents of which are incorporated by reference in their entireties, and to each of which priority is claimed.

### SEQUENCE LISTING

The present application contains a Sequence Listing which has been submitted via EFS-Web and is hereby incorporated by reference in its entirety. Said Sequence Listing, created on Feb. 26, 2024, is named 0727341572.xml and is 207,281 bytes in size.

### 1. INTRODUCTION

The presently disclosed subject matter relates generally to compositions and methods for in vivo diagnosis and treatment of diseases or disorders associated with DLL3. The presently disclosed subject matter provides novel anti-DLL3 antibodies-transcyclooctene (TCO) conjugates, which can form a radioimmunoconjugate with a radioligand comprising a radioactive isotope.

### 2. BACKGROUND OF THE INVENTION

Antibodies are an effective tool for the sensitive and specific delivery of radioactive isotopes to tumors. Radiolabeled antibodies and antigen-binding fragments thereof show several limitations for their clinical use (e.g., size, clearance, half-life, rapid pharmacokinetics, etc.). To address these limitations, pretargeting, or strategies to decouple the antibody from the radioactivity at the time of injection, have been developed as alternative. However, current pretargeting strategy has been hampered by its intrinsic limitations. For example, the immunogenicity of the streptavidin-bearing immunoconjugates or the slow clearance of the radioligand limit their use in clinical practice. There is a need to develop radioimmunoconjugates that delineate cancer from healthy tissue in small amounts of time, possess favorable pharmacokinetic profiles, can be cleared rapidly from the body to enable high contrast PET imaging of cancer and that can specifically target and kill cancer cells (e.g., radioimmunotherapy).

DLL3 is selectively expressed in high grade pulmonary neuroendocrine tumors of the lung (LU-NETs). Lu-NETs embrace a heterogeneous family of neoplasms classified into four histological variants, namely typical carcinoid (TC), atypical carcinoid (AC), large cell neuroendocrine carcinoma (LCNEC) and small cell lung carcinoma (SCLC). Increased expression of DLL3 was observed in SCLC and LCNEC patient-derived xenograft tumors and was also confirmed in primary tumors. See Saunders et al., Sci Translational Medicine (302): 302ra136 (2015). Both SCLC and pulmonary LCNEC are high-grade and poor-prognosis tumors, with higher incidence in smokers. Pulmonary LCNEC exhibits biologically aggressive behavior, similarly to SCLC. Stage by stage, survival curves of pulmonary LCNEC and SCLC overlap, and in addition, survival is lower than other NSCLCs. Prognosis is poor even in patients with potentially resectable stage I lung cancer with 5-year survival rates ranging from 27% to 67%. See Iyoda A. et al., J Thorac Cardiovasc Surg. 138:446-453 (2009).

Given the significant role for DLL3 in various diseases or disorders, radioimmunoconjugates that recognize DLL3 are desired.

### 3. SUMMARY OF THE INVENTION

The presently disclosed subject matter provides anti-DLL3 antibody-transcyclooctene (TCO) conjugates, radioimmunoconjugates comprising the anti-DLL3 antibody-TCO conjugates and radioligand, and methods of using thereof.

In certain embodiments, the antibody-transcyclooctene (TCO) conjugate comprises an anti-DLL3 antibody or an antigen-binding fragment thereof, wherein the anti-DLL3 antibody-TCO conjugate comprises a heavy chain variable region that comprises CDR1, CDR2, and CDR3 domains; and a light chain variable region that comprises CDR1, CDR2, and CDR3 domains, wherein the heavy chain variable region and light chain variable region CDR3 domains are selected from the group consisting of:
a) a heavy chain variable region CDR3 comprising the amino acid sequence set forth in SEQ ID NO: 3 and a conservative modification thereof; and a light chain variable region CDR3 comprising the amino acid sequence set forth in SEQ ID NO: 6 and a conservative modification thereof;
b) a heavy chain variable region CDR3 comprising the amino acid sequence set forth in SEQ ID NO: 13 and a conservative modification thereof; and a light chain variable region CDR3 comprising the amino acid sequence set forth in SEQ ID NO: 16 and a conservative modification thereof;
c) a heavy chain variable region CDR3 comprising the amino acid sequence set forth in SEQ ID NO: 22 and a conservative modification thereof; and a light chain variable region CDR3 comprising the amino acid sequence set forth in SEQ ID NO: 23 and a conservative modification thereof;
d) a heavy chain variable region CDR3 comprising the amino acid sequence set forth in SEQ ID NO: 30 and a conservative modification thereof; and a light chain variable region CDR3 comprising the amino acid sequence set forth in SEQ ID NO: 33 and a conservative modification thereof;
e) a heavy chain variable region CDR3 comprising the amino acid sequence set forth in SEQ ID NO: 39 and a conservative modification thereof; and a light chain variable region CDR3 comprising the amino acid sequence set forth in SEQ ID NO: 41 and a conservative modification thereof;
f) a heavy chain variable region CDR3 comprising the amino acid sequence set forth in SEQ ID NO: 48 and a conservative modification thereof; and a light chain variable region CDR3 comprising the amino acid sequence set forth in SEQ ID NO: 51 and a conservative modification thereof;
g) a heavy chain variable region CDR3 comprising the amino acid sequence set forth in SEQ ID NO: 56 and a conservative modification thereof; and a light chain variable region CDR3 comprising the amino acid sequence set forth in SEQ ID NO: 59 and a conservative modification thereof;
h) a heavy chain variable region CDR3 comprising the amino acid sequence set forth in SEQ ID NO: 64 and a conservative modification thereof; and a light chain variable region CDR3 comprising the amino acid sequence set forth in SEQ ID NO: 65 and a conservative modification thereof;
i) a heavy chain variable region CDR3 comprising the amino acid sequence set forth in SEQ ID NO: 72 and a conservative modification thereof; and a light chain variable region CDR3 comprising the amino acid sequence set forth in SEQ ID NO: 75 and a conservative modification thereof;
j) a heavy chain variable region CDR3 comprising the amino acid sequence set forth in SEQ ID NO: 81 and a conservative modification thereof; and a light chain variable region CDR3 comprising the amino acid sequence set forth in SEQ ID NO: 82 and a conservative modification thereof;
k) a heavy chain variable region CDR3 comprising the amino acid sequence set forth in SEQ ID NO: 89 and a conservative modification thereof; and a light chain variable region CDR3 comprising the amino acid sequence set forth in SEQ ID NO: 91 and a conservative modification thereof;
l) a heavy chain variable region CDR3 comprising the amino acid sequence set forth in SEQ ID NO: 98 and a conservative modification thereof; and a light chain variable region CDR3 comprising the amino acid sequence set forth in SEQ ID NO: 101 and a conservative modification thereof;
m) a heavy chain variable region CDR3 comprising the amino acid sequence set forth in SEQ ID NO: 107 and a conservative modification thereof; and a light chain variable region CDR3 comprising the amino acid sequence set forth in SEQ ID NO: 82 and a conservative modification thereof;
n) a heavy chain variable region CDR3 comprising the amino acid sequence set forth in SEQ ID NO: 107 and a conservative modification thereof; and a light chain variable region CDR3 comprising the amino acid sequence set forth in SEQ ID NO: 112 and a conservative modification thereof;
o) a heavy chain variable region CDR3 comprising the amino acid sequence set forth in SEQ ID NO: 116 and a conservative modification thereof; and a light chain variable region CDR3 comprising the amino acid sequence set forth in SEQ ID NO: 118 and a conservative modification thereof;
p) a heavy chain variable region CDR3 comprising the amino acid sequence set forth in SEQ ID NO: 123 and a conservative modification thereof; and a light chain variable region CDR3 comprising the amino acid sequence set forth in SEQ ID NO: 125 and a conservative modification thereof;
q) a heavy chain variable region CDR3 comprising the amino acid sequence set forth in SEQ ID NO: 56 and a conservative modification thereof; and a light chain variable region CDR3 comprising the amino acid sequence set forth in SEQ ID NO: 130 and a conservative modification thereof;
r) a heavy chain variable region CDR3 comprising the amino acid sequence set forth in SEQ ID NO: 137 and a conservative modification thereof; and a light chain variable region CDR3 comprising the amino acid sequence set forth in SEQ ID NO: 140 and a conservative modification thereof;
s) a heavy chain variable region CDR3 comprising the amino acid sequence set forth in SEQ ID NO: 145 and a conservative modification thereof; and a light chain variable region CDR3 comprising the amino acid sequence set forth in SEQ ID NO: 125 and a conservative modification thereof;
t) a heavy chain variable region CDR3 comprising the amino acid sequence set forth in SEQ ID NO: 152 and a conservative modification thereof; and a light chain variable region CDR3 comprising the amino acid sequence set forth in SEQ ID NO: 82 and a conservative modification thereof;
u) a heavy chain variable region CDR3 comprising the amino acid sequence set forth in SEQ ID NO: 123 and a conservative modification thereof; and a light chain variable region CDR3 comprising the amino acid sequence set forth in SEQ ID NO: 59 and a conservative modification thereof;
v) a heavy chain variable region CDR3 comprising the amino acid sequence set forth in SEQ ID NO: 161 and a conservative modification thereof; and a light chain variable region CDR3 comprising the amino acid sequence set forth in SEQ ID NO: 162 and a conservative modification thereof;
w) a heavy chain variable region CDR3 comprising the amino acid sequence set forth in SEQ ID NO: 168 and a conservative modification thereof; and a light chain variable region CDR3 comprising the amino acid sequence set forth in SEQ ID NO: 171 and a conservative modification thereof;
x) a heavy chain variable region CDR3 comprising the amino acid sequence set forth in SEQ ID NO: 177 and a conservative modification thereof; and a light chain variable region CDR3 comprising the amino acid sequence set forth in SEQ ID NO: 179 and a conservative modification thereof;
y) a heavy chain variable region CDR3 comprising the amino acid sequence set forth in SEQ ID NO: 186 and a conservative modification thereof; and a light chain variable region CDR3 comprising the amino acid sequence set forth in SEQ ID NO: 189 and a conservative modification thereof;
z) a heavy chain variable region CDR3 comprising the amino acid sequence set forth in SEQ ID NO: 195 and a conservative modification thereof; and a light chain variable region CDR3 comprising the amino acid sequence set forth in SEQ ID NO: 196 and a conservative modification thereof; and
aa) a heavy chain variable region CDR3 comprising the amino acid sequence set forth in SEQ ID NO: 203 and a conservative modification thereof; and a light chain variable region CDR3 comprising the amino acid sequence set forth in SEQ ID NO: 204 and a conservative modification thereof.

In certain embodiments, the heavy chain variable region and light chain variable region CDR2 domains are selected from the group consisting of:
a) a heavy chain variable region CDR2 comprising the amino acid sequence set forth in SEQ ID NO: 2 and a conservative modification thereof; and a light chain variable region CDR2 comprising the amino acid sequence set forth in SEQ ID NO: 5 and a conservative modification thereof;
b) a heavy chain variable region CDR2 comprising the amino acid sequence set forth in SEQ ID NO: 12 and a conservative modification thereof; and a light chain variable region CDR2 comprising the amino acid sequence set forth in SEQ ID NO: 15 and a conservative modification thereof;
c) a heavy chain variable region CDR2 comprising the amino acid sequence set forth in SEQ ID NO: 29 and a conservative modification thereof; and a light chain variable region CDR2 comprising the amino acid sequence set forth in SEQ ID NO: 32 and a conservative modification thereof;
d) a heavy chain variable region CDR2 comprising the amino acid sequence set forth in SEQ ID NO: 38 and a conservative modification thereof; and a light chain variable region CDR2 comprising the amino acid sequence set forth in SEQ ID NO: 5 and a conservative modification thereof;
e) a heavy chain variable region CDR2 comprising the amino acid sequence set forth in SEQ ID NO: 47 and a conservative modification thereof; and a light chain variable region CDR2 comprising the amino acid sequence set forth in SEQ ID NO: 50 and a conservative modification thereof;
f) a heavy chain variable region CDR2 comprising the amino acid sequence set forth in SEQ ID NO: 2 and a conservative modification thereof; and a light chain variable region CDR2 comprising the amino acid sequence set forth in SEQ ID NO: 58 and a conservative modification thereof;
g) a heavy chain variable region CDR2 comprising the amino acid sequence set forth in SEQ ID NO: 71 and a conservative modification thereof; and a light chain variable region CDR2 comprising the amino acid sequence set forth in SEQ ID NO: 74 and a conservative modification thereof;
h) a heavy chain variable region CDR2 comprising the amino acid sequence set forth in SEQ ID NO: 80 and a conservative modification thereof; and a light chain variable region CDR2 comprising the amino acid sequence set forth in SEQ ID NO: 58 and a conservative modification thereof;
i) a heavy chain variable region CDR2 comprising the amino acid sequence set forth in SEQ ID NO: 88 and a conservative modification thereof; and a light chain variable region CDR2 comprising the amino acid sequence set forth in SEQ ID NO: 210 and a conservative modification thereof;
j) a heavy chain variable region CDR2 comprising the amino acid sequence set forth in SEQ ID NO: 97 and a conservative modification thereof; and a light chain variable region CDR2 comprising the amino acid sequence set forth in SEQ ID NO: 100 and a conservative modification thereof;
k) a heavy chain variable region CDR2 comprising the amino acid sequence set forth in SEQ ID NO: 106 and a conservative modification thereof; and a light chain variable region CDR2 comprising the amino acid sequence set forth in SEQ ID NO: 58 and a conservative modification thereof;
l) a heavy chain variable region CDR2 comprising the amino acid sequence set forth in SEQ ID NO: 106 and a conservative modification thereof; and a light chain variable region CDR2 comprising the amino acid sequence set forth in SEQ ID NO: 5 and a conservative modification thereof;
m) a heavy chain variable region CDR2 comprising the amino acid sequence set forth in SEQ ID NO: 115 and a conservative modification thereof; and a light chain variable region CDR2 comprising the amino acid sequence set forth in SEQ ID NO: 100 and a conservative modification thereof;
n) a heavy chain variable region CDR2 comprising the amino acid sequence set forth in SEQ ID NO: 2 and a conservative modification thereof; and a light chain variable region CDR2 comprising the amino acid sequence set forth in SEQ ID NO: 58 and a conservative modification thereof;
o) a heavy chain variable region CDR2 comprising the amino acid sequence set forth in SEQ ID NO: 136 and a conservative modification thereof; and a light chain variable region CDR2 comprising the amino acid sequence set forth in SEQ ID NO: 139 and a conservative modification thereof;
p) a heavy chain variable region CDR2 comprising the amino acid sequence set forth in SEQ ID NO: 2 and a conservative modification thereof; and a light chain variable region CDR2 comprising the amino acid sequence set forth in SEQ ID NO: 146 and a conservative modification thereof;
q) a heavy chain variable region CDR2 comprising the amino acid sequence set forth in SEQ ID NO: 136 and a conservative modification thereof; and a light chain variable region CDR2 comprising the amino acid sequence set forth in SEQ ID NO: 74 and a conservative modification thereof; and
r) a heavy chain variable region CDR2 comprising the amino acid sequence set forth in SEQ ID NO: 167 and a conservative modification thereof; and a light chain variable region CDR2 comprising the amino acid sequence set forth in SEQ ID NO: 170 and a conservative modification thereof;
s) a heavy chain variable region CDR2 comprising the amino acid sequence set forth in SEQ ID NO: 176 and a conservative modification thereof; and a light chain variable region CDR2 comprising the amino acid sequence set forth in SEQ ID NO: 50 and a conservative modification thereof;
t) a heavy chain variable region CDR2 comprising the amino acid sequence set forth in SEQ ID NO: 185 and a conservative modification thereof; and a light chain variable region CDR2 comprising the amino acid sequence set forth in SEQ ID NO: 188 and a conservative modification thereof;
u) a heavy chain variable region CDR2 comprising the amino acid sequence set forth in SEQ ID NO: 194 and a conservative modification thereof; and a light chain variable region CDR2 comprising the amino acid sequence set forth in SEQ ID NO: 5 and a conservative modification thereof; and
v) a heavy chain variable region CDR2 comprising the amino acid sequence set forth in SEQ ID NO: 202 and a conservative modification thereof; and a light chain variable region CDR2 comprising the amino acid sequence set forth in SEQ ID NO: 5 and a conservative modification thereof.

In certain embodiments, the heavy chain variable region and light chain variable region CDR1 domains are selected from the group consisting of:
a) a heavy chain variable region CDR1 comprising the amino acid sequence set forth in SEQ ID NO: 1 and a conservative modification thereof; and a light chain variable region CDR1 comprising the amino acid sequence set forth in SEQ ID NO: 4 and a conservative modification thereof;
b) a heavy chain variable region CDR1 comprising the amino acid sequence set forth in SEQ ID NO: 11 and a conservative modification thereof; and a light chain variable region CDR1 comprising the amino acid sequence set forth in SEQ ID NO: 14 and a conservative modification thereof;
c) a heavy chain variable region CDR1 comprising the amino acid sequence set forth in SEQ ID NO: 21 and a conservative modification thereof; and a light chain variable region CDR1 comprising the amino acid sequence set forth in SEQ ID NO: 4 and a conservative modification thereof;
d) a heavy chain variable region CDR1 comprising the amino acid sequence set forth in SEQ ID NO: 28 and a conservative modification thereof; and a light chain variable region CDR1 comprising the amino acid sequence set forth in SEQ ID NO: 31 and a conservative modification thereof;
e) a heavy chain variable region CDR1 comprising the amino acid sequence set forth in SEQ ID NO: 21 and a conservative modification thereof; and a light chain variable region CDR1 comprising the amino acid sequence set forth in SEQ ID NO: 40 and a conservative modification thereof;
f) a heavy chain variable region CDR1 comprising the amino acid sequence set forth in SEQ ID NO: 46 and a conservative modification thereof; and a light chain variable region CDR1 comprising the amino acid sequence set forth in SEQ ID NO: 49 and a conservative modification thereof;
g) a heavy chain variable region CDR1 comprising the amino acid sequence set forth in SEQ ID NO: 21 and a conservative modification thereof; and a light chain variable region CDR1 comprising the amino acid sequence set forth in SEQ ID NO: 57 and a conservative modification thereof;
h) a heavy chain variable region CDR1 comprising the amino acid sequence set forth in SEQ ID NO: 70 and a conservative modification thereof; and a light chain variable region CDR1 comprising the amino acid sequence set forth in SEQ ID NO: 73 and a conservative modification thereof;
i) a heavy chain variable region CDR1 comprising the amino acid sequence set forth in SEQ ID NO: 87 and a conservative modification thereof; and a light chain variable region CDR1 comprising the amino acid sequence set forth in SEQ ID NO: 90 and a conservative modification thereof;
j) a heavy chain variable region CDR1 comprising the amino acid sequence set forth in SEQ ID NO: 96 and a conservative modification thereof; and a light chain variable region CDR1 comprising the amino acid sequence set forth in SEQ ID NO: 99 and a conservative modification thereof;
k) a heavy chain variable region CDR1 comprising the amino acid sequence set forth in SEQ ID NO: 96 and a conservative modification thereof; and a light chain variable region CDR1 comprising the amino acid sequence set forth in SEQ ID NO: 117 and a conservative modification thereof;
l) a heavy chain variable region CDR1 comprising the amino acid sequence set forth in SEQ ID NO: 21 and a conservative modification thereof; and a light chain variable region CDR1 comprising the amino acid sequence set forth in SEQ ID NO: 124 and a conservative modification thereof;
m) a heavy chain variable region CDR1 comprising the amino acid sequence set forth in SEQ ID NO: 135 and a conservative modification thereof; and a light chain variable region CDR1 comprising the amino acid sequence set forth in SEQ ID NO: 138 and a conservative modification thereof;
n) a heavy chain variable region CDR1 comprising the amino acid sequence set forth in SEQ ID NO: 151 and a conservative modification thereof; and a light chain variable region CDR1 comprising the amino acid sequence set forth in SEQ ID NO: 57 and a conservative modification thereof;
o) a heavy chain variable region CDR1 comprising the amino acid sequence set forth in SEQ ID NO: 11 and a conservative modification thereof; and a light chain variable region CDR1 comprising the amino acid sequence set forth in SEQ ID NO: 73 and a conservative modification thereof;
p) a heavy chain variable region CDR1 comprising the amino acid sequence set forth in SEQ ID NO: 96 and a conservative modification thereof; and a light chain variable region CDR1 comprising the amino acid sequence set forth in SEQ ID NO: 169 and a conservative modification thereof;
q) a heavy chain variable region CDR1 comprising the amino acid sequence set forth in SEQ ID NO: 21 and a conservative modification thereof; and a light chain variable region CDR1 comprising the amino acid sequence set forth in SEQ ID NO: 178 and a conservative modification thereof;
r) a heavy chain variable region CDR1 comprising the amino acid sequence set forth in SEQ ID NO: 184 and a conservative modification thereof; and a light chain variable region CDR1 comprising the amino acid sequence set forth in SEQ ID NO: 187 and a conservative modification thereof;
s) a heavy chain variable region CDR1 comprising the amino acid sequence set forth in SEQ ID NO: 11 and a conservative modification thereof; and a light chain variable region CDR1 comprising the amino acid sequence set forth in SEQ ID NO: 4 and a conservative modification thereof; and
t) a heavy chain variable region CDR1 comprising the amino acid sequence set forth in SEQ ID NO: 201 and a conservative modification thereof; and a light chain variable region CDR1 comprising the amino acid sequence set forth in SEQ ID NO: 57 and a conservative modification thereof.

In certain embodiments, the anti-DLL3 antibody or antigen-binding fragment thereof comprises:
a) a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3;
b) a heavy chain variable region comprising a CDR1 comprising the amino acid sequence set forth in SEQ ID NO: 11, a CDR2 comprising the amino acid sequence set forth in SEQ ID NO: 12, and a CDR3 comprising the amino acid sequence set forth in SEQ ID NO: 13;
c) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 22;
d) a heavy chain variable region comprising a CDR1 comprising the amino acid sequence set forth in SEQ ID NO: 28, a CDR2 comprising the amino acid sequence set forth in SEQ ID NO: 29, and a CDR3 comprising the amino acid sequence set forth in SEQ ID NO: 30;
e) a heavy chain variable region comprising a CDR1 comprising the amino acid sequence set forth in SEQ ID NO: 21, a CDR2 comprising the amino acid sequence set forth in SEQ ID NO: 38, and a CDR3 comprising the amino acid sequence set forth in SEQ ID NO: 39;
f) a heavy chain variable region comprising a CDR1 comprising the amino acid sequence set forth in SEQ ID NO: 46, a CDR2 comprising the amino acid sequence set forth in SEQ ID NO: 47, and a CDR3 comprising the amino acid sequence set forth in SEQ ID NO: 48;
g) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 56;
h) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 64;
i) a heavy chain variable region comprising a CDR1 comprising the amino acid sequence set forth in SEQ ID NO: 70, a CDR2 comprising the amino acid sequence set forth in SEQ ID NO: 71, and a CDR3 comprising the amino acid sequence set forth in SEQ ID NO: 72;
j) a heavy chain variable region comprising a CDR1 comprising the amino acid sequence set forth in SEQ ID NO: 21, a CDR2 comprising the amino acid sequence set forth in SEQ ID NO: 80, and a CDR3 comprising the amino acid sequence set forth in SEQ ID NO: 81;
k) a heavy chain variable region comprising a CDR1 comprising the amino acid sequence set forth in SEQ ID NO: 87, a CDR2 comprising the amino acid sequence set forth in SEQ ID NO: 88, and a CDR3 comprising the amino acid sequence set forth in SEQ ID NO: 89;
l) a heavy chain variable region comprising a CDR1 comprising the amino acid sequence set forth in SEQ ID NO: 96, a CDR2 comprising the amino acid sequence set forth in SEQ ID NO: 97, and a CDR3 comprising the amino acid sequence set forth in SEQ ID NO: 98;
m) a heavy chain variable region comprising a CDR1 comprising the amino acid sequence set forth in SEQ ID NO: 21, a CDR2 comprising the amino acid sequence set forth in SEQ ID NO: 106, and a CDR3 comprising the amino acid sequence set forth in SEQ ID NO: 107;
n) a heavy chain variable region comprising a CDR1 comprising the amino acid sequence set forth in SEQ ID NO: 96, a CDR2 comprising the amino acid sequence set forth in SEQ ID NO: 115, and a CDR3 comprising the amino acid sequence set forth in SEQ ID NO: 116;
o) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 123;
p) a heavy chain variable region comprising a CDR1 comprising the amino acid sequence set forth in SEQ ID NO: 135, a CDR2 comprising the amino acid sequence set forth in SEQ ID NO: 136, and a CDR3 comprising the amino acid sequence set forth in SEQ ID NO: 137;
q) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 145;
r) a heavy chain variable region comprising a CDR1 comprising the amino acid sequence set forth in SEQ ID NO: 151, a CDR2 comprising the amino acid sequence set forth in SEQ ID NO: 2, and a CDR3 comprising the amino acid sequence set forth in SEQ ID NO: 152;
s) a heavy chain variable region comprising a CDR1 comprising the amino acid sequence set forth in SEQ ID NO: 11, a CDR2 comprising the amino acid sequence set forth in SEQ ID NO: 136, and a CDR3 comprising the amino acid sequence set forth in SEQ ID NO: 161;
t) a heavy chain variable region comprising a CDR1 comprising the amino acid sequence set forth in SEQ ID NO: 96, a CDR2 comprising the amino acid sequence set forth in SEQ ID NO: 167, and a CDR3 comprising the amino acid sequence set forth in SEQ ID NO: 168;
u) a heavy chain variable region comprising a CDR1 comprising the amino acid sequence set forth in SEQ ID NO: 21, a CDR2 comprising the amino acid sequence set forth in SEQ ID NO: 176, and a CDR3 comprising the amino acid sequence set forth in SEQ ID NO: 177;
v) a heavy chain variable region comprising a CDR1 comprising the amino acid sequence set forth in SEQ ID NO: 184, a CDR2 comprising the amino acid sequence set forth in SEQ ID NO: 185, and a CDR3 comprising the amino acid sequence set forth in SEQ ID NO: 186;
w) a heavy chain variable region comprising a CDR1 comprising the amino acid sequence set forth in SEQ ID NO: 11, a CDR2 comprising the amino acid sequence set forth in SEQ ID NO: 194, and a CDR3 comprising the amino acid sequence set forth in SEQ ID NO: 195; or
x) a heavy chain variable region comprising a CDR1 comprising the amino acid sequence set forth in SEQ ID NO: 201, a CDR2 comprising the amino acid sequence set forth in SEQ ID NO: 202, and a CDR3 comprising the amino acid sequence set forth in SEQ ID NO: 203.

In certain embodiments, the anti-DLL3 antibody or antigen-binding fragment thereof comprises:
a) a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6;
b) a light chain variable region comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16;
c) a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 23;
d) a light chain variable region comprising a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33;
e) a light chain variable region comprising a CDR1 comprising the amino acid sequence set forth in SEQ ID NO: 40, a CDR2 comprising the amino acid sequence set forth in SEQ ID NO: 5, and a CDR3 comprising the amino acid sequence set forth in SEQ ID NO: 41;
f) a light chain variable region comprising a CDR1 comprising the amino acid sequence set forth in SEQ ID NO: 49, CDR2 comprising the amino acid sequence set forth in SEQ ID NO: 50, and a CDR3 comprising the amino acid sequence set forth in SEQ ID NO: 51;
g) a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 59;
h) a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 65;
i) a light chain variable region comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 75;
j) a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82;
k) a light chain variable region comprising a CDR1 comprising the amino acid sequence set forth in SEQ ID NO: 90, a CDR2 comprising the amino acid sequence set forth in SEQ ID NO: 210, and a CDR3 comprising the amino acid sequence set forth in SEQ ID NO: 91;
l) a light chain variable region comprising a CDR1 comprising the amino acid sequence set forth in SEQ ID NO: 99, a CDR2 comprising the amino acid sequence set forth in SEQ ID NO: 100, and a CDR3 comprising the amino acid sequence set forth in SEQ ID NO: 101;
m) a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 112;
n) a light chain variable region comprising a CDR1 comprising the amino acid sequence set forth in SEQ ID NO: 117, a CDR2 comprising the amino acid sequence set forth in SEQ ID NO: 100, and a CDR3 comprising the amino acid sequence set forth in SEQ ID NO: 118;
o) a light chain variable region comprising a CDR1 comprising the amino acid sequence set forth in SEQ ID NO: 124, a CDR2 comprising the amino acid sequence set forth in SEQ ID NO: 58, and a CDR3 comprising the amino acid sequence set forth in SEQ ID NO: 125;
p) a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 130;
q) a light chain variable region comprising a CDR1 comprising the amino acid sequence set forth in SEQ ID NO: 138, a CDR2 comprising the amino acid sequence set forth in SEQ ID NO: 139, and a CDR3 comprising the amino acid sequence set forth in SEQ ID NO: 140;
r) a light chain variable region comprising a CDR1 comprising the amino acid sequence set forth in SEQ ID NO: 57, a CDR2 comprising the amino acid sequence set forth in SEQ ID NO: 146, and a CDR3 comprising the amino acid sequence set forth in SEQ ID NO: 125;
s) a light chain variable region comprising a CDR1 comprising the amino acid sequence set forth in SEQ ID NO: 124, a CDR2 comprising the amino acid sequence set forth in SEQ ID NO: 58, and a CDR3 comprising the amino acid sequence set forth in SEQ ID NO: 59;
t) a light chain variable region comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 162;
u) a light chain variable region comprising a CDR1 comprising the amino acid sequence set forth in SEQ ID NO: 169, a CDR2 comprising the amino acid sequence set forth in SEQ ID NO: 170, and a CDR3 comprising the amino acid sequence set forth in SEQ ID NO: 171;
v) a light chain variable region comprising a CDR1 comprising the amino acid sequence set forth in SEQ ID NO: 178, a CDR2 comprising the amino acid sequence set forth in SEQ ID NO: 50, and a CDR3 comprising the amino acid sequence set forth in SEQ ID NO: 179;
w) a light chain variable region comprising a CDR1 comprising the amino acid sequence set forth in SEQ ID NO: 187, a CDR2 comprising the amino acid sequence set forth in SEQ ID NO: 188, and a CDR3 comprising the amino acid sequence set forth in SEQ ID NO: 189;
x) a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 196; or
y) a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 204.

In certain embodiments, the anti-DLL3 antibody or antigen-binding fragment thereof comprises:
a) a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6;
b) a heavy chain variable region comprising a CDR1 comprising the amino acid sequence set forth in SEQ ID NO: 11, a CDR2 comprising an amino acid sequence set forth in SEQ ID NO: 12, and a CDR3 comprising the amino acid sequence set forth in SEQ ID NO: 13; and a light chain variable region comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16;
c) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 22; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 23;
d) a heavy chain variable region comprising a CDR1 comprising the amino acid sequence set forth in SEQ ID NO: 28, a CDR2 comprising the amino acid sequence set forth in SEQ ID NO: 29, and a CDR3 comprising the amino acid sequence set forth in SEQ ID NO: 30; and a light chain variable region comprising a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33;
e) a heavy chain variable region comprising a CDR1 comprising the amino acid sequence set forth in SEQ ID NO: 21, a CDR2 comprising the amino acid sequence set forth in SEQ ID NO: 38, and a CDR3 comprising the amino acid sequence set forth in SEQ ID NO: 39; and a light chain variable region comprising a CDR1 comprising the amino acid sequence set forth in SEQ ID NO: 40, a CDR2 comprising the amino acid sequence set forth in SEQ ID NO: 5, and a CDR3 comprising the amino acid sequence set forth in SEQ ID NO: 41;
f) a heavy chain variable region comprising a CDR1 comprising the amino acid sequence set forth in SEQ ID NO: 46, a CDR2 comprising the amino acid sequence set forth in SEQ ID NO: 47, and a CDR3 comprising the amino acid sequence set forth in SEQ ID NO: 48; and a light chain variable region comprising a CDR1 comprising the amino acid sequence set forth in SEQ ID NO: 49, a CDR2 comprising the amino acid sequence set forth in SEQ ID NO: 50, and a CDR3 comprising the amino acid sequence set forth in SEQ ID NO: 51;
g) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 56; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 59;
h) a heavy chain variable region comprising a CDR1 comprising the amino acid sequence set forth in SEQ ID NO: 21, a CDR2 comprising the amino acid sequence set forth in SEQ ID NO: 2, and CDR3 comprising the amino acid sequence set forth in SEQ ID NO: 64; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 65;
i) a heavy chain variable region comprising a CDR1 comprising the amino acid sequence set forth in SEQ ID NO: 70, a CDR2 comprising the amino acid sequence set forth in SEQ ID NO: 71, and a CDR3 comprising the amino acid sequence set forth in SEQ ID NO: 72; and a light chain variable region comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 75;
j) a heavy chain variable region comprising a CDR1 comprising the amino acid sequence set forth in SEQ ID NO: 21, a CDR2 comprising the amino acid sequence set forth in SEQ ID NO: 80, a CDR3 comprising the amino acid sequence set forth in SEQ ID NO: 81; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82;
k) a heavy chain variable region comprising a CDR1 comprising the amino acid sequence set forth in SEQ ID NO: 87, a CDR2 comprising the amino acid sequence set forth in SEQ ID NO: 88, and a CDR3 comprising the amino acid sequence set forth in SEQ ID NO: 89; and a light chain variable region comprising a CDR1 comprising the amino acid sequence set forth in SEQ ID NO: 90, a CDR2 comprising the amino acid sequence set forth in SEQ ID NO: 210, and a CDR3 comprising the amino acid sequence set forth in SEQ ID NO: 91;
l) a heavy chain variable region comprising a CDR1 comprising the amino acid sequence set forth in SEQ ID NO: 96, a CDR2 comprising the amino acid sequence set forth in SEQ ID NO: 97, and a CDR3 comprising the amino acid sequence set forth in SEQ ID NO: 98; and a light chain variable region comprising a CDR1 comprising the amino acid sequence set forth in SEQ ID NO: 99, a CDR2 comprising the amino acid sequence set forth in SEQ ID NO: 100, and a CDR3 comprising the amino acid sequence set forth in SEQ ID NO: 101;
m) a heavy chain variable region comprising a CDR1 comprising the amino acid sequence set forth in SEQ ID NO: 21, a CDR2 comprising the amino acid sequence set forth in SEQ ID NO: 106, and a CDR3 comprising the amino acid sequence set forth in SEQ ID NO: 107; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82;
n) a heavy chain variable region comprising a CDR1 comprising the amino acid sequence set forth in SEQ ID NO: 21, a CDR2 comprising the amino acid sequence set forth in SEQ ID NO: 106, and a CDR3 comprising the amino acid sequence set forth in SEQ ID NO: 107; and a light chain variable region comprising a CDR1 comprising the amino acid sequence set forth in SEQ ID NO: 4, a CDR2 comprising the amino acid sequence set forth in SEQ ID NO: 5, and a lCDR3 comprising the amino acid sequence set forth in SEQ ID NO: 112;
o) a heavy chain variable region comprising a CDR1 comprising the amino acid sequence set forth in SEQ ID NO: 96, a CDR2 comprising the amino acid sequence set forth in SEQ ID NO: 115, and a CDR3 comprising the amino acid sequence set forth in SEQ ID NO: 116; and a light chain variable region comprising a CDR1 comprising the amino acid sequence set forth in SEQ ID NO: 117, a CDR2 comprising the amino acid sequence set forth in SEQ ID NO: 100, and a CDR3 comprising the amino acid sequence set forth in SEQ ID NO: 118;
p) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 123; and a light chain variable region comprising a CDR1 comprising the amino acid sequence set forth in SEQ ID NO: 124, a CDR2 comprising the amino acid sequence set forth in SEQ ID NO: 58, and a CDR3 comprising the amino acid sequence set forth in SEQ ID NO: 125;
q) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 56; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 130;
r) a heavy chain variable region comprising a CDR1 comprising the amino acid sequence set forth in SEQ ID NO: 135, a CDR2 comprising the amino acid sequence set forth in SEQ ID NO: 136, and a CDR3 comprising the amino acid sequence set forth in SEQ ID NO: 137; and a light chain variable region comprising a CDR1 comprising the amino acid sequence set forth in SEQ ID NO: 138, a CDR2 comprising the amino acid sequence set forth in SEQ ID NO: 139, and a CDR3 comprising the amino acid sequence set forth in SEQ ID NO: 140;
s) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 145; and a light chain variable region comprising a CDR1 comprising the amino acid sequence set forth in SEQ ID NO: 57, a CDR2 comprising the amino acid sequence set forth in SEQ ID NO: 146, and a CDR3 comprising the amino acid sequence set forth in SEQ ID NO: 125;
t) a heavy chain variable region comprising a CDR1 comprising the amino acid sequence set forth in SEQ ID NO: 151, a CDR2 comprising the amino acid sequence set forth in SEQ ID NO: 2, and a CDR3 comprising the amino acid sequence set forth in SEQ ID NO: 152; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82;
u) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 123; and a light chain variable region comprising a CDR1 comprising the amino acid sequence set forth in SEQ ID NO: 124, a CDR2 comprising the amino acid sequence set forth in SEQ ID NO: 58, and a CDR3 comprising the amino acid sequence set forth in SEQ ID NO: 59;
v) a heavy chain variable region comprising a CDR1 comprising the amino acid sequence set forth in SEQ ID NO: 11, a CDR2 comprising the amino acid sequence set forth in SEQ ID NO: 136, a CDR3 comprising the amino acid sequence set forth in SEQ ID NO: 161; and a light chain variable region comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 162;
w) a heavy chain variable region comprising a CDR1 comprising the amino acid sequence set forth in SEQ ID NO: 96, a CDR2 comprising the amino acid sequence set forth in SEQ ID NO: 167 and a CDR3 comprising the amino acid sequence set forth in SEQ ID NO: 168; and a light chain variable region comprising a CDR1 comprising the amino acid sequence set forth in SEQ ID NO: 169, a CDR2 comprising the amino acid sequence set forth in SEQ ID NO: 170, and a CDR3 comprising the amino acid sequence set forth in SEQ ID NO: 171;
x) a heavy chain variable region comprising a CDR1 comprising the amino acid sequence set forth in SEQ ID NO: 21, a CDR2 comprising the amino acid sequence set forth in SEQ ID NO: 176, and a CDR3 comprising the amino acid sequence set forth in SEQ ID NO: 177; and a light chain variable region comprising a CDR1 comprising the amino acid sequence set forth in SEQ ID NO: 178, a CDR2 comprising the amino acid sequence set forth in SEQ ID NO: 50, and a CDR3 comprising the amino acid sequence set forth in SEQ ID NO: 179;
y) a heavy chain variable region comprising a CDR1 comprising the amino acid sequence set forth in SEQ ID NO: 184, a CDR2 comprising the amino acid sequence set forth in SEQ ID NO: 185, and a CDR3 comprising the amino acid sequence set forth in SEQ ID NO: 186; and a light chain variable region comprising a CDR1 comprising the amino acid sequence set forth in SEQ ID NO: 187, a CDR2 comprising the amino acid sequence set forth in SEQ ID NO: 188, and a CDR3 comprising the amino acid sequence set forth in SEQ ID NO: 188;
z) a heavy chain variable region comprising a CDR1 comprising the amino acid sequence set forth in SEQ ID NO: 11, a CDR2 comprising the amino acid sequence set forth in SEQ ID NO: 194, and a CDR3 comprising the amino acid sequence set forth in SEQ ID NO: 195; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 196; or
aa) a heavy chain variable region comprising a CDR1 comprising the amino acid sequence set forth in SEQ ID NO: 201, a CDR2 comprising the amino acid sequence set forth in SEQ ID NO: 202, and a CDR3 comprising the amino acid sequence set forth in SEQ ID NO: 203; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 204.

In certain embodiments, the anti-DLL3 antibody or antigen-binding fragment thereof comprises:
a) a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and CDR3 comprising the amino acid sequence set forth in SEQ ID NO: 6;
b) a heavy chain variable region comprising a CDR1 comprising the amino acid sequence set forth in SEQ ID NO: 28, a CDR2 comprising the amino acid sequence set forth in SEQ ID NO: 29, and a CDR3 comprising the amino acid sequence set forth in SEQ ID NO: 30; and a light chain variable region comprising a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33; or
c) a heavy chain variable region comprising a CDR1 comprising the amino acid sequence set forth in SEQ ID NO: 21, a CDR2 comprising the amino acid sequence set forth in SEQ ID NO: 38, and a CDR3 comprising the amino acid sequence set forth in SEQ ID NO: 39; and a light chain variable region comprising a CDR1 comprising the amino acid sequence set forth in SEQ ID NO: 40, a CDR2 comprising the amino acid sequence set forth in SEQ ID NO: 5, and a CDR3 comprising the amino acid sequence set forth in SEQ ID NO: 41.

In certain embodiments, the anti-DLL3 antibody or antigen-binding fragment thereof comprises a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 7, SEQ ID NO: 17, SEQ ID NO: 24, SEQ ID NO: 34, SEQ ID NO: 42, SEQ ID NO: 52, SEQ ID NO: 60, SEQ ID NO: 66, SEQ ID NO: 76, SEQ ID NO: 83, SEQ ID NO: 92, SEQ ID NO: 102, SEQ ID NO: 108, SEQ ID NO: 119, SEQ ID NO: 126, SEQ ID NO: 131, SEQ ID NO: 141, SEQ ID NO: 147, SEQ ID NO: 153, SEQ ID NO: 157, SEQ ID NO: 163, SEQ ID NO: 172, SEQ ID NO: 180, SEQ ID NO: 190, SEQ ID NO: 197, or SEQ ID NO: 205.

In certain embodiments, the anti-DLL3 antibody or antigen-binding fragment thereof comprises a heavy chain variable region comprising the amino acid sequence set forth in SEQ ID NO: 7, SEQ ID NO: 17, SEQ ID NO: 24, SEQ ID NO: 34, SEQ ID NO: 42, SEQ ID NO: 52, SEQ ID NO: 60, SEQ ID NO: 66, SEQ ID NO: 76, SEQ ID NO: 83, SEQ ID NO: 92, SEQ ID NO: 102, SEQ ID NO: 108, SEQ ID NO: 119, SEQ ID NO: 126, SEQ ID NO: 131, SEQ ID NO: 141, SEQ ID NO: 147, SEQ ID NO: 153, SEQ ID NO: 157, SEQ ID NO: 163, SEQ ID NO: 172, SEQ ID NO: 180, SEQ ID NO: 190, SEQ ID NO: 197, or SEQ ID NO: 205.

In certain embodiments, the anti-DLL3 antibody or antigen-binding fragment thereof comprises a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 8, SEQ ID NO: 18, SEQ ID NO: 25, SEQ ID NO: 35, SEQ ID NO: 43, SEQ ID NO: 53, SEQ ID NO: 61, SEQ ID NO: 67, SEQ ID NO: 77, SEQ ID NO: 84, SEQ ID NO: 93, SEQ ID NO: 103, SEQ ID NO: 109, SEQ ID NO: 113, SEQ ID NO: 120, SEQ ID NO: 127, SEQ ID NO: 132, SEQ ID NO: 142, SEQ ID NO: 148, SEQ ID NO: 154, SEQ ID NO: 158, SEQ ID NO: 164, SEQ ID NO: 173, SEQ ID NO: 181, SEQ ID NO: 191, SEQ ID NO: 198, or SEQ ID NO: 206.

In certain embodiments, the anti-DLL3 antibody or antigen-binding fragment thereof comprises a light chain variable region comprising the amino acid sequence set forth in SEQ ID NO: 8, SEQ ID NO: 18, SEQ ID NO: 25, SEQ ID NO: 35, SEQ ID NO: 43, SEQ ID NO: 53, SEQ ID NO: 61, SEQ ID NO: 67, SEQ ID NO: 77, SEQ ID NO: 84, SEQ ID NO: 93, SEQ ID NO: 103, SEQ ID NO: 109, SEQ ID NO: 113, SEQ ID NO: 120, SEQ ID NO: 127, SEQ ID NO: 132, SEQ ID NO: 142, SEQ ID NO: 148, SEQ ID NO: 154, SEQ ID NO: 158, SEQ ID NO: 164, SEQ ID NO: 173, SEQ ID NO: 181, SEQ ID NO: 191, SEQ ID NO: 198, or SEQ ID NO: 206.

In certain embodiments, the anti-DLL3 antibody or antigen-binding fragment thereof comprises:
a) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 7, SEQ ID NO: 17, SEQ ID NO: 24, SEQ ID NO: 34, SEQ ID NO: 42, SEQ ID NO: 52, SEQ ID NO: 60, SEQ ID NO: 66, SEQ ID NO: 76, SEQ ID NO: 83, SEQ ID NO: 92, SEQ ID NO: 102, SEQ ID NO: 108, SEQ ID NO: 119, SEQ ID NO: 126, SEQ ID NO: 131, SEQ ID NO: 141, SEQ ID NO: 147, SEQ ID NO: 153, SEQ ID NO: 157, SEQ ID NO: 163, SEQ ID NO: 172, SEQ ID NO: 180, SEQ ID NO: 190, SEQ ID NO: 197, or SEQ ID NO: 205; and
b) a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 8, SEQ ID NO: 18, SEQ ID NO: 25, SEQ ID NO: 35, SEQ ID NO: 43, SEQ ID NO: 53, SEQ ID NO: 61, SEQ ID NO: 67, SEQ ID NO: 77, SEQ ID NO: 84, SEQ ID NO: 93, SEQ ID NO: 103, SEQ ID NO: 109, SEQ ID NO: 113, SEQ ID NO: 120, SEQ ID NO: 127, SEQ ID NO: 132, SEQ ID NO: 142, SEQ ID NO: 148, SEQ ID NO: 154, SEQ ID NO: 158, SEQ ID NO: 164, SEQ ID NO: 173, SEQ ID NO: 181, SEQ ID NO: 191, SEQ ID NO: 198, or SEQ ID NO: 206.

In certain embodiments, the anti-DLL3 antibody or antigen-binding fragment thereof comprises:
a) a heavy chain variable region comprising the amino acid sequence set forth in SEQ ID NO: 7, SEQ ID NO: 17, SEQ ID NO: 24, SEQ ID NO: 34, SEQ ID NO: 42, SEQ ID NO: 52, SEQ ID NO: 60, SEQ ID NO: 66, SEQ ID NO: 76, SEQ ID NO: 83, SEQ ID NO: 92, SEQ ID NO: 102, SEQ ID NO: 108, SEQ ID NO: 119, SEQ ID NO: 126, SEQ ID NO: 131, SEQ ID NO: 141, SEQ ID NO: 147, SEQ ID NO: 153, SEQ ID NO: 157, SEQ ID NO: 163, SEQ ID NO: 172, SEQ ID NO: 180, SEQ ID NO: 190, SEQ ID NO: 197, or SEQ ID NO: 205; and
b) a light chain variable region comprising the amino acid sequence set forth in SEQ ID NO: 8, SEQ ID NO: 18, SEQ ID NO: 25, SEQ ID NO: 35, SEQ ID NO: 43, SEQ ID NO: 53, SEQ ID NO: 61, SEQ ID NO: 67, SEQ ID NO: 77, SEQ ID NO: 84, SEQ ID NO: 93, SEQ ID NO: 103, SEQ ID NO: 109, SEQ ID NO: 113, SEQ ID NO: 120, SEQ ID NO: 127, SEQ ID NO: 132, SEQ ID NO: 142, SEQ ID NO: 148, SEQ ID NO: 154, SEQ ID NO: 158, SEQ ID NO: 164, SEQ ID NO: 173, SEQ ID NO: 181, SEQ ID NO: 191, SEQ ID NO: 198, or SEQ ID NO: 206.

In certain embodiments, the anti-DLL3 antibody or antigen-binding fragment thereof comprises:
a) a heavy chain variable region comprising the amino acid sequence set forth in SEQ ID NO: 7, and a light chain variable region comprising the amino acid sequence set forth in SEQ ID NO: 8;
b) a heavy chain variable region comprising the amino acid sequence set forth in SEQ ID NO: 17, and a light chain variable region comprising the amino acid sequence set forth in SEQ ID NO: 18;
c) a heavy chain variable region comprising the amino acid sequence set forth in SEQ ID NO: 24, and a light chain variable region comprising the amino acid sequence set forth in SEQ ID NO: 25;
d) a heavy chain variable region comprising the amino acid sequence set forth in SEQ ID NO: 34, and a light chain variable region comprising the amino acid sequence set forth in SEQ ID NO: 35;
e) a heavy chain variable region comprising the amino acid sequence set forth in SEQ ID NO: 42, and a light chain variable region comprising the amino acid sequence set forth in SEQ ID NO: 43;
f) a heavy chain variable region comprising the amino acid sequence set forth in SEQ ID NO: 52, and a light chain variable region comprising the amino acid sequence set forth in SEQ ID NO: 53;
g) a heavy chain variable region comprising the amino acid sequence set forth in SEQ ID NO: 60, and a light chain variable region comprising the amino acid sequence set forth in SEQ ID NO: 61;
h) a heavy chain variable region comprising the amino acid sequence set forth in SEQ ID NO: 66, and a light chain variable region comprising the amino acid sequence set forth in SEQ ID NO: 67;
i) a heavy chain variable region comprising the amino acid sequence set forth in SEQ ID NO: 76, and a light chain variable region comprising the amino acid sequence set forth in SEQ ID NO: 77;
j) a heavy chain variable region comprising the amino acid sequence set forth in SEQ ID NO: 83, and a light chain variable region comprising the amino acid sequence set forth in SEQ ID NO: 84;
k) a heavy chain variable region comprising the amino acid sequence set forth in SEQ ID NO: 92, and a light chain variable region comprising the amino acid sequence set forth in SEQ ID NO: 93;
l) a heavy chain variable region comprising the amino acid sequence set forth in SEQ ID NO: 102, and a light chain variable region comprising the amino acid sequence set forth in SEQ ID NO: 103;
m) a heavy chain variable region comprising the amino acid sequence set forth in SEQ ID NO: 108, and a light chain variable region comprising the amino acid sequence set forth in SEQ ID NO: 109;
n) a heavy chain variable region comprising the amino acid sequence set forth in SEQ ID NO: 108, and a light chain variable region comprising the amino acid sequence set forth in SEQ ID NO: 113;
o) a heavy chain variable region comprising the amino acid sequence set forth in SEQ ID NO: 119, and a light chain variable region comprising the amino acid sequence set forth in SEQ ID NO: 120;
p) a heavy chain variable region comprising the amino acid sequence set forth in SEQ ID NO: 126, and a light chain variable region comprising the amino acid sequence set forth in SEQ ID NO: 127;
q) a heavy chain variable region comprising the amino acid sequence set forth in SEQ ID NO: 131, and a light chain variable region comprising the amino acid sequence set forth in SEQ ID NO: 132;
r) a heavy chain variable region comprising the amino acid sequence set forth in SEQ ID NO: 141, and a light chain variable region comprising the amino acid sequence set forth in SEQ ID NO: 142;
s) a heavy chain variable region comprising the amino acid sequence set forth in SEQ ID NO: 147, and a light chain variable region comprising the amino acid sequence set forth in SEQ ID NO: 148;
t) a heavy chain variable region comprising the amino acid sequence set forth in SEQ ID NO: 153, and a light chain variable region comprising the amino acid sequence set forth in SEQ ID NO: 154;
u) a heavy chain variable region comprising the amino acid sequence set forth in SEQ ID NO: 157, and a light chain variable region comprising the amino acid sequence set forth in SEQ ID NO: 158;
v) a heavy chain variable region comprising the amino acid sequence set forth in SEQ ID NO: 163, and a light chain variable region comprising the amino acid sequence set forth in SEQ ID NO: 164;
w) a heavy chain variable region comprising the amino acid sequence set forth in SEQ ID NO: 172, and a light chain variable region comprising the amino acid sequence set forth in SEQ ID NO: 173;
x) a heavy chain variable region comprising the amino acid sequence set forth in SEQ ID NO: 180, and a light chain variable region comprising the amino acid sequence set forth in SEQ ID NO: 181;
y) a heavy chain variable region comprising the amino acid sequence set forth in SEQ ID NO: 190, and a light chain variable region comprising the amino acid sequence set forth in SEQ ID NO: 191;
z) a heavy chain variable region comprising the amino acid sequence set forth in SEQ ID NO: 197, and a light chain variable region comprising the amino acid sequence set forth in SEQ ID NO: 198; or
aa) a heavy chain variable region comprising the amino acid sequence set forth in SEQ ID NO: 205, and a light chain variable region comprising the amino acid sequence set forth in SEQ ID NO: 206.

In certain embodiments, the anti-DLL3 antibody or antigen-binding fragment thereof comprises:
a) a heavy chain variable region comprising the amino acid sequence set forth in SEQ ID NO: 7, and a light chain variable region comprising the amino acid sequence set forth in SEQ ID NO: 8;
b) a heavy chain variable region comprising the amino acid sequence set forth in SEQ ID NO: 34, and a light chain variable region comprising the amino acid sequence set forth in SEQ ID NO: 35; or
c) a heavy chain variable region comprising the amino acid sequence set forth in SEQ ID NO: 42, and a light chain variable region comprising the amino acid sequence set forth in SEQ ID NO: 43.

In certain embodiments, the TCO has formula

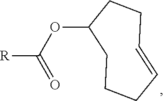

wherein R is N, C, or an ester.

In certain embodiments, the TCO has formula

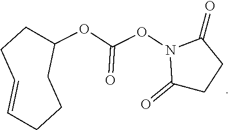 [SMILES: O=C(OC1CC/C=C/CCC1)ON1C(=O)CCC1=O]

The presently disclosed subject matter also provides a composition comprising the antibody-TCO conjugate disclosed herein. In certain embodiments, the composition is pharmaceutical composition further comprising a pharmaceutically acceptable carrier.

Furthermore, the presently disclosed subject matter provides a radioimmunoconjugate comprising the antibody-TCO conjugate disclosed herein and a radioligand comprising a tetrazine moiety (Tz) and a radiolabel. In certain embodiments, the radiolabel comprises a radioactive isotope selected from the group consisting of ^{99m}Tc, ¹¹¹In, ⁴⁴Sc, ⁴⁷Sc, ⁶⁴Cu, ⁶⁷Ga, ⁶⁸Ga, ⁷²As, ⁷⁷As, ⁵²Mn, ¹⁸⁶Re, ¹⁵³Sm ¹⁷⁷Lu, ⁶⁷Cu, ¹²³I, ¹²⁴I, ¹²⁵I, ¹¹C, ¹³N, ¹⁵O, ¹⁸F, ¹⁶⁶Ho, ¹⁴⁹Pm, ⁹⁰Y, ⁸⁶Y, ²¹³Bi, ¹⁰³Pd, ¹⁰⁹Pd, ¹⁵⁹Gd, ¹⁴⁰La, ¹⁹⁸Au, ¹⁹⁹Au, ¹⁷⁵Yb, ¹⁶⁹Yb, ¹⁶⁵Dy, ¹⁶⁶Dy, ¹⁰⁵Ph, ¹¹¹Ag, ⁸⁹Zr, ¹⁹²Ir, ²²⁵Ac, ²¹²Bi ²¹³Bi, ⁷⁶Br, ⁵⁵Co, ⁶⁰Cu, ⁶¹Cu, ⁶²Cu, ⁶⁶Ga, ⁹⁰Nb, ²¹²Pb, ¹⁵²Tb, ⁷⁷Br, ²¹¹At, ²²⁷Th, ²²⁴Ra and ¹⁴⁹Tb. In certain embodiments, the radiolabel comprises a ⁶⁴Cu radioactive isotope. In certain embodiments, the radiolabel comprises a ²²⁵Ac radioactive isotope.

In certain embodiments, the radioimmunoconjugate further comprises a chelator. In certain embodiments, the chelator is a sarcophagine chelator. In certain embodiments, the chelator is 1,4,7-triazonane-1,4,7-triyl-triacetic acid (NOTA) or 1,4,7-triazacyclononane-1,4-diacetate (NODA).

In certain embodiments, the radioimmunoconjugate further comprises a linker attaching the tetrazine moiety (Tz) to the chelator. In certain embodiments, the linker comprises is polyethylene glycol (PEG) or (poly)-L-lysine. In certain embodiments, the polyethylene glycol (PEG) comprises from about 1 unit to about 100 units. In certain embodiments, the (poly)-L-lysine comprises from about 1 unit to about 200 units.

In certain embodiments, the tetrazine moiety (Tz) and the chelator form a compound selected from the group consisting of:
a) 2,2′,2″-(3-(4-(3-(1-(4-(1,2,4,5-tetrazin-3-yl)phenyl)-1-oxo-5,8,11,14,17,20,23-heptaoxa-2-azapentacosan-25-yl)thioureido)benzyl)-1,4,7-triazonane-2,5,8-triyl)triacetic acid;
b) 2,2′,2″-(3-(4-(3-(1-(4-(1,2,4,5-tetrazin-3-yl)phenyl)-1-oxo-5,8,11,14,17,20,23,26,29,32,35-undecaoxa-2-azaheptatriacontan-37-yl)thioureido)benzyl)-1,4,7-triazonane-2,5,8-triyl)triacetic acid;
c) 2,2′-(7-(4-(3-(1-(4-(1,2,4,5-tetrazin-3-yl)phenyl)-1-oxo-5,8,11,14,17,20,23,26,29,32,35-undecaoxa-2-azaheptatriacontan-37-yl)thioureido)benzyl)-1,4,7-triazonane-1,4-diyl)diacetic acid;
d) 2,2′,2″-(3-(4-(3-(1-(4-(1,2,4,5-tetrazin-3-yl)phenyl)-3,7-dioxo-11,14,17,20,23,26,29-heptaoxa-2,8-diazahentriacontan-31-yl)thioureido)benzyl)-1,4,7-triazonane-2,5,8-triyl)triacetic acid;
e) 2,2′,2″-(3-(4-(3-(1-(4-(1,2,4,5-tetrazin-3-yl)phenyl)-3,7-dioxo-11,14,17,20,23,26,29,32,35,38,41-undecaoxa-2,8-diazatritetracontan-43-yl)thioureido)benzyl)-1,4,7-triazonane-2,5,8-triyl)triacetic acid;
f) 2,2′,2″-(3-(4-(3-(25,28-dioxo-28-((6-(6-(pyridin-2-yl)-1,2,4,5-tetrazin-3-yl)pyridin-3-yl)amino)-3,6,9,12,15,18,21-heptaoxa-24-azaoctacosyl)thioureido)benzyl)-1,4,7-triazonane-2,5,8-triyl)triacetic acid;
g) 2,2′,2″-(3-(4-(3-(37,40-dioxo-40-((6-(6-(pyridin-2-yl)-1,2,4,5-tetrazin-3-yl)pyridin-3-yl)amino)-3,6,9,12,15,18,21,24,27,30,33-undecaoxa-36-azatetracontyl)thioureido)benzyl)-1,4,7-triazonane-2,5,8-triyl)triacetic acid;
h) 2,2′,2″-(3-(4-(1-(4-(6-methyl-1,2,4,5-tetrazin-3-yl)phenyl)-3-oxo-6,9,12,15,18,21,24-heptaoxa-2-azaheptacosan-27-amido)benzyl)-1,4,7-triazonane-2,5,8-triyl)triacetic acid;
i) 2,2′,2″-(2-(4-(1-(4-(6-methyl-1,2,4,5-tetrazin-3-yl)phenoxy)-3,6,9,12,15,18,21,24,27,30,33-undecaoxahexatriacontan-36-amido)benzyl)-1,4,7-triazonane-1,4,7-triyl)triacetic acid;
j) 2,2′,2″-(3-(4-(3-(5-amino-6-((4-(6-methyl-1,2,4,5-tetrazin-3-yl)benzyl)amino)-6-oxohexyl)thioureido)benzyl)-1,4,7-triazonane-2,5,8-triyl)triacetic acid;
k) 2,2′-(7-(4-(3-(5-amino-6-((4-6-methyl-1,2,4,5-tetrazin-3-yl)benzyl)amino)-6-oxohexyl)thioureido)benzyl)-1,4,7-triazonane-1,4-diyl)diacetic acid;
l) 2,2′,2″-(3-(4-(3-(5-amino-6-((5-amino-6-((4-(6-methyl-1,2,4,5-tetrazin-3-yl)benzyl)amino)-6-oxohexyl)amino)-6-oxohexyl)thioureido)benzyl)-1,4,7-triazonane-2,5,8-triyl)triacetic acid; and
m) 2,2′,2″-(3-(4-(3-(5-amino-6-((5-amino-6-((5-amino-6-((4-(6-methyl-1,2,4,5-tetrazin-3-yl)benzyl)amino)-6-oxohexyl)amino)-6-oxohexyl)amino)-6-oxohexyl)thioureido)benzyl)-1,4,7-triazonane-2,5,8-triyl)triacetic acid.

In certain embodiments, the antibody-TCO conjugate and the radioligand bind together to form the radioimmunoconjugate via an in vivo click reaction.

The presently disclosed subject matter also provides methods of in vivo diagnosis of a disease or disorder associated with DLL3 in a subject. In certain embodiments, the methods comprise administering to the subject the antibody-TCO conjugate disclosed herein or the composition thereof. In certain embodiments, a portion of the antibody-TCO conjugate localizes at cells of the disease or disorder and unbound antibody-TCO conjugate is cleared from blood, from renal system, and/or from the subject after an accumulation interval. In certain embodiments, the methods further comprise administering to the subject a radioligand comprising a tetrazine moiety (Tz) and a radiolabel or a composition thereof. In certain embodiments, the antibody-TCO conjugate and the radioligand bind together to form a radioimmunoconjugate via an in vivo click reaction at the cells of the disease or disorder within a region of the subject. In certain embodiments, the methods further comprise imaging the radioimmunoconjugate accumulated in the region of the subject.

In certain embodiments, the radioligand is delivered to the subject after the accumulation interval. In certain embodiments, the imaging occurs via positron emission tomography (PET). In certain embodiments, the imaging occurs within a time period up to about 7 days from the administering of the radioligand. In certain embodiments, the imaging occurs between about 24 hours and up to about 7 days from the administering of the radioligand.

In certain embodiments, the radiolabel comprises a radioactive isotope selected from the group consisting of ^{99m}Tc, ¹¹¹In, ⁴⁴Sc, ⁴⁷Sc, ⁶⁴Cu, ⁶⁷Ga, ⁶⁸Ga, ⁷²As, ⁷⁷As, ⁵²Mn, ¹⁸⁶Re, ¹⁵³Sm, ¹⁷⁷Lu, ⁶⁷Cu, ¹²³I, ¹²⁴I, ¹²⁵I, ¹¹C, ¹³N, ¹⁵O, ¹⁸F, ¹⁶⁶Ho, ¹⁴⁹Pm, ⁹⁰Y, ⁸⁶Y, ²¹³Bi, ¹⁰³Pd, ¹⁰⁹Pd, ¹⁵⁹Gd, ¹⁴⁰La, ¹⁹⁸Au, ¹⁹⁹Au, ¹⁷⁵Yb, ¹⁶⁹Yb, ¹⁶⁵Dy, ¹⁶⁶Dy, ¹⁰⁵Ph, ¹¹¹Ag, ⁸⁹Zr ¹⁹²Ir, ²²⁵Ac, ²¹²Bi, ²¹³Bi, ⁷⁶Br, ⁵⁵Co, ⁶⁰Cu, ⁶¹Cu, ⁶²Cu, ⁶⁶Ga, ⁹⁰Nb, ²¹²Pb, ¹⁵²Tb, ⁷⁷Br, ²¹¹At, ²²⁷Th, ²²⁴Ra and ¹⁴⁹Tb In certain embodiments, the radiolabel comprises a ⁶⁴Cu radioactive isotope. In certain embodiments, the radiolabel comprises a ¹⁸F radioactive isotope. In certain embodiments, the radioligand further comprises a chelator. In certain embodiments, the chelator is a sarcophagine chelator. In certain embodiments, the chelator is 1,4,7-triazonane-1,4,7-triyl-triacetic acid (NOTA) or 1,4,7-triazacyclononane-1,4-diacetate (NODA). In certain embodiments, the radioligand further comprises a linker attaching the tetrazine moiety (Tz) to the chelator. In certain embodiments, the linker comprises is polyethylene glycol (PEG) or (poly)-L-lysine. In certain embodiments, the polyethylene glycol (PEG) comprises from about 1 unit to about 100 units. In certain embodiments, the (poly)-L-lysine comprises from about 1 unit to about 200 units.

In certain embodiments, the tetrazine moiety (Tz) and the chelator form a compound selected from the group consisting of:
a) 2,2′,2″-(3-(4-(3-(1-(4-(1,2,4,5-tetrazin-3-yl)phenyl)-1-oxo-5,8,11,14,17,20,23-heptaoxa-2-azapentacosan-25-yl)thioureido)benzyl)-1,4,7-triazonane-2,5,8-triyl)triacetic acid;
b) 2,2′,2″-(3-(4-(3-(1-(4-(1,2,4,5-tetrazin-3-yl)phenyl)-1-oxo-5,8,11,14,17,20,23,26,29,32,35-undecaoxa-2-azaheptatriacontan-37-yl)thioureido)benzyl)-1,4,7-triazonane-2,5,8-triyl)triacetic acid;
c) 2,2′-(7-(4-(3-(1-(4-(1,2,4,5-tetrazin-3-yl)phenyl)-1-oxo-5,8,11,14,17,20,23,26,29,32,35-undecaoxa-2-azaheptatriacontan-37-yl)thioureido)benzyl)-1,4,7-triazonane-1,4-diyl)diacetic acid;
d) 2,2′,2″-(3-(4-(3-(1-(4-(1,2,4,5-tetrazin-3-yl)phenyl)-3,7-dioxo-11,14,17,20,23,26,29-heptaoxa-2,8-diazahentriacontan-31-yl)thioureido)benzyl)-1,4,7-triazonane-2,5,8-triyl)triacetic acid;
e) 2,2′,2″-(3-(4-(3-(1-(4-(1,2,4,5-tetrazin-3-yl)phenyl)-3,7-dioxo-11,14,17,20,23,26,29,32,35,38,41-undecaoxa-2,8-diazatritetracontan-43-yl)thioureido)benzyl)-1,4,7-triazonane-2,5,8-triyl)triacetic acid;
f) 2,2′,2″-(3-(4-(3-(25,28-dioxo-28-((6-(6-(pyridin-2-yl)-1,2,4,5-tetrazin-3-yl)pyridin-3-yl)amino)-3,6,9,12,15,18,21-heptaoxa-24-azaoctacosyl)thioureido)benzyl)-1,4,7-triazonane-2,5,8-triyl)triacetic acid;
g) 2,2′,2″-(3-(4-(3-(37,40-dioxo-40-((6-(6-(pyridin-2-yl)-1,2,4,5-tetrazin-3-yl)pyridin-3-yl)amino)-3,6,9,12,15,18,21,24,27,30,33-undecaoxa-36-azatetracontyl)thioureido)benzyl)-1,4,7-triazonane-2,5,8-triyl)triacetic acid;
h) 2,2′,2″-(3-(4-(1-(4-(6-methyl-1,2,4,5-tetrazin-3-yl)phenyl)-3-oxo-6,9,12,15,18,21,24-heptaoxa-2-azaheptacosan-27-amido)benzyl)-1,4,7-triazonane-2,5,8-triyl)triacetic acid;
i) 2,2′,2″-(2-(4-(1-(4-(6-methyl-1,2,4,5-tetrazin-3-yl)phenoxy)-3,6,9,12,15,18,21,24,27,30,33-undecaoxahexatriacontan-36-amido)benzyl)-1,4,7-triazonane-1,4,7-triyl)triacetic acid;
j) 2,2′,2″-(3-(4-(3-(5-amino-6-((4-(6-methyl-1,2,4,5-tetrazin-3-yl)benzyl)amino)-6-oxohexyl)thioureido)benzyl)-1,4,7-triazonane-2,5,8-triyl)triacetic acid;
k) 2,2′-(7-(4-(3-(5-amino-6-((4-6-methyl-1,2,4,5-tetrazin-3-yl)benzyl)amino)-6-oxohexyl)thioureido)benzyl)-1,4,7-triazonane-1,4-diyl)diacetic acid;
l) 2,2′,2″-(3-(4-(3-(5-amino-6-((5-amino-6-((4-(6-methyl-1,2,4,5-tetrazin-3-yl)benzyl)amino)-6-oxohexyl)amino)-6-oxohexyl)thioureido)benzyl)-1,4,7-triazonane-2,5,8-triyl)triacetic acid; and
m) 2,2′,2″-(3-(4-(3-(5-amino-6-((5-amino-6-((5-amino-6-((4-(6-methyl-1,2,4,5-tetrazin-3-yl)benzyl)amino)-6-oxohexyl)amino)-6-oxohexyl)amino)-6-oxohexyl)thioureido)benzyl)-1,4,7-triazonane-2,5,8-triyl)triacetic acid.

In certain embodiments, the imaging comprises detecting the radioactive levels emitted by the radioimmunoconjugate. In certain embodiments, the radioactive levels emitted by the radioimmunoconjugate are expressed as the percentage injected dose per gram tissue (% ID/g).

The presently disclosed subject matter also provides methods for treating a disease or disorder associated with DLL3 in a subject. In certain embodiments, the methods comprise administering to the subject the antibody-TCO conjugate disclosed herein or the composition thereof. In certain embodiments, a portion of the antibody-TCO conjugate localizes at cells of the disease or disorder and unbound antibody-TCO conjugate is cleared from blood, from renal system, and/or from the subject after an accumulation interval. In certain embodiments, the methods further comprise administering to the subject a radioligand comprising a tetrazine moiety (Tz) and a radiolabel or a composition comprising the radioligand. In certain embodiments, the antibody-TCO conjugate and the radioligand bind together to form a radioimmunoconjugate via an in vivo click reaction at the cells of the disease or disorder within a region of the subject. In certain embodiments, the radiolabel comprises a radioactive isotope selected from the group consisting of ^{99m}Tc, ¹¹¹In, ⁴⁴Sc, ⁴⁷Sc, ⁶⁴Cu, ⁶⁷Ga, ⁶⁸Ga, ⁷²As, ⁷⁷As, ⁵²Mn, ¹⁸⁶Re, ¹⁵³Sm, ¹⁷⁷Lu, ⁶⁷Cu, ¹²³I, ¹²⁴I, ¹²⁵I, ¹¹C, ¹³N, ¹⁵O, ¹⁸F, ¹⁶⁶Ho, ¹⁴⁹Pm, ⁹⁰Y, ⁸⁶Y, ²¹³Bi, ¹⁰³Pd, ¹⁰⁹Pd, ¹⁵⁹Gd, ¹⁴⁰La, ¹⁹⁸Au, ¹⁹⁹Au, ¹⁷⁵Yb ¹⁶⁹Yb ¹⁶⁵Dy, ¹⁶⁶Dy, ¹⁰⁵Rh, ¹¹¹Ag, ⁸⁹Zr, ¹⁹²Ir, ²²⁵Ac, ²¹²Bi, ²¹³Bi, ⁷⁶Br, ⁵⁵Co, ⁶⁰Cu, ⁶¹Cu, ⁶²Cu, ⁶⁶Ga, ⁹⁰Nb, ²¹²Pb, ¹⁵²Tb, ⁷⁷Br, ²¹¹At, ²²⁷Th, ²²⁴Ra and ¹⁴⁹Tb.

In certain embodiments, the radioligand is delivered to the subject after the accumulation interval. In certain embodiments, the radioligand further comprises a chelator. In certain embodiments, the chelator is a sarcophagine chelator. In certain embodiments, the chelator is 1,4,7-triazonane-1,4,7-triyl-triacetic acid (NOTA) or 1,4,7-triazacyclononane-1,4-diacetate (NODA). In certain embodiments, the radioligand further comprises a linker attaching the tetrazine moiety (Tz) to the chelator. In certain embodiments, the linker comprises is polyethylene glycol (PEG) or (poly)-L-lysine. In certain embodiments, the polyethylene glycol (PEG) comprises from about 1 unit to about 100 units. In certain embodiments, the (poly)-L-lysine comprises from about 1 unit to about 200 units. In certain embodiments, the tetrazine moiety (Tz) and the chelator form a compound selected from the group consisting of:
a) 2,2′,2″-(3-(4-(3-(1-(4-(1,2,4,5-tetrazin-3-yl)phenyl)-1-oxo-5,8,11,14,17,20,23-heptaoxa-2-azapentacosan-25-yl)thioureido)benzyl)-1,4,7-triazonane-2,5,8-triyl)triacetic acid; b) 2,2′,2″-(3-(4-(3-(1-(4-(1,2,4,5-tetrazin-3-yl)phenyl)-1-oxo-5,8,11,14,17,20,23,26,29,32,35-undecaoxa-2-azaheptatriacontan-37-yl)thioureido)benzyl)-1,4,7-triazonane-2,5,8-triyl)triacetic acid;
c) 2,2′-(7-(4-(3-(1-(4-(1,2,4,5-tetrazin-3-yl)phenyl)-1-oxo-5,8,11,14,17,20,23,26,29,32,35-undecaoxa-2-azaheptatriacontan-37-yl)thioureido)benzyl)-1,4,7-triazonane-1,4-diyl)diacetic acid;
d) 2,2′,2″-(3-(4-(3-(1-(4-(1,2,4,5-tetrazin-3-yl)phenyl)-3,7-dioxo-11,14,17,20,23,26,29-heptaoxa-2,8-diazahentriacontan-31-yl)thioureido)benzyl)-1,4,7-triazonane-2,5,8-triyl)triacetic acid;
e) 2,2′,2″-(3-(4-(3-(1-(4-(1,2,4,5-tetrazin-3-yl)phenyl)-3,7-dioxo-11,14,17,20,23,26,29,32,35,38,41-undecaoxa-2,8-diazatritetracontan-43-yl)thioureido)benzyl)-1,4,7-triazonane-2,5,8-triyl)triacetic acid;
f) 2,2′,2″-(3-(4-(3-(25,28-dioxo-28-((6-(6-(pyridin-2-yl)-1,2,4,5-tetrazin-3-yl)pyridin-3-yl)amino)-3,6,9,12,15,18,21-heptaoxa-24-azaoctacosyl)thioureido)benzyl)-1,4,7-triazonane-2,5,8-triyl)triacetic acid;
g) 2,2′,2″-(3-(4-(3-(37,40-dioxo-40-((6-(6-(pyridin-2-yl)-1,2,4,5-tetrazin-3-yl)pyridin-3-yl)amino)-3,6,9,12,15,18,21,24,27,30,33-undecaoxa-36-azatetracontyl)thioureido)benzyl)-1,4,7-triazonane-2,5,8-triyl)triacetic acid;
h) 2,2′,2″-(3-(4-(1-(4-(6-methyl-1,2,4,5-tetrazin-3-yl)phenyl)-3-oxo-6,9,12,15,18,21,24-heptaoxa-2-azaheptacosan-27-amido)benzyl)-1,4,7-triazonane-2,5,8-triyl)triacetic acid;
i) 2,2′,2″-(2-(4-(1-(4-(6-methyl-1,2,4,5-tetrazin-3-yl)phenoxy)-3,6,9,12,15,18,21,24,27,30,33-undecaoxahexatriacontan-36-amido)benzyl)-1,4,7-triazonane-1,4,7-triyl)triacetic acid;
j) 2,2′,2″-(3-(4-(3-(5-amino-6-((4-(6-methyl-1,2,4,5-tetrazin-3-yl)benzyl)amino)-6-oxohexyl)thioureido)benzyl)-1,4,7-triazonane-2,5,8-triyl)triacetic acid;
k) 2,2′-(7-(4-(3-(5-amino-6-((4-6-methyl-1,2,4,5-tetrazin-3-yl)benzyl)amino)-6-oxohexyl)thioureido)benzyl)-1,4,7-triazonane-1,4-diyl)diacetic acid;
l) 2,2′,2″-(3-(4-(3-(5-amino-6-((5-amino-6-((4-(6-methyl-1,2,4,5-tetrazin-3-yl)benzyl)amino)-6-oxohexyl)amino)-6-oxohexyl)thioureido)benzyl)-1,4,7-triazonane-2,5,8-triyl)triacetic acid; and
m) 2,2′,2″-(3-(4-(3-(5-amino-6-((5-amino-6-((5-amino-6-((4-(6-methyl-1,2,4,5-tetrazin-3-yl)benzyl)amino)-6-oxohexyl)amino)-6-oxohexyl)amino)-6-oxohexyl)thioureido)benzyl)-1,4,7-triazonane-2,5,8-triyl)triacetic acid.

In certain embodiments, the disease or disorder associated with DLL3 is a tumor. In certain embodiments, the tumor is a cancer. In certain embodiments, the disease or disorder associated with DLL3 is selected from neuroendocrine tumors of the lung, extrapulmonary neuroendocrine carcinomas, melanoma, neuroendocrine prostate cancer, and breast cancer. In certain embodiments, the neuroendocrine tumors of the lung are selected from the group consisting of pulmonary neuroendocrine cancer, large cell neuroendocrine carcinoma, and small-cell lung cancer. In certain embodiments, the subject is a human.

The presently disclosed subject matter further provides kits for in vivo diagnosis and/or treatment of a disease or disorder associated with DLL3 in a subject. In certain embodiments, the kits comprise the antibody-TCO conjugate disclosed herein or the composition thereof, and a radioligand comprising a tetrazine moiety (Tz) and a radiolabel or a composition thereof. In certain embodiments, the kits further comprise written instructions for using the antibody-TCO conjugate and the radioligand for in vivo diagnosis and/or treatment of a disease or disorder associated with DLL3 in a subject.

### 4. BRIEF DESCRIPTION OF THE DRAWINGS

The following Detailed Description, given by way of example, but not intended to limit the invention to specific embodiments described, may be understood in conjunction with the accompanying drawings.

FIG. 1 depicts a schematic of the pretargeted PET imaging strategy.

FIGS. 2A and 2B depict representative molecules used in the presently disclosed subject matter. FIG. 2A shows the structure of TCO. FIG. 2B shows the structure of ⁶⁴Cu-Tz-SarAr.

FIGS. 3A and 3B depict imagining experiments using directly labeled antibodies. FIG. 3A shows results using [⁸⁹Zr]Zr-DFO-A18 antibody. FIG. 3B shows results using [⁸⁹Zr]Zr-DFO-J8 antibody.

FIG. 4 depicts a pretargeting study setup for detection of small cell lung cancer (SCLC).

FIGS. 5A-5E depict maximum intensity projections (MIPs) at different time points.

FIG. 5A shows MIPs at 4 h post-injection (p.i.). FIG. 5B shows MIPs at 12 h p.i. FIG. 5C shows MIPs at 24 h p.i. FIG. 5D shows MIPs at 48 h p.i. FIG. 5E shows imaging slices obtained at 48 h p.i.

FIGS. 6A and 6B depict biodistribution data. FIG. 6A shows tissue uptake at 48 h p.i. FIG. 6B shows tumor:tissue uptake ratios at 48 h p.i.

### 5. DETAILED DESCRIPTION OF THE INVENTION

The presently disclosed subject matter relates generally to compositions and methods for in vivo diagnosis and treatment of diseases or disorders associated with DLL3. The presently disclosed subject matter provides novel anti-DLL3 antibodies-transcyclooctene (TCO) conjugates, which can form a radioimmunoconjugate with a radioligand comprising a radioactive isotope. The presently disclosed subject matter provides pretargeting strategies using anti-DLL3 antibody-TCO conjugates and radioligands comprising a radioactive isotope (e.g., a sarcophagine-based tetrazine) for radioimmunotherapy and pretargeted PET imaging with more rapid excretion of the excess radioligand through the bladder and kidneys. These strategies enable (i) targeted radioimmunotherapy, and (ii) delineation of cancer at earlier time points compared to other imaging strategies. Furthermore, these strategies decrease the radiation dose to healthy tissues compared to directly labeled antibodies.

The presently disclosed subject matter provides pretargeting strategies comprising two main components: the anti-DLL3 antibody-transcyclooctene conjugate (e.g., anti-DLL3 antibody-TCO conjugate) and the radioligand. In certain embodiments, the radioligand comprises a tetrazine. In certain embodiments, the tetrazine binds to the transcyclooctene via click chemistry reaction.

Non-limiting embodiments of the present disclosure are described by the present specification and Examples. For purposes of clarity of disclosure and not by way of limitation, the detailed description is divided into the following subsections:
5.1. Definitions;
5.2. Pretargeting Systems;
5.3. Pharmaceutical Compositions; and
5.4. Methods of Uses;
5.5. Kits; and
5.6. Exemplary Embodiments.

### 5.1. Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

In the description that follows, certain conventions will be followed as regards the usage of terminology. Generally, terms used herein are intended to be interpreted consistently with the meaning of those terms as they are known to those of skill in the art.

An “antigen-binding protein” is a protein or polypeptide that comprises an antigen-binding region or antigen-binding fragment, that is, has a strong affinity to another molecule to which it binds. Antigen-binding proteins encompass antibodies.

“Antibody” and “antibodies” as those terms are known in the art refer to antigen binding proteins of the immune system. The term “antibody” as referred to herein includes whole, full-length antibodies having an antigen-binding region, and any fragment thereof in which the ‘antigen-binding fragment” or “antigen-binding region” is retained, or single chains, for example, single-chain variable fragment (scFv), thereof. A naturally occurring “antibody” is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (CH) region. The heavy chain constant region is comprised of three domains, CH1, CH2, and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1 q) of the classical complement system.

The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the presently disclosed subject matter may include amino acid residues not encoded by human germline immunoglobulin sequences (e.g., mutations introduced by random or site-specific mutagenesis in vitro or by somatic mutation in vivo).

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the presently disclosed subject matter may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

As used herein, an antibody that “specifically binds to DLL3” is intended to refer to an antibody that binds to DLL3 (e.g., human DLL3) with a dissociation constant (K_{D}) of about 1×10⁻⁸ M or less, about 5×10⁻⁹ M or less, about 1×10⁻⁹ M or less, about 5×10⁻¹⁰ M or less, about 1×10⁻¹⁰ M or less, about 5×10⁻¹¹ M or less, about 1×10⁻¹¹ M or less, about 5×10⁻¹² M or less, or about 1×10⁻¹² M or less.

The phrases “an antibody recognizing an antigen” and “an antibody specific for an antigen” are used interchangeably herein with the term” an antibody which binds specifically to an antigen (e.g., a DLL3 polypeptide).”

The term “antigen-binding fragment” or “antigen-binding region” of an antibody, as used herein, refers to that region or fragment of the antibody that binds to the antigen and which confers antigen specificity to the antibody; fragments of antigen-binding proteins, for example, antibodies including one or more fragments of an antibody that retain the ability to specifically bind to an antigen (e.g., a DLL3 polypeptide). It has been shown that the antigen-binding function of an antibody can be performed by fragments of a full-length antibody. Examples of antigen-binding fragments encompassed within the term “antibody fragments” of an antibody include a Fab fragment, a monovalent fragment consisting of the VL, VH, CL, and CH1 domains; an F(ab)2 fragment, a bivalent fragment comprising two Fab fragments linked by a disulfide bridge at the hinge region; an Fd fragment consisting of the VH and CH1 domains; an Fv fragment consisting of the VL and VH domains of a single arm of an antibody; a dAb fragment (Ward et al., Nature 1989; 341:544-546), which consists of a VH domain; and an isolated complementarity determining region (CDR).

Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules. These are known as single chain Fv (scFv); see e.g., Bird et al., Science (1988); 242:423-426; and Huston et al., Proc Natl Acad Sci (1998); 85:5879-5883. These antibody fragments are obtained using conventional techniques known to those of skill in the art, and the fragments are screened for utility in the same manner as are intact antibodies.

An “antibody” or “antigen-binding protein” is one which has been identified and separated and/or recovered from a component of its natural environment. “Synthetic antibodies” or “recombinant antibodies” are generally generated using recombinant technology or using peptide synthetic techniques known to those of skill in the art.

As used herein, the term “single-chain variable fragment” or “scFv” is a fusion protein of the variable regions of the heavy (VH) and light chains (VL) of an immunoglobulin (e.g., mouse or human) covalently linked to form a VH::VL heterodimer. The heavy (VH) and light chains (VL) are either joined directly or joined by a peptide-encoding linker (e.g., 10, 15, 20, 25 amino acids), which connects the N-terminus of the VH with the C-terminus of the VL, or the C-terminus of the VH with the N-terminus of the VL. The linker is usually rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal Chem (2008); 80(6):1910-1917 and WO 2014/087010, the contents of which are hereby incorporated by reference in their entireties.

As used herein, “F(ab)” refers to a fragment of an antibody structure that binds to an antigen but is monovalent and does not have an Fc portion, for example, an antibody digested by the enzyme papain yields two F(ab) fragments and an Fc fragment (e.g., a heavy (H) chain constant region; Fc region that does not bind to an antigen).

As used herein, “F(ab′)₂” refers to an antibody fragment generated by pepsin digestion of whole IgG antibodies, wherein this fragment has two antigen binding (ab′) (bivalent) regions, wherein each (ab′) region comprises two separate amino acid chains, a part of an H chain and a light (L) chain linked by an S—S bond for binding an antigen and where the remaining H chain portions are linked together. A “F(ab′)₂” fragment can be split into two individual Fab′ fragments.

“CDRs” are defined as the complementarity determining region amino acid sequences of an antibody which are the hypervariable regions of immunoglobulin heavy and light chains. See, e. g., Kabat et al., Sequences of Proteins of Immunological Interest, 4th U. S. Department of Health and Human Services, National Institutes of Health (1987), or IMGT numbering system (Lefranc, The Immunologist (1999); 7:132-136; Lefranc et al., Dev. Comp. Immunol. (2003); 27:55-77). The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise three heavy chain and three light chain CDRs or CDR regions in the variable region. CDRs provide the majority of contact residues for the binding of the antibody to the antigen or epitope region. In certain embodiments, the CDRs are identified according to the IMGT system. In certain embodiments, the CDRs are identified using the IMGT numbering system accessible at http://www.imgt.org/IMGT_vquest/input.

The terms “isolated” denotes a degree of separation from original source or surroundings.

An “isolated antibody” is one which has been separated from a component of its natural environment. In certain embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr (2007); B 848:79-87.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

By “substantially identical” or “substantially homologous” is meant a polypeptide or nucleic acid molecule exhibiting at least about 50% homologous or identical to a reference amino acid sequence (for example, any of the amino acid sequences described herein) or a reference nucleic acid sequence (for example, any of the nucleic acid sequences described herein). In certain embodiments, such a sequence is at least about 60%, at least about 65%, at least about 70%, at least about 75%, at least about 80%, at least about 85%, at least about 90%, at least about 95%, at least about 99%, or at least about 100% homologous or identical to the sequence of the amino acid or nucleic acid used for comparison. Sequence identity can be measured by using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

As used herein, the percent homology between two amino acid sequences is equivalent to the percent identity between the two sequences. The percent identity between the two sequences is a function of the number of identical positions shared by the sequences (i.e., % homology=#of identical positions/total #of positions×100), taking into account the number of gaps, and the length of each gap, which need to be introduced for optimal alignment of the two sequences. The comparison of sequences and determination of percent identity between two sequences can be accomplished using a mathematical algorithm. The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences (e.g., heavy and light chain variable region sequences of scFv m903, m904, m905, m906, and m900) disclosed herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

As used herein, the term “a conservative sequence modification” refers to an amino acid modification that does not significantly affect or alter the binding characteristics of the presently disclosed antibodies (e.g., anti-DLL3 antibodies). Conservative modifications can include amino acid substitutions, additions and deletions. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (1) above) using the functional assays described herein. In certain embodiments, no more than one, no more than two, no more than three, no more than four, no more than five residues within a specified sequence or a CDR region are altered.

As used herein, the term “administration” refers to introducing a substance into a subject. In general, any route of administration may be utilized including, for example, parenteral (e.g., intravenous), oral, topical, subcutaneous, peritoneal, intraarterial, inhalation, vaginal, rectal, nasal, introduction into the cerebrospinal fluid, or instillation into body compartments. In certain embodiments, administration is oral. Additionally or alternatively, administration is parenteral. In certain embodiments, administration is intravenous.

The term “biocompatible”, as used herein is intended to describe materials that do not elicit a substantial detrimental response in vivo. In certain embodiments, the materials are “biocompatible” if they are not toxic to cells. In certain embodiments, materials are “biocompatible” if their addition to cells in vitro results in less than or equal to 20% cell death, and/or their administration in vivo does not induce inflammation or other such adverse effects. In certain embodiments, materials are biodegradable.

As used herein, “biodegradable” materials are those that, when introduced into cells, are broken down by cellular machinery (e.g., enzymatic degradation) or by hydrolysis into components that cells can either reuse or dispose of without significant toxic effects on the cells. In certain embodiments, components generated by breakdown of a biodegradable material do not induce inflammation and/or other adverse effects in vivo. In certain embodiments, biodegradable materials are enzymatically broken down. Alternatively or additionally, biodegradable materials are broken down by hydrolysis. In certain embodiments, biodegradable polymeric materials break down into their component polymers. In certain embodiments, breakdown of biodegradable materials (including, for example, biodegradable polymeric materials) includes hydrolysis of ester bonds. In certain embodiments, breakdown of materials (including, for example, biodegradable polymeric materials) includes cleavage of urethane linkages.

As used herein, “biomolecule” refers to bioactive, diagnostic, and prophylactic molecules. Biomolecules that can be used in the present invention include, but are not limited to, synthetic, recombinant or isolated peptides and proteins such as antibodies and antigens, receptor ligands, enzymes, and adhesion peptides; nucleotides and polynucleotides such as DNA and antisense nucleic acid molecule; activated sugars and polysaccharides; bacteria; viruses; and chemical drugs such as antibiotics, anti-inflammatories, and antifungal agents.

As used herein, “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

As used herein, the term “chelator” refers to a chemical compound in the form of a heterocyclic ring or surrounding structure containing a metal ion attached by coordinate bonds to at least two nonmetal ions. Non-limiting examples of chelator include 1,4,7-Triazacyclononane-1,4,7-triacetic acid (NOTA), 2,2′-(7-(1-carboxy-4-((4-isothiocyanatobenzyl)amino)-4-oxobutyl)-1,4,7-triazonane-1,4-diyl)diacetic acid (NODA), 2,2′,2″,2′″-(1,4,7,10-Tetraazacyclododecane-1,4,7,10-tetrayl)tetraacetic acid (DOTA), Diethylenetriamine-N,N,N′,N,N-pentaacetic acid, pentetic acid, (Carboxymethyl)imino]bis(ethylenenitrilo)-tetra-acetic acid (DTPA), 1-Hydroxy-2-pyridone; 2-Pyridinol-1-oxide (HOPO), N-(5-(3-((5-Aminopentyl)hydroxycarbamoyl)propionamido)pentyl)-3-((5-(N-hydroxyacetamido)pentyl)carbamoyl)propionohydroxamic acid (DFO), and 2-[1,4,7-Triazacyclononan-1-yl-4,7-bis(tBu-ester)]-1,5-pentanedioic acid (NODAGA).

As used herein, “radiolabel” refers to a moiety comprising a radioactive isotope of at least one element. Exemplary suitable radiolabels include but are not limited to those described herein. In certain embodiments, a radiolabel is one used in radioimmunotherapy. In certain embodiments, a radiolabel is one used in positron emission tomography (PET). In certain embodiments, a radiolabel is one used in single-photon emission computed tomography (SPECT). In certain embodiments, the radioactive isotope cam be ^{99m}Tc, ¹¹¹In, ⁴⁴Sc, ⁴⁷Sc, ⁶⁴Cu, ⁶⁷Ga, ⁶⁸Ga, ⁷²As, ⁷⁷As, ⁵²Mn, ¹⁸⁶Re, ¹⁵³Sm ¹⁷⁷Lu, ⁶⁷Cu, ¹²³I, ¹²⁴I, ¹²⁵I, ¹¹C, ¹³N, ¹⁵O, ¹⁸F, ¹⁶⁶Ho, ¹⁴⁹Pm, ⁹⁰Y, ⁸⁶Y, ²¹³Bi, ¹⁰³Pd, ¹⁰⁹Pd, ¹⁵⁹Gd, ¹⁴⁰La, ¹⁹⁸Au, ¹⁹⁹Au, ¹⁷⁵Yb, ¹⁶⁹Yb, ¹⁶⁵Dy, ¹⁶⁶Dy, ¹⁰⁵Rh ¹¹¹Ag, ⁸⁹Zr, ¹⁹²Ir, ²²⁵Ac, ²¹²Bi, ²¹³Bi, ⁷⁶Br, ⁵⁵Co, ⁶⁰Cu, ⁶¹Cu, ⁶²Cu, ⁶⁶Ga, ⁹⁰Nb, ²¹²Pb, ¹⁵²Tb, ⁷⁷Br, ²¹¹At, ²²⁷Th, ²²⁴Ra, or ¹⁴⁹Tb.

An “individual” or “subject” herein is a vertebrate, such as a human or non-human animal, for example, a mammal. Mammals include, but are not limited to, humans, primates, farm animals, sport animals, rodents, and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “small molecule” can refer to a non-polymeric molecule, for example, or a species less than 5000 Da.

As used herein, the phrase “therapeutic agent” refers to any agent that has a therapeutic effect and/or elicits a desired biological and/or pharmacological effect, when administered to a subject.

An “effective amount” is an amount sufficient to affect a beneficial or desired clinical result upon treatment. An effective amount can be administered to a subject in one or more doses. In certain embodiments, an effective amount can be an amount that is sufficient to palliate, ameliorate, stabilize, reverse or slow the progression of the disease, or otherwise reduce the pathological consequences of the disease. The effective amount can be determined by a physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the cells administered.

As used herein, “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

The terms “comprises”, “comprising”, and are intended to have the broad meaning ascribed to them in U.S. Patent Law and can mean “includes”, “including” and the like.

Other aspects of the presently disclosed subject matter are described in the following disclosure and are within the ambit of the presently disclosed subject matter.

### 5.2. Pretargeting Systems

In certain embodiments, the presently disclosed subject matter provides an anti-DLL3 antibody-TCO conjugate (e.g., an anti-DLL3 antibody bound to TCO) and a radioligand (e.g., a Tz-SarAr) for the visualization of cancers (e.g., DLL3-expressing cancers) and/or for the radioimmunotherapy of the same. It was found that the radioligand disclosed herein, in combination with the anti-DLL3 antibody-TCO conjugate, exhibited higher activity concentrations in the tumor and was rapidly cleared through the renal system allowing for higher tumor-to-background activity concentration ratios at early time points. In addition, the use of alternative radioactive isotopes (e.g., Ac²²⁵) allows the use of this system for radioimmunotherapy.

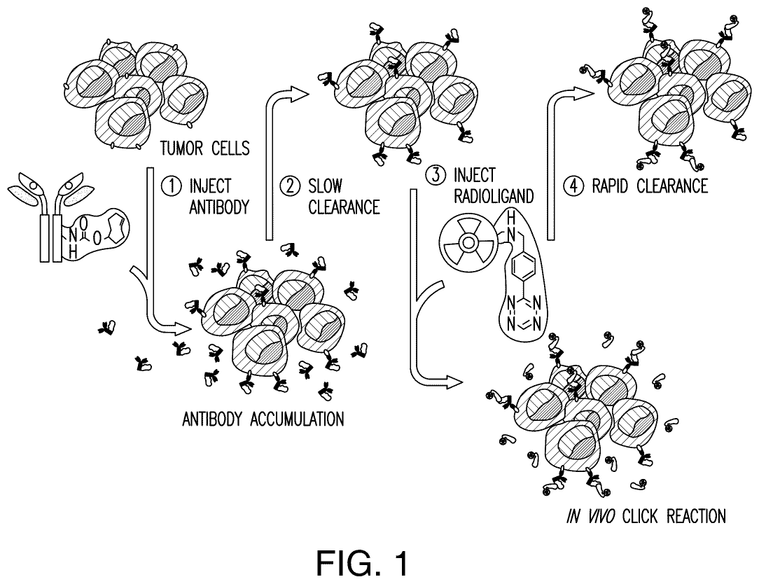

The presently disclosed subject matter provides a pretargeting system including (i) injection of the anti-DLL3 antibody-TCO conjugate; (ii) accumulation of the antibody at the target site and allowing unbound antibody to clear from the blood; (iii) injection of the radioligand; and (iv) an in vivo click ligation of the two components, followed by clearance of the excess radioligand (FIG. 1). Importantly, this pretargeting strategy has significant advantages. First, it effectively delineates tumor tissue at much earlier time points than directly radiolabeled antibodies and significantly reduces the overall radiation burden to the patient, permitting safer and more accurate diagnoses in shorter time frames. Second, it allows the use of different radioactive isotopes (e.g., Ac²²⁵) which can be used for radioimmunotherapy at the tumor site and reducing significantly side effects related to off-target radiation.

### 5.2.1. Anti-DLL3 Antibodies

The presently disclosed anti-DLL3 antibodies bind specifically to DLL3 (e.g., human DLL3).

DLL3 (also known as Delta-like 3 or SCDO1) is a member of the Delta-like family of Notch DSL ligands. Aberrant DLL3 expression (genotypic and/or phenotypic) is associated with various tumorigenic cell subpopulations such as cancer stem cells and tumor initiating cells. Accordingly, the present disclosure provides DLL3 antibodies that can be particularly useful for the disclosed pretargeting strategies disclosed herein, thereby facilitating the treatment, management, prevention, or imaging of neoplastic disorders.

In certain embodiments, the presently disclosed anti-DLL3 antibody binds to a DLL3 polypeptide. In certain embodiments, the presently disclosed anti-DLL3 antibody binds to a human DLL3 polypeptide. In certain embodiments, the human DLL3 polypeptide comprises or consists of the amino acid sequence with a UniProt Reference No: Q9NYJ7-1 (SEQ ID NO: 209) or a fragment thereof. SEQ ID NO: 209 is provided below. In certain embodiments, the DLL3 comprises an extracellular domain, a transmembrane domain, and a cytoplasmic domain. In certain embodiments, the extracellular domain comprises or consists of amino acids 27 to 492 of SEQ ID NO: 209. In certain embodiments, the transmembrane domain comprises or consists of amino acids 493 to 513 of SEQ ID NO: 209. In certain embodiments, the cytoplasmic domain comprises or consists of amino acids 514 to 618 of SEQ ID NO: 209.

In certain embodiments, the extracellular domain of DLL3 comprises a DSL domain, an EGF-like 1 domain, an EGF-like 2 domain, an EGF-like 3 domain, an EGF-like 4 domain, and EGF-like 5 domain, and an EGF-like 6 domain. In certain embodiments, the DSL domain comprises or consists of amino acids 176 to 215 of SEQ ID NO: 209. In certain embodiments, the EGF-like 1 domain comprises or consists of amino acids 216 to 249 of SEQ ID NO: 209. In certain embodiments, the EGF-like 2 domain comprises or consists of amino acids 274 to 310 of SEQ ID NO: 209. In certain embodiments, the EGF-like 3 domain comprises or consists of amino acids 312 to 351 of SEQ ID NO: 209. In certain embodiments, the EGF-like 4 domain comprises or consists of amino acids 353 to 389 of SEQ ID NO: 209. In certain embodiments, the EGF-like 5 domain comprises or consists of amino acids 391 to 427 of SEQ ID NO: 209. In certain embodiments, the EGF-like 6 domain comprises or consists of amino acids 429 to 465 of SEQ ID NO: 209.

[SEQ ID NO: 209]
MVSPRMSGLL SQTVILALIF LPQTRPAGVF ELQIHSFGPG
PGPGAPRSPC SARLPCRLFF RVCLKPGLSE EAAESPCALG
AALSARGPVY TEQPGAPAPD LPLPDGLLQV PFRDAWPGTF
SFIIETWREE LGDQIGGPAW SLLARVAGRR RLAAGGPWAR
DIQRAGAWEL RFSYRARCEP PAVGTACTRL CRPRSAPSRC
GPGLRPCAPL EDECEAPLVC RAGCSPEHGF CEQPGECRCL
EGWTGPLCTV PVSTSSCLSP RGPSSATTGC LVPGPGPCDG
NPCANGGSCS ETPRSFECTC PRGFYGLRCE VSGVTCADGP
CFNGGLCVGG ADPDSAYICH CPPGFQGSNC EKRVDRCSLQ
PCRNGGLCLD LGHALRCRCR AGFAGPRCEH DLDDCAGRAC
ANGGTCVEGG GAHRCSCALG FGGRDCRERA DPCAARPCAH
GGRCYAHFSG LVCACAPGYM GARCEFPVHP DGASALPAAP
PGLRPGDPQR YLLPPALGLL VAAGVAGAAL LLVHVRRRGH
SQDAGSRLLA GTPEPSVHAL PDALNNLRTQ EGSGDGPSSS
VDWNRPEDVD PQGIYVISAP SIYAREVATP LFPPLHTGRA
GQRQHLLFPY PSSILSVK

In certain embodiments, the DLL3 polypeptide comprises or consists of an amino acid sequence that is at least about 80%, at least about 85%, at least about 90%, at least about 95%, at least about 96%, at least about 97%, at least about 98%, or at least about 99%, at least about 100% identical to the amino acid sequence set forth in SEQ ID NO: 209 or a fragment thereof.

In certain embodiments, the anti-DLL3 antibodies or antigen-binding fragments thereof bind to a portion of human DLL3. In certain embodiments, the anti-DLL3 antibodies or antigen-binding fragments thereof bind to the extracellular domain of DLL3. In certain embodiments, the anti-DLL3 antibodies or antigen-binding fragments thereof bind to amino acids 27 to 492 of SEQ ID NO: 209.

In certain embodiments, the antigen recognizing receptor binds to EGF-like 3 domain of DLL3. In certain embodiments, the antigen recognizing receptor binds to amino acids 312 to 351 of SEQ ID NO: 209. In certain embodiments, the antigen recognizing receptor binds to EGF-like 4 domain of DLL3. In certain embodiments, the antigen recognizing receptor binds to amino acids 353 to 389 of SEQ ID NO: 209. In certain embodiments, the antigen recognizing receptor binds to EGF-like 5 domain of DLL3. In certain embodiments, the antigen recognizing receptor binds to amino acids 391 to 427 of SEQ ID NO: 209. In certain embodiments, the antigen recognizing receptor binds to EGF-like 6 domain of DLL3. In certain embodiments, the antigen recognizing receptor binds to amino acids 429 to 465 of SEQ ID NO: 209.

In certain embodiments, a presently disclosed antibody or antigen-binding fragment thereof binds to DLL3 (e.g., human DLL3) with a binding affinity, for example with a dissociation constant (K_{D}) of about 1×10⁻⁸ M or less, about 5×10⁻⁹ M or less, about 1×10⁻⁹ M or less, about 5×10⁻¹⁰ M or less, about 1×10⁻¹⁰ M or less, about 5×10⁻¹¹ M or less, or about 1×10⁻¹¹ M or less, about 5×10⁻¹² M or less, or about 1×10⁻¹² M or less. In certain embodiments, extracellular antigen-binding domain (embodied, for example, an scFv) binds to DLL3 (e.g., human DLL3) with a binding affinity, for example with a dissociation constant (K_{D}) of about 5×10⁻⁹ M or less. In certain embodiments, a presently disclosed antibody or antigen-binding fragment binds to DLL3 (e.g., human DLL3) with a binding affinity, for example with a dissociation constant (K_{D}) of about 1×10⁻⁹ M or less. In certain embodiments, extracellular antigen-binding domain (embodied, for example, an scFv) binds to DLL3 (e.g., human DLL3) with a binding affinity, for example with a dissociation constant (K_{D}) of about 3.5×10⁻⁹ M. In certain embodiments, extracellular antigen-binding domain (embodied, for example, an scFv) binds to DLL3 (e.g., human DLL3) with a binding affinity, for example with a dissociation constant (K_{D}) of about 1.5×10⁻⁹ M. In certain embodiments, a presently disclosed antibody or antigen-binding fragment binds to DLL3 (e.g., human DLL3) with a binding affinity, for example with a dissociation constant (K_{D}) of about 1×10⁻¹² M.

The heavy and light chains of a presently disclosed antibody or antigen-binding fragment thereof can be full-length (e.g., an antibody can include at least one (e.g., one or two) complete heavy chains, and at least one (e.g., one or two) complete light chains) or can include an antigen-binding fragment (a Fab, F(ab′)₂, Fv or a single chain Fv fragment (“scFv”)). In certain embodiments, the antibody heavy chain constant region is chosen from, e.g., IgG1, IgG2, IgG3, IgG4, IgM, IgA1, IgA2, IgD, and IgE, particularly chosen from, e.g., IgG1, IgG2, IgG3, and IgG4. In certain embodiments, the immunoglobulin isotype is IgG1 (e.g., human IgG1). The choice of antibody isotype can depend on the immune effector function that the antibody is designed to elicit. In certain embodiments, the antibody light chain constant region is chosen from, e.g., kappa or lambda, particularly kappa.

In certain embodiments, the presently disclosed subject matter includes antibodies or antigen-binding fragments thereof that have the scFv sequence fused to one or more constant domains to form an antibody with an Fc region of a human immunoglobulin to yield a bivalent protein, increasing the overall avidity and stability of the antibody.

In constructing a recombinant immunoglobulin, appropriate amino acid sequences for constant regions of various immunoglobulin isotypes and methods for the production of a wide array of antibodies are known to those of skill in the art.

In certain embodiments, the anti-DLL3 antibody or antigen-binding fragment thereof comprises V_{H} and V_{L} regions or CDRs selected from Table 1. In certain embodiments, the anti-DLL3 antibody or antigen-binding fragment thereof comprises a V_{H} comprising the amino acid sequence set forth in SEQ ID NO: 7. An exemplary nucleotide sequence encoding the amino acid sequence of SEQ ID NO: 7 is set forth in SEQ ID NO: 9. In certain embodiments, the anti-DLL3 antibody or antigen-binding fragment thereof comprises a V_{L} comprising the amino acid sequence set forth in SEQ ID NO: 8. An exemplary nucleotide sequence encoding the amino acid sequence of SEQ ID NO: 8 is set forth in SEQ ID NO: 10. SEQ ID NO: 7-10 are provided in Table 1. In certain embodiments, the antibody or antigen-binding fragment thereof is designated as “J8”.

In certain embodiments, the anti-DLL3 antibody or antigen-binding fragment thereof comprises a V_{H} comprising the amino acid sequence set forth in SEQ ID NO: 7 and a V_{L} comprising the amino acid sequence set forth in SEQ ID NO: 8.

In certain embodiments, the anti-DLL3 antibody or antigen-binding fragment thereof comprises a V_{H} comprising a CDR1 comprising the amino acid sequence set forth in SEQ ID NO: 1 or a conservative modification thereof, a CDR2 comprising the amino acid sequence set forth in SEQ ID NO: 2 or a conservative modification thereof, and a CDR3 comprising the amino acid sequence set forth in SEQ ID NO: 3 or a conservative modification thereof. SEQ ID NOs: 1-3 are provided in Table 1.

In certain embodiments, the anti-DLL3 antibody or antigen-binding fragment thereof comprises a V_{L} comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 6 or a conservative modification thereof. SEQ ID NOs: 4-6 are provided in Table 1.

In certain embodiments, the anti-DLL3 antibody or antigen-binding fragment thereof comprises a V_{H} comprising a CDR1 comprising the amino acid sequence set forth in SEQ ID NO: 1 or a conservative modification thereof, a CDR2 comprising the amino acid sequence set forth in SEQ ID NO: 2 or a conservative modification thereof, and a CDR3 comprising the amino acid sequence set forth in SEQ ID NO: 3 or a conservative modification thereof; and a V_{L} comprising a CDR1 comprising the amino acid sequence set forth in SEQ ID NO: 4 or a conservative modification thereof, a CDR2 comprising the amino acid sequence set forth in SEQ ID NO: 5 or a conservative modification, and a CDR3 comprising the amino acid sequence set forth in SEQ ID NO: 6 or a conservative modification thereof.

In certain embodiments, the anti-DLL3 antibody or antigen-binding fragment thereof comprises a V_{H} comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and a V_{L} Comprising CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6.

TABLE 1

| CDRs | 1 | 2 | 3 |
|---|---|---|---|
| V_{H} | GYTFTIY [SEQ ID | NPSGGS [SEQ ID | NDYVDYVTTDYYGMDV |
| | NO: 1] | NO: 2] | [SEQ ID NO: 3] |
| V_{L} | RASQGISNYLA [SEQ | AASSLQS [SEQ ID | QQYNSSPYT [SEQ |
| | ID NO: 4] | NO: 5] | ID NO: 6] |

| | |
|---|---|
| Full V_{H} | QVHLVQSGAEVKKPGASVKVSCKASGYTFTIYYIHWVRQAPGQGLEWMGIINPS |
| | GGSTSYAQKFQGRVTMTRDTSTSTVYMELSSLRSEDTAVYYCSGNDYVDYVTTD |
| | YYGMDVWGQGTTVTVSS [SEQ ID NO: 7] |
| Full V_{L} | DIQMTQSPSSLSASVGDRVTITCRASQGISNYLAWFQQKPGKAPKSLIYAASSL |
| | QSGVPSKFSGSGSGTDFTLTISSLQPEDFATYYCQQYNSSPYTFGQGTKLEIK |
| | [SEQ ID NO: 8] |
| DNA for | CAGGTGCATCTGGTGCAGTCTGGGGCTGAGGTGAAGAAGCCTGGGGCCTCAGTG |
| Full V_{H} | AAGGTTTCTTGCAAGGCATCTGGATACACCTTCACCATCTACTATATTCACTGG |
| | GTGCGACAGGCCCCTGGACAGGGGCTTGAGTGGATGGGAATAATCAACCCTAGT |
| | GGTGGTAGCACAAGCTACGCACAGAAGTTCCAGGGCAGAGTCACCATGACCAGG |
| | GACACGTCCACGAGCACAGTCTACATGGAGCTGAGCAGCCTGAGATCTGAGGAC |
| | ACGGCCGTATATTACTGTTCGGGTAATGACTACGTTGACTACGTAACTACTGAC |
| | TACTACGGTATGGACGTCTGGGGCCAAGGGACCACGGTCACCGTCTCCTCA |
| | [SEQ ID NO: 9] |
| DNA for | GACATCCAGATGACCCAGTCTCCATCCTCACTGTCTGCATCTGTAGGAGATAGA |
| Full V_{L} | GTCACCATCACTTGTCGGGCGAGTCAGGGCATTAGCAATTATTTAGCCTGGTTT |
| | CAGCAGAAACCAGGGAAAGCCCCTAAGTCCCTGATCTATGCTGCATCCAGTTTG |
| | CAAAGTGGGGTCCCATCAAAGTTCAGCGGCAGTGGATCTGGGACAGATTTCACA |
| | CTCACCATCAGCAGCCTGCAGCCTGAAGATTTTGCAACTTATTACTGCCAACAG |
| | TATAATAGTTCCCCGTACACTTTTGGCCAGGGGACCAAGCTGGAGATCAAA |
| | [SEQ ID NO: 10] |

In certain embodiments, the anti-DLL3 antibody or antigen-binding fragment thereof comprises V_{H} and V_{L} regions or CDRs selected from Table 2. In certain embodiments, the anti-DLL3 antibody or antigen-binding fragment thereof comprises a V_{H} comprising the amino acid sequence set forth in SEQ ID NO: 17, as shown in Table 2. An exemplary nucleotide sequence encoding the amino acid sequence of SEQ ID NO: 17 is set forth in SEQ ID NO: 19. In certain embodiments, the anti-DLL3 antibody or antigen-binding fragment thereof comprises a V_{L} comprising the amino acid sequence set forth in SEQ ID NO: 18. An exemplary nucleotide sequence encoding the amino acid sequence of SEQ ID NO: 18 is set forth in SEQ ID NO: 20. In certain embodiments, the anti-DLL3 antibody or antigen-binding fragment thereof comprises a V_{H} comprising the amino acid sequence set forth in SEQ ID NO: 17 and a V_{L} comprising the amino acid sequence set forth in SEQ ID NO: 18. SEQ ID NO: 17-20 are provided in Table 2. In certain embodiments, the antibody or antigen-binding fragment thereof is designated as “L22”.

In certain embodiments, the anti-DLL3 antibody or antigen-binding fragment thereof comprises a V_{H} comprising a CDR1 comprising the amino acid sequence set forth in SEQ ID NO: 11 or a conservative modification thereof, a CDR2 comprising the amino acid sequence set forth in SEQ ID NO: 12 or a conservative modification thereof, and a CDR3 comprising the amino acid sequence set forth in SEQ ID NO: 13 or a conservative modification thereof. SEQ ID NOs: 11-13 are provided in Table 2.

In certain embodiments, the anti-DLL3 antibody or antigen-binding fragment thereof comprises a V_{L} comprising a CDR1 comprising the amino acid sequence set forth in SEQ ID NO: 14 or a conservative modification thereof, a CDR2 comprising the amino acid sequence set forth in SEQ ID NO: 15 or a conservative modification thereof, and a CDR3 comprising the amino acid sequence set forth in SEQ ID NO: 16 or a conservative modification thereof. SEQ ID NOs: 14-16 are provided in Table 2.

In certain embodiments, the anti-DLL3 antibody or antigen-binding fragment thereof comprises a V_{H} comprising a CDR1 comprising the amino acid sequence set forth in SEQ ID NO: 11 or a conservative modification thereof, a CDR2 comprising the amino acid sequence set forth in SEQ ID NO: 12 or a conservative modification thereof, and a CDR3 comprising the amino acid sequence set forth in SEQ ID NO: 13 or a conservative modification thereof; and a V_{L} comprising a CDR1 comprising the amino acid sequence set forth in SEQ ID NO: 14 or a conservative modification thereof, a CDR2 comprising the amino acid sequence set forth in SEQ ID NO: 15 or a conservative modification thereof, and a CDR3 comprising the amino acid sequence set forth in SEQ ID NO: 16 or a conservative modification.

In certain embodiments, the anti-DLL3 antibody or antigen-binding fragment thereof comprises a V_{H} comprising a CDR1 comprising the amino acid sequence set forth in SEQ ID NO: 11, a CDR2 comprising the amino acid sequence set forth in SEQ ID NO: 12, and a CDR3 comprising the amino acid sequence set forth in SEQ ID NO: 13; and a V_{L} Comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16.

TABLE 2

| CDRs | 1 | 2 | 3 |
|---|---|---|---|
| V_{H} | GFTFSSY | SGRGGD | ERGLGFDY |
| | [SEQ ID NO: 11] | [SEQ ID NO: 12] | [SEQ ID NO: 13] |
| V_{L} | RSSQSLLDSDDGNTYLD | TLSYRAS | MQHLEFPLT |
| | [SEQ ID NO: 14] | [SEQ ID NO: 15] | [SEQ ID NO: 16] |

| | |
|---|---|
| Full V_{H} | EVQLLESGGGLVQPGGSLRLSCAASGFTFSSYAMGWVRQTPGKGLEWVSTISGR |
| | GGDTYYADSVKGRFTISRDNSKNTLYLQMNSLRAEDTAIYYCAKERGLGFDYWG |
| | QGTLVTVSS |
| | [SEQ ID NO: 17] |
| Full V_{L} | DIVMTQTPLSLPVTPGEPASISCRSSQSLLDSDDGNTYLDWYLQKPGQSPQLLI |
| | YTLSYRASGVPDRFSGSGSGTDFTLKISRVEAEDVGVYYCMQHLEFPLTFGGGT |
| | KVEIK |
| | [SEQ ID NO: 18] |
| DNA for | GAGGTGCAGCTGTTGGAGTCTGGGGGAGGCTTGGTACAGCCTGGGGGGTCCCTG |
| Full V_{H} | AGACTCTCCTGTGCAGCCTCTGGATTCACCTTTAGCAGCTATGCCATGGGCTGG |
| | GTCCGCCAGACTCCAGGGAAGGGGCTGGAGTGGGTCTCAACTATTAGTGGTCGT |
| | GGTGGTGACACATACTACGCAGACTCCGTGAAGGGCCGGTTCACCATCTCCAGA |
| | GACAATTCCAAGAACACGCTGTATCTGCAAATGAACAGCCTGAGAGCCGAGGAC |
| | ACGGCCATATATTACTGTGCGAAAGAGAGGGGACTGGGGTTTGACTACTGGGGC |
| | CAGGGAACCCTGGTCACCGTCTCCTCA |
| | [SEQ ID NO: 19] |
| DNA for | GATATTGTGATGACCCAGACTCCACTCTCCCTGCCCGTCACCCCTGGAGAGCCG |
| Full V_{L} | GCCTCCATCTCCTGCAGGTCTAGTCAGAGCCTCTTGGATAGTGATGATGGAAAC |
| | ACCTATTTGGACTGGTACCTGCAGAAGCCAGGGCAGTCTCCACAGCTCCTGATC |
| | TATACGCTTTCCTATCGGGCCTCTGGAGTCCCAGACAGGTTCAGTGGCAGTGGG |
| | TCAGGCACTGATTTCACACTGAAAATCAGCAGGGTGGAGGCTGAGGATGTTGGA |
| | GTTTATTACTGCATGCAACATCTAGAGTTTCCGCTCACTTTCGGCGGAGGGACC |
| | AAGGTGGAGATCAAA |
| | [SEQ ID NO: 20] |

In certain embodiments, the anti-DLL3 antibody or antigen-binding fragment thereof comprises V_{H} and V_{L} regions or CDRs selected from Table 3. In certain embodiments, the anti-DLL3 antibody or antigen-binding fragment thereof comprises a V_{H} comprising the amino acid sequence set forth in SEQ ID NO: 24, as shown in Table 3. An exemplary nucleotide sequence encoding the amino acid sequence of SEQ ID NO: 24 is set forth in SEQ ID NO: 26. In certain embodiments, the anti-DLL3 antibody or antigen-binding fragment thereof comprises a V_{L} comprising the amino acid sequence set forth in SEQ ID NO: 25. An exemplary nucleotide sequence encoding the amino acid sequence of SEQ ID NO: 25 is set forth in SEQ ID NO: 27. SEQ ID NO: 24-27 are provided in Table 3. In certain embodiments, the antibody or antigen-binding fragment thereof is designated as “B2”.

In certain embodiments, the anti-DLL3 antibody or antigen-binding fragment thereof comprises a V_{H} comprising the amino acid sequence set forth in SEQ ID NO: 24 and a V_{L} comprising the amino acid sequence set forth in SEQ ID NO: 25.

In certain embodiments, the anti-DLL3 antibody or antigen-binding fragment thereof comprises a V_{H} comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 22 or a conservative modification thereof. SEQ ID NOs: 2, 21, and 22 are provided in Table 3.

In certain embodiments, the anti-DLL3 antibody or antigen-binding fragment thereof comprises a V_{L} comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 23 or a conservative modification thereof. SEQ ID NOs: 4, 5, and 23 are provided in Table 3.

In certain embodiments, the anti-DLL3 antibody or antigen-binding fragment thereof comprises a V_{H} comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 22 or a conservative modification thereof; and a V_{L} comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 23 or a conservative modification thereof.

In certain embodiments, the anti-DLL3 antibody or antigen-binding fragment thereof comprises a V_{H} comprising a CDR1 comprising the amino acid sequence set forth in SEQ ID NO: 21, a CDR2 comprising the amino acid sequence set forth in SEQ ID NO: 2, and a V_{H} CDR3 comprising the amino acid sequence set forth in SEQ ID NO: 22; and a V_{L} Comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 23.

TABLE 3

| CDRs | 1 | 2 | 3 |
|---|---|---|---|
| V_{H} | GYTFTSY | NPSGGS | GDYDILTDYYYNMDV |
| | [SEQ ID NO: 21] | [SEQ ID NO: 2] | [SEQ ID NO: 22] |
| V_{L} | RASQGISNYLA | AASSLQS | LQHNSYPYT |
| | [SEQ ID NO: 4] | [SEQ ID NO: 5] | [SEQ ID NO: 23] |

| | |
|---|---|
| Full V_{H} | QVQLVQSGAEVKKPGASVKVSCKASGYTFTSYYMHWVRQAPGQGLEWMGIINPS |
| | GGSTSYAQKFRGRVTMTRDTSTSTVYMELSSLRSEDTAVYYCARGDYDILTDYY |
| | YNMDVWGQGTTVTVSS |
| | [SEQ ID NO: 24] |
| Full V_{L} | DIQMTQSPSAMSASVGDRVTITCRASQGISNYLAWFQQKPGKVPKRLIYAASSL |
| | QSGVPSRFSGSGSGTEFTLTISSLQPEDFATYYCLQHNSYPYTFGQGTKLEIK |
| | [SEQ ID NO: 25] |
| DNA for | CAGGTGCAGCTGGTGCAGTCTGGGGCTGAGGTGAAGAAGCCTGGGGCCTCAGTG |
| Full V_{H} | AAGGTTTCCTGCAAGGCATCTGGATACACCTTCACCAGCTACTATATGCACTGG |
| | GTGCGACAGGCCCCTGGACAAGGGCTTGAGTGGATGGGAATAATCAACCCTAGT |
| | GGTGGTAGCACAAGCTACGCACAGAAGTTCCGGGGCAGAGTCACCATGACCAGG |
| | GACACGTCCACGAGCACAGTCTACATGGAGCTGAGCAGCCTGAGATCTGAGGAC |
| | ACGGCCGTTTATTACTGTGCGAGAGGGGATTACGATATTTTGACGGACTACTAC |
| | TACAATATGGACGTCTGGGGCCAAGGGACCACGGTCACCGTCTCCTCA |
| | [SEQ ID NO: 26] |
| DNA for | GACATCCAGATGACCCAGTCTCCATCTGCCATGTCTGCATCTGTAGGAGACAGA |
| Full V_{L} | GTCACCATCACTTGTCGGGCGAGTCAGGGCATTAGCAATTATTTAGCCTGGTTT |
| | CAGCAGAAACCAGGGAAAGTCCCTAAGCGCCTGATCTATGCTGCATCCAGTTTG |
| | CAAAGTGGGGTCCCATCAAGGTTCAGCGGCAGTGGATCTGGGACAGAATTCACT |
| | CTCACAATCAGCAGCCTGCAGCCTGAAGATTTTGCAACTTATTACTGTCTACAG |
| | CATAATAGTTACCCGTACACTTTCGGCCAGGGGACCAAGCTGGAGATCAAA |
| | [SEQ ID NO: 27] |

In certain embodiments, the anti-DLL3 antibody or antigen-binding fragment thereof comprises V_{H} and V_{L} regions or CDRs selected from Table 4. In certain embodiments, the anti-DLL3 antibody or antigen-binding fragment thereof comprises a V_{H} comprising the amino acid sequence set forth in SEQ ID NO: 34, as shown in Table 4. An exemplary nucleotide sequence encoding the amino acid sequence of SEQ ID NO: 34 is set forth in SEQ ID NO: 36. In certain embodiments, the anti-DLL3 antibody or antigen-binding fragment thereof comprises a V_{L} comprising the amino acid sequence set forth in SEQ ID NO: 35. An exemplary nucleotide sequence encoding the amino acid sequence of SEQ ID NO: 35 is set forth in SEQ ID NO: 37. SEQ ID NO: 34-37 are provided in Table 4. In certain embodiments, the antibody or antigen-binding fragment thereof is designated as “A18”.

In certain embodiments, the anti-DLL3 antibody or antigen-binding fragment thereof comprises a V_{H} comprising the amino acid sequence set forth in SEQ ID NO: 34 and a V_{L} comprising the amino acid sequence set forth in SEQ ID NO: 35. SEQ ID NOs: 34 and 35 are provided in Table 4.

In certain embodiments, the anti-DLL3 antibody or antigen-binding fragment thereof comprises a V_{H} comprising a CDR1 comprising the amino acid sequence set forth in SEQ ID NO: 28 or a conservative modification thereof, a CDR2 comprising the amino acid sequence set forth in SEQ ID NO: 29 or a conservative modification thereof, and a CDR3 comprising the amino acid sequence set forth in SEQ ID NO: 30 or a conservative modification thereof. SEQ ID NOs: 28, 29, and 30 are provided in Table 4.

In certain embodiments, the anti-DLL3 antibody or antigen-binding fragment thereof comprises a V_{L} comprising a CDR1 comprising the amino acid sequence set forth in SEQ ID NO: 31 or a conservative modification thereof, a CDR2 comprising the amino acid sequence set forth in SEQ ID NO: 32 or a conservative modification thereof, and a CDR3 comprising the amino acid sequence set forth in SEQ ID NO: 33 or a conservative modification thereof. SEQ ID NOs: 31, 32, and 33 are provided in Table 4.

In certain embodiments, the anti-DLL3 antibody or antigen-binding fragment thereof comprises a V_{H} comprising a CDR1 comprising the amino acid sequence set forth in SEQ ID NO: 28 or a conservative modification thereof, a CDR2 comprising the amino acid sequence set forth in SEQ ID NO: 29 or a conservative modification thereof, and a CDR3 comprising the amino acid sequence set forth in SEQ ID NO: 30 or a conservative modification thereof; and a V_{L} comprising a CDR1 comprising the amino acid sequence set forth in SEQ ID NO: 31 or a conservative modification thereof, a CDR2 comprising the amino acid sequence set forth in SEQ ID NO: 32 or a conservative modification thereof, and a CDR3 comprising the amino acid sequence set forth in SEQ ID NO: 33 or a conservative modification thereof.

In certain embodiments, the anti-DLL3 antibody or antigen-binding fragment thereof comprises a V_{H} comprising a CDR1 comprising the amino acid sequence set forth in SEQ ID NO: 28, a CDR2 comprising the amino acid sequence set forth in SEQ ID NO: 29, and a CDR3 comprising the amino acid sequence set forth in SEQ ID NO: 30; and a V_{L} Comprising a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33.

TABLE 4

| CDRs | 1 | 2 | 3 |
|---|---|---|---|
| V_{H} | GFSLSTSGM | DWDDN | IRGYSGSYDAFDI |
| | [SEQ ID NO: 28] | [SEQ ID NO: 29] | [SEQ ID NO: 30] |
| V_{L} | RSSQSLLHSNGYNYLD | LGSNRAP | MQALQTPFT |
| | [SEQ ID NO: 31] | [SEQ ID NO: 32] | [SEQ ID NO: 33] |

| | |
|---|---|
| Full V_{H} | QVTLRESGPALVKSTQTLTLTCTFSGFSLSTSGMCVSWIRQTPGKALEWLALID |
| | WDDNKYYSTSLKTRLIISKDTSKNQVVLTMTNMEPVDTATYYCARIRGYSGSYD |
| | AFDIWGQGTMVTVSS |
| | [SEQ ID NO: 34] |
| Full V_{L} | VDIKDIVMTQSPLSLPVTPGEPASISCRSSQSLLHSNGYNYLDWYLQKPGQSPHILLY |
| | LGSNRAPGVPDRFSGSGSGTDFTLKISRVEAEDVGVYYCMQALQTP |
| | FTFGPGTK |
| | [SEQ ID NO: 35] |
| DNA for | CAGGTCACCTTGAGGGAGTCTGGTCCTGCGCTGGTGAAATCCACACAGACCCTC |
| Full V_{H} | ACACTGACCTGCACCTTCTCTGGGTTCTCACTCAGTACTAGTGGAATGTGTGTG |
| | AGCTGGATCCGTCAGACCCCAGGGAAGGCCCTGGAGTGGCTTGCACTCATTGAT |
| | TGGGATGATAATAAATACTACAGCACATCTCTGAAGACCAGGCTCATCATCTCC |
| | AAGGACACCTCCAAAAACCAGGTGGTCCTTACAATGACCAATATGGAACCTGTG |
| | GACACAGCCACGTATTATTGTGCACGGATACGGGGGTATAGTGGGAGCTACGAT |
| | GCTTTTGATATCTGGGGCCAAGGGACAATGGTCACCGTCTCTTCA |
| | [SEQ ID NO: 36] |
| DNA for | GATATTGTGATGACTCAGTCTCCACTCTCCCTGCCCGTCACCCCTGGAGAGCCG |
| Full V_{L} | GCCTCCATCTCCTGCAGGTCTAGTCAGAGCCTCCTGCATAGTAATGGATACAAC |
| | TATTTGGATTGGTACCTGCAGAAGCCAGGGCAGTCTCCACACATCCTGCTCTAT |
| | TTGGGTTCTAATCGGGCCCCCGGGGTCCCTGACAGGTTCAGTGGCAGTGGATCA |
| | GGCACAGATTTTACACTGAAAATCAGCAGAGTGGAGGCTGAGGATGTTGGGGTT |
| | TATTACTGCATGCAAGCTCTACAAACTCCATTCACTTTCGGCCCTGGGACCAAA |
| | GTGGATATCAAA |
| | [SEQ ID NO: 37] |

In certain embodiments, the anti-DLL3 antibody or antigen-binding fragment thereof comprises V_{H} and V_{L} regions or CDRs selected from Table 5. In certain embodiments, the anti-DLL3 antibody or antigen-binding fragment thereof comprises a V_{H} comprising the amino acid sequence set forth in SEQ ID NO: 42, as shown in Table 5. An exemplary nucleotide sequence encoding the amino acid sequence of SEQ ID NO: 42 is set forth in SEQ ID NO: 44. In certain embodiments, the anti-DLL3 antibody or antigen-binding fragment thereof comprises a V_{L} comprising the amino acid sequence set forth in SEQ ID NO: 43. An exemplary nucleotide sequence encoding the amino acid sequence of SEQ ID NO: 43 is set forth in SEQ ID NO: 45. SEQ ID NO: 42-45 are provided in Table 5. In certain embodiments, the antibody or antigen-binding fragment thereof is designated as “E9”.

In certain embodiments, the anti-DLL3 antibody or antigen-binding fragment thereof comprises a V_{H} comprising the amino acid sequence set forth in SEQ ID NO: 42 and a V_{L} comprising the amino acid sequence set forth in SEQ ID NO: 43.

In certain embodiments, the anti-DLL3 antibody or antigen-binding fragment thereof comprises a V_{H} comprising a CDR1 comprising the amino acid sequence set forth in SEQ ID NO: 21 or a conservative modification thereof, a CDR2 comprising the amino acid sequence set forth in SEQ ID NO: 38 or a conservative modification thereof, and a CDR3 comprising the amino acid sequence set forth in SEQ ID NO: 39 or a conservative modification thereof. SEQ ID NOs: 21, 38, and -39 are provided in Table 5.

In certain embodiments, the anti-DLL3 antibody or antigen-binding fragment thereof comprises a V_{L} comprising a CDR1 comprising the amino acid sequence set forth in SEQ ID NO: 40 or a conservative modification thereof, a CDR2 comprising the amino acid sequence set forth in SEQ ID NO: 5 or a conservative modification thereof, and a CDR3 comprising the amino acid sequence set forth in SEQ ID NO: 41 or a conservative modification thereof. SEQ ID NOs: 40, 5, and 41 are provided in Table 5.

In certain embodiments, the anti-DLL3 antibody or antigen-binding fragment thereof comprises a V_{H} comprising a CDR1 comprising the amino acid sequence set forth in SEQ ID NO: 21 or a conservative modification thereof, a CDR2 comprising the amino acid sequence set forth in SEQ ID NO: 38 or a conservative modification thereof, and a CDR3 comprising the amino acid sequence set forth in SEQ ID NO: 39 or a conservative modification thereof; and a V_{L} comprising a CDR1 comprising the amino acid sequence set forth in SEQ ID NO: 40 or a conservative modification thereof, a CDR2 comprising the amino acid sequence set forth in SEQ ID NO: 5 or a conservative modification thereof, and a CDR3 comprising the amino acid sequence set forth in SEQ ID NO: 41 or a conservative modification thereof.

In certain embodiments, the anti-DLL3 antibody or antigen-binding fragment thereof comprises a V_{H} comprising a CDR1 comprising the amino acid sequence set forth in SEQ ID NO: 21, a CDR2 comprising the amino acid sequence set forth in SEQ ID NO: 38, and a CDR3 comprising the amino acid sequence set forth in SEQ ID NO: 39; and a V_{L} Comprising a CDR1 comprising the amino acid sequence set forth in SEQ ID NO: 40, a CDR2 comprising the amino acid sequence set forth in SEQ ID NO: 5, and a CDR3 comprising the amino acid sequence set forth in SEQ ID NO: 41.

TABLE 5

| CDRs | 1 | 2 | 3 |
|---|---|---|---|
| V_{H} | GYTFTSY | NSSGGS | GYCTNGVYYDEDFDY |
| | [SEQ ID NO: 21] | [SEQ ID NO: 38] | [SEQ ID NO: 39] |
| V_{L} | RASQGISHYLA | AASSLQS | QQYNSYPLT |
| | [SEQ ID NO: 40] | [SEQ ID NO: 5] | [SEQ ID NO: 41] |

| | |
|---|---|
| Full V_{H} | QVQLVQSGAEVKKPGASVKVSCKASGYTFTSYYMHWVRQAPGQGLEWMGVINSS |
| | GGSTSYAQKFQGRVTMTRDTSTSTVYMDLSSLRSEDTAVYYCARGYCTNGVYYD |
| | EDFDYWGQGTLVTVSS |
| | [SEQ ID NO: 42] |
| Full V_{L} | DIQMTQSPSSLSASVGDRVTITCRASQGISHYLAWFQQKPGKAPTSLIYAASSL |
| | QSGVPSKFSGSGSGTDFTLTISSLQPEDFATYYCQQYNSYPLTFGGGTKVEIK |
| | [SEQ ID NO: 43] |
| DNA for | CAGGTGCAGCTGGTGCAGTCTGGGGCTGAGGTGAAGAAGCCTGGGGCCTCAGTG |
| Full V_{H} | AAGGTTTCCTGCAAGGCATCTGGATACACCTTCACCAGCTACTATATGCACTGG |
| | GTGCGACAGGCCCCTGGACAAGGGCTTGAGTGGATGGGAGTAATCAATTCTAGT |
| | GGTGGTAGCACAAGCTACGCACAGAAGTTCCAGGGCAGAGTCACCATGACCAGG |
| | GACACGTCCACGAGCACAGTCTACATGGACCTGAGCAGCCTGAGATCTGAGGAC |
| | ACGGCCGTGTATTACTGTGCGAGAGGGTATTGTACTAATGGTGTTTACTATGAT |
| | GAGGACTTTGACTACTGGGGCCAGGGAACCCTGGTCACCGTCTCCTCA |
| | [SEQ ID NO: 44] |
| DNA for | GACATCCAGATGACCCAGTCTCCATCCTCACTGTCTGCATCTGTAGGAGACAGA |
| Full V_{L} | GTCACCATCACTTGTCGGGCGAGTCAGGGCATTAGCCATTATTTAGCCTGGTTT |
| | CAGCAGAAACCAGGGAAAGCCCCTACGTCCCTGATCTATGCTGCATCCAGTTTG |
| | CAAAGTGGGGTCCCATCAAAGTTCAGCGGCAGTGGATCTGGGACAGATTTCACT |
| | CTCACCATCAGCAGCCTGCAGCCTGAAGATTTTGCAACTTATTACTGCCAACAG |
| | TATAATAGTTACCCTCTCACTTTCGGCGGAGGGACCAAGGTGGAGATCAAA |
| | [SEQ ID NO: 45] |

In certain embodiments, the anti-DLL antibody or antigen-binding fragment thereof comprises V_{H} and V_{L} regions or CDRs selected from Table 6. In certain embodiments, the anti-DLL3 antibody or antigen-binding fragment thereof comprises a V_{H} comprising the amino acid sequence set forth in SEQ ID NO: 52, as shown in Table 6. An exemplary nucleotide sequence encoding the amino acid sequence of SEQ ID NO: 52 is set forth in SEQ ID NO: 54. In certain embodiments, the anti-DLL3 antibody or antigen-binding fragment thereof comprises a V_{L} comprising the amino acid sequence set forth in SEQ ID NO: 53. An exemplary nucleotide sequence encoding the amino acid sequence of SEQ ID NO: 53 is set forth in SEQ ID NO: 55. SEQ ID NO: 52-55 are provided in Table 6. In certain embodiments, the antibody or antigen-binding fragment thereof is designated as “G3”.

In certain embodiments, the anti-DLL3 antibody or antigen-binding fragment thereof comprises a V_{H} comprising the amino acid sequence set forth in SEQ ID NO: 52 and a V_{L} comprising the amino acid sequence set forth in SEQ ID NO: 53.

In certain embodiments, the anti-DLL3 antibody or antigen-binding fragment thereof comprises a V_{H} comprising a CDR1 comprising the amino acid sequence set forth in SEQ ID NO: 46 or a conservative modification thereof, a CDR2 comprising the amino acid sequence set forth in SEQ ID NO: 47 or a conservative modification thereof, and a CDR3 comprising the amino acid sequence set forth in SEQ ID NO: 48 or a conservative modification thereof. SEQ ID NOs: 46-48 are provided in Table 6.

In certain embodiments, the anti-DLL3 antibody or antigen-binding fragment thereof comprises a V_{L} comprising a CDR1 comprising the amino acid sequence set forth in SEQ ID NO: 49 or a conservative modification thereof, a CDR2 comprising the amino acid sequence set forth in SEQ ID NO: 50 or a conservative modification thereof, and a CDR3 comprising the amino acid sequence set forth in SEQ ID NO: 51 or a conservative modification thereof. SEQ ID NOs: 49, 50, and 51 are provided in Table 6.

In certain embodiments, the anti-DLL3 antibody or antigen-binding fragment thereof comprises a V_{H} comprising a CDR1 comprising the amino acid sequence set forth in SEQ ID NO: 46 or a conservative modification thereof, a CDR2 comprising the amino acid sequence set forth in SEQ ID NO: 47 or a conservative modification thereof, and a CDR3 comprising the amino acid sequence set forth in SEQ ID NO: 48 or a conservative modification thereof; and a V_{L} comprising a CDR1 comprising the amino acid sequence set forth in SEQ ID NO: 49 or a conservative modification thereof, a CDR2 comprising the amino acid sequence set forth in SEQ ID NO: 50 or a conservative modification thereof, and a CDR3 comprising the amino acid sequence set forth in SEQ ID NO: 51 or a conservative modification thereof.

In certain embodiments, the anti-DLL3 antibody or antigen-binding fragment thereof comprises a V_{H} comprising a CDR1 comprising the amino acid sequence set forth in SEQ ID NO: 46, a CDR2 comprising the amino acid sequence set forth in SEQ ID NO: 47, and a CDR3 comprising the amino acid sequence set forth in SEQ ID NO: 48; and a V_{L} Comprising a CDR1 comprising the amino acid sequence set forth in SEQ ID NO: 49, a CDR2 comprising the amino acid sequence set forth in SEQ ID NO: 50, and a CDR3 comprising the amino acid sequence set forth in SEQ ID NO: 51.

TABLE 6

| CDRs | 1 | 2 | 3 |
|---|---|---|---|
| V_{H} | GGSISSY | YTSGS | EDWESWTFDI |
| | [SEQ ID NO: 46] | [SEQ ID NO: 47] | [SEQ ID NO: 48] |
| V_{L} | RSGQSLVYSDGDTYLN | KVSNRDS | MQGTHWPLS |
| | [SEQ ID NO: 49] | [SEQ ID NO: 50] | [SEQ ID NO: 51] |

| | |
|---|---|
| Full V_{H} | QVQLQESGPGLVKPSETLFLTCTVSGGSISSYYWTWIRQPAGKGLEWIGRIYTS |
| | GSTNYNPSLKSRVTMSVDTSKNQFSLKLSSVTAADTAVYYCAREDWESWTFDIW |
| | GQGTMVTVSS |
| | [SEQ ID NO: 52] |
| Full V_{L} | DVVMTQSPLSLPVTLGQPASISCRSGQSLVYSDGDTYLNWFQQRPGQSPRRLIY |
| | KVSNRDSGVPDRFSGSGSGTDFTLKISRVEAEDVGVYYCMQGTHWPLSFGGGTK |
| | VEIK |
| | [SEQ ID NO: 53] |
| DNA for | CAGGTGCAGCTGCAGGAGTCGGGCCCAGGACTGGTGAAGCCTTCGGAGACCCTG |
| Full V_{H} | TTCCTCACCTGCACTGTCTCTGGTGGCTCCATCAGTAGTTACTACTGGACCTGG |
| | ATCCGGCAGCCCGCCGGGAAGGGACTGGAGTGGATTGGGCGTATCTATACCAGT |
| | GGGAGCACCAACTACAACCCCTCCCTCAAGAGTCGAGTCACCATGTCAGTAGAC |
| | ACGTCCAAGAACCAGTTCTCCCTGAAGCTGAGCTCTGTGACCGCCGCGGACACG |
| | GCCGTGTATTACTGTGCGAGAGAGGACTGGGAATCGTGGACTTTTGATATCTGG |
| | GGCCAAGGGACAATGGTCACCGTCTCTTCA |
| | [SEQ ID NO: 54] |
| DNA for | GATGTTGTGATGACTCAGTCTCCACTCTCCCTGCCCGTCACCCTTGGACAGCCG |
| Full V_{L} | GCCTCCATCTCCTGCAGGTCTGGTCAAAGCCTCGTTTACAGTGATGGAGACACC |
| | TACTTGAATTGGTTTCAGCAGAGGCCAGGCCAATCTCCAAGGCGCCTAATTTAT |
| | AAGGTTTCTAACCGGGACTCTGGGGTCCCAGACAGATTCAGCGGCAGTGGGTCA |
| | GGCACTGATTTCACACTGAAAATCAGCAGGGTGGAGGCTGAGGATGTTGGGGTT |
| | TATTACTGCATGCAAGGTACACACTGGCCGCTCTCTTTCGGCGGAGGGACCAAG |
| | GTGGAGATCAAA |
| | [SEQ ID NO: 55] |

In certain embodiments, the anti-DLL3 antibody or antigen-binding fragment thereof comprises V_{H} and V_{L} regions or CDRs selected from Table 7. In certain embodiments, the anti-DLL3 antibody or antigen-binding fragment thereof comprises a V_{H} comprising the amino acid sequence set forth in SEQ ID NO: 60, as shown in Table 7. An exemplary nucleotide sequence encoding the amino acid sequence of SEQ ID NO: 60 is set forth in SEQ ID NO: 62. In certain embodiments, the anti-DLL3 antibody or antigen-binding fragment thereof comprises a V_{L} comprising the amino acid sequence set forth in SEQ ID NO: 61. An exemplary nucleotide sequence encoding the amino acid sequence of SEQ ID NO: 61 is set forth in SEQ ID NO: 63. SEQ ID NO: 60-63 are provided in Table 7. In certain embodiments, the antibody or antigen-binding fragment thereof is designated as “M11”.

In certain embodiments, the anti-DLL3 antibody or antigen-binding fragment thereof comprises a V_{H} comprising the amino acid sequence set forth in SEQ ID NO: 60 and a V_{L} comprising the amino acid sequence set forth in SEQ ID NO: 61.

In certain embodiments, the anti-DLL3 antibody or antigen-binding fragment thereof comprises a V_{H} comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 56 or a conservative modification thereof. SEQ ID NOs: 2, 21, and 56 are provided in Table 7.

In certain embodiments, the anti-DLL3 antibody or antigen-binding fragment thereof comprises a V_{L} comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 59 or a conservative modification thereof. SEQ ID NOs: 57-59 are provided in Table 7.

In certain embodiments, the anti-DLL3 antibody or antigen-binding fragment thereof comprises a V_{H} comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 56 or a conservative modification thereof; and a V_{L} comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 59 or a conservative modification thereof.

In certain embodiments, the anti-DLL3 antibody or antigen-binding fragment thereof comprises a V_{H} comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 56; and a V_{L} Comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 59.

TABLE 7

| CDRs | 1 | 2 | 3 |
|---|---|---|---|
| V_{H} | GYTFTSY | NPSGGS | QLELLGNAFDI |
| | [SEQ ID NO: 21] | [SEQ ID NO: 2] | [SEQ ID NO: 56] |
| V_{L} | RASQSVSSSYLA | GASSRAT | QQYGSSPYT |
| | [SEQ ID NO: 57] | [SEQ ID NO: 58] | [SEQ ID NO: 59] |

| | |
|---|---|
| Full V_{H} | QVQLVQSGAEVKKPGASVKVSCKASGYTFTSYYIHWVRQAPGQGLEWMGIINPS |
| | GGSKSYAQKFQGRVTMTRDTSTGTVYMELSSLRSEDTAVYYCAGQLELLGNAFD |
| | IWGQGTMVTVSS |
| | [SEQ ID NO: 60] |
| Full V_{L} | EIVLTQSPGTLSLSPGERATLSCRASQSVSSSYLAWYQQKPGQAPRLLIYGASS |
| | RATGIPDRFSGSGSGTDFTLTISRLEPEDFAVYYCQQYGSSPYTFGQGTKLEIK |
| | [SEQ ID NO: 61] |
| DNA for | CAGGTGCAGCTGGTGCAGTCTGGGGCTGAGGTGAAGAAGCCTGGGGCCTCAGTG |
| Full V_{H} | AAGGTTTCCTGCAAGGCATCTGGATACACCTTCACCAGCTACTATATACACTGG |
| | GTGCGACAGGCCCCTGGACAAGGGCTTGAGTGGATGGGAATAATCAACCCTAGT |
| | GGTGGTAGTAAAAGCTACGCACAGAAGTTCCAGGGCAGAGTCACCATGACCAGG |
| | GACACGTCCACGGGAACAGTCTATATGGAGCTGAGCAGCCTGAGATCTGAGGAC |
| | ACGGCCGTGTATTACTGTGCGGGACAACTGGAACTTTTGGGGAATGCTTTTGAT |
| | ATCTGGGGCCAAGGGACAATGGTCACCGTCTCTTCA |
| | [SEQ ID NO: 62] |
| DNA for | GAAATTGTGTTGACGCAGTCTCCAGGCACCCTGTCTTTGTCTCCAGGGGAAAGA |
| Full V_{L} | GCCACCCTCTCCTGCAGGGCCAGTCAGAGTGTTAGCAGCAGCTACTTAGCCTGG |
| | TACCAGCAGAAACCTGGCCAGGCTCCCAGGCTCCTCATCTATGGTGCATCCAGC |
| | AGGGCCACTGGCATCCCAGACAGGTTCAGTGGCAGTGGGTCTGGGACAGACTTC |
| | ACTCTCACCATCAGCAGACTGGAGCCTGAAGATTTTGCAGTGTATTACTGTCAG |
| | CAGTATGGTAGCTCACCGTACACTTTTGGCCAGGGGACCAAGCTGGAGATCAAA |
| | [SEQ ID NO: 63] |

In certain embodiments, the anti-DLL3 antibody or antigen-binding fragment thereof comprises V_{H} and V_{L} regions or CDRs selected from Table 8. In certain embodiments, the anti-DLL3 antibody or antigen-binding fragment thereof comprises a V_{H} comprising the amino acid sequence set forth in SEQ ID NO: 66, as shown in Table 8. An exemplary nucleotide sequence encoding the amino acid sequence of SEQ ID NO: 66 is set forth in SEQ ID NO: 68. In certain embodiments, the anti-DLL3 antibody or antigen-binding fragment thereof comprises a V_{L} comprising the amino acid sequence set forth in SEQ ID NO: 67. An exemplary nucleotide sequence encoding the amino acid sequence of SEQ ID NO: 67 is set forth in SEQ ID NO: 69. SEQ ID NO: 66-69 are provided in Table 8. In certain embodiments, the antibody or antigen-binding fragment thereof is designated as “024”.

In certain embodiments, the anti-DLL3 antibody or antigen-binding fragment thereof comprises a V_{H} comprising the amino acid sequence set forth in SEQ ID NO: 66 and a V_{L} comprising the amino acid sequence set forth in SEQ ID NO: 67.

In certain embodiments, the anti-DLL3 antibody or antigen-binding fragment thereof comprises a V_{H} comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 64 or a conservative modification thereof. SEQ ID NOs: 2, 21, and 64 are provided in Table 8.

In certain embodiments, the anti-DLL3 antibody or antigen-binding fragment thereof comprises a V_{L} comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 65 or a conservative modification thereof. SEQ ID NOs: 4, 5 and 65 are provided in Table 8.

In certain embodiments, the anti-DLL3 antibody or antigen-binding fragment thereof comprises a V_{H} comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 64 or a conservative modification thereof; and a V_{L} comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 65 or a conservative modification thereof.

In certain embodiments, the anti-DLL3 antibody or antigen-binding fragment thereof comprises a V_{H} comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 64; and a V_{L} comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 65.

TABLE 8

| CDRs | 1 | 2 | 3 |
|---|---|---|---|
| V_{H} | GYTFTSY | NPSGGS | ERQQLDYYYFAMDV |
| | [SEQ ID NO: 21] | [SEQ ID NO: 2] | [SEQ ID NO: 64] |
| V_{L} | RASQGISNYLA | AASSLQS | QQYNSYPPT |
| | [SEQ ID NO: 4] | [SEQ ID NO: 5] | [SEQ ID NO: 65] |

| | |
|---|---|
| Full V_{H} | QVQLVQSGAEVKKPGASVKVSCKASGYTFTSYYLHWVRQAPGHGLEWMAIINPS |
| | GGSTSYAQKFLGRVTMTRDTSTNTVYMELSSLRSEDTAVYYCARERQQLDYYYF |
| | AMDVWGQGTTVTVSS |
| | [SEQ ID NO: 66] |
| Full V_{L} | DIQMTQSPSSLSASVGDRVTITCRASQGISNYLAWFQQKPGKAPKSLIYAASSL |
| | QSGVPSKFSGSGSGTDFTLTISSLQPEDFATYYCQQYNSYPPTFGQGTKVEIK |
| | [SEQ ID NO: 67] |
| DNA for | CAGGTGCAGCTGGTGCAGTCTGGGGCTGAGGTGAAGAAGCCTGGGGCCTCAGTG |
| Full V_{H} | AAGGTTTCCTGCAAGGCATCTGGATACACCTTCACCAGCTACTATTTGCACTGG |
| | GTGCGACAGGCCCCTGGACACGGGCTTGAGTGGATGGCAATAATCAACCCTAGT |
| | GGTGGTAGTACAAGCTACGCACAGAAGTTCCTGGGCAGAGTCACCATGACCAGG |
| | GACACGTCCACGAACACAGTCTACATGGAGCTGAGCAGCCTGAGATCTGAGGAC |
| | ACGGCCGTGTATTACTGTGCGAGAGAACGGCAGCAGCTGGACTACTACTACTTC |
| | GCTATGGACGTCTGGGGCCAAGGGACCACGGTCACCGTCTCCTCA |
| | [SEQ ID NO: 68] |
| DNA for | GACATCCAGATGACCCAGTCTCCATCCTCACTGTCTGCATCTGTAGGAGACAGA |
| Full V_{L} | GTCACCATCACTTGTCGGGCGAGTCAGGGCATTAGCAATTATTTAGCCTGGTTT |
| | CAGCAGAAACCAGGGAAAGCCCCTAAGTCCCTGATCTATGCTGCATCCAGTTTG |
| | CAAAGTGGGGTCCCATCAAAGTTCAGCGGCAGTGGATCTGGGACAGATTTCACT |
| | CTCACCATCAGCAGCCTGCAGCCTGAAGATTTTGCAACTTATTACTGCCAACAG |
| | TATAATAGTTACCCTCCGACGTTCGGCCAAGGGACCAAGGTGGAAATCAAA |
| | [SEQ ID NO: 69] |

In certain embodiments, the anti-DLL3 antibody or antigen-binding fragment thereof comprises V_{H} and V_{L} regions or CDRs selected from Table 9. In certain embodiments, the anti-DLL3 antibody or antigen-binding fragment thereof comprises a V_{H} comprising the amino acid sequence set forth in SEQ ID NO: 76, as shown in Table 9. An exemplary nucleotide sequence encoding the amino acid sequence of SEQ ID NO: 76 is set forth in SEQ ID NO: 78. In certain embodiments, the anti-DLL3 antibody or antigen-binding fragment thereof comprises a V_{L} comprising the amino acid sequence set forth in SEQ ID NO: 77. An exemplary nucleotide sequence encoding the amino acid sequence of SEQ ID NO: 77 is set forth in SEQ ID NO: 79. SEQ ID NO: 76-79 are provided in Table 9. In certain embodiments, the antibody or antigen-binding fragment thereof is designated as “P4”.

In certain embodiments, the anti-DLL3 antibody or antigen-binding fragment thereof comprises a V_{H} comprising the amino acid sequence set forth in SEQ ID NO: 76 and a V_{L} comprising the amino acid sequence set forth in SEQ ID NO: 77.

In certain embodiments, the anti-DLL3 antibody or antigen-binding fragment thereof comprises a V_{H} comprising a CDR1 comprising the amino acid sequence set forth in SEQ ID NO: 70 or a conservative modification thereof, a CDR2 comprising the amino acid sequence set forth in SEQ ID NO: 71 or a conservative modification thereof, and a CDR3 comprising the amino acid sequence set forth in SEQ ID NO: 72 or a conservative modification thereof. SEQ ID NOs: 70-72 are provided in Table 9.

In certain embodiments, the anti-DLL3 antibody or antigen-binding fragment thereof comprises a V_{L} comprising a CDR1 comprising the amino acid sequence set forth in SEQ ID NO: 73 or a conservative modification thereof, a CDR2 comprising the amino acid sequence set forth in SEQ ID NO: 74 or a conservative modification thereof, and a CDR3 comprising the amino acid sequence set forth in SEQ ID NO: 75 or a conservative modification thereof. SEQ ID NOs: 73-75 are provided in Table 9.

In certain embodiments, the anti-DLL3 antibody or antigen-binding fragment thereof comprises a V_{H} comprising a CDR1 comprising the amino acid sequence set forth in SEQ ID NO: 70 or a conservative modification thereof, a CDR2 comprising the amino acid sequence set forth in SEQ ID NO: 71 or a conservative modification thereof, and a CDR3 comprising the amino acid sequence set forth in SEQ ID NO: 72 or a conservative modification thereof; and a V_{L} comprising a CDR1 comprising the amino acid sequence set forth in SEQ ID NO: 73 or a conservative modification thereof, a CDR2 comprising the amino acid sequence set forth in SEQ ID NO: 74 or a conservative modification thereof, and a CDR3 comprising the amino acid sequence set forth in SEQ ID NO: 75 or a conservative modification thereof.

In certain embodiments, the anti-DLL3 antibody or antigen-binding fragment thereof comprises a V_{H} comprising a CDR1 comprising the amino acid sequence set forth in SEQ ID NO: 70, a CDR2 comprising the amino acid sequence set forth in SEQ ID NO: 71, and a CDR3 comprising the amino acid sequence set forth in SEQ ID NO: 72; and a V_{L} Comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 75.

TABLE 9

| CDRs | 1 | 2 | 3 |
|---|---|---|---|
| V_{H} | GFTFSFY | SSSSSY | NYDFWRGAFDI |
| | [SEQ ID NO: 70] | [SEQ ID NO: 71] | [SEQ ID NO: 72] |
| V_{L} | RASQSVSSNLA | GASTRAT | QQYNNWPTWT |
| | [SEQ ID NO: 73] | [SEQ ID NO: 74] | [SEQ ID NO: 75] |

| | |
|---|---|
| Full V_{H} | EVQLVESGGGLVKPGGSLRLSCAASGFTFSFYSMNWVRQAPGKGLEWVSSISSSSSYIY |
| | YADSVKGRFTISRDNAKNSLYLQMNSLRAEDTAVYYCARNYDFWRGAFDIWGQGTMVTV |
| | SS |
| | [SEQ ID NO: 76] |
| Full V_{L} | EIVMTQSPATLSVSPGERATLSCRASQSVSSNLAWYQQKPGQAPRLLIYGASTRATGIP |
| | ARFSGSGSGTEFTLTISSLQSEDFAVYYCQQYNNWPTWTFGQGTKVEIK |
| | [SEQ ID NO: 77] |
| DNA for | GAGGTGCAGCTGGTGGAGTCTGGGGGGGGCCTGGTCAAGCCTGGAGGGTCCCTGAGACT |
| Full V_{H} | CTCCTGTGCAGCCTCTGGATTCACCTTCAGTTTCTATAGCATGAACTGGGTCCGCCAGG |
| | CTCCAGGGAAGGGGCTGGAGTGGGTCTCATCCATTAGTAGTAGTAGTAGTTACATATAC |
| | TACGCAGACTCAGTGAAGGGCCGATTCACCATCTCCAGAGACAACGCCAAGAACTCACT |
| | GTATCTGCAAATGAACAGCCTGAGAGCCGAGGACACGGCTGTGTATTACTGTGCGAGAA |
| | ATTACGATTTTTGGAGAGGTGCTTTTGATATCTGGGGCCAAGGGACAATGGTCACCGTC |
| | TCTTCA |
| | [SEQ ID NO: 78] |
| DNA for | GAAATAGTGATGACGCAGTCTCCAGCCACCCTGTCTGTGTCTCCAGGGGAAAGAGCCAC |
| Full V_{L} | CCTCTCCTGCAGGGCCAGTCAGAGTGTTAGCAGCAACTTAGCCTGGTACCAGCAGAAAC |
| | CTGGCCAGGCTCCCAGGCTCCTCATCTATGGTGCATCCACCAGGGCCACTGGTATCCCA |
| | GCCAGGTTCAGTGGCAGTGGGTCTGGGACAGAGTTCACTCTCACCATCAGCAGCCTGCA |
| | GTCTGAAGATTTTGCAGTTTATTACTGTCAGCAGTATAATAACTGGCCCACGTGGACGT |
| | TCGGCCAAGGGACCAAGGTGGAAATCAAA |
| | [SEQ ID NO: 79] |

In certain embodiments, the anti-DLL3 antibody or antigen-binding fragment thereof comprises V_{H} and V_{L} regions or CDRs selected from Table 10. In certain embodiments, the anti-DLL3 antibody or antigen-binding fragment thereof comprises a V_{H} comprising the amino acid sequence set forth in SEQ ID NO: 83, as shown in Table 10. An exemplary nucleotide sequence encoding the amino acid sequence of SEQ ID NO: 83 is set forth in SEQ ID NO: 85. In certain embodiments, the anti-DLL3 antibody or antigen-binding fragment thereof comprises a V_{L} comprising the amino acid sequence set forth in SEQ ID NO: 84. An exemplary nucleotide sequence encoding the amino acid sequence of SEQ ID NO: 84 is set forth in SEQ ID NO: 86. SEQ ID NO: 83-86 are provided in Table 10. In certain embodiments, the antibody or antigen-binding fragment thereof is designated as “J23”.

In certain embodiments, the anti-DLL3 antibody or antigen-binding fragment thereof comprises a V_{H} comprising the amino acid sequence set forth in SEQ ID NO: 83 and a V_{L} comprising the amino acid sequence set forth in SEQ ID NO: 84.

In certain embodiments, the anti-DLL3 antibody or antigen-binding fragment thereof comprises a V_{H} comprising a CDR1 comprising the amino acid sequence set forth in SEQ ID NO: 21 or a conservative modification thereof, a CDR2 comprising the amino acid sequence set forth in SEQ ID NO: 80 or a conservative modification thereof, and a CDR3 comprising the amino acid sequence set forth in SEQ ID NO: 81 or a conservative modification thereof. SEQ ID NOs: 21, 80, and 81 are provided in Table 10.

In certain embodiments, the anti-DLL3 antibody or antigen-binding fragment thereof comprises a V_{L} comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 82 or a conservative modification thereof. SEQ ID NOs: 57, 58, and 82 are provided in Table 10.

In certain embodiments, the anti-DLL3 antibody or antigen-binding fragment thereof comprises a V_{H} comprising a CDR1 comprising the amino acid sequence set forth in SEQ ID NO: 21 or a conservative modification thereof, a CDR2 comprising the amino acid sequence set forth in SEQ ID NO: 80 or a conservative modification thereof, and a CDR3 comprising the amino acid sequence set forth in SEQ ID NO: 81 or a conservative modification thereof; and a V_{L} comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 82 or a conservative modification thereof.

In certain embodiments, the anti-DLL3 antibody or antigen-binding fragment thereof comprises a V_{H} comprising a CDR1 comprising the amino acid sequence set forth in SEQ ID NO: 21, a CDR2 comprising the amino acid sequence set forth in SEQ ID NO: 80, and a CDR3 comprising the amino acid sequence set forth in SEQ ID NO: 81; and a V_{L} Comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82.

TABLE 10

| CDRs | 1 | 2 | 3 |
|---|---|---|---|
| V_{H} | GYTFTSY | NYSGGS | QGRYSGSYLDY |
| | [SEQ ID NO: 21] | [SEQ ID NO: 80] | [SEQ ID NO: 81] |
| V_{L} | RASQSVSSSYLA | GASSRAT | QQYGSSP |
| | [SEQ ID NO: 57] | [SEQ ID NO: 58] | [SEQ ID NO: 82] |

| | |
|---|---|
| Full V_{H} | QVQLVQSGAEVKKPGASVKVSCKASGYTFTSYYMHWVRQAPGQGLEWMGIINYS |
| | GGSKSYAQKFQGRVTMTRDTSTSTVYMELSSLRSEDTGVYYCARQGRYSGSYLD |
| | YWGQGTLVTVSS |
| | [SEQ ID NO: 83] |
| Full V_{L} | EIVLTQSPGTLSLSPGERATLSCRASQSVSSSYLAWYQQKPGQAPRLLIYGASS |
| | RATGIPDRFSGSGSGTDFTLTISRLEPEDFAVYYCQQYGSSPFGQGTKLEIK |
| | [SEQ ID NO: 84] |
| DNA for | CAGGTGCAGCTGGTGCAGTCTGGGGCTGAGGTGAAGAAGCCTGGGGCCTCAGTG |
| Full V_{H} | AAGGTTTCCTGCAAGGCATCTGGATACACCTTCACCAGCTACTATATGCACTGG |
| | GTGCGACAGGCCCCTGGACAAGGGCTTGAGTGGATGGGAATAATCAACTATAGT |
| | GGTGGTAGCAAAAGCTACGCACAGAAGTTCCAGGGCAGAGTCACCATGACCCGG |
| | GACACGTCCACGAGCACAGTCTACATGGAGCTGAGCAGCCTGAGATCTGAGGAC |
| | ACGGGCGTGTATTACTGTGCGAGACAGGGGCGGTATAGTGGGAGCTACCTTGAC |
| | TACTGGGGCCAGGGAACCCTGGTCACCGTCTCCTCA |
| | [SEQ ID NO: 85] |
| DNA for | GAAATTGTGTTGACGCAGTCTCCAGGCACCCTGTCTTTGTCTCCAGGGGAAAGA |
| Full V_{L} | GCCACCCTCTCCTGCAGGGCCAGTCAGAGTGTTAGCAGCAGCTACTTAGCCTGG |
| | TACCAGCAGAAACCTGGCCAGGCTCCCAGGCTCCTCATCTATGGTGCATCCAGC |
| | AGGGCCACTGGCATCCCAGACAGGTTCAGTGGCAGTGGGTCTGGGACAGACTTC |
| | ACTCTCACCATCAGCAGACTGGAGCCTGAAGATTTTGCAGTGTATTACTGTCAG |
| | CAGTATGGTAGCTCACCTTTTGGCCAGGGGACCAAGCTGGAGATCAAA |
| | [SEQ ID NO: 86] |

In certain embodiments, the anti-DLL3 antibody or antigen-binding fragment thereof comprises V_{H} and V_{L} regions or CDRs selected from Table 11. In certain embodiments, the anti-DLL3 antibody or antigen-binding fragment thereof comprises a V_{H} comprising the amino acid sequence set forth in SEQ ID NO: 92, as shown in Table 1. An exemplary nucleotide sequence encoding the amino acid sequence of SEQ ID NO: 92 is set forth in SEQ ID NO: 94. In certain embodiments, the anti-DLL3 antibody or antigen-binding fragment thereof comprises a V_{L} comprising the amino acid sequence set forth in SEQ ID NO: 93. An exemplary nucleotide sequence encoding the amino acid sequence of SEQ ID NO: 93 is set forth in SEQ ID NO: 95. SEQ ID NO: 92-95 are provided in Table 11. In certain embodiments, the antibody or antigen-binding fragment thereof is designated as “K19”.

In certain embodiments, the anti-DLL3 antibody or antigen-binding fragment thereof comprises a V_{H} comprising the amino acid sequence set forth in SEQ ID NO: 92 and a V_{L} comprising the amino acid sequence set forth in SEQ ID NO: 93.

In certain embodiments, the anti-DLL3 antibody or antigen-binding fragment thereof comprises a V_{H} comprising a CDR1 comprising the amino acid sequence set forth in SEQ ID NO: 87 or a conservative modification thereof, a CDR2 comprising the amino acid sequence set forth in SEQ ID NO: 88 or a conservative modification thereof, and a CDR3 comprising the amino acid sequence set forth in SEQ ID NO: 89 or a conservative modification thereof. SEQ ID NOs: 87-89 are provided in Table 11.

In certain embodiments, the anti-DDL3 antibody or antigen-binding fragment thereof comprises a V_{L} comprising a CDR1 comprising the amino acid sequence set forth in SEQ ID NO: 90 or a conservative modification thereof, a CDR2 comprising the amino acid sequence set forth in SEQ ID NO: 210 or a conservative modification thereof, and a CDR3 comprising the amino acid sequence set forth in SEQ ID NO: 91 or a conservative modification thereof. SEQ ID NOs: 210, 90, and 91 are provided in Table 11.

In certain embodiments, the anti-DLL3 antibody or antigen-binding fragment thereof comprises a V_{H} comprising a CDR1 comprising the amino acid sequence set forth in SEQ ID NO: 87 or a conservative modification thereof, a CDR2 comprising the amino acid sequence set forth in SEQ ID NO: 88 or a conservative modification thereof, and a CDR3 comprising the amino acid sequence set forth in SEQ ID NO: 89 or a conservative modification thereof; and a V_{L} comprising a CDR1 comprising the amino acid sequence set forth in SEQ ID NO: 90 or a conservative modification thereof, a CDR2 comprising the amino acid sequence set forth in SEQ ID NO: 210 or a conservative modification thereof, and a CDR3 comprising the amino acid sequence set forth in SEQ ID NO: 91 or a conservative modification thereof.

In certain embodiments, the anti-DLL3 antibody or antigen-binding fragment thereof comprises a V_{H} comprising a CDR1 comprising the amino acid sequence set forth in SEQ ID NO: 87, a CDR2 comprising the amino acid sequence set forth in SEQ ID NO: 88, and a CDR3 comprising the amino acid sequence set forth in SEQ ID NO: 89; and a V_{L} Comprising a CDR1 comprising the amino acid sequence set forth in SEQ ID NO: 90, a CDR2 comprising the amino acid sequence set forth in SEQ ID NO: 210, and a CDR3 comprising the amino acid sequence set forth in SEQ ID NO: 91.

TABLE 11

| CDRs | 1 | 2 | 3 |
|---|---|---|---|
| V_{H} | GYTENSY | NPSGGT | DWGEQYGMDV |
| | [SEQ ID NO: 87] | [SEQ ID NO: 88] | [SEQ ID NO: 89] |
| V_{L} | RSSQSLLHSNGYNYLD | LGSNRAS | MQALQTPLT |
| | [SEQ ID NO: 90] | [SEQ ID NO: 210] | [SEQ ID NO: 91] |

| | |
|---|---|
| Full V_{H} | QVQLVQSGAEVKKPGASLKVSCKTSGYTFNSYYMHWVRQAPGQGLEWMGINNPS |
| | GGTTSYAQKFQGRVTMTRDTSTSTVYMELSSLRSEDTAVYYCARDWGEQYGMDV |
| | WGQGTTVTVSS |
| | [SEQ ID NO: 92] |
| Full V_{L} | DIVMTQSPLSLPVTPGEPASISCRSSQSLLHSNGYNYLDWYLQKPGQSPQLLIY |
| | LGSNRASGVPDRFSGSGSGTDFTLKISRVEAEDVGVYYCMQALQTPLTFGGGTK |
| | VEIK |
| | [SEQ ID NO: 93] |
| DNA for | CAGGTGCAGCTGGTGCAGTCTGGGGCTGAGGTGAAGAAGCCTGGGGCCTCACTG |
| Full V_{H} | AAGGTTTCCTGCAAGACATCTGGATACACCTTCAACAGCTACTATATGCACTGG |
| | GTGCGACAGGCCCCTGGACAAGGGCTTGAGTGGATGGGAATAAACAACCCTAGT |
| | GGTGGTACCACAAGCTACGCACAGAAGTTCCAGGGCAGAGTCACCATGACCAGG |
| | GACACGTCCACGAGCACAGTCTACATGGAGCTGAGCAGCCTGAGATCTGAGGAC |
| | ACGGCCGTGTATTACTGTGCGAGAGATTGGGGGGAGCAGTACGGTATGGACGTC |
| | TGGGGCCAAGGGACCACGGTCACCGTCTCCTCA |
| | [SEQ ID NO: 94] |
| DNA for | GATATTGTGATGACTCAGTCTCCACTCTCCCTGCCCGTCACCCCTGGAGAGCCG |
| Full V_{L} | GCCTCCATCTCCTGCAGGTCTAGTCAGAGCCTCCTGCATAGTAATGGATACAAC |
| | TATTTGGATTGGTACCTGCAGAAGCCAGGACAGTCTCCACAGCTCCTGATCTAT |
| | TTGGGTTCTAATCGGGCCTCCGGGGTCCCTGACAGGTTCAGTGGCAGTGGATCA |
| | GGCACAGATTTTACACTGAAAATCAGCAGAGTGGAGGCTGAGGATGTTGGGGTT |
| | TATTACTGCATGCAAGCTCTACAAACTCCGCTCACTTTCGGCGGAGGGACCAAG |
| | GTGGAGATCAAA |
| | [SEQ ID NO: 95] |

In certain embodiments, the anti-DLL3 antibody or antigen-binding fragment thereof comprises V_{H} and V_{L} regions or CDRs selected from Table 12. In certain embodiments, the anti-DLL3 antibody or antigen-binding fragment thereof comprises a V_{H} comprising the amino acid sequence set forth in SEQ ID NO: 102, as shown in Table 12. An exemplary nucleotide sequence encoding the amino acid sequence of SEQ ID NO: 102 is set forth in SEQ ID NO: 104. In certain embodiments, the anti-DLL3 antibody or antigen-binding fragment thereof comprises a V_{L} comprising the amino acid sequence set forth in SEQ ID NO: 103. An exemplary nucleotide sequence encoding the amino acid sequence of SEQ ID NO: 103 is set forth in SEQ ID NO: 105. SEQ ID NO: 102-105 are provided in Table 12. In certain embodiments, the antibody or antigen-binding fragment thereof is designated as “N10”.

In certain embodiments, the anti-DLL3 antibody or antigen-binding fragment thereof comprises a V_{H} comprising the amino acid sequence set forth in SEQ ID NO: 102 and a V_{L} comprising the amino acid sequence set forth in SEQ ID NO: 103.

In certain embodiments, the anti-DLL3 antibody or antigen-binding fragment thereof comprises a V_{H} comprising a CDR1 comprising the amino acid sequence set forth in SEQ ID NO: 96 or a conservative modification thereof, a CDR2 comprising the amino acid sequence set forth in SEQ ID NO: 97 or a conservative modification thereof, and a CDR3 comprising the amino acid sequence set forth in SEQ ID NO: 98 or a conservative modification thereof. SEQ ID NOs: 96-98 are provided in Table 12.

In certain embodiments, the anti-DLL3 antibody or antigen-binding fragment thereof comprises a V_{L} comprising a CDR1 comprising the amino acid sequence set forth in SEQ ID NO: 99 or a conservative modification thereof, a CDR2 comprising the amino acid sequence set forth in SEQ ID NO: 100 or a conservative modification thereof, and a CDR3 comprising the amino acid sequence set forth in SEQ ID NO: 101 or a conservative modification thereof. SEQ ID NOs: 99-101 are provided in Table 12.

In certain embodiments, the anti-DLL3 antibody or antigen-binding fragment thereof comprises a V_{H} comprising a CDR1 comprising the amino acid sequence set forth in SEQ ID NO: 96 or a conservative modification thereof, a CDR2 comprising the amino acid sequence set forth in SEQ ID NO: 97 or a conservative modification thereof, and a CDR3 comprising the amino acid sequence set forth in SEQ ID NO: 98 or a conservative modification thereof; and a V_{L} comprising a CDR1 comprising the amino acid sequence set forth in SEQ ID NO: 99 or a conservative modification thereof, a CDR2 comprising the amino acid sequence set forth in SEQ ID NO: 100 or a conservative modification thereof, and a CDR3 comprising the amino acid sequence set forth in SEQ ID NO: 101 or a conservative modification thereof.

In certain embodiments, the anti-DLL3 antibody or antigen-binding fragment thereof comprises a V_{H} comprising a CDR1 comprising the amino acid sequence set forth in SEQ ID NO: 96, a CDR2 comprising the amino acid sequence set forth in SEQ ID NO: 97, and a CDR3 comprising the amino acid sequence set forth in SEQ ID NO: 98; and a V_{L} Comprising a CDR1 comprising the amino acid sequence set forth in SEQ ID NO: 99, a CDR2 comprising the amino acid sequence set forth in SEQ ID NO: 100, and a CDR3 comprising the amino acid sequence set forth in SEQ ID NO: 101.

TABLE 12

| CDRs | 1 | 2 | 3 |
|---|---|---|---|
| V_{H} | GYTFTGY | NPHSGA | DSSGWYGDAFDI |
| | [SEQ ID NO: 96] | [SEQ ID NO: 97] | [SEQ ID NO: 98] |
| V_{L} | RSSQSLVHSDGNTYLS | KISNRFS | MQATQFPLT |
| | [SEQ ID NO: 99] | [SEQ ID NO: 100] | [SEQ ID NO: 101] |

| | |
|---|---|
| Full V_{H} | QVQLVQSGAEVKKPGASVKVSCKASGYTFTGYYMHWVRQAPGQGLEWMGWINPH |
| | SGAPNSAQKFQVRVTMTRDTSISTAYMELSRLRSDDTAVYYCARDSSGWYGDAF |
| | DIWGQGTMVTVSS |
| | [SEQ ID NO: 102] |
| Full V_{L} | DIVMTQTPLSSPVTLGQPASISCRSSQSLVHSDGNTYLSWLQQRPGQPPRLLIY |
| | KISNRFSGVPDRFSGSGAGTDFTLKISRVEAEDVGVYYCMQATQFPLTFGGGTK |
| | VEIK |
| | [SEQ ID NO: 103] |
| DNA for | CAGGTGCAGCTGGTGCAGTCTGGGGCTGAGGTGAAGAAGCCTGGGGCCTCAGTG |
| Full V_{H} | AAGGTCTCCTGCAAGGCTTCTGGATACACCTTCACCGGCTACTATATGCACTGG |
| | GTGCGACAGGCCCCTGGACAAGGGCTTGAGTGGATGGGATGGATCAACCCTCAC |
| | AGTGGTGCCCCAAACTCTGCACAGAAGTTTCAGGTCAGGGTCACCATGACCAGG |
| | GACACGTCCATCAGCACAGCCTACATGGAGCTGAGCAGGCTGAGATCTGACGAC |
| | ACGGCCGTGTATTACTGTGCGAGGGATAGCAGCGGCTGGTACGGTGATGCTTTT |
| | GATATCTGGGGCCAAGGGACAATGGTCACCGTCTCTTCA |
| | [SEQ ID NO: 104] |
| DNA for | GATATTGTGATGACCCAGACTCCACTCTCCTCACCTGTCACCCTTGGACAGCCG |
| Full V_{L} | GCCTCCATCTCCTGCAGGTCTAGTCAAAGCCTCGTACACAGTGATGGCAACACC |
| | TACTTGAGTTGGCTTCAGCAGAGGCCAGGCCAGCCTCCAAGACTCCTAATTTAT |
| | AAGATTTCTAACCGGTTCTCTGGGGTCCCAGACAGATTCAGTGGCAGTGGGGCA |
| | GGGACAGATTTCACACTGAAAATCAGCAGGGTGGAAGCTGAGGATGTCGGGGTT |
| | TATTACTGCATGCAAGCTACACAATTTCCGCTCACTTTCGGCGGAGGGACCAAG |
| | GTGGAGATCAAA |
| | [SEQ ID NO: 105] |

In certain embodiments, the anti-DLL3 antibody or antigen-binding fragment thereof comprises V_{H} and V_{L} regions or CDRs selected from Table 13. In certain embodiments, the anti-DLL3 scFv comprises a V_{H} comprising the amino acid sequence set forth in SEQ ID NO: 108, as shown in Table 13. An exemplary nucleotide sequence encoding the amino acid sequence of SEQ ID NO: 108 is set forth in SEQ ID NO: 110. In certain embodiments, the anti-DLL3 antibody or antigen-binding fragment thereof comprises a V_{L} comprising the amino acid sequence set forth in SEQ ID NO: 109. An exemplary nucleotide sequence encoding the amino acid sequence of SEQ ID NO: 109 is set forth in SEQ ID NO: 111. SEQ ID NO: 108-111 are provided in Table 13. In certain embodiments, the antibody or antigen-binding fragment thereof is designated as “B16-v1”.

In certain embodiments, the anti-DLL3 antibody or antigen-binding fragment thereof comprises a V_{H} comprising the amino acid sequence set forth in SEQ ID NO: 108 and a V_{L} comprising the amino acid sequence set forth in SEQ ID NO: 109.

In certain embodiments, the anti-DLL3 antibody or antigen-binding fragment thereof comprises a V_{H} comprising a CDR1 comprising the amino acid sequence set forth in SEQ ID NO: 21 or a conservative modification thereof, a CDR2 comprising the amino acid sequence set forth in SEQ ID NO: 106 or a conservative modification thereof, and a CDR3 comprising the amino acid sequence set forth in SEQ ID NO: 107 or a conservative modification thereof. SEQ ID NOs: 105-107 are provided in Table 13.

In certain embodiments, the anti-DLL3 antibody or antigen-binding fragment thereof comprises a V_{L} comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 82 or a conservative modification thereof. SEQ ID NOs: 57, 58, and 82 are provided in Table 13.

In certain embodiments, the anti-DLL3 antibody or antigen-binding fragment thereof comprises a V_{H} comprising a CDR1 comprising the amino acid sequence set forth in SEQ ID NO: 21 or a conservative modification thereof, a CDR2 comprising the amino acid sequence set forth in SEQ ID NO: 106 or a conservative modification thereof, and a CDR3 comprising the amino acid sequence set forth in SEQ ID NO: 107 or a conservative modification thereof; and a V_{L} comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 82 or a conservative modification thereof.

In certain embodiments, the anti-DLL3 antibody or antigen-binding fragment thereof comprises a V_{H} comprising a CDR1 comprising the amino acid sequence set forth in SEQ ID NO: 21, a CDR2 comprising the amino acid sequence set forth in SEQ ID NO: 106, and a CDR3 comprising the amino acid sequence set forth in SEQ ID NO: 107; and a V_{L} comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82.

TABLE 13

| CDRs | 1 | 2 | 3 |
|---|---|---|---|
| V_{H} | GYTFTSY | DPSGGS | QGRYVGSYLDY |
| | [SEQ ID NO: 21] | [SEQ ID NO: 106] | [SEQ ID NO: 107] |
| V_{L} | RASQSVSSSYLA | GASSRAT | QQYGSSP |
| | [SEQ ID NO: 57] | [SEQ ID NO: 58] | [SEQ ID NO: 82] |

| | |
|---|---|
| Full V_{H} | QVQLVQSGAEVKKPGASVKVSCKASGYTFTSYYMHWVRQAPGOGLEWMGIIDPS |
| | GGSTSYAQKFQGRVTMTRDTSTNTVYMDLSSLRSEDTAVYYCARQGRYVGSYLD |
| | YWGQGTLVTVSS |
| | [SEQ ID NO: 108] |
| Full V_{L} | EIVLTQSPGTLSLSPGERATLSCRASQSVSSSYLAWYQQKPGQAPRLLIYGASS |
| | RATGIPDRESGSGSGTDFTLTISRLEPEDFAVYYCQQYGSSPFGQGTKLEIK |
| | [SEQ ID NO: 109] |
| DNA for | CAGGTGCAGCTGGTGCAGTCTGGGGCTGAGGTGAAGAAGCCTGGGGCCTCAGTG |
| Full V_{H} | AAGGTTTCCTGCAAGGCATCTGGATACACCTTCACCAGCTACTATATGCACTGG |
| | GTGCGACAGGCCCCTGGACAAGGGCTTGAGTGGATGGGAATAATCGACCCTAGT |
| | GGTGGTAGCACAAGCTACGCACAGAAGTTCCAGGGCAGAGTCACCATGACCAGG |
| | GACACGTCCACGAACACAGTCTACATGGACCTGAGCAGCCTGAGATCTGAGGAC |
| | ACGGCCGTGTATTACTGTGCGAGACAGGGGCGGTATGTTGGGAGCTACCTTGAC |
| | TACTGGGGCCAGGGAACCCTGGTCACCGTCTCCTCA |
| | [SEQ ID NO: 110] |
| DNA for | GAAATTGTGTTGACGCAGTCTCCAGGCACCCTGTCTTTGTCTCCAGGGGAAAGA |
| Full V_{L} | GCCACCCTCTCCTGCAGGGCCAGTCAGAGTGTTAGCAGCAGCTACTTAGCCTGG |
| | TACCAGCAGAAACCTGGCCAGGCTCCCAGGCTCCTCATCTATGGTGCATCCAGC |
| | AGGGCCACTGGCATCCCAGACAGGTTCAGTGGCAGTGGGTCTGGGACAGACTTC |
| | ACTCTCACCATCAGCAGACTGGAGCCTGAAGATTTTGCAGTGTATTACTGTCAG |
| | CAGTATGGTAGCTCACCTTTTGGCCAGGGGACCAAGCTGGAGATCAAA |
| | [SEQ ID NO: 111] |

In certain embodiments, the anti-DLL3 antibody or antigen-binding fragment thereof comprises V_{H} and V_{L} regions or CDRs selected from Table 14. In certain embodiments, the anti-DLL3 antibody or antigen-binding fragment thereof comprises a V_{H} comprising the amino acid sequence set forth in SEQ ID NO: 108, as shown in Table 14. An exemplary nucleotide sequence encoding the amino acid sequence of SEQ ID NO: 108 is set forth in SEQ ID NO: 110. In certain embodiments, the anti-DLL3 antibody or antigen-binding fragment thereof comprises a V_{L} comprising the amino acid sequence set forth in SEQ ID NO: 113. An exemplary nucleotide sequence encoding the amino acid sequence of SEQ ID NO: 113 is set forth in SEQ ID NO: 114. SEQ ID NO: 108, 110, 113, and 114 are provided in Table 14. In certain embodiments, the antibody or antigen-binding fragment thereof is designated as “B16-v2”.

In certain embodiments, the anti-DLL3 antibody or antigen-binding fragment thereof comprises a V_{H} comprising the amino acid sequence set forth in SEQ ID NO: 108 and a V_{L} comprising the amino acid sequence set forth in SEQ ID NO: 113.

In certain embodiments, the anti-DLL3 antibody or antigen-binding fragment thereof comprises a V_{H} comprising a CDR1 comprising the amino acid sequence set forth in SEQ ID NO: 21 or a conservative modification thereof, a CDR2 comprising the amino acid sequence set forth in SEQ ID NO: 106 or a conservative modification thereof, and a CDR3 comprising the amino acid sequence set forth in SEQ ID NO: 107 or a conservative modification thereof. SEQ ID NOs: 21, 106, and 107 are provided in Table 14.

In certain embodiments, the anti-DLL3 antibody or antigen-binding fragment thereof comprises a V_{L} comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 112 or a conservative modification thereof. SEQ ID NOs: 4, 5, and 112 are provided in Table 14.

In certain embodiments, the anti-DLL3 antibody or antigen-binding fragment thereof comprises a V_{H} comprising a CDR1 comprising the amino acid sequence set forth in SEQ ID NO: 21 or a conservative modification thereof, a CDR2 comprising the amino acid sequence set forth in SEQ ID NO: 106 or a conservative modification thereof, and a CDR3 comprising the amino acid sequence set forth in SEQ ID NO: 107 or a conservative modification thereof; and a V_{L} comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 112 or a conservative modification.

In certain embodiments, the anti-DLL3 antibody or antigen-binding fragment thereof comprises a V_{H} comprising a CDR1 comprising the amino acid sequence set forth in SEQ ID NO: 21, a CDR2 comprising the amino acid sequence set forth in SEQ ID NO: 106, and a CDR3 comprising the amino acid sequence set forth in SEQ ID NO: 107; and a V_{L} comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 112.

TABLE 14

| CDRs | 1 | 2 | 3 |
|---|---|---|---|
| V_{H} | GYTFTSY | DPSGGS | QGRYVGSYLDY |
| | [SEQ ID NO: 21] | [SEQ ID NO: 106] | [SEQ ID NO: 107] |
| V_{L} | RASQGISNYLA | AASSLOS | QQYNSYPYT |
| | [SEQ ID NO: 4] | [SEQ ID NO: 5] | [SEQ ID NO: 112] |

| | |
|---|---|
| Full V_{H} | QVQLVQSGAEVKKPGASVKVSCKASGYTFTSYYMHWVRQAPGQGLEWMGIIDPSGGSTS |
| | YAQKFQGRVTMTRDTSTNTVYMDLSSLRSEDTAVYYCARQGRYVGSYLDYWGQGTLVTV |
| | SS |
| | [SEQ ID NO: 108] |
| Full V_{L} | DIQMTQSPSSLSASVGDRVTITCRASQGISNYLAWFQQKPGKAPKSVIYAASSLQSGVP |
| | SKFSGSGSGTDFTLTISSMOPEDFATYYCQQYNSYPYTFGQGTKLEIK |
| | [SEQ ID NO: 113] |
| DNA for | CAGGTGCAGCTGGTGCAGTCTGGGGCTGAGGTGAAGAAGCCTGGGGCCTCAGTGAAGGT |
| Full V_{H} | TTCCTGCAAGGCATCTGGATACACCTTCACCAGCTACTATATGCACTGGGTGCGACAGG |
| | CCCCTGGACAAGGGCTTGAGTGGATGGGAATAATCGACCCTAGTGGTGGTAGCACAAGC |
| | TACGCACAGAAGTTCCAGGGCAGAGTCACCATGACCAGGGACACGTCCACGAACACAGT |
| | CTACATGGACCTGAGCAGCCTGAGATCTGAGGACACGGCCGTGTATTACTGTGCGAGAC |
| | AGGGGCGGTATGTTGGGAGCTACCTTGACTACTGGGGCCAGGGAACCCTGGTCACCGTC |
| | TCCTCA |
| | [SEQ ID NO: 110] |
| DNA for | GACATCCAGATGACCCAGTCTCCATCCTCACTGTCTGCATCTGTAGGAGACAGAGTCAC |
| Full V_{L} | CATCACTTGTCGGGCGAGTCAGGGCATTAGCAATTATTTAGCCTGGTTTCAGCAGAAAC |
| | CAGGGAAAGCCCCTAAGTCCGTGATCTATGCTGCATCCAGTTTGCAAAGTGGGGTCCCA |
| | TCAAAGTTCAGCGGCAGTGGATCTGGGACAGATTTCACTCTCACCATCAGCAGCATGCA |
| | GCCTGAAGATTTTGCAACTTATTACTGCCAACAGTATAATAGTTACCCGTACACTTTTG |
| | GCCAGGGGACCAAGCTGGAGATCAAA |
| | [SEQ ID NO: 114] |

In certain embodiments, the anti-DLL3 antibody or antigen-binding fragment thereof comprises V_{H} and V_{L} regions or CDRs selected from Table 15. In certain embodiments, the anti-DLL3 antibody or antigen-binding fragment thereof comprises a V_{H} comprising the amino acid sequence set forth in SEQ ID NO: 119, as shown in Table 15. An exemplary nucleotide sequence encoding the amino acid sequence of SEQ ID NO: 119 is set forth in SEQ ID NO: 121. In certain embodiments, the anti-DLL3 antibody or antigen-binding fragment thereof comprises a V_{L} comprising the amino acid sequence set forth in SEQ ID NO: 120. An exemplary nucleotide sequence encoding the amino acid sequence of SEQ ID NO: 120 is set forth in SEQ ID NO: 122. In certain embodiments, the anti-DLL3 antibody or antigen-binding fragment thereof comprises a V_{H} comprising the amino acid sequence set forth in SEQ ID NO: 119 and a V_{L} Comprising the amino acid sequence set forth in SEQ ID NO: 120. SEQ ID NO: 119-122 are provided in Table 15. In certain embodiments, the antibody or antigen-binding fragment thereof is designated as “E23”.

In certain embodiments, the anti-DLL3 antibody or antigen-binding fragment thereof comprises a V_{H} comprising a CDR1 comprising the amino acid sequence set forth in SEQ ID NO: 96 or a conservative modification thereof, a CDR2 comprising the amino acid sequence set forth in SEQ ID NO: 115 or a conservative modification thereof, and a CDR3 comprising the amino acid sequence set forth in SEQ ID NO: 116 or a conservative modification thereof. SEQ ID NOs: 96, 115, and 116 are provided in Table 15.

In certain embodiments, the anti-DLL3 antibody or antigen-binding fragment thereof comprises a V_{L} comprising a CDR1 comprising the amino acid sequence set forth in SEQ ID NO: 117 or a conservative modification thereof, a CDR2 comprising the amino acid sequence set forth in SEQ ID NO: 100 or a conservative modification thereof, and a CDR3 comprising the amino acid sequence set forth in SEQ ID NO: 118 or a conservative modification thereof. SEQ ID NOs: 100, 117, and 118 are provided in Table 15.

In certain embodiments, the anti-DLL3 antibody or antigen-binding fragment thereof comprises a V_{H} comprising a CDR1 comprising the amino acid sequence set forth in SEQ ID NO: 96 or a conservative modification thereof, a CDR2 comprising the amino acid sequence set forth in SEQ ID NO: 115 or a conservative modification thereof, and a CDR3 comprising the amino acid sequence set forth in SEQ ID NO: 116 or a conservative modification thereof; and a V_{L} comprising a CDR1 comprising the amino acid sequence set forth in SEQ ID NO:117 or a conservative modification thereof, a CDR2 comprising the amino acid sequence set forth in SEQ ID NO: 100 or a conservative modification thereof, and a CDR3 comprising the amino acid sequence set forth in SEQ ID NO: 118 or a conservative modification.

In certain embodiments, the anti-DLL3 antibody or antigen-binding fragment thereof comprises a V_{H} comprising a CDR1 comprising the amino acid sequence set forth in SEQ ID NO: 96, a CDR2 comprising the amino acid sequence set forth in SEQ ID NO: 115, and a CDR3 comprising the amino acid sequence set forth in SEQ ID NO: 116; and a V_{L} comprising a CDR1 comprising the amino acid sequence set forth in SEQ ID NO: 117, a CDR2 comprising the amino acid sequence set forth in SEQ ID NO: 100, and a CDR3 comprising the amino acid sequence set forth in SEQ ID NO: 118.

TABLE 15

| CDRs | 1 | 2 | 3 |
|---|---|---|---|
| V_{H} | GYTFTGY | NSYSGG | DGSGWYGSYFDE |
| | [SEQ ID NO: 96] | [SEQ ID NO: 115] | [SEQ ID NO: 116] |
| V_{L} | RSSQSLVHSDGNTYLS | KISNRFS | IQTTQFPLT |
| | [SEQ ID NO: 117] | [SEQ ID NO: 100] | [SEQ ID NO: 118] |

| | |
|---|---|
| Full V_{H} | QVQLVQSGAEVRKPGASVKVSCKASGYTFTGYYMHWVRQAPGQGLEWMGWINSY |
| | SGGANYAQKFQDRVTMTRDTSVSTASMELSRLRSDDTAVYYCARDGSGWYGSYF |
| | DFWGQGTLVTVSS |
| | [SEQ ID NO: 119] |
| Full V_{L} | DIVMTQTPLSSPVTLGQPASISCRSSQSLVHSDGNTYLSWLQQRPGQPPRLLIY |
| | KISNRFSGVPDRESGSGAGTDFTLKISRVEAEDVGFYYCIQTTQFPLTFGGGTK |
| | VEIK |
| | [SEQ ID NO: 120] |
| DNA for | CAGGTGCAGCTGGTGCAGTCTGGGGCTGAGGTGAGGAAGCCTGGGGCCTCAGTG |
| Full V_{H} | AAGGTCTCCTGCAAGGCTTCTGGATACACCTTCACCGGCTACTATATGCACTGG |
| | GTGCGACAGGCCCCTGGACAAGGGCTTGAGTGGATGGGATGGATCAACTCTTAC |
| | AGTGGTGGCGCAAACTATGCACAGAAGTTTCAGGACAGGGTCACCATGACCAGG |
| | GACACGTCCGTCAGCACAGCCTCCATGGAGCTGAGCAGGCTGAGATCTGACGAC |
| | ACGGCCGTGTATTACTGTGCGAGAGATGGCAGTGGCTGGTACGGGTCCTACTTT |
| | GACTTCTGGGGCCAGGGAACCCTGGTCACCGTCTCCTCA |
| | [SEQ ID NO: 121] |
| DNA for | GATATTGTGATGACCCAGACTCCACTCTCCTCACCTGTCACCCTTGGACAGCCG |
| Full V_{L} | GCCTCCATCTCCTGCAGGTCTAGTCAAAGCCTCGTACACAGTGATGGAAACACC |
| | TACTTGAGTTGGCTTCAGCAGAGGCCAGGCCAGCCTCCAAGACTCCTAATTTAT |
| | AAGATTTCTAACCGGTTCTCTGGGGTCCCAGACAGATTCAGTGGCAGTGGGGCA |
| | GGGACAGATTTCACACTGAAAATCAGCAGGGTGGAAGCTGAGGATGTCGGGTTT |
| | TATTACTGCATACAAACTACACAATTTCCGCTCACTTTCGGCGGAGGGACCAAG |
| | GTGGAGATCAAA |
| | [SEQ ID NO: 122] |

In certain embodiments, the anti-DLL3 antibody or antigen-binding fragment thereof comprises V_{H} and V_{L} regions or CDRs selected from Table 16. In certain embodiments, the anti-DLL3 scFv comprises a V_{H} comprising the amino acid sequence set forth in SEQ ID NO: 126, as shown in Table 16. An exemplary nucleotide sequence encoding the amino acid sequence of SEQ ID NO: 126 is set forth in SEQ ID NO: 128. In certain embodiments, the anti-DLL3 antibody or antigen-binding fragment thereof comprises a V_{L} comprising the amino acid sequence set forth in SEQ ID NO: 127. An exemplary nucleotide sequence encoding the amino acid sequence of SEQ ID NO: 127 is set forth in SEQ ID NO: 129. In certain embodiments, the anti-DLL3 antibody or antigen-binding fragment thereof comprises a V_{H} comprising the amino acid sequence set forth in SEQ ID NO: 126 and a V_{L} comprising the amino acid sequence set forth in SEQ ID NO: 127. SEQ ID NO: 126-129 are provided in Table 16. In certain embodiments, the antibody or antigen-binding fragment thereof is designated as “1F9”.

In certain embodiments, the anti-DLL3 antibody or antigen-binding fragment thereof comprises a V_{H} comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 123 or a conservative modification thereof. SEQ ID NOs: 2, 21, and 123 are provided in Table 16.

In certain embodiments, the anti-DLL3 antibody or antigen-binding fragment thereof comprises a V_{L} comprising a CDR1 comprising the amino acid sequence set forth in SEQ ID NO: 124 or a conservative modification thereof, a CDR2 comprising the amino acid sequence set forth in SEQ ID NO: 58 or a conservative modification thereof, and a CDR3 comprising the amino acid sequence set forth in SEQ ID NO: 125 or a conservative modification thereof. SEQ ID NOs: 58, 124, and 125 are provided in Table 16.

In certain embodiments, the anti-DLL3 antibody or antigen-binding fragment thereof comprises a V_{H} comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 123 or a conservative modification thereof; and a V_{L} comprising a CDR1 comprising the amino acid sequence set forth in SEQ ID NO: 124 or a conservative modification thereof, a CDR2 comprising the amino acid sequence set forth in SEQ ID NO: 58 or a conservative modification thereof, and a CDR3 comprising the amino acid sequence set forth in SEQ ID NO: 125 or a conservative modification.

In certain embodiments, the anti-DLL3 antibody or antigen-binding fragment thereof comprises a V_{H} comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 123; and a V_{L} comprising a CDR1 comprising the amino acid sequence set forth in SEQ ID NO: 124, a CDR2 comprising the amino acid sequence set forth in SEQ ID NO: 58, and a CDR3 comprising the amino acid sequence set forth in SEQ ID NO: 125.

TABLE 16

| CDRs | 1 | 2 | 3 |
|---|---|---|---|
| V_{H} | GYTFTSY | NPSGGS | QGGAQWLVLAFDI |
| | [SEQ ID NO: 21] | [SEQ ID NO: 2] | [SEQ ID NO: 123] |
| V_{L} | RASQSVSSYLA | GASSRAT | QQYDSSPYT |
| | [SEQ ID NO: 124] | [SEQ ID NO: 58] | [SEQ ID NO: 125] |

| | |
|---|---|
| Full V_{H} | QVQLVQSGAEVKKPGASVQVSCKASGYT FTSYYMHWVRQAPGQGLEWMGIINPS |
| | GGSTSYAQKFQGRVTMTRDTSTSTVYMELSSLRSEDTAVYYCARQGGAQWLVLA |
| | FDIWGQGTMVTVSS [SEQ ID NO: 126] |
| Full V_{L} | EIVLTQSPGTLSLSPGERATLSCRASQSVSSYLAWYQQKPGQPPRLLIYGASSR |
| | ATGIPDRESGGGSGTDETLTISRLEPEDFAVYYCQQYDSSPYTFGQGTKLEIK |
| | [SEQ ID NO: 127] |
| DNA for | CAGGTGCAGCTGGTGCAGTCTGGGGCTGAGGTGAAGAAGCCTGGGGCCTCAGTG |
| Full V_{H} | CAGGTTTCCTGCAAGGCATCTGGATACACCTTCACCAGCTACTATATGCACTGG |
| | GTGCGACAGGCCCCTGGACAAGGGCTTGAGTGGATGGGAATAATCAACCCTAGT |
| | GGTGGTAGCACAAGCTACGCACAGAAGTTCCAGGGCAGAGTCACCATGACCAGG |
| | GACACGTCCACGAGCACAGTCTACATGGAGCTGAGCAGCCTGAGATCTGAGGAC |
| | ACGGCCGTGTATTACTGTGCGAGACAGGGGGGAGCCCAGTGGCTGGTACTTGCT |
| | TTTGATATCTGGGGCCAAGGGACAATGGTCACCGTCTCTTCA |
| | [SEQ ID NO: 128] |
| DNA for | GAAATTGTGTTGACGCAGTCTCCAGGCACCCTGTCTTTGTCTCCAGGGGAAAGA |
| Full V_{L} | GCCACCCTCTCCTGCAGGGCCAGTCAGAGTGTTAGCAGCTACTTAGCCTGGTAC |
| | CAGCAGAAACCTGGCCAGCCTCCCAGGCTCCTCATCTATGGTGCATCCAGCAGG |
| | GCCACTGGCATCCCAGACAGGTTCAGTGGCGGTGGGTCTGGGACAGACTTCACT |
| | CTCACCATCAGCAGACTGGAGCCTGAAGATTTTGCAGTGTATTACTGTCAGCAG |
| | TATGATAGCTCACCGTACACTTTTGGCCAGGGGACCAAGCTGGAGATCAAA |
| | [SEQ ID NO: 129] |

In certain embodiments, the anti-DLL3 antibody or antigen-binding fragment thereof comprises V_{H} and V_{L} regions or CDRs selected from Table 17. In certain embodiments, the anti-DLL3 antibody or antigen-binding fragment thereof comprises a V_{H} comprising the amino acid sequence set forth in SEQ ID NO: 131, as shown in Table 17. An exemplary nucleotide sequence encoding the amino acid sequence of SEQ ID NO: 131 is set forth in SEQ ID NO: 133. In certain embodiments, the anti-DLL3 antibody or antigen-binding fragment thereof comprises a V_{L} comprising the amino acid sequence set forth in SEQ ID NO: 132. An exemplary nucleotide sequence encoding the amino acid sequence of SEQ ID NO: 132 is set forth in SEQ ID NO: 134. In certain embodiments, the anti-DLL3 antibody or antigen-binding fragment thereof comprises a V_{H} comprising the amino acid sequence set forth in SEQ ID NO: 131 and a V_{L} Comprising the amino acid sequence set forth in SEQ ID NO: 132. SEQ ID NO: 131-134 are provided in Table 17. In certain embodiments, the antibody or antigen-binding fragment thereof is designated as “L12”.

In certain embodiments, the anti-DLL3 antibody or antigen-binding fragment thereof comprises a V_{H} comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 56 or a conservative modification thereof. SEQ ID NOs: 2, 21, and 56 are provided in Table 17.

In certain embodiments, the anti-DLL3 antibody or antigen-binding fragment thereof comprises a V_{L} comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 130 or a conservative modification thereof. SEQ ID NOs: 57, 58, and 130 are provided in Table 17.

In certain embodiments, the anti-DLL3 antibody or antigen-binding fragment thereof comprises a V_{H} comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 56 or a conservative modification thereof; and a V_{L} comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 130 or a conservative modification.

In certain embodiments, the anti-DLL3 antibody or antigen-binding fragment thereof comprises a V_{H} comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 56; and a V_{L} Comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 130.

TABLE 17

| CDRs | 1 | 2 | 3 |
|---|---|---|---|
| V_{H} | GYTFTSY | NPSGGS | QLELLGNAFDI |
| | [SEQ ID NO: 21] | [SEQ ID NO: 2] | [SEQ ID NO: 56] |
| V_{L} | RASQSVSSSYLA | GASSRAT | QQYGNSPYT |
| | [SEQ ID NO: 57] | [SEQ ID NO: 58] | [SEQ ID NO: 130] |

| | |
|---|---|
| Full V_{H} | QVQLVQSGAEVKKPGASVKVSCKASGYTFTSYYMHWVRQAPGQGLEWMGIINPS |
| | GGSRSYAQKFQGRVTMTRDTSTSTVYMELSSLRSEDTAVYYCARQLELLGNAFD |
| | IWGQGTMVTVSS |
| | [SEQ ID NO: 131] |
| Full V_{L} | EIVLTQSPGTLSLSPGERATLSCRASQSVSSSYLAWYQQKPGQAPRLLIYGASS |
| | RATGIPDRESGSGSGTDFTLTISRLEPEDFAVYYCQQYGNSPYTFGQGTKLEIK |
| | [SEQ ID NO: 132] |
| DNA for | CAGGTGCAGCTGGTGCAGTCTGGGGCTGAGGTGAAGAAGCCTGGGGCCTCAGTG |
| Full V_{H} | AAGGTTTCCTGCAAGGCATCTGGATACACCTTCACCAGCTACTATATGCACTGG |
| | GTGCGACAGGCCCCTGGACAAGGGCTTGAGTGGATGGGAATAATCAACCCTAGT |
| | GGTGGTAGTAGAAGCTACGCACAGAAGTTCCAGGGCAGAGTCACTATGACCAGG |
| | GACACGTCCACGAGCACAGTCTACATGGAGCTGAGCAGCCTGAGATCTGAGGAC |
| | ACGGCCGTGTATTACTGTGCGAGACAACTGGAACTTTTGGGGAATGCTTTTGAT |
| | ATCTGGGGCCAAGGGACAATGGTCACCGTCTCTTCA |
| | [SEQ ID NO: 133] |
| DNA for | GAAATTGTGTTGACGCAGTCTCCAGGCACCCTGTCTTTGTCTCCAGGGGAAAGA |
| Full V_{L} | GCCACCCTCTCCTGCAGGGCCAGTCAGAGTGTTAGCAGCAGCTACTTAGCCTGG |
| | TACCAGCAGAAACCTGGCCAGGCTCCCAGGCTCCTCATCTATGGTGCATCCAGC |
| | AGGGCCACTGGCATCCCAGACAGGTTCAGTGGCAGTGGGTCTGGGACAGACTTC |
| | ACTCTCACCATCAGCAGACTGGAGCCTGAAGATTTTGCAGTGTATTACTGTCAG |
| | CAGTATGGTAACTCGCCGTACACTTTTGGCCAGGGGACCAAGCTGGAGATCAAA |
| | [SEQ ID NO: 134] |

In certain embodiments, the anti-DLL3 antibody or antigen-binding fragment thereof comprises V_{H} and V_{L} regions or CDRs selected from Table 18. In certain embodiments, the anti-DLL3 antibody or antigen-binding fragment thereof comprises a V_{H} comprising the amino acid sequence set forth in SEQ ID NO: 141, as shown in Table 18. An exemplary nucleotide sequence encoding the amino acid sequence of SEQ ID NO: 141 is set forth in SEQ ID NO: 143. In certain embodiments, the anti-DLL3 antibody or antigen-binding fragment thereof comprises a V_{L} comprising the amino acid sequence set forth in SEQ ID NO: 142. An exemplary nucleotide sequence encoding the amino acid sequence of SEQ ID NO: 142 is set forth in SEQ ID NO: 144. In certain embodiments, the anti-DLL3 antibody or antigen-binding fragment thereof comprises a V_{H} comprising the amino acid sequence set forth in SEQ ID NO: 141 and a V_{L} Comprising the amino acid sequence set forth in SEQ ID NO: 142. SEQ ID NO: 141-144 are provided in Table 18. In certain embodiments, the antibody or antigen-binding fragment thereof is designated as “B22”.

In certain embodiments, the anti-DLL3 antibody or antigen-binding fragment thereof comprises a V_{H} comprising a CDR1 comprising the amino acid sequence set forth in SEQ ID NO: 135 or a conservative modification thereof, a CDR2 comprising the amino acid sequence set forth in SEQ ID NO: 136 or a conservative modification thereof, and a CDR3 comprising the amino acid sequence set forth in SEQ ID NO: 137 or a conservative modification thereof. SEQ ID NOs: 135-137 are provided in Table 18.

In certain embodiments, the anti-DLL3 antibody or antigen-binding fragment thereof comprises a V_{L} comprising a CDR1 comprising the amino acid sequence set forth in SEQ ID NO: 138 or a conservative modification thereof, a CDR2 comprising the amino acid sequence set forth in SEQ ID NO: 139 or a conservative modification thereof, and a CDR3 comprising the amino acid sequence set forth in SEQ ID NO: 140 or a conservative modification thereof. SEQ ID NOs: 138-140 are provided in Table 18.

In certain embodiments, the anti-DLL3 antibody or antigen-binding fragment thereof comprises a V_{H} comprising a CDR1 comprising the amino acid sequence set forth in SEQ ID NO: 135 or a conservative modification thereof, a CDR2 comprising the amino acid sequence set forth in SEQ ID NO: 136 or a conservative modification thereof, and a CDR3 comprising the amino acid sequence set forth in SEQ ID NO: 137 or a conservative modification thereof; and a V_{L} comprising a CDR1 comprising the amino acid sequence set forth in SEQ ID NO: 138 or a conservative modification thereof, a CDR2 comprising the amino acid sequence set forth in SEQ ID NO: 139 or a conservative modification thereof, and a CDR3 comprising the amino acid sequence set forth in SEQ ID NO: 140 or a conservative modification.

In certain embodiments, the anti-DLL3 antibody or antigen-binding fragment thereof comprises a V_{H} comprising a CDR1 comprising the amino acid sequence set forth in SEQ ID NO: 135, a CDR2 comprising the amino acid sequence set forth in SEQ ID NO: 136, and a CDR3 comprising the amino acid sequence set forth in SEQ ID NO: 137; and a V_{L} comprising a CDR1 comprising the amino acid sequence set forth in SEQ ID NO: 138, a CDR2 comprising the amino acid sequence set forth in SEQ ID NO: 139, and a CDR3 comprising the amino acid sequence set forth in SEQ ID NO: 140.

TABLE 18

| CDRs | 1 | 2 | 3 |
|---|---|---|---|
| V_{H} | GFTENNF | KQDGSE | ESYFDL |
| | [SEQ ID NO: 135] | [SEQ ID NO: 136] | [SEQ ID NO: 137] |
| V_{L} | RSSTGAVTTSNYAN | GTNNRAP | ALWYSNRWV |
| | [SEQ ID NO: 138] | [SEQ ID NO: 139] | [SEQ ID NO: 140] |

| | |
|---|---|
| Full V_{H} | EVQLVESGGGLVQPGGSLRLSCAASGFTENNFWMSWVRQAPGKGLEWVANIKQD |
| | GSEKYYVDSVKGRFTISRDNAKNSLYLQMNSLRAEDTAVYYCARESYFDLWGRG |
| | TLVTVSS |
| | [SEQ ID NO: 141] |
| Full V_{L} | QAVVTQESALTTSPGETVTLTCRSSTGAVTTSNYANWVQEKPDHLFTGLIGGTN |
| | NRAPGVPARESGSLIGDKAALTITGAQTEDEAIYFCALWYSNRWVEGGGTKLTV |
| | L [SEQ ID NO: 142] |
| DNA for | GAGGTGCAGCTGGTGGAGTCTGGGGGAGGCTTGGTCCAGCCTGGGGGGTCCCTG |
| Full V_{H} | AGACTCTCCTGTGCAGCCTCTGGATTCACCTTTAATAACTTTTGGATGAGCTGG |
| | GTCCGCCAGGCTCCAGGGAAGGGGCTGGAGTGGGTGGCCAACATAAAGCAAGAT |
| | GGAAGTGAGAAATACTATGTGGACTCTGTGAAGGGCCGATTCACCATCTCCAGA |
| | GACAACGCCAAGAACTCACTGTATCTGCAAATGAACAGCCTGAGAGCCGAGGAC |
| | ACGGCTGTGTATTACTGTGCGAGAGAGAGTTACTTCGATCTCTGGGGCCGTGGC |
| | ACCCTGGTCACTGTCTCCTCA |
| | [SEQ ID NO: 143] |
| DNA for | CAGGCTGTTGTGACTCAGGAATCTGCACTCACCACATCACCTGGTGAAACAGTC |
| Full V_{L} | ACACTCACTTGTCGCTCAAGTACTGGGGCTGTTACAACTAGTAACTATGCCAAC |
| | TGGGTCCAAGAAAAACCAGATCATTTATTCACTGGTCTAATAGGTGGTACCAAC |
| | AACCGAGCTCCAGGTGTTCCTGCCAGATTCTCAGGCTCCCTGATTGGAGACAAG |
| | GCTGCCCTCACCATCACAGGGGCACAGACTGAGGATGAGGCAATATATTTCTGT |
| | GCTCTATGGTACAGCAACCGCTGGGTGTTCGGTGGAGGAACCAAACTGACTGTC |
| | CTA |
| | [SEQ ID NO: 144] |

In certain embodiments, the anti-DLL3 antibody or antigen-binding fragment thereof comprises V_{H} and V_{L} regions or CDRs selected from Table 19. In certain embodiments, the anti-DLL3 antibody or antigen-binding fragment thereof comprises a V_{H} comprising the amino acid sequence set forth in SEQ ID NO: 147, as shown in Table 19. An exemplary nucleotide sequence encoding the amino acid sequence of SEQ ID NO: 147 is set forth in SEQ ID NO: 149. In certain embodiments, the anti-DLL3 antibody or antigen-binding fragment thereof comprises a V_{L} comprising the amino acid sequence set forth in SEQ ID NO: 148. An exemplary nucleotide sequence encoding the amino acid sequence of SEQ ID NO: 148 is set forth in SEQ ID NO: 150. In certain embodiments, the anti-DLL3 antibody or antigen-binding fragment thereof comprises a V_{H} comprising the amino acid sequence set forth in SEQ ID NO: 147 and a V_{L} Comprising the amino acid sequence set forth in SEQ ID NO: 148. SEQ ID NO: 147-150 are provided in Table 19. In certain embodiments, the antibody or antigen-binding fragment thereof is designated as “C22”.

In certain embodiments, the anti-DLL3 antibody or antigen-binding fragment thereof comprises a V_{H} comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 145 or a conservative modification thereof. SEQ ID NOs: 2, 21, and 145 are provided in Table 19.

In certain embodiments, the anti-DLL3 antibody or antigen-binding fragment thereof comprises a V_{L} comprising a CDR1 comprising the amino acid sequence set forth in SEQ ID NO: 57 or a conservative modification thereof, a CDR2 comprising the amino acid sequence set forth in SEQ ID NO: 146 or a conservative modification thereof, and a CDR3 comprising the amino acid sequence set forth in SEQ ID NO: 125 or a conservative modification thereof. SEQ ID NOs: 57, 125, and 146 are provided in Table 19.

In certain embodiments, the anti-DLL3 antibody or antigen-binding fragment thereof comprises a V_{H} comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 145 or a conservative modification thereof; and a V_{L} comprising a CDR1 comprising the amino acid sequence set forth in SEQ ID NO: 57 or a conservative modification thereof, a CDR2 comprising the amino acid sequence set forth in SEQ ID NO: 146 or a conservative modification thereof, and a CDR3 comprising the amino acid sequence set forth in SEQ ID NO: 125 or a conservative modification.

In certain embodiments, the anti-DLL3 antibody or antigen-binding fragment thereof comprises a V_{H} comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 145; and a V_{L} comprising a CDR1 comprising the amino acid sequence set forth in SEQ ID NO: 57, a CDR2 comprising the amino acid sequence set forth in SEQ ID NO: 146, and a CDR3 comprising the amino acid sequence set forth in SEQ ID NO: 125.

TABLE 19

| CDRs | 1 | 2 | 3 |
|---|---|---|---|
| V_{H} | GYTFTSY | NPSGGS | VADIVGATEDY |
| | [SEQ ID NO: 21] | [SEQ ID NO: 2] | [SEQ ID NO: 145] |
| V_{L} | RASQSVSSSYLA | GASSWAT | QQYDSSPYT |
| | [SEQ ID NO: 57] | [SEQ ID NO: 146] | [SEQ ID NO: 125] |

| | |
|---|---|
| Full V_{H} | QVQLVQSGAEVKKPGASVKVSCKASGYTFTSYYMHWVRQAPGQGLEWMGIINPS |
| | GGSTSYAQKFQGRVTMTRDTSTSTVYMELSSLRSEDTAVYYCARVADIVGATED |
| | YWGQGTLVTVSS |
| | [SEQ ID NO: 147] |
| Full V_{L} | EIVLTQSPGTLSLSPGERATLSCRASQSVSSSYLAWYQQKPGQAPRLLIYGASS |
| | WATGIPDRESGSGSGTDETLTISRLEPEDFAVYYCQQYDSSPYTFGQGTKLEIK |
| | [SEQ ID NO: 148] |
| DNA for | CAGGTGCAGCTGGTGCAGTCTGGGGCTGAGGTGAAGAAGCCTGGGGCCTCAGTG |
| Full V_{H} | AAAGTTTCCTGCAAGGCATCTGGATACACCTTCACCAGCTACTATATGCACTGG |
| | GTGCGACAGGCCCCTGGACAAGGGCTTGAGTGGATGGGAATAATCAACCCTAGT |
| | GGTGGTAGCACTAGCTACGCACAGAAGTTCCAGGGCAGAGTCACCATGACCAGG |
| | GACACGTCCACGAGCACAGTCTACATGGAGCTGAGCAGCCTGAGATCTGAGGAC |
| | ACGGCCGTGTATTACTGTGCGAGAGTGGCGGACATAGTGGGAGCTACTTTTGAC |
| | TACTGGGGCCAGGGAACCCTGGTCACCGTCTCCTCA |
| | [SEQ ID NO: 149] |
| DNA for | GAAATTGTGTTGACGCAGTCTCCAGGCACCCTGTCTTTGTCTCCAGGGGAAAGA |
| Full V_{L} | GCCACCCTCTCCTGCAGGGCCAGTCAGAGTGTTAGCAGCAGCTACTTAGCCTGG |
| | TACCAGCAGAAACCTGGCCAGGCTCCCAGGCTCCTCATCTATGGTGCATCCAGC |
| | TGGGCCACTGGCATCCCAGACAGGTTCAGTGGCAGTGGGTCTGGGACAGACTTC |
| | ACTCTCACCATCAGCAGACTGGAACCTGAAGATTTTGCAGTGTATTACTGTCAG |
| | CAGTATGATAGCTCACCCTACACTTTTGGCCAGGGGACCAAGCTGGAGATCAAA |
| | [SEQ ID NO: 150] |

In certain embodiments, the anti-DLL3 antibody or antigen-binding fragment thereof comprises V_{H} and V_{L} regions or CDRs selected from Table 20. In certain embodiments, the anti-DLL3 antibody or antigen-binding fragment thereof comprises a V_{H} comprising the amino acid sequence set forth in SEQ ID NO: 153, as shown in Table 20. An exemplary nucleotide sequence encoding the amino acid sequence of SEQ ID NO: 153 is set forth in SEQ ID NO: 155. In certain embodiments, the anti-DLL3 antibody or antigen-binding fragment thereof comprises a V_{L} comprising the amino acid sequence set forth in SEQ ID NO: 154. An exemplary nucleotide sequence encoding the amino acid sequence of SEQ ID NO: 154 is set forth in SEQ ID NO: 156. In certain embodiments, the anti-DLL3 antibody or antigen-binding fragment thereof comprises a V_{H} comprising the amino acid sequence set forth in SEQ ID NO: 153 and a V_{L} Comprising the amino acid sequence set forth in SEQ ID NO: 154. SEQ ID NO: 153-156 are provided in Table 20. In certain embodiments, the antibody or antigen-binding fragment thereof is designated as “D8”.

In certain embodiments, the anti-DLL3 antibody or antigen-binding fragment thereof comprises a V_{H} comprising a CDR1 comprising the amino acid sequence set forth in SEQ ID NO: 151 or a conservative modification thereof, a CDR2 comprising the amino acid sequence set forth in SEQ ID NO: 2 or a conservative modification thereof, and a CDR3 comprising the amino acid sequence set forth in SEQ ID NO: 152 or a conservative modification thereof. SEQ ID NOs: 2, 151, and 152 are provided in Table 20.

In certain embodiments, the anti-DLL3 antibody or antigen-binding fragment thereof comprises a V_{L} comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 82 or a conservative modification thereof. SEQ ID NOs: 57, 58, and 82 are provided in Table 20.

In certain embodiments, the anti-DLL3 antibody or antigen-binding fragment thereof comprises a V_{H} comprising a CDR1 comprising the amino acid sequence set forth in SEQ ID NO: 151 or a conservative modification thereof, a CDR2 comprising the amino acid sequence set forth in SEQ ID NO: 2 or a conservative modification thereof, and a V_{H} CDR3 comprising the amino acid sequence set forth in SEQ ID NO: 152 or a conservative modification thereof; and a V_{L} comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 82 or a conservative modification.

In certain embodiments, the anti-DLL3 antibody or antigen-binding fragment thereof comprises a V_{H} comprising a CDR1 comprising the amino acid sequence set forth in SEQ ID NO: 151, a CDR2 comprising the amino acid sequence set forth in SEQ ID NO: 2, and a CDR3 comprising the amino acid sequence set forth in SEQ ID NO: 152; and a V_{L} comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82.

TABLE 20

| CDRs | 1 | 2 | 3 |
|---|---|---|---|
| V_{H} | GYTFTTY | NPSGGS | QGHYIGNYLDY |
| | [SEQ ID NO: 151] | [SEQ ID NO: 2] | [SEQ ID NO: 152] |
| V_{L} | RASQSVSSSYLA | GASSRAT | QQYGSSP |
| | [SEQ ID NO: 57] | [SEQ ID NO: 58] | [SEQ ID NO: 82] |

| | |
|---|---|
| Full V_{H} | QVQLVQSGAEVKKPGASVKVSCKASGYTFTTYYMHWVRQAPGQGLEWMGIINPS |
| | GGSTSYAQKFQGRVTMTRDTSTSTVYMELSSLRSEDTAVYYCARQGHYIGNYLD |
| | YWGQGTLVTVSS |
| | [SEQ ID NO: 153] |
| Full V_{L} | EIVLTQSPGTLSLSPGERATLSCRASQSVSSSYLAWYQQKPGQAPRLLIYGASS |
| | RATGIPDRESGSGSGTDETLTISRLEPEDFAVYYCQQYGSSPFGQGTKLEIK |
| | [SEQ ID NO: 154] |
| DNA for | CAGGTGCAGCTGGTGCAGTCTGGGGCTGAGGTGAAGAAGCCTGGGGCCTCAGTG |
| Full V_{H} | AAGGTTTCCTGCAAGGCATCTGGATACACCTTCACCACCTACTATATGCACTGG |
| | GTGCGACAGGCCCCTGGACAAGGGCTTGAGTGGATGGGAATAATCAACCCTAGT |
| | GGTGGTAGCACAAGCTACGCACAGAAGTTCCAGGGCAGAGTCACCATGACCAGG |
| | GACACGTCCACGAGCACAGTCTACATGGAGCTGAGCAGCCTGAGATCTGAGGAC |
| | ACGGCCGTGTATTACTGTGCGAGACAGGGCCATTATATTGGGAACTACCTTGAC |
| | TACTGGGGCCAGGGAACCCTGGTCACCGTCTCCTCA |
| | [SEQ ID NO: 155] |
| DNA for | GAAATTGTGTTGACGCAGTCTCCAGGCACCCTGTCTTTGTCTCCAGGGGAAAGA |
| Full V_{L} | GCCACCCTCTCCTGCAGGGCCAGTCAGAGTGTTAGCAGCAGCTACTTAGCCTGG |
| | TACCAGCAGAAACCTGGCCAGGCTCCCAGGCTCCTCATCTATGGTGCATCCAGC |
| | AGGGCCACTGGCATCCCAGACAGGTTCAGTGGCAGTGGGTCTGGGACAGACTTC |
| | ACTCTCACCATCAGCAGACTGGAGCCTGAAGATTTTGCAGTGTATTACTGTCAG |
| | CAGTATGGTAGCTCACCTTTTGGCCAGGGGACCAAGCTGGAGATCAAA |
| | [SEQ ID NO: 156] |

In certain embodiments, the anti-DLL3 antibody or antigen-binding fragment thereof comprises V_{H} and V_{L} regions or CDRs selected from Table 21. In certain embodiments, the anti-DLL3 antibody or antigen-binding fragment thereof comprises a V_{H} comprising the amino acid sequence set forth in SEQ ID NO: 157, as shown in Table 21. An exemplary nucleotide sequence encoding the amino acid sequence of SEQ ID NO: 157 is set forth in SEQ ID NO: 159. In certain embodiments, the anti-DLL3 antibody or antigen-binding fragment thereof comprises a V_{L} comprising the amino acid sequence set forth in SEQ ID NO: 158. An exemplary nucleotide sequence encoding the amino acid sequence of SEQ ID NO: 158 is set forth in SEQ ID NO: 160. In certain embodiments, the anti-DLL3 antibody or antigen-binding fragment thereof comprises a V_{H} comprising the amino acid sequence set forth in SEQ ID NO: 157 and a V_{L} Comprising the amino acid sequence set forth in SEQ ID NO: 158. SEQ ID NO: 157-160 are provided in Table 21. In certain embodiments, the antibody or antigen-binding fragment thereof is designated as “G16”.

In certain embodiments, the anti-DLL3 antibody or antigen-binding fragment thereof comprises a V_{H} comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 123 or a conservative modification thereof. SEQ ID NOs: 2, 21, and 123 are provided in Table 21.

In certain embodiments, the anti-DLL3 antibody or antigen-binding fragment thereof comprises a V_{L} comprising a CDR1 comprising the amino acid sequence set forth in SEQ ID NO: 124 or a conservative modification thereof, a CDR2 comprising the amino acid sequence set forth in SEQ ID NO: 58 or a conservative modification thereof, and a CDR3 comprising the amino acid sequence set forth in SEQ ID NO: 59 or a conservative modification thereof. SEQ ID NOs: 58, 59, and 124 are provided in Table 21.

In certain embodiments, the anti-DLL3 antibody or antigen-binding fragment thereof comprises a V_{H} comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 123 or a conservative modification thereof; and a V_{L} comprising a CDR1 comprising the amino acid sequence set forth in SEQ ID NO: 124 or a conservative modification thereof, a CDR2 comprising the amino acid sequence set forth in SEQ ID NO: 58 or a conservative modification thereof, and a CDR3 comprising the amino acid sequence set forth in SEQ ID NO: 59 or a conservative modification.

In certain embodiments, the anti-DLL3 antibody or antigen-binding fragment thereof comprises a V_{H} comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 123; and a V_{L} comprising a CDR1 comprising the amino acid sequence set forth in SEQ ID NO: 124, a CDR2 comprising the amino acid sequence set forth in SEQ ID NO: 58, and a CDR3 comprising the amino acid sequence set forth in SEQ ID NO: 59.

TABLE 21

| CDRs | 1 | 2 | 3 |
|---|---|---|---|
| V_{H} | GYTFTSY | NPSGGS | QGGAQWLVLAFDI |
| | [SEQ ID NO: 21] | [SEQ ID NO: 2] | [SEQ ID NO: 123] |
| V_{L} | RASQSVSSYLA | GASSRAT | QQYGSSPYT |
| | [SEQ ID NO: 124] | [SEQ ID NO: 58] | [SEQ ID NO: 59] |

| | |
|---|---|
| Full V_{H} | QVQLVQSGAEVKKPGASVQVSCKASGYTFTSYYMHWVRQAPGQGLEWMGIINPS |
| | GGSTSYAQKFQGRVTMTRDTSTSTVYMELSSLRSEDTAVYYCARQGGAQWLVLA |
| | FDIWGQGTMVTVSS |
| | [SEQ ID NO: 157] |
| Full V_{L} | EIVLTQSPGTLSLSPGERATLSCRASQSVSSYLAWYQQKPGQAPRLLIYGASSR |
| | ATGIPDRESGGGSGTDFTLTISRLEPEDFAVYYCQQYGSSPYTFGQGTKLEIK |
| | [SEQ ID NO: 158] |
| DNA for | CAGGTGCAGCTGGTGCAGTCTGGGGCTGAGGTGAAGAAGCCTGGGGCCTCAGTG |
| Full V_{H} | CAGGTTTCCTGCAAGGCATCTGGATACACCTTCACCAGCTACTATATGCACTGG |
| | GTGCGACAGGCCCCTGGACAAGGGCTTGAGTGGATGGGAATAATCAACCCTAGT |
| | GGTGGTAGCACAAGCTACGCACAGAAGTTCCAGGGCAGAGTCACCATGACCAGG |
| | GACACGTCCACGAGCACAGTCTACATGGAGCTGAGCAGCCTGAGATCTGAGGAC |
| | ACGGCCGTGTATTACTGTGCGAGACAGGGGGGAGCCCAGTGGCTGGTACTTGCT |
| | TTTGATATCTGGGGCCAAGGGACAATGGTCACCGTCTCTTCA |
| | [SEQ ID NO: 159] |
| DNA for | GAAATTGTGTTGACGCAGTCTCCAGGCACCCTGTCTTTGTCTCCAGGGGAAAGA |
| Full V_{L} | GCCACCCTCTCCTGCAGGGCCAGTCAGAGTGTTAGCAGCTACTTAGCCTGGTAC |
| | CAGCAGAAACCTGGCCAGGCTCCCAGGCTCCTCATCTATGGTGCATCCAGCAGG |
| | GCCACTGGCATCCCAGACAGGTTCAGTGGCGGTGGGTCTGGGACAGACTTCACT |
| | CTCACCATCAGCAGACTGGAGCCTGAAGATTTTGCAGTGTATTACTGTCAGCAG |
| | TATGGTAGCTCACCGTACACTTTTGGCCAGGGGACCAAGCTGGAGATCAAA |
| | [SEQ ID NO: 160] |

In certain embodiments, the anti-DLL3 antibody or antigen-binding fragment thereof comprises V_{H} and V_{L} regions or CDRs selected from Table 22. In certain embodiments, the anti-DLL3 antibody or antigen-binding fragment thereof comprises a V_{H} comprising the amino acid sequence set forth in SEQ ID NO: 163, as shown in Table 22. An exemplary nucleotide sequence encoding the amino acid sequence of SEQ ID NO: 163 is set forth in SEQ ID NO: 165. In certain embodiments, the anti-DLL3 antibody or antigen-binding fragment thereof comprises a V_{L} comprising the amino acid sequence set forth in SEQ ID NO: 164. An exemplary nucleotide sequence encoding the amino acid sequence of SEQ ID NO: 164 is set forth in SEQ ID NO: 166. In certain embodiments, the anti-DLL3 antibody or antigen-binding fragment thereof comprises a V_{H} comprising the amino acid sequence set forth in SEQ ID NO: 163 and a V_{L} Comprising the amino acid sequence set forth in SEQ ID NO: 164. SEQ ID NO: 163-166 are provided in Table 22. In certain embodiments, the antibody or antigen-binding fragment thereof is designated as “F21”.

In certain embodiments, the anti-DLL3 antibody or antigen-binding fragment thereof comprises a V_{H} comprising a CDR1 comprising the amino acid sequence set forth in SEQ ID NO: 11 or a conservative modification thereof, a CDR2 comprising the amino acid sequence set forth in SEQ ID NO: 136 or a conservative modification thereof, and a CDR3 comprising the amino acid sequence set forth in SEQ ID NO: 161 or a conservative modification thereof. SEQ ID NOs: 11, 136, and 161 are provided in Table 22.

In certain embodiments, the anti-DLL3 antibody or antigen-binding fragment thereof comprises a V_{L} comprising a CDR1 comprising the amino acid sequence set forth in SEQ ID NO: 73 or a conservative modification thereof, a CDR2 comprising the amino acid sequence set forth in SEQ ID NO: 74 or a conservative modification thereof, and a CDR3 comprising the amino acid sequence set forth in SEQ ID NO: 162 or a conservative modification thereof. SEQ ID NOs: 73, 74, and 162 are provided in Table 22.

In certain embodiments, the anti-DLL3 antibody or antigen-binding fragment thereof comprises a V_{H} comprising a CDR1 comprising the amino acid sequence set forth in SEQ ID NO: 11 or a conservative modification thereof, a CDR2 comprising the amino acid sequence set forth in SEQ ID NO: 136 or a conservative modification thereof, and a CDR3 comprising the amino acid sequence set forth in SEQ ID NO: 161 or a conservative modification thereof; and a V_{L} comprising a CDR1 comprising the amino acid sequence set forth in SEQ ID NO: 73 or a conservative modification thereof, a CDR2 comprising the amino acid sequence set forth in SEQ ID NO: 74 or a conservative modification thereof, and a CDR3 comprising the amino acid sequence set forth in SEQ ID NO: 162 or a conservative modification.

In certain embodiments, the anti-DLL3 antibody or antigen-binding fragment thereof comprises a V_{H} comprising a CDR1 comprising the amino acid sequence set forth in SEQ ID NO: 11, a CDR2 comprising the amino acid sequence set forth in SEQ ID NO: 136, and a CDR3 comprising the amino acid sequence set forth in SEQ ID NO: 161; and a V_{L} comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 162.

TABLE 22

| CDRs | 1 | 2 | 3 |
|---|---|---|---|
| V_{H} | GFTESSY | KQDGSE | EWLRFGGLVY |
| | [SEQ ID NO: 11] | [SEQ ID NO: 136] | [SEQ ID NO: 161] |
| V_{L} | RASQSVSSNLA | GASTRAT | QQYNNWPLT |
| | [SEQ ID NO: 73] | [SEQ ID NO: 74] | [SEQ ID NO: 162] |

| | |
|---|---|
| Full V_{H} | EVQLVESGGGLVQPGGSLRLSCAASGFTFSSYWMSWVRQAPGKGLEWVANIKQD |
| | GSEKYYVDSVKGRFTISRDNAKNSLYLQMNSLRAEDTAVYYCAREWLREGGLVY |
| | WGQGTLVTVSS |
| | [SEQ ID NO: 163] |
| Full V_{L} | EIVMTQSPATLSVSPGERATLSCRASQSVSSNLAWYQQKPGQAPRLLIYGASTR |
| | ATGIPARFSGSGSGTEFTLTISSLQSEDFAVYYCQQYNNWPLTFGGGTKVEIK |
| | [SEQ ID NO: 164] |
| DNA for | GAGGTGCAGCTGGTGGAGTCTGGGGGAGGCTTGGTCCAGCCTGGGGGGTCCCTG |
| Full V_{H} | AGACTCTCCTGTGCAGCCTCTGGATTCACCTTTAGTAGCTATTGGATGAGCTGG |
| | GTCCGCCAGGCTCCAGGGAAGGGGCTGGAGTGGGTGGCCAACATAAAGCAAGAT |
| | GGAAGTGAGAAATACTATGTGGACTCTGTGAAGGGCCGATTCACCATCTCCAGA |
| | GACAACGCCAAGAACTCACTGTATCTGCAAATGAACAGCCTGAGAGCCGAGGAC |
| | ACGGCTGTGTATTACTGTGCGAGAGAGTGGCTACGATTTGGGGGCTTAGTGTAC |
| | TGGGGCCAGGGAACCCTGGTCACCGTCTCCTCA |
| | [SEQ ID NO: 165] |
| DNA for | GAAATAGTGATGACGCAGTCTCCAGCCACCCTGTCTGTGTCTCCAGGGGAAAGA |
| Full V_{L} | GCCACCCTCTCCTGCAGGGCCAGTCAGAGTGTTAGCAGCAACTTAGCCTGGTAC |
| | CAGCAGAAACCTGGCCAGGCTCCCAGGCTCCTCATCTATGGTGCATCCACCAGG |
| | GCCACTGGTATCCCAGCCAGGTTCAGTGGCAGTGGGTCTGGGACAGAGTTCACT |
| | CTCACCATCAGCAGCCTGCAGTCTGAAGATTTTGCAGTTTATTACTGTCAGCAG |
| | TATAATAACTGGCCGCTCACTTTCGGCGGAGGGACCAAGGTGGAGATCAAA |
| | [SEQ ID NO: 166] |

In certain embodiments, the anti-DLL3 antibody or antigen-binding fragment thereof comprises V_{H} and V_{L} regions or CDRs selected from Table 23. In certain embodiments, the anti-DLL3 antibody or antigen-binding fragment thereof comprises a V_{H} comprising the amino acid sequence set forth in SEQ ID NO: 172, as shown in Table 23. An exemplary nucleotide sequence encoding the amino acid sequence of SEQ ID NO: 172 is set forth in SEQ ID NO: 174. In certain embodiments, the anti-DLL3 antibody or antigen-binding fragment thereof comprises a V_{L} comprising the amino acid sequence set forth in SEQ ID NO: 173. An exemplary nucleotide sequence encoding the amino acid sequence of SEQ ID NO: 173 is set forth in SEQ ID NO: 175. In certain embodiments, the anti-DLL3 antibody or antigen-binding fragment thereof comprises a V_{H} comprising the amino acid sequence set forth in SEQ ID NO: 172 and a V_{L} Comprising the amino acid sequence set forth in SEQ ID NO: 173. SEQ ID NO: 172-175 are provided in Table 23. In certain embodiments, the antibody or antigen-binding fragment thereof is designated as “N12”.

In certain embodiments, the anti-DLL3 antibody or antigen-binding fragment thereof comprises a V_{H} comprising a CDR1 comprising the amino acid sequence set forth in SEQ ID NO: 96 or a conservative modification thereof, a CDR2 comprising the amino acid sequence set forth in SEQ ID NO: 167 or a conservative modification thereof, and a CDR3 comprising the amino acid sequence set forth in SEQ ID NO: 168 or a conservative modification thereof. SEQ ID NOs: 96, 167, and 168 are provided in Table 23.

In certain embodiments, the anti-DLL3 antibody or antigen-binding fragment thereof comprises a V_{L} comprising a CDR1 comprising the amino acid sequence set forth in SEQ ID NO: 169 or a conservative modification thereof, a CDR2 comprising the amino acid sequence set forth in SEQ ID NO: 170 or a conservative modification thereof, and a CDR3 comprising the amino acid sequence set forth in SEQ ID NO: 171 or a conservative modification thereof. SEQ ID NOs: 169-171 are provided in Table 23.

In certain embodiments, the anti-DLL3 antibody or antigen-binding fragment thereof comprises a V_{H} comprising a CDR1 comprising the amino acid sequence set forth in SEQ ID NO: 96 or a conservative modification thereof, a CDR2 comprising the amino acid sequence set forth in SEQ ID NO: 167 or a conservative modification thereof, and a CDR3 comprising the amino acid sequence set forth in SEQ ID NO: 168 or a conservative modification thereof; and a V_{L} comprising a CDR1 comprising the amino acid sequence set forth in SEQ ID NO: 169 or a conservative modification thereof, a CDR2 comprising the amino acid sequence set forth in SEQ ID NO: 170 or a conservative modification thereof, and a CDR3 comprising the amino acid sequence set forth in SEQ ID NO: 171 or a conservative modification.

In certain embodiments, the anti-DLL3 antibody or antigen-binding fragment thereof comprises a V_{H} comprising a CDR1 comprising the amino acid sequence set forth in SEQ ID NO: 96, a CDR2 comprising the amino acid sequence set forth in SEQ ID NO: 167, and a CDR3 comprising the amino acid sequence set forth in SEQ ID NO: 168; and a V_{L} comprising a CDR1 comprising the amino acid sequence set forth in SEQ ID NO: 169, a CDR2 comprising the amino acid sequence set forth in SEQ ID NO: 170, and a CDR3 comprising the amino acid sequence set forth in SEQ ID NO: 171.

TABLE 23

| CDRs | 1 | 2 | 3 |
|---|---|---|---|
| V_{H} | GYTFTGY | NPNSGG | DGVITSEDY |
| | [SEQ ID NO: 96] | [SEQ ID NO: 167] | [SEQ ID NO: 168] |
| V_{L} | KSSQSVLYSSNIKTYLA | WASTRES | QQYY SAPYT |
| | [SEQ ID NO: 169] | [SEQ ID NO: 170] | [SEQ ID NO: 171] |

| | |
|---|---|
| Full V_{H} | QVQLVQSGAEVKKPGASVKVSCKASGYTFTGYYMHWVRQAPGQGLEWMGWINPN |
| | SGGTNYAQKFQGRVTMTRDTSISTAYMELSRLRSDDTAVYYCARDGVITSFDYW |
| | GQGTLVTVSS [SEQ ID NO: 172] |
| Full V_{L} | DIVMTQSPDSLAVSLGERATINCKSSQSVLYSSNIKTYLAWYQQKPGQPPKLLIY |
| | WASTRESGVPDRESGGGSGTDFTLTITSLQAEDVAVYYCQQYYSAPYTFGQGTKL |
| | EIK[SEQ ID NO: 173] |
| DNA for | CAGGTGCAGCTGGTGCAGTCTGGGGCTGAGGTGAAGAAGCCTGGGGCCTCAGTG |
| Full V_{H} | AAGGTCTCCTGCAAGGCTTCTGGATACACTTTCACCGGCTACTATATGCACTGG |
| | GTGCGACAGGCCCCTGGACAAGGGCTTGAGTGGATGGGATGGATCAACCCTAAC |
| | AGTGGTGGCACAAACTATGCACAGAAGTTTCAGGGCAGGGTCACCATGACCAGG |
| | GACACGTCCATCAGCACAGCCTACATGGAGCTGAGCAGGCTGAGATCTGACGAC |
| | ACGGCCGTGTATTACTGTGCGAGAGACGGGGTGATTACGTCTTTTGACTACTGG |
| | GGCCAGGGAACCCTGGTCACCGTCTCCTCA |
| | [SEQ ID NO: 174] |
| DNA for | GACATCGTGATGACCCAGTCTCCAGACTCCCTGGCTGTGTCTCTGGGCGAGAGG |
| Full V_{L} | GCCACCATCAACTGCAAGTCCAGCCAGAGTGTTTTATACAGCTCCAACATTAAG |
| | ACCTACTTAGCTTGGTACCAGCAGAAACCAGGACAGCCTCCTAAGCTGCTCATT |
| | TACTGGGCATCTACCCGGGAATCCGGAGTCCCTGACCGATTCAGTGGCGGCGGG |
| | TCTGGGACAGATTTCACTCTCACCATCACCAGCCTGCAGGCTGAAGATGTGGCA |
| | GTTTATTACTGTCAGCAGTATTATAGTGCTCCGTACACTTTTGGCCAGGGGACC |
| | AAGCTGGAGATCAAA |
| | [SEQ ID NO: 175] |

In certain embodiments, the anti-DLL3 antibody or antigen-binding fragment thereof comprises V_{H} and V_{L} regions or CDRs selected from Table 24. In certain embodiments, the anti-DLL3 antibody or antigen-binding fragment thereof comprises a V_{H} comprising the amino acid sequence set forth in SEQ ID NO: 180, as shown in Table 24. An exemplary nucleotide sequence encoding the amino acid sequence of SEQ ID NO: 180 is set forth in SEQ ID NO: 182. In certain embodiments, the anti-DLL3 antibody or antigen-binding fragment thereof comprises a V_{L} comprising the amino acid sequence set forth in SEQ ID NO: 181. An exemplary nucleotide sequence encoding the amino acid sequence of SEQ ID NO: 181 is set forth in SEQ ID NO: 183. In certain embodiments, the anti-DLL3 antibody or antigen-binding fragment thereof comprises a V_{H} comprising the amino acid sequence set forth in SEQ ID NO: 180 and a V_{L} Comprising the amino acid sequence set forth in SEQ ID NO: 181. SEQ ID NO: 180-183 are provided in Table 24. In certain embodiments, the antibody or antigen-binding fragment thereof is designated as “G23”.

In certain embodiments, the anti-DLL3 antibody or antigen-binding fragment thereof comprises a V_{H} comprising a CDR1 comprising the amino acid sequence set forth in SEQ ID NO: 21 or a conservative modification thereof, a CDR2 comprising the amino acid sequence set forth in SEQ ID NO: 176 or a conservative modification thereof, and a CDR3 comprising the amino acid sequence set forth in SEQ ID NO: 177 or a conservative modification thereof. SEQ ID NOs: 21, 176, and 177 are provided in Table 24.

In certain embodiments, the anti-DLL3 antibody or antigen-binding fragment thereof comprises a V_{L} comprising a CDR1 comprising the amino acid sequence set forth in SEQ ID NO: 178 or a conservative modification thereof, a CDR2 comprising the amino acid sequence set forth in SEQ ID NO: 50 or a conservative modification thereof, and a CDR3 comprising the amino acid sequence set forth in SEQ ID NO: 179 or a conservative modification thereof. SEQ ID NOs: 50, 178, and 179 are provided in Table 24.

In certain embodiments, the anti-DLL3 antibody or antigen-binding fragment thereof comprises a V_{H} comprising a CDR1 comprising the amino acid sequence set forth in SEQ ID NO: 21 or a conservative modification thereof, a CDR2 comprising the amino acid sequence set forth in SEQ ID NO: 176 or a conservative modification thereof, and a CDR3 comprising the amino acid sequence set forth in SEQ ID NO: 177 or a conservative modification thereof; and a V_{L} comprising a CDR1 comprising the amino acid sequence set forth in SEQ ID NO: 178 or a conservative modification thereof, a CDR2 comprising the amino acid sequence set forth in SEQ ID NO: 50 or a conservative modification thereof, and a CDR3 comprising the amino acid sequence set forth in SEQ ID NO: 179 or a conservative modification.

In certain embodiments, the anti-DLL3 antibody or antigen-binding fragment thereof comprises a V_{H} comprising a CDR1 comprising the amino acid sequence set forth in SEQ ID NO: 21, a CDR2 comprising the amino acid sequence set forth in SEQ ID NO: 176, and a CDR3 comprising the amino acid sequence set forth in SEQ ID NO: 177; and a V_{L} comprising a CDR1 comprising the amino acid sequence set forth in SEQ ID NO: 178, a CDR2 comprising the amino acid sequence set forth in SEQ ID NO: 50, and a CDR3 comprising the amino acid sequence set forth in SEQ ID NO: 179.

TABLE 24

| CDRs | 1 | 2 | 3 |
|---|---|---|---|
| V_{H} | GYTFTSY | DPSDGS | DREYNYYGLDV |
| | [SEQ ID NO: 21] | [SEQ ID NO: 176] | [SEQ ID NO: 177] |
| V_{L} | RSSQSLVYRDGNTYLN | KVSNRDS | MQGTHWPPT |
| | [SEQ ID NO: 178] | [SEQ ID NO: 50] | [SEQ ID NO: 179] |

| | |
|---|---|
| Full V_{H} | QVQLVQSGAEVKKPGASVKVSCKASGYTFTSYYIHWVRQAPGQGLEWMGIIDPS |
| | DGSTNYAQKFQGRVTMTRDTSTSTVYMELSSLRSEDTAVYYCARDREYNYYGLD |
| | VWGQGTTVTVSS |
| | [SEQ ID NO: 180] |
| Full V_{L} | DVVMTQSPLSLPVTLGQPASISCRSSQSLVYRDGNTYLNWFQQRPGQSPRRLIY |
| | KVSNRDSGVPDRFRGSGSGTDFTLKISRVEAEDVGVYYCMQGTHWPPTFGQGTK |
| | VEIK [SEQ ID NO: 181] |
| DNA for | CAGGTGCAGCTGGTGCAGTCTGGGGCTGAGGTGAAGAAGCCTGGGGCCTCAGTG |
| Full V_{H} | AAGGTTTCCTGCAAGGCATCTGGATACACCTTCACCAGCTACTATATACACTGG |
| | GTGCGACAGGCCCCTGGACAAGGGCTTGAGTGGATGGGAATAATCGACCCAAGT |
| | GATGGTAGCACAAACTACGCACAGAAGTTCCAGGGCAGAGTCACCATGACCAGG |
| | GACACGTCCACGAGCACAGTCTACATGGAGCTGAGCAGCCTGAGATCTGAGGAC |
| | ACGGCCGTGTATTACTGTGCGAGAGATCGGGAATATAACTACTACGGTTTGGAC |
| | GTCTGGGGCCAAGGGACCACGGTCACCGTCTCCTCA |
| | [SEQ ID NO: 182] |
| DNA for | GATGTTGTGATGACTCAGTCTCCACTCTCCCTGCCCGTCACCCTTGGACAGCCG |
| Full V_{L} | GCCTCCATCTCCTGCAGGTCTAGTCAAAGCCTCGTATACCGTGATGGAAACACC |
| | TACTTGAATTGGTTTCAGCAGAGGCCAGGCCAATCTCCAAGGCGCCTAATTTAT |
| | AAGGTTTCTAACCGGGACTCTGGGGTCCCAGACAGATTCCGCGGCAGTGGGTCA |
| | GGCACTGATTTCACACTGAAAATCAGCCGGGTGGAGGCTGAGGATGTTGGGGTT |
| | TATTACTGCATGCAAGGTACACACTGGCCTCCGACGTTCGGCCAAGGGACCAAG |
| | GTGGAAATCAAA |
| | SEQ ID NO: 183] |

In certain embodiments, the anti-DLL3 antibody or antigen-binding fragment thereof comprises V_{H} and V_{L} regions or CDRs selected from Table 25. In certain embodiments, the anti-DLL3 antibody or antigen-binding fragment thereof comprises a V_{H} comprising the amino acid sequence set forth in SEQ ID NO: 190, as shown in Table 25. An exemplary nucleotide sequence encoding the amino acid sequence of SEQ ID NO: 190 is set forth in SEQ ID NO: 192. In certain embodiments, the anti-DLL3 antibody or antigen-binding fragment thereof comprises a V_{L} comprising the amino acid sequence set forth in SEQ ID NO: 191. An exemplary nucleotide sequence encoding the amino acid sequence of SEQ ID NO: 191 is set forth in SEQ ID NO: 193. In certain embodiments, the anti-DLL3 antibody or antigen-binding fragment thereof comprises a V_{H} comprising the amino acid sequence set forth in SEQ ID NO: 190 and a V_{L} Comprising the amino acid sequence set forth in SEQ ID NO: 191. SEQ ID NO: 190-193 are provided in Table 25. In certain embodiments, the antibody or antigen-binding fragment thereof is designated as “I1”.

In certain embodiments, the anti-DLL3 antibody or antigen-binding fragment thereof comprises a V_{H} comprising a CDR1 comprising the amino acid sequence set forth in SEQ ID NO: 184 or a conservative modification thereof, a CDR2 comprising the amino acid sequence set forth in SEQ ID NO: 185 or a conservative modification thereof, and a CDR3 comprising the amino acid sequence set forth in SEQ ID NO: 186 or a conservative modification thereof. SEQ ID NOs: 184-186 are provided in Table 25.

In certain embodiments, the anti-DLL3 antibody or antigen-binding fragment thereof comprises a V_{L} comprising a CDR1 comprising the amino acid sequence set forth in SEQ ID NO: 187 or a conservative modification thereof, a CDR2 comprising the amino acid sequence set forth in SEQ ID NO: 188 or a conservative modification thereof, and a CDR3 comprising the amino acid sequence set forth in SEQ ID NO: 189 or a conservative modification thereof. SEQ ID NOs: 187-189 are provided in Table 25.

In certain embodiments, the anti-DLL3 antibody or antigen-binding fragment thereof comprises a V_{H} comprising a CDR1 comprising the amino acid sequence set forth in SEQ ID NO: 184 or a conservative modification thereof, a CDR2 comprising the amino acid sequence set forth in SEQ ID NO: 185 or a conservative modification thereof, and a CDR3 comprising the amino acid sequence set forth in SEQ ID NO: 186 or a conservative modification thereof; and a V_{L} comprising a CDR1 comprising the amino acid sequence set forth in SEQ ID NO: 187 or a conservative modification thereof, a CDR2 comprising the amino acid sequence set forth in SEQ ID NO: 188 or a conservative modification thereof, and a CDR3 comprising the amino acid sequence set forth in SEQ ID NO: 189 or a conservative modification.

In certain embodiments, the anti-DLL3 antibody or antigen-binding fragment thereof comprises a V comprising a CDR1 comprising the amino acid sequence set forth in SEQ ID NO: 184, a CDR2 comprising the amino acid sequence set forth in SEQ ID NO: 185, and a CDR3 comprising the amino acid sequence set forth in SEQ ID NO: 186; and a V_{L} comprising a CDR1 comprising the amino acid sequence set forth in SEQ ID NO: 187, a CDR2 comprising the amino acid sequence set forth in SEQ ID NO: 188, and a CDR3 comprising the amino acid sequence set forth in SEQ ID NO: 189.

TABLE 25

| CDRs | 1 | 2 | 3 |
|---|---|---|---|
| V_{H} | GFTFSNTW | IKSKSDGGTT | TQYYWNSFDY |
| | [SEQ ID NO: 184] | [SEQ ID NO: 185] | [SEQ ID NO: 186] |
| V_{L} | QASQDISNYLN | DASNLET | QQYDNLPLT |
| | [SEQ ID NO: 187] | [SEQ ID NO: 188] | [SEQ ID NO: 189] |

| | |
|---|---|
| Full V_{H} | EVQLVESGGGLVKPGGSLRLSCAASGFTFSNTWMSWVRQAPGKGLEWVGRIKSK |
| | SDGGTTDYAAPVKGRFTISRDDSKNTLYLQMNSLKTEDTAVYYCTQYYWNSFDY |
| | WGQGTLVTVSS |
| | [SEQ ID NO: 190] |
| Full V_{L} | DIQMTQSPSSLSASVGDRVTITCQASQDISNYLNWYQQKPGKAPKLLIYDASNL |
| | ETGVPSRESGSGSGTDFTFTISSLQPEDIATYYCQQYDNLPLTFGGGTKVEIK |
| | [SEQ ID NO: 191] |
| DNA for | GAGGTGCAGCTGGTGGAGTCTGGGGGGGGCTTGGTAAAGCCTGGGGGGTCCCTT |
| Full V_{H} | AGACTCTCCTGTGCAGCCTCTGGATTCACTTTCAGTAACACCTGGATGAGCTGG |
| | GTCCGCCAGGCTCCAGGGAAGGGGCTGGAGTGGGTTGGCCGTATTAAAAGCAAA |
| | TCTGATGGTGGGACAACAGACTACGCTGCACCCGTGAAAGGCAGATTCACCATC |
| | TCAAGAGATGATTCAAAAAACACGCTGTATCTGCAAATGAACAGCCTGAAAACC |
| | GAGGACACAGCCGTGTATTACTGTACCCAGTATTATTGGAACTCCTTTGACTAC |
| | TGGGGCCAGGGAACCCTGGTCACCGTCTCCTCA |
| | [SEQ ID NO: 192] |
| DNA for | GACATCCAGATGACCCAGTCTCCATCCTCCCTGTCTGCATCTGTAGGAGACAGA |
| Full V_{L} | GTCACCATCACTTGCCAGGCGAGTCAGGACATTAGCAACTATTTAAATTGGTAT |
| | CAGCAGAAACCAGGGAAAGCCCCTAAGCTCCTGATCTACGATGCATCCAATTTG |
| | GAAACAGGGGTCCCATCAAGGTTCAGTGGAAGTGGATCTGGGACAGATTTTACT |
| | TTCACCATCAGCAGCCTGCAGCCTGAAGATATTGCAACATATTACTGTCAACAG |
| | TATGATAATCTCCCGCTCACTTTCGGCGGAGGGACCAAGGTGGAGATCAAA |
| | [SEQ ID NO: 193] |

In certain embodiments, the anti-DLL3 antibody or antigen-binding fragment thereof comprises V_{H} and V_{L} regions or CDRs selected from Table 26. In certain embodiments, the anti-DLL3 antibody or antigen-binding fragment thereof comprises a V_{H} comprising the amino acid sequence set forth in SEQ ID NO: 197, as shown in Table 26. An exemplary nucleotide sequence encoding the amino acid sequence of SEQ ID NO: 197 is set forth in SEQ ID NO: 199. In certain embodiments, the anti-DLL3 antibody or antigen-binding fragment thereof comprises a V_{L} comprising the amino acid sequence set forth in SEQ ID NO: 198. An exemplary nucleotide sequence encoding the amino acid sequence of SEQ ID NO: 198 is set forth in SEQ ID NO: 200. In certain embodiments, the anti-DLL3 antibody or antigen-binding fragment thereof comprises a V_{H} comprising the amino acid sequence set forth in SEQ ID NO: 197 and a V_{L} Comprising the amino acid sequence set forth in SEQ ID NO: 198. SEQ ID NO: 197-200 are provided in Table 26. In certain embodiments, the antibody or antigen-binding fragment thereof is designated as “C8”.

In certain embodiments, the anti-DLL3 antibody or antigen-binding fragment thereof comprises a V_{H} comprising a CDR1 comprising the amino acid sequence set forth in SEQ ID NO: 11 or a conservative modification thereof, a CDR2 comprising the amino acid sequence set forth in SEQ ID NO: 194 or a conservative modification thereof, and a CDR3 comprising the amino acid sequence set forth in SEQ ID NO: 195 or a conservative modification thereof. SEQ ID NOs: 11, 194, and 195 are provided in Table 26.

In certain embodiments, the anti-DLL3 antibody or antigen-binding fragment thereof comprises a V_{L} comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 196 or a conservative modification thereof. SEQ ID NOs: 4, 5, and 196 are provided in Table 26.

In certain embodiments, the anti-DLL3 antibody or antigen-binding fragment thereof comprises a V_{H} comprising a CDR1 comprising the amino acid sequence set forth in SEQ ID NO: 11 or a conservative modification thereof, a CDR2 comprising the amino acid sequence set forth in SEQ ID NO: 194 or a conservative modification thereof, and a CDR3 comprising the amino acid sequence set forth in SEQ ID NO: 195 or a conservative modification thereof; and a V_{L} comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 196 or a conservative modification.

In certain embodiments, the anti-DLL3 antibody or antigen-binding fragment thereof comprises a V_{H} comprising a CDR1 comprising the amino acid sequence set forth in SEQ ID NO: 11, a CDR2 comprising the amino acid sequence set forth in SEQ ID NO: 194, and a CDR3 comprising the amino acid sequence set forth in SEQ ID NO: 195; and a V_{L} comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 196.

TABLE 26

| CDRs | 1 | 2 | 3 |
|---|---|---|---|
| V_{H} | GFTESSY | KEDGSE | DPGWAPEDY |
| | [SEQ ID NO: 11] | [SEQ ID NO: 194] | [SEQ ID NO: 195] |
| V_{L} | RASQGISNYLA | AASSLOS | QQYNSFPYT |
| | [SEQ ID NO: 4] | [SEQ ID NO: 5] | [SEQ ID NO: 196] |

| | |
|---|---|
| Full V_{H} | EVQLVESGGGLVQPGGSQRLSCAASGFTFSSYWMNWVRQAPGKGLEWVANIKED |
| | GSEKYYVDSVKGRFTISRDNAKNSLYLQMNSLRAEDTAVYYCARDPGWAPFDYW |
| | GQGTLVTVSS |
| | [SEQ ID NO: 197] |
| Full V_{L} | DIQMSQSPSSLSASVGDRVTITCRASQGISNYLAWFQQKPGKAPKSLIYAASSL |
| | QSGVPSKFSGSGSGTDFTLAISSLQPEDFATYYCQQYNSFPYTFGQGTTLEIK |
| | [SEQ ID NO: 198] |
| DNA for | GAGGTGCAGCTGGTGGAGTCTGGGGGAGGCTTGGTCCAGCCTGGGGGGTCCCAG |
| Full V_{H} | AGACTCTCCTGTGCAGCCTCTGGATTCACCTTTAGTAGCTATTGGATGAACTGG |
| | GTCCGCCAGGCTCCAGGGAAGGGGCTGGAGTGGGTGGCCAACATAAAGGAAGAT |
| | GGAAGTGAGAAATACTATGTGGACTCTGTGAAGGGCCGATTCACCATCTCCAGA |
| | GACAACGCCAAGAACTCACTGTATCTGCAAATGAACAGCCTGAGAGCCGAGGAC |
| | ACGGCTGTGTATTACTGTGCGAGAGATCCGGGCTGGGCTCCCTTTGACTACTGG |
| | GGCCAGGGAACCCTGGTCACCGTCTCCTCA |
| | [SEQ ID NO: 199] |
| DNA for | GACATCCAGATGTCCCAGTCTCCATCCTCACTGTCTGCATCTGTAGGAGACAGA |
| Full V_{L} | GTCACCATCACTTGTCGGGCGAGTCAGGGCATTAGCAATTATTTAGCCTGGTTT |
| | CAGCAGAAACCAGGGAAAGCCCCTAAGTCCCTGATCTATGCTGCATCCAGTTTG |
| | CAAAGTGGGGTCCCATCAAAGTTCAGCGGCAGTGGATCTGGGACAGATTTCACT |
| | CTCGCCATCAGCAGCCTGCAGCCTGAAGATTTTGCAACTTATTACTGCCAACAG |
| | TATAATAGTTTCCCGTACACTTTTGGCCAGGGGACCACGCTGGAGATCAAA |
| | [SEQ ID NO: 200] |

In certain embodiments, the anti-DLL3 antibody or antigen-binding fragment thereof comprises V_{H} and V_{L} regions or CDRs selected from Table 27. In certain embodiments, the anti-DLL3 antibody or antigen-binding fragment thereof comprises a V_{H} comprising the amino acid sequence set forth in SEQ ID NO: 205, as shown in Table 27. An exemplary nucleotide sequence encoding the amino acid sequence of SEQ ID NO: 205 is set forth in SEQ ID NO: 207. In certain embodiments, the anti-DLL3 antibody or antigen-binding fragment thereof comprises a V_{L} comprising the amino acid sequence set forth in SEQ ID NO: 206. An exemplary nucleotide sequence encoding the amino acid sequence of SEQ ID NO: 206 is set forth in SEQ ID NO: 208. In certain embodiments, the anti-DLL3 antibody or antigen-binding fragment thereof comprises a V_{H} comprising the amino acid sequence set forth in SEQ ID NO: 205 and a V_{L} Comprising the amino acid sequence set forth in SEQ ID NO: 206. SEQ ID NO: 205-208 are provided in Table 27. In certain embodiments, the antibody or antigen-binding fragment thereof is designated as “018”.

In certain embodiments, the anti-DLL3 antibody or antigen-binding fragment thereof comprises a V_{H} comprising a CDR1 comprising the amino acid sequence set forth in SEQ ID NO: 201 or a conservative modification thereof, a CDR2 comprising the amino acid sequence set forth in SEQ ID NO: 202 or a conservative modification thereof, and a CDR3 comprising the amino acid sequence set forth in SEQ ID NO: 203 or a conservative modification thereof. SEQ ID NOs: 201-203 are provided in Table 27.

In certain embodiments, the anti-DLL3 antibody or antigen-binding fragment thereof comprises a V_{L} comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 204 or a conservative modification thereof. SEQ ID NOs: 57, 58, and 204 are provided in Table 27.

In certain embodiments, the anti-DLL3 antibody or antigen-binding fragment thereof comprises a V_{H} comprising a CDR1 comprising the amino acid sequence set forth in SEQ ID NO: 201 or a conservative modification thereof, a CDR2 comprising the amino acid sequence set forth in SEQ ID NO: 202 or a conservative modification thereof, and a CDR3 comprising the amino acid sequence set forth in SEQ ID NO: 203 or a conservative modification thereof; and a V_{L} comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 204 or a conservative modification.

In certain embodiments, the anti-DLL3 antibody or antigen-binding fragment thereof comprises a V_{H} comprising a CDR1 comprising the amino acid sequence set forth in SEQ ID NO: 201 a CDR2 comprising the amino acid sequence set forth in SEQ ID NO: 202, a CDR3 comprising the amino acid sequence set forth in SEQ ID NO: 203; and a V_{L} comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 204.

TABLE 27

| CDRs | 1 | 2 | 3 |
|---|---|---|---|
| V_{H} | GGSINSY | FYSGI | IGVAGFYFDY |
| | [SEQ ID NO: 201] | [SEQ ID NO: 202] | [SEQ ID NO: 203] |
| V_{L} | RASQSVSSSYLA | GASSRAT | QQYGTSPLT |
| | [SEQ ID NO: 57] | [SEQ ID NO: 58] | [SEQ ID NO: 204] |

| | |
|---|---|
| Full V_{H} | QVQLQESGPGLVKPSETLSLTCTVSGGSINSYYWSWIRQPPGKGLEWIGYIFYS |
| | GITNYNPSLKSRVTISLDTSKNQFSLKLSSVTAADTAVYYCARIGVAGFYFDYW |
| | GQGTLVTVSS |
| | [SEQ ID NO: 205] |
| Full V_{L} | EIVLTQSPGTLSLSPGERATLSCRASQSVSSSYLAWYQQKPGQAPRLLIYGASS |
| | RATGIPDRESGSGSGTDETLTISRLEPEDFAVYYCQQYGTSPLTFGGGTKVEIK |
| | [SEQ ID NO: 206] |
| DNA for | CAGGTGCAGCTGCAGGAGTCGGGCCCAGGACTGGTGAAGCCTTCGGAGACCCTG |
| Full V_{H} | TCCCTCACCTGCACTGTCTCTGGTGGCTCCATCAATAGTTACTACTGGAGCTGG |
| | ATCCGGCAGCCCCCAGGGAAGGGACTGGAGTGGATTGGGTATATCTTTTACAGT |
| | GGGATCACCAACTACAACCCCTCCCTCAAGAGTCGAGTCACCATATCATTAGAC |
| | ACGTCCAAGAACCAGTTCTCCCTGAAGCTGAGCTCTGTGACCGCTGCGGACACG |
| | GCCGTGTATTACTGTGCGAGAATCGGCGTGGCTGGTTTTTACTTTGACTACTGG |
| | GGCCAGGGAACCCTGGTCACCGTCTCCTCA |
| | [SEQ ID NO: 207] |
| DNA for | GAAATTGTGTTGACGCAGTCTCCAGGCACCCTGTCTTTGTCTCCAGGGGAAAGA |
| Full V_{L} | GCCACCCTCTCCTGCAGGGCCAGTCAGAGTGTTAGCAGCAGCTACTTAGCCTGG |
| | TACCAGCAGAAACCTGGCCAGGCTCCCAGGCTCCTCATCTATGGTGCATCCAGC |
| | AGGGCCACTGGCATCCCAGACAGGTTCAGTGGCAGTGGGTCTGGGACAGACTTC |
| | ACTCTCACCATCAGCAGACTGGAGCCTGAAGATTTTGCAGTGTATTACTGTCAG |
| | CAGTATGGTACCTCACCGCTCACTTTCGGCGGAGGGACCAAGGTGGAGATCAAA |
| | [SEQ ID NO: 208] |

### 5.2.2. Antibody-TCO Conjugates and Radioligands

The presently disclosed subject matter is based, in part, on a rapid and selective in vivo biorthogonal reaction between trans-cyclooctene (TCO) and tetrazine (Tz). The use of antibody-TCO conjugates and small molecule tetrazine-based radioligands allows radioactive labeling of antibodies after accumulation at the target site in vivo.

In certain embodiments, an inverse electron demand Diels-Alder (IEDDA) reaction occurs between the tetrazine and transcyclooctene. The IEDDA cycloaddition as a click chemistry conjugation reaction has been shown to be extraordinarily rapid, with a second order rate constant greater than 30,000 M⁻¹s⁻¹. Furthermore, both components have been shown to be sufficiently stable in physiological settings, and amine-reactive variants (e.g., TCO-NHS and Tz-NHS) of both are commercially available.

In certain embodiments, the anti-DLL3 antibody-TCO conjugate comprises an anti-DLL3 antibody disclosed herein in Section 5.2.1. In certain embodiments, the anti-DLL3 antibody-TCO conjugate comprises a TCO moiety. In certain embodiments, the TCO moiety has formula:

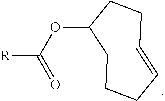

In certain embodiments, R is N, C, or an ester. In certain embodiments, R is a moiety capable of binding a biomolecule (e.g., an anti-DLL3 antibody disclosed herein). Non-limiting examples of moiety include NHS-ester, NCS, maleimide, PODS, DIBO, or DBCO. In certain embodiments, R comprises a linker. In certain embodiments, the linker attaches the TCO to the biomolecule (e.g., anti-DLL3 antibody). In certain embodiments, the linker comprises polyethylene glycol (PEG). In certain embodiments, the PEG comprises from about 1 unit to about 100 units. In certain embodiments, the linker comprises (poly)-L-lysine. In certain embodiments, the (poly)-L-lysine comprises from about 1 unit to about 200 units.

In certain embodiments, the TCO moiety has formula:

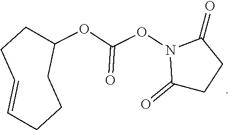 [SMILES: O=C(OC1CC/C=C/CCC1)ON1C(=O)CCC1=O]

Additional information on the synthesis and characterization of the TCOs used in the presently disclosed subject matter can be found in International Patent Publication No. WO2016/182804, the content of which is incorporated herein in its entirety.

In certain embodiments, the radioligand comprises a tetrazine moiety. A tetrazine is a compound that has a six-membered aromatic ring containing four nitrogen atoms with the molecular formula C₂H₂N₄. In certain embodiments, tetrazine moieties are tetrazines modified with different electron withdrawing groups at the three and six position of the 1,2,4,5-tetrazine core to increase their reactivity and at the same time ensuring modest stability in aqueous solutions. Additional information on the synthesis and characterization of the tetrazines used in the presently disclosed subject matter can be found in International Patent Publication No. WO2016/182804, the content of which is incorporated herein in its entirety.

In certain embodiments, the radioligand comprises a radiolabel. In certain embodiments, the radiolabel comprises a radioactive isotope. In certain embodiments, the radioactive isotope is selected from the group consisting of ^{99m}Tc, ¹¹¹In, ⁴⁴Sc, ⁴⁷Sc, ⁶⁴Cu, ⁶⁷Ga, ⁶⁸Ga, ⁷²As, ⁷⁷As, ⁵²Mn, ¹⁸⁶Re, ¹⁵³Sm, ¹⁷⁷Lu, ⁶⁷Cu, ¹²³I, ¹²⁴I, ¹²⁵I, ¹¹C, ¹³N, ¹⁵O, ¹⁸F, ¹⁶⁶Ho, ¹⁴⁹Pm, ⁹⁰Y, ⁸⁶Y, ²¹³Bi, ¹⁰³Pd, ¹⁰⁹Pd, ¹⁵⁹Gd, ¹⁴⁰La, ¹⁹⁸Au, ¹⁹⁹Au, ¹⁷⁵Yb, ¹⁶⁹Yb, ¹⁶⁵Dy, ¹⁶⁶Dy, ¹⁰⁵Ph, ¹¹¹Ag, ⁸⁹Zr, ¹⁹²Ir, ²²⁵Ac, ²¹²Bi, ²¹³Bi, ⁷⁶Br, ⁵⁵Co, ⁶⁰Cu, ⁶¹Cu, ⁶²Cu, ⁶⁶Ga, ⁹⁰Nb, ²¹²Pb, ¹⁵²Tb, ⁷⁷Br, ²¹¹At, ²²⁷Th, ²²⁴Ra and ¹⁴⁹Tb. In certain embodiments, the radiolabel comprises a ⁶⁴Cu radioactive isotope. In certain embodiments, the radiolabel comprises a ²²⁵Ac radioactive isotope. In certain embodiments, the radiolabel comprises a ¹⁸F radioactive isotope. In certain embodiments, the radiolabel comprises a ¹⁷⁷Lu radioactive isotope. In certain embodiments, the radiolabel comprises a ⁸⁹Zr radioactive isotope.

In certain embodiments, the radiolabel has a decay energy in the range of 20 to 6,000 keV. Decay energies can be within the range of 60 to 200 keV for an Auger emitter, 100-2,500 keV for a beta emitter, and 4,000-6,000 keV for an alpha emitter. Maximum decay energies of useful beta-particle-emitting nuclides can range from 20-5,000 keV, 100-4,000 keV, or 500-2,500 keV. Decay energies of useful Auger-emitters can be <1,000 keV, <100 keV, or <70 keV. Decay energies of useful alpha-particle-emitting radionuclides can range from 2,000-10,000 keV, 3,000-8,000 keV, or 4,000-7,000 keV.

In certain embodiments, the radioligand comprises a chelator. In certain embodiments, the chelator is a sarcophagine-based chelator (SarAr). In certain embodiments, the chelator comprises sarcophagine or a derivative thereof. In certain embodiments, the chelator is a 1,4,7-triazonane-1,4,7-triyl-triacetic acid (NOTA) or a derivative thereof. In certain embodiments, the chelator is a 1,4,7-triazacyclononane-1,4-diacetate (NODA) or a derivative thereof.

In certain embodiments, the radioligand comprises a linker. In certain embodiments, the linker attaches the tetrazine moiety to the chelator. In certain embodiments, the linker comprises polyethylene glycol (PEG). In certain embodiments, the PEG comprises from about 1 unit to about 100 units. In certain embodiments, the linker comprises (poly)-L-lysine. In certain embodiments, the (poly)-L-lysine comprises from about 1 unit to about 200 units.

In certain embodiments, the tetrazine moiety and the chelator form a compound selected from the group consisting of:
2,2′,2″-(3-(4-(3-(1-(4-(1,2,4,5-tetrazin-3-yl)phenyl)-1-oxo-5,8,11,14,17,20,23-heptaoxa-2-azapentacosan-25-yl)thioureido)benzyl)-1,4,7-triazonane-2,5,8-triyl)triacetic acid;
2,2′,2″-(3-(4-(3-(1-(4-(1,2,4,5-tetrazin-3-yl)phenyl)-1-oxo-5,8,11,14,17,20,23,26,29,32,35-undecaoxa-2-azaheptatriacontan-37-yl)thioureido)benzyl)-1,4,7-triazonane-2,5,8-triyl)triacetic acid;
2,2′-(7-(4-(3-(1-(4-(1,2,4,5-tetrazin-3-yl)phenyl)-1-oxo-5,8,11,14,17,20,23,26,29,32,35-undecaoxa-2-azaheptatriacontan-37-yl)thioureido)benzyl)-1,4,7-triazonane-1,4-diyl)diacetic acid;
2,2′,2″-(3-(4-(3-(1-(4-(1,2,4,5-tetrazin-3-yl)phenyl)-3,7-dioxo-11,14,17,20,23,26,29-heptaoxa-2,8-diazahentriacontan-31-yl)thioureido)benzyl)-1,4,7-triazonane-2,5,8-triyl)triacetic acid;
2,2′,2″-(3-(4-(3-(1-(4-(1,2,4,5-tetrazin-3-yl)phenyl)-3,7-dioxo-11,14,17,20,23,26,29,32,35,38,41-undecaoxa-2,8-diazatritetracontan-43-yl)thioureido)benzyl)-1,4,7-triazonane-2,5,8-triyl)triacetic acid;
2,2′,2″-(3-(4-(3-(25,28-dioxo-28-((6-(6-(pyridin-2-yl)-1,2,4,5-tetrazin-3-yl)pyridin-3-yl)amino)-3,6,9,12,15,18,21-heptaoxa-24-azaoctacosyl)thioureido)benzyl)-1,4,7-triazonane-2,5,8-triyl)triacetic acid;
2,2′,2″-(3-(4-(3-(37,40-dioxo-40-((6-(6-(pyridin-2-yl)-1,2,4,5-tetrazin-3-yl)pyridin-3-yl)amino)-3,6,9,12,15,18,21,24,27,30,33-undecaoxa-36-azatetracontyl)thioureido)benzyl)-1,4,7-triazonane-2,5,8-triyl)triacetic acid;
2,2′,2″-(3-(4-(1-(4-(6-methyl-1,2,4,5-tetrazin-3-yl)phenyl)-3-oxo-6,9,12,15,18,21,24-heptaoxa-2-azaheptacosan-27-amido)benzyl)-1,4,7-triazonane-2,5,8-triyl)triacetic acid;
2,2′,2″-(2-(4-(1-(4-(6-methyl-1,2,4,5-tetrazin-3-yl)phenoxy)-3,6,9,12,15,18,21,24,27,30,33-undecaoxahexatriacontan-36-amido)benzyl)-1,4,7-triazonane-1,4,7-triyl)triacetic acid;
2,2′,2″-(3-(4-(3-(5-amino-6-((4-(6-methyl-1,2,4,5-tetrazin-3-yl)benzyl)amino)-6-oxohexyl)thioureido)benzyl)-1,4,7-triazonane-2,5,8-triyl)triacetic acid;
2,2′-(7-(4-(3-(5-amino-6-((4-6-methyl-1,2,4,5-tetrazin-3-yl)benzyl)amino)-6-oxohexyl)thioureido)benzyl)-1,4,7-triazonane-1,4-diyl)diacetic acid;
2,2′,2″-(3-(4-(3-(5-amino-6-((5-amino-6-((4-(6-methyl-1,2,4,5-tetrazin-3-yl)benzyl)amino)-6-oxohexyl)amino)-6-oxohexyl)thioureido)benzyl)-1,4,7-triazonane-2,5,8-triyl)triacetic acid; and
2,2′,2″-(3-(4-(3-(5-amino-6-((5-amino-6-((5-amino-6-((4-(6-methyl-1,2,4,5-tetrazin-3-yl)benzyl)amino)-6-oxohexyl)amino)-6-oxohexyl)amino)-6-oxohexyl)thioureido)benzyl)-1,4,7-triazonane-2,5,8-triyl)triacetic acid.

Additional information on the synthesis and characterization of the radioligands used in the presently disclosed subject matter can be found in International Patent Publication No. WO2016/182804, the content of which is incorporated herein in its entirety.

### 5.3. Pharmaceutical Compositions

The presently disclosed subject matter provides compositions comprising a presently disclosed anti-DLL3 antibody-TCO conjugate (e.g., one disclosed in Section 5.2.2). The presently disclosed subject matter provides compositions comprising a presently disclosed radioligand (e.g., one disclosed in Section 5.2.2). In certain embodiments, the composition is a pharmaceutical composition further comprising a pharmaceutically acceptable carrier.

Suitable pharmaceutically acceptable carriers include, for example, one or more of water, saline, phosphate buffered saline, dextrose, glycerol, ethanol and the like, as well as combinations thereof.

Pharmaceutically acceptable carriers may further comprise minor amounts of auxiliary substances such as wetting or emulsifying agents, preservatives or buffers, which enhance the shelf life or effectiveness of the binding proteins. The compositions of the injection can, as is well known in the art, be formulated so as to provide quick, sustained or delayed release of the active ingredient after administration to the mammal.

The presently disclosed antibody-TCO conjugates and the radioligands can be incorporated into pharmaceutical compositions suitable for administration. The pharmaceutical compositions generally comprise recombinant or substantially purified anti-DLL3 antibody-TCO conjugates and a pharmaceutically acceptable carrier in a form suitable for administration to a subject. The pharmaceutical compositions also comprise substantially purified radioligands and a pharmaceutically acceptable carrier in a form suitable for administration to a subject. Pharmaceutically acceptable carriers are determined in part by the particular composition being administered, as well as by the particular method used to administer the composition. Accordingly, there is a wide variety of suitable formulations of pharmaceutical compositions for administering the antibody compositions. The pharmaceutical compositions can be formulated as sterile, substantially isotonic and in full compliance with all Good Manufacturing Practice (GMP) regulations of the U.S. Food and Drug Administration.

The terms “pharmaceutically acceptable,” “physiologically tolerable,” and grammatical variations thereof, as they refer to compositions, carriers, diluents and reagents, are used interchangeably and represent that the materials are capable of administration to or upon a subject without the production of undesirable physiological effects to a degree that would prohibit administration of the composition. For example, “pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous. The term “pharmaceutically acceptable salts and esters” means salts and esters that are pharmaceutically acceptable and have the desired pharmacological properties. Such salts include salts that can be formed where acidic protons present in the composition are capable of reacting with inorganic or organic bases. Suitable inorganic salts include those formed with the alkali metals, e.g., sodium and potassium, magnesium, calcium, and aluminum. Suitable organic salts include those formed with organic bases such as the amine bases, e.g., ethanolamine, diethanolamine, triethanolamine, tromethamine, N-methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically acceptable esters include esters formed from carboxy, sulfonyloxy, and phosphonoxy groups present in the antibody-TCO conjugates, e.g., C1-6 alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. An anti-DDL3 antibody-TCO conjugate named in this technology can be present in unsalified or unesterified form, or in salified and/or esterified form, and the naming of such anti-DDL3 antibody-TCO conjugate is intended to include both the original (unsalified and unesterified) compound and its pharmaceutically-acceptable salts and esters.

A presently pharmaceutical composition is formulated to be compatible with its intended and/or suitable route of administration. The presently disclosed antibody-TCO conjugates and the radioligands can be administered by parenteral, topical, intravenous, oral, subcutaneous, intraarterial, intradermal, transdermal, rectal, intracranial, intrathecal, intraperitoneal, intranasal; or intramuscular routes, or as inhalants.

### 5.4. Methods of Uses

The presently disclosed subject matter provides various methods of using the antibody-TCO conjugates, the radioligands, and compositions disclosed herein.

The presently disclosed subject matter provides methods of in vivo diagnosis of a disease or disorder in a subject. In certain embodiments, the method comprises administering to the subject the antibody-TCO conjugate disclosed herein or a composition comprising thereof. In certain embodiments, a portion of the antibody-TCO conjugate localizes at cells of the disease or disorder and unbound antibody-TCO conjugate is cleared from blood, from renal system, and/or from the subject after an accumulation interval. In certain embodiments, the method further comprises administering to the subject the radioligand disclosed herein or a composition comprising thereof, wherein the radioligand comprises a tetrazine moiety and a radiolabel. In certain embodiments, the antibody-TCO conjugate and the radioligand bind together to form a radioimmunoconjugate via an in vivo click reaction at the cells of the disease or disorder within a region of the subject. In certain embodiments, the method further comprises imaging the radioimmunoconjugate accumulated in the region of the subject. In certain embodiments, the imaging occurs via positron emission tomography (PET). In certain embodiments, the imaging occurs via single photon emission computed tomography. In certain embodiments, the imaging occurs within a time period of about 4 hours, about 8 hours, about 12 hours, about 24 hours, about 48 hours, up to about 72 hours, up to about 4 days, up to about 5 days, up to about 6 days, or up to about 7 days from the administering of the radioligand. In certain embodiments, the imaging occurs within a time period of between about 24 hours and up to about 7 days from the administering of the radioligand. In certain embodiments, the imaging occurs within a time period of less than about 9 hours from the administering of the radioligand.

In certain embodiments, the radioligand is administered to the subject after an accumulation interval. In certain embodiments, the accumulation interval is up to and including one day, up to and including two days, up to and including three days, up to and including four days, up to and including five days, up to and including six days, up to and including seven days, up to and including eight days, up to and including nine days, up to and including ten days, up to and including eleven days, up to and including twelve days, up to and including thirteen days, up to and including fourteen days, or up to and including fifteen days after administration of the antibody-TCO targeting probe. In certain embodiments, the accumulation interval is between about one day and about two days, between about one day and about three days, between about one day and about four days, between about one day and about five days, between about one day and about six days, between about one day and about seven days, between about one and about ten days, or between about one and about fourteen days after administration of the antibody-TCO conjugate. In certain embodiments, the accumulation interval depends on the body clearance and half-life of the antibody-TCO conjugate.

In certain embodiments, the radiolabel comprises a radioactive isotope that is selected from the group consisting of ⁶⁴Cu, ¹⁸F, ⁴⁴Sc, ⁷²As, ⁵²Mn, ¹²⁴I, ¹¹C, ¹³N, ¹⁵O, ⁸⁶Y, ⁷⁶Br, ⁵⁵Co, ⁶⁰Cu, ⁶¹Cu, ⁶²Cu, ⁶⁶Ga, ⁹⁰Nb, and ¹⁵²Tb. In certain embodiments, the radiolabel comprises a ⁶⁴Cu radioactive isotope. In certain embodiments, the imaging comprises detecting the radioactive levels emitted by the radioimmunoconjugate. In certain embodiments, the radioactive levels emitted by the radioimmunoconjugate are expressed as the percentage injected dose per gram tissue (% ID/g).

Furthermore, the presently disclosed subject matter provides radiotherapies for treating a disease or disorder in a subject. In certain embodiments, the method comprises administering to the subject the antibody-TCO conjugate disclosed herein or a composition comprising thereof. In certain embodiments, a portion of the antibody-TCO conjugate localizes at cells of the disease or disorder and unbound antibody-TCO conjugate is cleared from blood, from renal system, and/or from the subject after an accumulation interval. In certain embodiments, the method further comprises administering to the subject the radioligand disclosed herein or a composition comprising thereof, wherein the radioligand comprises a tetrazine moiety and a radiolabel. In certain embodiments, the antibody-TCO conjugate and the radioligand bind together to form a radioimmunoconjugate via an in vivo click reaction at the cells of the disease or disorder within a region of the subject. In certain embodiments, the radioligand is administered to the subject after an accumulation interval. In certain embodiments, the accumulation interval is up to and including one day, up to and including two days, up to and including three days, up to and including four days, up to and including five days, up to and including six days, up to and including seven days, up to and including eight days, up to and including nine days, up to and including ten days, up to and including eleven days, up to and including twelve days, up to and including thirteen days, up to and including fourteen days, or up to and including fifteen days after administration of the antibody-TCO targeting probe. In certain embodiments, the accumulation interval is between about one day and about two days, between about one day and about three days, between about one day and about four days, between about one day and about five days, between about one day and about six days, between about one day and about seven days, between about one day and about ten days, or between about one day and about fourteen days after administration of the antibody-TCO conjugate. In certain embodiments, the accumulation interval depends on the body clearance and half-life of the antibody-TCO conjugate.

In certain embodiments, the radiolabel comprises an alpha-particle emitter radioactive isotope. In certain embodiments, the radiolabel comprises a beta-particle emitter radioactive isotope. In certain embodiments, the radiolabel comprises an auger emitter radioactive isotope. In certain embodiments, the radiolabel comprises a radioactive isotope that is selected from the group consisting of ²²⁵Ac, ^{99m}Tc, ¹¹¹In, ⁴⁴Sc, ⁴⁷Sc, ⁶⁴Cu, ⁶⁷Ga, ⁶⁸Ga, ⁷²As, ⁷⁷As, ⁵²Mn, ¹⁸⁶Re, ¹⁵³Sm, ¹⁷⁷Lu, ⁶⁷Cu, ¹²³I, ¹²⁴I, ¹²⁵I, ¹¹C, ¹³N, ¹⁵O, ¹⁸F, ¹⁶⁶Ho, ¹⁴⁹Pm, ⁹⁰Y, ⁸⁶Y, ²¹³Bi, ¹⁰³Pd, ¹⁰⁹Pd, ¹⁵⁹Gd, ¹⁴⁰La, ¹⁹⁸Au, ¹⁹⁹Au, ¹⁷⁵Yb, ¹⁶⁹Yb, ¹⁶⁵Dy, ¹⁶⁶Dy, ¹⁰⁵Ph, ¹¹¹Ag, ⁸⁹Zr, ¹⁹²Ir, ²¹²Bi, ²¹³Bi, ⁷⁶Br, ⁵⁵Co, ⁶⁰Cu, ⁶¹Cu, ⁶²Cu, ⁶⁶Ga, ⁹⁰Nb, ²¹²Pb, ¹⁵²Tb, ⁷⁷Br, ²¹¹At, ²²⁷Th, ²²⁴Ra and ¹⁴⁹Tb. In certain embodiments, the radiolabel comprises a ²²⁵Ac radioactive isotope. In certain embodiments, the radiolabel is an alpha-particle emitter. Since certain tumors are generally radio-resistant, alpha-particle radiotherapy (e.g., using alpha-particle emitter radioligands such as ²²⁵Ac) results in highly potent cell-killing activity with minimal collateral damage via high linear energy transfer alpha particle emissions with a range of 50-80 microns and 5-8 MeV energy. Unlike beta-particles that can deposit their energy over a longer distance, alpha-particle radiotherapy has a high therapeutic potential against small-volume tumors, including minimal residual disease which can be a major cause of cancer relapse.

In certain embodiments, the disease or disorder is associated with DLL3. In certain embodiments, the disease or disorder is associated with overexpression of DLL3. In certain embodiments, the disease or disorder is tumor. In certain embodiments, the tumor is cancer. In certain embodiments, the disease or disorder is selected from neuroendocrine tumors of the lung, extrapulmonary neuroendocrine carcinomas, melanoma, neuroendocrine prostate cancer, and breast cancer. In certain embodiments, the neuroendocrine tumors of the lung are selected from the group consisting of pulmonary neuroendocrine cancer (including typical carcinoid tumors, and atypical carcinoid tumors), large cell neuroendocrine carcinoma (LCNEC), and small-cell lung cancer. In certain embodiments, the melanoma is uveal melanoma. In certain embodiments, the breast cancer is triple negative breast cancer.

### 5.5. Kits

The presently disclosed subject matter provides kits for performing various methods disclosed herein (e.g., those disclosed in Section 5.4). In certain embodiments, the kit comprises the anti-DLL3 antibody-TCO conjugates (e.g., those disclosed in Section 5.2.2) or compositions comprising thereof disclosed herein (e.g., these disclosed in Section 5.3). In certain embodiments, the kit comprises the radioligands (e.g., one disclosed in Section 5.2.2) or compositions thereof disclosed herein. In certain embodiments, the kit comprises a sterile container which contains an anti-DDL3 antibody-TCO conjugate. In certain embodiments, the kit further comprises a sterile container which contains radioligand. Such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

In certain embodiments, the kit further comprises instructions for administering the antibody-TCO conjugates and the radioligands disclosed herein to a subject. The instructions can generally include information about the use of the antibody-TCO conjugates and the radioligands disclosed herein for in vivo diagnosis of a disease or disorder in a subject. In certain embodiments, instructions can include information about the use of the antibody-TCO conjugates and the radioligands disclosed herein for radiotherapies for treating a disease or disorder disclosed herein. In certain non-limiting embodiments, the instructions include at least one of the following: description of the therapeutic agents; dosage schedule and administration for treatment and/or prevention of a disease or disorder or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### 5.6. Exemplary Embodiments

A1. In certain embodiments, the presently disclosed subject matter provides an antibody-transcyclooctene (TCO) conjugate comprising an anti-DLL3 antibody or an antigen-binding fragment thereof comprising a heavy chain variable region that comprises CDR1, CDR2, and CDR3 domains; and a light chain variable region that comprises CDR1, CDR2, and CDR3 domains, wherein the heavy chain variable region and light chain variable region CDR3 domains are selected from the group consisting of:
a) a heavy chain variable region CDR3 comprising the amino acid sequence set forth in SEQ ID NO: 3 and a conservative modification thereof; and a light chain variable region CDR3 comprising the amino acid sequence set forth in SEQ ID NO: 6 and a conservative modification thereof;
b) a heavy chain variable region CDR3 comprising the amino acid sequence set forth in SEQ ID NO: 13 and a conservative modification thereof; and a light chain variable region CDR3 comprising the amino acid sequence set forth in SEQ ID NO: 16 and a conservative modification thereof;
c) a heavy chain variable region CDR3 comprising the amino acid sequence set forth in SEQ ID NO: 22 and a conservative modification thereof; and a light chain variable region CDR3 comprising the amino acid sequence set forth in SEQ ID NO: 23 and a conservative modification thereof;
d) a heavy chain variable region CDR3 comprising the amino acid sequence set forth in SEQ ID NO: 30 and a conservative modification thereof; and a light chain variable region CDR3 comprising the amino acid sequence set forth in SEQ ID NO: 33 and a conservative modification thereof;
e) a heavy chain variable region CDR3 comprising the amino acid sequence set forth in SEQ ID NO: 39 and a conservative modification thereof; and a light chain variable region CDR3 comprising the amino acid sequence set forth in SEQ ID NO: 41 and a conservative modification thereof;
f) a heavy chain variable region CDR3 comprising the amino acid sequence set forth in SEQ ID NO: 48 and a conservative modification thereof; and a light chain variable region CDR3 comprising the amino acid sequence set forth in SEQ ID NO: 51 and a conservative modification thereof;
g) a heavy chain variable region CDR3 comprising the amino acid sequence set forth in SEQ ID NO: 56 and a conservative modification thereof; and a light chain variable region CDR3 comprising the amino acid sequence set forth in SEQ ID NO: 59 and a conservative modification thereof;
h) a heavy chain variable region CDR3 comprising the amino acid sequence set forth in SEQ ID NO: 64 and a conservative modification thereof; and a light chain variable region CDR3 comprising the amino acid sequence set forth in SEQ ID NO: 65 and a conservative modification thereof;
i) a heavy chain variable region CDR3 comprising the amino acid sequence set forth in SEQ ID NO: 72 and a conservative modification thereof; and a light chain variable region CDR3 comprising the amino acid sequence set forth in SEQ ID NO: 75 and a conservative modification thereof;
j) a heavy chain variable region CDR3 comprising the amino acid sequence set forth in SEQ ID NO: 81 and a conservative modification thereof; and a light chain variable region CDR3 comprising the amino acid sequence set forth in SEQ ID NO: 82 and a conservative modification thereof;
k) a heavy chain variable region CDR3 comprising the amino acid sequence set forth in SEQ ID NO: 89 and a conservative modification thereof; and a light chain variable region CDR3 comprising the amino acid sequence set forth in SEQ ID NO: 91 and a conservative modification thereof;
l) a heavy chain variable region CDR3 comprising the amino acid sequence set forth in SEQ ID NO: 98 and a conservative modification thereof; and a light chain variable region CDR3 comprising the amino acid sequence set forth in SEQ ID NO: 101 and a conservative modification thereof;
m) a heavy chain variable region CDR3 comprising the amino acid sequence set forth in SEQ ID NO: 107 and a conservative modification thereof; and a light chain variable region CDR3 comprising the amino acid sequence set forth in SEQ ID NO: 82 and a conservative modification thereof;
n) a heavy chain variable region CDR3 comprising the amino acid sequence set forth in SEQ ID NO: 107 and a conservative modification thereof; and a light chain variable region CDR3 comprising the amino acid sequence set forth in SEQ ID NO: 112 and a conservative modification thereof;
o) a heavy chain variable region CDR3 comprising the amino acid sequence set forth in SEQ ID NO: 116 and a conservative modification thereof; and a light chain variable region CDR3 comprising the amino acid sequence set forth in SEQ ID NO: 118 and a conservative modification thereof;
p) a heavy chain variable region CDR3 comprising the amino acid sequence set forth in SEQ ID NO: 123 and a conservative modification thereof; and a light chain variable region CDR3 comprising the amino acid sequence set forth in SEQ ID NO: 125 and a conservative modification thereof;
q) a heavy chain variable region CDR3 comprising the amino acid sequence set forth in SEQ ID NO: 56 and a conservative modification thereof; and a light chain variable region CDR3 comprising the amino acid sequence set forth in SEQ ID NO: 130 and a conservative modification thereof;
r) a heavy chain variable region CDR3 comprising the amino acid sequence set forth in SEQ ID NO: 137 and a conservative modification thereof; and a light chain variable region CDR3 comprising the amino acid sequence set forth in SEQ ID NO: 140 and a conservative modification thereof;
s) a heavy chain variable region CDR3 comprising the amino acid sequence set forth in SEQ ID NO: 145 and a conservative modification thereof; and a light chain variable region CDR3 comprising the amino acid sequence set forth in SEQ ID NO: 125 and a conservative modification thereof;
t) a heavy chain variable region CDR3 comprising the amino acid sequence set forth in SEQ ID NO: 152 and a conservative modification thereof; and a light chain variable region CDR3 comprising the amino acid sequence set forth in SEQ ID NO: 82 and a conservative modification thereof;
u) a heavy chain variable region CDR3 comprising the amino acid sequence set forth in SEQ ID NO: 123 and a conservative modification thereof; and a light chain variable region CDR3 comprising the amino acid sequence set forth in SEQ ID NO: 59 and a conservative modification thereof;
v) a heavy chain variable region CDR3 comprising the amino acid sequence set forth in SEQ ID NO: 161 and a conservative modification thereof; and a light chain variable region CDR3 comprising the amino acid sequence set forth in SEQ ID NO: 162 and a conservative modification thereof;
w) a heavy chain variable region CDR3 comprising the amino acid sequence set forth in SEQ ID NO: 168 and a conservative modification thereof; and a light chain variable region CDR3 comprising the amino acid sequence set forth in SEQ ID NO: 171 and a conservative modification thereof;
x) a heavy chain variable region CDR3 comprising the amino acid sequence set forth in SEQ ID NO: 177 and a conservative modification thereof; and a light chain variable region CDR3 comprising the amino acid sequence set forth in SEQ ID NO: 179 and a conservative modification thereof;
y) a heavy chain variable region CDR3 comprising the amino acid sequence set forth in SEQ ID NO: 186 and a conservative modification thereof; and a light chain variable region CDR3 comprising the amino acid sequence set forth in SEQ ID NO: 189 and a conservative modification thereof;
z) a heavy chain variable region CDR3 comprising the amino acid sequence set forth in SEQ ID NO: 195 and a conservative modification thereof; and a light chain variable region CDR3 comprising the amino acid sequence set forth in SEQ ID NO: 196 and a conservative modification thereof; and
aa) a heavy chain variable region CDR3 comprising the amino acid sequence set forth in SEQ ID NO: 203 and a conservative modification thereof; and a light chain variable region CDR3 comprising the amino acid sequence set forth in SEQ ID NO: 204 and a conservative modification thereof.

A2. The foregoing antibody-TCO conjugate of A1, wherein the heavy chain variable region and light chain variable region CDR2 domains are selected from the group consisting of:
a) a heavy chain variable region CDR2 comprising the amino acid sequence set forth in SEQ ID NO: 2 and a conservative modification thereof; and a light chain variable region CDR2 comprising the amino acid sequence set forth in SEQ ID NO: 5 and a conservative modification thereof;
b) a heavy chain variable region CDR2 comprising the amino acid sequence set forth in SEQ ID NO: 12 and a conservative modification thereof; and a light chain variable region CDR2 comprising the amino acid sequence set forth in SEQ ID NO: 15 and a conservative modification thereof;
c) a heavy chain variable region CDR2 comprising the amino acid sequence set forth in SEQ ID NO: 29 and a conservative modification thereof; and a light chain variable region CDR2 comprising the amino acid sequence set forth in SEQ ID NO: 32 and a conservative modification thereof;
d) a heavy chain variable region CDR2 comprising the amino acid sequence set forth in SEQ ID NO: 38 and a conservative modification thereof; and a light chain variable region CDR2 comprising the amino acid sequence set forth in SEQ ID NO: 5 and a conservative modification thereof;
e) a heavy chain variable region CDR2 comprising the amino acid sequence set forth in SEQ ID NO: 47 and a conservative modification thereof; and a light chain variable region CDR2 comprising the amino acid sequence set forth in SEQ ID NO: 50 and a conservative modification thereof;
f) a heavy chain variable region CDR2 comprising the amino acid sequence set forth in SEQ ID NO: 2 and a conservative modification thereof; and a light chain variable region CDR2 comprising the amino acid sequence set forth in SEQ ID NO: 58 and a conservative modification thereof;
g) a heavy chain variable region CDR2 comprising the amino acid sequence set forth in SEQ ID NO: 71 and a conservative modification thereof; and a light chain variable region CDR2 comprising the amino acid sequence set forth in SEQ ID NO: 74 and a conservative modification thereof;
h) a heavy chain variable region CDR2 comprising the amino acid sequence set forth in SEQ ID NO: 80 and a conservative modification thereof; and a light chain variable region CDR2 comprising the amino acid sequence set forth in SEQ ID NO: 58 and a conservative modification thereof;
i) a heavy chain variable region CDR2 comprising the amino acid sequence set forth in SEQ ID NO: 88 and a conservative modification thereof; and a light chain variable region CDR2 comprising the amino acid sequence set forth in SEQ ID NO: 210 and a conservative modification thereof;
j) a heavy chain variable region CDR2 comprising the amino acid sequence set forth in SEQ ID NO: 97 and a conservative modification thereof; and a light chain variable region CDR2 comprising the amino acid sequence set forth in SEQ ID NO: 100 and a conservative modification thereof;
k) a heavy chain variable region CDR2 comprising the amino acid sequence set forth in SEQ ID NO: 106 and a conservative modification thereof; and a light chain variable region CDR2 comprising the amino acid sequence set forth in SEQ ID NO: 58 and a conservative modification thereof;
l) a heavy chain variable region CDR2 comprising the amino acid sequence set forth in SEQ ID NO: 106 and a conservative modification thereof; and a light chain variable region CDR2 comprising the amino acid sequence set forth in SEQ ID NO: 5 and a conservative modification thereof;
m) a heavy chain variable region CDR2 comprising the amino acid sequence set forth in SEQ ID NO: 115 and a conservative modification thereof; and a light chain variable region CDR2 comprising the amino acid sequence set forth in SEQ ID NO: 100 and a conservative modification thereof;
n) a heavy chain variable region CDR2 comprising the amino acid sequence set forth in SEQ ID NO: 2 and a conservative modification thereof; and a light chain variable region CDR2 comprising the amino acid sequence set forth in SEQ ID NO: 58 and a conservative modification thereof;
o) a heavy chain variable region CDR2 comprising the amino acid sequence set forth in SEQ ID NO: 136 and a conservative modification thereof; and a light chain variable region CDR2 comprising the amino acid sequence set forth in SEQ ID NO: 139 and a conservative modification thereof;
p) a heavy chain variable region CDR2 comprising the amino acid sequence set forth in SEQ ID NO: 2 and a conservative modification thereof; and a light chain variable region CDR2 comprising the amino acid sequence set forth in SEQ ID NO: 146 and a conservative modification thereof;
q) a heavy chain variable region CDR2 comprising the amino acid sequence set forth in SEQ ID NO: 136 and a conservative modification thereof; and a light chain variable region CDR2 comprising the amino acid sequence set forth in SEQ ID NO: 74 and a conservative modification thereof; and
r) a heavy chain variable region CDR2 comprising the amino acid sequence set forth in SEQ ID NO: 167 and a conservative modification thereof; and a light chain variable region CDR2 comprising the amino acid sequence set forth in SEQ ID NO: 170 and a conservative modification thereof;
s) a heavy chain variable region CDR2 comprising the amino acid sequence set forth in SEQ ID NO: 176 and a conservative modification thereof; and a light chain variable region CDR2 comprising the amino acid sequence set forth in SEQ ID NO: 50 and a conservative modification thereof;
t) a heavy chain variable region CDR2 comprising the amino acid sequence set forth in SEQ ID NO: 185 and a conservative modification thereof; and a light chain variable region CDR2 comprising the amino acid sequence set forth in SEQ ID NO: 188 and a conservative modification thereof;
u) a heavy chain variable region CDR2 comprising the amino acid sequence set forth in SEQ ID NO: 194 and a conservative modification thereof; and a light chain variable region CDR2 comprising the amino acid sequence set forth in SEQ ID NO: 5 and a conservative modification thereof; and
v) a heavy chain variable region CDR2 comprising the amino acid sequence set forth in SEQ ID NO: 202 and a conservative modification thereof; and a light chain variable region CDR2 comprising the amino acid sequence set forth in SEQ ID NO: 5 and a conservative modification thereof.

A3. The foregoing antibody-TCO conjugate of A1 or A2, wherein, wherein the heavy chain variable region and light chain variable region CDR1 domains are selected from the group consisting of:
a) a heavy chain variable region CDR1 comprising the amino acid sequence set forth in SEQ ID NO: 1 and a conservative modification thereof; and a light chain variable region CDR1 comprising the amino acid sequence set forth in SEQ ID NO: 4 and a conservative modification thereof;
b) a heavy chain variable region CDR1 comprising the amino acid sequence set forth in SEQ ID NO: 11 and a conservative modification thereof; and a light chain variable region CDR1 comprising the amino acid sequence set forth in SEQ ID NO: 14 and a conservative modification thereof;
c) a heavy chain variable region CDR1 comprising the amino acid sequence set forth in SEQ ID NO: 21 and a conservative modification thereof; and a light chain variable region CDR1 comprising the amino acid sequence set forth in SEQ ID NO: 4 and a conservative modification thereof;
d) a heavy chain variable region CDR1 comprising the amino acid sequence set forth in SEQ ID NO: 28 and a conservative modification thereof; and a light chain variable region CDR1 comprising the amino acid sequence set forth in SEQ ID NO: 31 and a conservative modification thereof;
e) a heavy chain variable region CDR1 comprising the amino acid sequence set forth in SEQ ID NO: 21 and a conservative modification thereof; and a light chain variable region CDR1 comprising the amino acid sequence set forth in SEQ ID NO: 40 and a conservative modification thereof;
f) a heavy chain variable region CDR1 comprising the amino acid sequence set forth in SEQ ID NO: 46 and a conservative modification thereof; and a light chain variable region CDR1 comprising the amino acid sequence set forth in SEQ ID NO: 49 and a conservative modification thereof;
g) a heavy chain variable region CDR1 comprising the amino acid sequence set forth in SEQ ID NO: 21 and a conservative modification thereof; and a light chain variable region CDR1 comprising the amino acid sequence set forth in SEQ ID NO: 57 and a conservative modification thereof;
h) a heavy chain variable region CDR1 comprising the amino acid sequence set forth in SEQ ID NO: 70 and a conservative modification thereof; and a light chain variable region CDR1 comprising the amino acid sequence set forth in SEQ ID NO: 73 and a conservative modification thereof;
i) a heavy chain variable region CDR1 comprising the amino acid sequence set forth in SEQ ID NO: 87 and a conservative modification thereof; and a light chain variable region CDR1 comprising the amino acid sequence set forth in SEQ ID NO: 90 and a conservative modification thereof;
j) a heavy chain variable region CDR1 comprising the amino acid sequence set forth in SEQ ID NO: 96 and a conservative modification thereof; and a light chain variable region CDR1 comprising the amino acid sequence set forth in SEQ ID NO: 99 and a conservative modification thereof;
k) a heavy chain variable region CDR1 comprising the amino acid sequence set forth in SEQ ID NO: 96 and a conservative modification thereof; and a light chain variable region CDR1 comprising the amino acid sequence set forth in SEQ ID NO: 117 and a conservative modification thereof;
l) a heavy chain variable region CDR1 comprising the amino acid sequence set forth in SEQ ID NO: 21 and a conservative modification thereof; and a light chain variable region CDR1 comprising the amino acid sequence set forth in SEQ ID NO: 124 and a conservative modification thereof;
m) a heavy chain variable region CDR1 comprising the amino acid sequence set forth in SEQ ID NO: 135 and a conservative modification thereof; and a light chain variable region CDR1 comprising the amino acid sequence set forth in SEQ ID NO: 138 and a conservative modification thereof;
n) a heavy chain variable region CDR1 comprising the amino acid sequence set forth in SEQ ID NO: 151 and a conservative modification thereof; and a light chain variable region CDR1 comprising the amino acid sequence set forth in SEQ ID NO: 57 and a conservative modification thereof;
o) a heavy chain variable region CDR1 comprising the amino acid sequence set forth in SEQ ID NO: 11 and a conservative modification thereof; and a light chain variable region CDR1 comprising the amino acid sequence set forth in SEQ ID NO: 73 and a conservative modification thereof;
p) a heavy chain variable region CDR1 comprising the amino acid sequence set forth in SEQ ID NO: 96 and a conservative modification thereof; and a light chain variable region CDR1 comprising the amino acid sequence set forth in SEQ ID NO: 169 and a conservative modification thereof;
q) a heavy chain variable region CDR1 comprising the amino acid sequence set forth in SEQ ID NO: 21 and a conservative modification thereof; and a light chain variable region CDR1 comprising the amino acid sequence set forth in SEQ ID NO: 178 and a conservative modification thereof;
r) a heavy chain variable region CDR1 comprising the amino acid sequence set forth in SEQ ID NO: 184 and a conservative modification thereof; and a light chain variable region CDR1 comprising the amino acid sequence set forth in SEQ ID NO: 187 and a conservative modification thereof;
s) a heavy chain variable region CDR1 comprising the amino acid sequence set forth in SEQ ID NO: 11 and a conservative modification thereof; and a light chain variable region CDR1 comprising the amino acid sequence set forth in SEQ ID NO: 4 and a conservative modification thereof; and
t) a heavy chain variable region CDR1 comprising the amino acid sequence set forth in SEQ ID NO: 201 and a conservative modification thereof; and a light chain variable region CDR1 comprising the amino acid sequence set forth in SEQ ID NO: 57 and a conservative modification thereof.

A4. The foregoing antibody-TCO conjugate of any one of A1-A3, wherein the anti-DLL3 antibody or antigen-binding fragment thereof comprises:
a) a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3;
b) a heavy chain variable region comprising a CDR1 comprising the amino acid sequence set forth in SEQ ID NO: 11, a CDR2 comprising the amino acid sequence set forth in SEQ ID NO: 12, and a CDR3 comprising the amino acid sequence set forth in SEQ ID NO: 13;
c) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 22;
d) a heavy chain variable region comprising a CDR1 comprising the amino acid sequence set forth in SEQ ID NO: 28, a CDR2 comprising the amino acid sequence set forth in SEQ ID NO: 29, and a CDR3 comprising the amino acid sequence set forth in SEQ ID NO: 30;
e) a heavy chain variable region comprising a CDR1 comprising the amino acid sequence set forth in SEQ ID NO: 21, a CDR2 comprising the amino acid sequence set forth in SEQ ID NO: 38, and a CDR3 comprising the amino acid sequence set forth in SEQ ID NO: 39;
f) a heavy chain variable region comprising a CDR1 comprising the amino acid sequence set forth in SEQ ID NO: 46, a CDR2 comprising the amino acid sequence set forth in SEQ ID NO: 47, and a CDR3 comprising the amino acid sequence set forth in SEQ ID NO: 48;
g) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 56;
h) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 64;
i) a heavy chain variable region comprising a CDR1 comprising the amino acid sequence set forth in SEQ ID NO: 70, a CDR2 comprising the amino acid sequence set forth in SEQ ID NO: 71 and a CDR3 comprising the amino acid sequence set forth in SEQ ID NO: 72;
j) a heavy chain variable region comprising a CDR1 comprising the amino acid sequence set forth in SEQ ID NO: 21, a CDR2 comprising the amino acid sequence set forth in SEQ ID NO: 80, and a CDR3 comprising the amino acid sequence set forth in SEQ ID NO: 81;
k) a heavy chain variable region comprising a CDR1 comprising the amino acid sequence set forth in SEQ ID NO: 87, a CDR2 comprising the amino acid sequence set forth in SEQ ID NO: 88, and a CDR3 comprising the amino acid sequence set forth in SEQ ID NO: 89;
l) a heavy chain variable region comprising a CDR1 comprising the amino acid sequence set forth in SEQ ID NO: 96, a CDR2 comprising the amino acid sequence set forth in SEQ ID NO: 97, and a CDR3 comprising the amino acid sequence set forth in SEQ ID NO: 98;
m) a heavy chain variable region comprising a CDR1 comprising the amino acid sequence set forth in SEQ ID NO: 21, a CDR2 comprising the amino acid sequence set forth in SEQ ID NO: 106, and a CDR3 comprising the amino acid sequence set forth in SEQ ID NO: 107;
n) a heavy chain variable region comprising a CDR1 comprising the amino acid sequence set forth in SEQ ID NO: 96, a CDR2 comprising the amino acid sequence set forth in SEQ ID NO: 115, and a CDR3 comprising the amino acid sequence set forth in SEQ ID NO: 116;
o) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 123;
p) a heavy chain variable region comprising a CDR1 comprising the amino acid sequence set forth in SEQ ID NO: 135, a CDR2 comprising the amino acid sequence set forth in SEQ ID NO: 136, and a CDR3 comprising the amino acid sequence set forth in SEQ ID NO: 137;
q) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 145;
r) a heavy chain variable region comprising a CDR1 comprising the amino acid sequence set forth in SEQ ID NO: 151, a CDR2 comprising the amino acid sequence set forth in SEQ ID NO: 2, and a CDR3 comprising the amino acid sequence set forth in SEQ ID NO: 152;
s) a heavy chain variable region comprising a CDR1 comprising the amino acid sequence set forth in SEQ ID NO: 11, a CDR2 comprising the amino acid sequence set forth in SEQ ID NO: 136, and a CDR3 comprising the amino acid sequence set forth in SEQ ID NO: 161;
t) a heavy chain variable region comprising a CDR1 comprising the amino acid sequence set forth in SEQ ID NO: 96, a CDR2 comprising the amino acid sequence set forth in SEQ ID NO: 167, and CDR3 comprising the amino acid sequence set forth in SEQ ID NO: 168;
u) a heavy chain variable region comprising a CDR1 comprising the amino acid sequence set forth in SEQ ID NO: 21, a CDR2 comprising the amino acid sequence set forth in SEQ ID NO: 176, and a CDR3 comprising the amino acid sequence set forth in SEQ ID NO: 177;
v) a heavy chain variable region comprising a CDR1 comprising the amino acid sequence set forth in SEQ ID NO: 184, a CDR2 comprising the amino acid sequence set forth in SEQ ID NO: 185, and a CDR3 comprising the amino acid sequence set forth in SEQ ID NO: 186;
w) a heavy chain variable region comprising a CDR1 comprising the amino acid sequence set forth in SEQ ID NO: 11, a CDR2 comprising the amino acid sequence set forth in SEQ ID NO: 194, and a CDR3 comprising the amino acid sequence set forth in SEQ ID NO: 195; or
x) a heavy chain variable region comprising a CDR1 comprising the amino acid sequence set forth in SEQ ID NO: 201, a CDR2 comprising the amino acid sequence set forth in SEQ ID NO: 202, and a CDR3 comprising the amino acid sequence set forth in SEQ ID NO: 203.

A5. The foregoing antibody-TCO conjugate of any one of A1-A4, wherein the anti-DLL3 antibody or antigen-binding fragment thereof comprises:
a) a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6;
b) a light chain variable region comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16;
c) a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 23;
d) a light chain variable region comprising a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33;
e) a light chain variable region comprising a CDR1 comprising the amino acid sequence set forth in SEQ ID NO: 40, a CDR2 comprising the amino acid sequence set forth in SEQ ID NO: 5, and a CDR3 comprising the amino acid sequence set forth in SEQ ID NO: 41;
f) a light chain variable region comprising a CDR1 comprising the amino acid sequence set forth in SEQ ID NO: 49, a CDR2 comprising the amino acid sequence set forth in SEQ ID NO: 50, and a CDR3 comprising the amino acid sequence set forth in SEQ ID NO: 51;
g) a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 59;
h) a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 65;
i) a light chain variable region comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 75;
j) a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82;
k) a light chain variable region comprising a CDR1 comprising the amino acid sequence set forth in SEQ ID NO: 90, a CDR2 comprising the amino acid sequence set forth in SEQ ID NO: 210; and a CDR3 comprising the amino acid sequence set forth in SEQ ID NO: 91;
l) a light chain variable region comprising a CDR1 comprising the amino acid sequence set forth in SEQ ID NO: 99, a CDR2 comprising the amino acid sequence set forth in SEQ ID NO: 100, and a CDR3 comprising the amino acid sequence set forth in SEQ ID NO: 101;
m) a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 112;
n) a light chain variable region comprising a CDR1 comprising the amino acid sequence set forth in SEQ ID NO: 117, a CDR2 comprising the amino acid sequence set forth in SEQ ID NO: 100, and a CDR3 comprising the amino acid sequence set forth in SEQ ID NO: 118;
o) a light chain variable region comprising a CDR1 comprising the amino acid sequence set forth in SEQ ID NO: 124, a CDR2 comprising the amino acid sequence set forth in SEQ ID NO: 58, and a CDR3 comprising the amino acid sequence set forth in SEQ ID NO: 125;
p) a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 130;
q) a light chain variable region comprising a CDR1 comprising the amino acid sequence set forth in SEQ ID NO: 138, a CDR2 comprising the amino acid sequence set forth in SEQ ID NO: 139, and a CDR3 comprising the amino acid sequence set forth in SEQ ID NO: 140;
r) a light chain variable region comprising a CDR1 comprising the amino acid sequence set forth in SEQ ID NO: 57, a CDR2 comprising the amino acid sequence set forth in SEQ ID NO: 146, and a CDR3 comprising the amino acid sequence set forth in SEQ ID NO: 125;
s) a light chain variable region comprising a CDR1 comprising the amino acid sequence set forth in SEQ ID NO: 124, a CDR2 comprising the amino acid sequence set forth in SEQ ID NO: 58, and CDR3 comprising the amino acid sequence set forth in SEQ ID NO: 59;
t) a light chain variable region comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 162;
u) a light chain variable region comprising a CDR1 comprising the amino acid sequence set forth in SEQ ID NO: 169, a light chain variable region CDR2 comprising the amino acid sequence set forth in SEQ ID NO: 170, and a CDR3 comprising the amino acid sequence set forth in SEQ ID NO: 171;
v) a light chain variable region comprising a CDR1 comprising the amino acid sequence set forth in SEQ ID NO: 178, a CDR2 comprising the amino acid sequence set forth in SEQ ID NO: 50, and a CDR3 comprising the amino acid sequence set forth in SEQ ID NO: 179;
w) a light chain variable region comprising a CDR1 comprising the amino acid sequence set forth in SEQ ID NO: 187, a CDR2 comprising the amino acid sequence set forth in SEQ ID NO: 188, and a CDR3 comprising the amino acid sequence set forth in SEQ ID NO: 189;
x) a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 196; or
y) a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 204.

A6. The foregoing antibody-TCO conjugate of any one of A1-A5, wherein the anti-DLL3 antibody or antigen-binding fragment thereof comprises:
a) a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a region CDR3 comprising the amino acid sequence set forth in SEQ ID NO: 6;
b) a heavy chain variable region comprising a CDR1 comprising the amino acid sequence set forth in SEQ ID NO: 11, a CDR2 comprising an amino acid sequence set forth in SEQ ID NO: 12, and a CDR3 comprising the amino acid sequence set forth in SEQ ID NO: 13; and a light chain variable region comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16;
c) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 22; and a light chain variable region comprising a CDR1 comprising the amino acid sequence set forth in SEQ ID NO: 4, a CDR2 comprising the amino acid sequence set forth in SEQ ID NO: 5, and a region CDR3 comprising the amino acid sequence set forth in SEQ ID NO: 23;
d) a heavy chain variable region comprising a CDR1 comprising the amino acid sequence set forth in SEQ ID NO: 28, a CDR2 comprising the amino acid sequence set forth in SEQ ID NO: 29, and a CDR3 comprising the amino acid sequence set forth in SEQ ID NO: 30; and a light chain variable region comprising a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33;
e) a heavy chain variable region comprising a CDR1 comprising the amino acid sequence set forth in SEQ ID NO: 21, a CDR2 comprising the amino acid sequence set forth in SEQ ID NO: 38, and a CDR3 comprising the amino acid sequence set forth in SEQ ID NO: 39; and a light chain variable region comprising a CDR1 comprising the amino acid sequence set forth in SEQ ID NO: 40, a CDR2 comprising the amino acid sequence set forth in SEQ ID NO: 5, and a CDR3 comprising the amino acid sequence set forth in SEQ ID NO: 41;
f) a heavy chain variable region comprising a CDR1 comprising the amino acid sequence set forth in SEQ ID NO: 46, a CDR2 comprising the amino acid sequence set forth in SEQ ID NO: 47, and a CDR3 comprising the amino acid sequence set forth in SEQ ID NO: 48; and a light chain variable region comprising a CDR1 comprising the amino acid sequence set forth in SEQ ID NO: 49, a CDR2 comprising the amino acid sequence set forth in SEQ ID NO: 50, and a CDR3 comprising the amino acid sequence set forth in SEQ ID NO: 51;
g) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 56; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 59;
h) a heavy chain variable region comprising a CDR1 comprising the amino acid sequence set forth in SEQ ID NO: 21, CDR2 comprising the amino acid sequence set forth in SEQ ID NO: 2 and a CDR3 comprising the amino acid sequence set forth in SEQ ID NO: 64; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 65;
i) a heavy chain variable region comprising a CDR1 comprising the amino acid sequence set forth in SEQ ID NO: 70, a CDR2 comprising the amino acid sequence set forth in SEQ ID NO: 71, and a CDR3 comprising the amino acid sequence set forth in SEQ ID NO: 72; and a light chain variable region comprising a CDR1 comprising the amino acid sequence set forth in SEQ ID NO: 73, a CDR2 comprising the amino acid sequence set forth in SEQ ID NO: 74, and CDR3 comprising the amino acid sequence set forth in SEQ ID NO: 75;
j) a heavy chain variable region comprising a CDR1 comprising the amino acid sequence set forth in SEQ ID NO: 21, a CDR2 comprising the amino acid sequence set forth in SEQ ID NO: 80, and a CDR3 comprising the amino acid sequence set forth in SEQ ID NO: 81; and a light chain variable region comprising a CDR1 comprising the amino acid sequence set forth in SEQ ID NO: 57, a CDR2 comprising the amino acid sequence set forth in SEQ ID NO: 58, and CDR3 comprising the amino acid sequence set forth in SEQ ID NO: 82;
k) a heavy chain variable region comprising a CDR1 comprising the amino acid sequence set forth in SEQ ID NO: 87, a CDR2 comprising the amino acid sequence set forth in SEQ ID NO: 88, and a CDR3 comprising the amino acid sequence set forth in SEQ ID NO: 89; and a light chain variable region comprising a CDR1 comprising the amino acid sequence set forth in SEQ ID NO: 90, a CDR2 comprising the amino acid sequence set forth in SEQ ID NO: 210, and a CDR3 comprising the amino acid sequence set forth in SEQ ID NO: 91;
l) a heavy chain variable region comprising a CDR1 comprising the amino acid sequence set forth in SEQ ID NO: 96, a CDR2 comprising the amino acid sequence set forth in SEQ ID NO: 97, and a CDR3 comprising the amino acid sequence set forth in SEQ ID NO: 98; and a light chain variable region comprising a CDR1 comprising the amino acid sequence set forth in SEQ ID NO: 99, a CDR2 comprising the amino acid sequence set forth in SEQ ID NO: 100, and a CDR3 comprising the amino acid sequence set forth in SEQ ID NO: 101;
m) a heavy chain variable region comprising a CDR1 comprising the amino acid sequence set forth in SEQ ID NO: 21, a CDR2 comprising the amino acid sequence set forth in SEQ ID NO: 106, and a CDR3 comprising the amino acid sequence set forth in SEQ ID NO: 107; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82;
n) a heavy chain variable region comprising a CDR1 comprising the amino acid sequence set forth in SEQ ID NO: 21, CDR2 comprising the amino acid sequence set forth in SEQ ID NO: 106, and a CDR3 comprising the amino acid sequence set forth in SEQ ID NO: 107; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 112;
o) a heavy chain variable region comprising a CDR1 comprising the amino acid sequence set forth in SEQ ID NO: 96, a CDR2 comprising the amino acid sequence set forth in SEQ ID NO: 115, and a CDR3 comprising the amino acid sequence set forth in SEQ ID NO: 116; and a light chain variable region comprising a CDR1 comprising the amino acid sequence set forth in SEQ ID NO: 117, a CDR2 comprising the amino acid sequence set forth in SEQ ID NO: 100, and a CDR3 comprising the amino acid sequence set forth in SEQ ID NO: 118;
p) a heavy chain variable region comprising a CDR1 comprising the amino acid sequence set forth in SEQ ID NO: 21, CDR2 comprising the amino acid sequence set forth in SEQ ID NO: 2, and a CDR3 comprising the amino acid sequence set forth in SEQ ID NO: 123; and a light chain variable region comprising a CDR1 comprising the amino acid sequence set forth in SEQ ID NO: 124, a CDR2 comprising the amino acid sequence set forth in SEQ ID NO: 58, and a CDR3 comprising the amino acid sequence set forth in SEQ ID NO: 125;
q) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 56; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 130;
r) a heavy chain variable region comprising a CDR1 comprising the amino acid sequence set forth in SEQ ID NO: 135, a CDR2 comprising the amino acid sequence set forth in SEQ ID NO: 136, and a CDR3 comprising the amino acid sequence set forth in SEQ ID NO: 137; and a light chain variable region comprising a CDR1 comprising the amino acid sequence set forth in SEQ ID NO: 138, a CDR2 comprising the amino acid sequence set forth in SEQ ID NO: 139, and a CDR3 comprising the amino acid sequence set forth in SEQ ID NO: 140;
s) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 145; and a light chain variable region comprising a CDR1 comprising the amino acid sequence set forth in SEQ ID NO: 57, a CDR2 comprising the amino acid sequence set forth in SEQ ID NO: 146, and a CDR3 comprising the amino acid sequence set forth in SEQ ID NO: 125;
t) a heavy chain variable region comprising a CDR1 comprising the amino acid sequence set forth in SEQ ID NO: 151, a CDR2 comprising the amino acid sequence set forth in SEQ ID NO: 2, and a CDR3 comprising the amino acid sequence set forth in SEQ ID NO: 152; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82;
u) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 123; and a light chain variable region comprising a CDR1 comprising the amino acid sequence set forth in SEQ ID NO: 124, a CDR2 comprising the amino acid sequence set forth in SEQ ID NO: 58, and a CDR3 comprising the amino acid sequence set forth in SEQ ID NO: 59;
v) a heavy chain variable region comprising a CDR1 comprising the amino acid sequence set forth in SEQ ID NO: 11, a CDR2 comprising the amino acid sequence set forth in SEQ ID NO: 136, and a CDR3 comprising the amino acid sequence set forth in SEQ ID NO: 161; and a light chain variable region comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 162;
w) a heavy chain variable region comprising a CDR1 comprising the amino acid sequence set forth in SEQ ID NO: 96, a CDR2 comprising the amino acid sequence set forth in SEQ ID NO: 167, and a CDR3 comprising the amino acid sequence set forth in SEQ ID NO: 168; and a light chain variable region comprising a CDR1 comprising the amino acid sequence set forth in SEQ ID NO: 169, a CDR2 comprising the amino acid sequence set forth in SEQ ID NO: 170, and a CDR3 comprising the amino acid sequence set forth in SEQ ID NO: 171;
x) a heavy chain variable region comprising a CDR1 comprising the amino acid sequence set forth in SEQ ID NO: 21, a CDR2 comprising the amino acid sequence set forth in SEQ ID NO: 176, and a CDR3 comprising the amino acid sequence set forth in SEQ ID NO: 177; and a light chain variable region comprising a CDR1 comprising the amino acid sequence set forth in SEQ ID NO: 178, a CDR2 comprising the amino acid sequence set forth in SEQ ID NO: 50, and a CDR3 comprising the amino acid sequence set forth in SEQ ID NO: 179;
y) a heavy chain variable region comprising a CDR1 comprising the amino acid sequence set forth in SEQ ID NO: 184, a CDR2 comprising the amino acid sequence set forth in SEQ ID NO: 185, and a CDR3 comprising the amino acid sequence set forth in SEQ ID NO: 186; and a light chain variable region comprising a CDR1 comprising the amino acid sequence set forth in SEQ ID NO: 187, a CDR2 comprising the amino acid sequence set forth in SEQ ID NO: 188, and a CDR3 comprising the amino acid sequence set forth in SEQ ID NO: 188;
z) a heavy chain variable region comprising a CDR1 comprising the amino acid sequence set forth in SEQ ID NO: 11, a CDR2 comprising the amino acid sequence set forth in SEQ ID NO: 194, and a CDR3 comprising the amino acid sequence set forth in SEQ ID NO: 195; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 196; or aa) a heavy chain variable region comprising a CDR1 comprising the amino acid sequence set forth in SEQ ID NO: 201, a CDR2 comprising the amino acid sequence set forth in SEQ ID NO: 202, and a CDR3 comprising the amino acid sequence set forth in SEQ ID NO: 203; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 204.

A7. The foregoing antibody-TCO conjugate of any one of A1-A6, wherein the anti-DLL3 antibody or antigen-binding fragment thereof comprises:
a) a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6;
b) a heavy chain variable region comprising a CDR1 comprising the amino acid sequence set forth in SEQ ID NO: 28, a CDR2 comprising the amino acid sequence set forth in SEQ ID NO: 29, and a CDR3 comprising the amino acid sequence set forth in SEQ ID NO: 30; and a light chain variable region comprising a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33; or
c) a heavy chain variable region comprising a CDR1 comprising the amino acid sequence set forth in SEQ ID NO: 21, a CDR2 comprising the amino acid sequence set forth in SEQ ID NO: 38, and a CDR3 comprising the amino acid sequence set forth in SEQ ID NO: 39; and a light chain variable region comprising a CDR1 comprising the amino acid sequence set forth in SEQ ID NO: 40, a CDR2 comprising the amino acid sequence set forth in SEQ ID NO: 5, and a CDR3 comprising the amino acid sequence set forth in SEQ ID NO: 41.

A8. The foregoing antibody-TCO conjugate of any one of A1-A7, wherein the anti-DLL3 antibody or antigen-binding fragment thereof comprises a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 7, SEQ ID NO: 17, SEQ ID NO: 24, SEQ ID NO: 34, SEQ ID NO: 42, SEQ ID NO: 52, SEQ ID NO: 60, SEQ ID NO: 66, SEQ ID NO: 76, SEQ ID NO: 83, SEQ ID NO: 92, SEQ ID NO: 102, SEQ ID NO: 108, SEQ ID NO: 119, SEQ ID NO: 126, SEQ ID NO: 131, SEQ ID NO: 141, SEQ ID NO: 147, SEQ ID NO: 153, SEQ ID NO: 157, SEQ ID NO: 163, SEQ ID NO: 172, SEQ ID NO: 180, SEQ ID NO: 190, SEQ ID NO: 197, or SEQ ID NO: 205.

A9. The foregoing antibody-TCO conjugate of any one of A1-A8, wherein the anti-DLL3 antibody or antigen-binding fragment thereof comprises a heavy chain variable region comprising the amino acid sequence set forth in SEQ ID NO: 7, SEQ ID NO: 17, SEQ ID NO: 24, SEQ ID NO: 34, SEQ ID NO: 42, SEQ ID NO: 52, SEQ ID NO: 60, SEQ ID NO: 66, SEQ ID NO: 76, SEQ ID NO: 83, SEQ ID NO: 92, SEQ ID NO: 102, SEQ ID NO: 108, SEQ ID NO: 119, SEQ ID NO: 126, SEQ ID NO: 131, SEQ ID NO: 141, SEQ ID NO: 147, SEQ ID NO: 153, SEQ ID NO: 157, SEQ ID NO: 163, SEQ ID NO: 172, SEQ ID NO: 180, SEQ ID NO: 190, SEQ ID NO: 197, or SEQ ID NO: 205.

A10. The foregoing antibody-TCO conjugate of any one of A1-A9, wherein the anti-DLL3 antibody or antigen-binding fragment thereof comprises a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 8, SEQ ID NO: 18, SEQ ID NO: 25, SEQ ID NO: 35, SEQ ID NO: 43, SEQ ID NO: 53, SEQ ID NO: 61, SEQ ID NO: 67, SEQ ID NO: 77, SEQ ID NO: 84, SEQ ID NO: 93, SEQ ID NO: 103, SEQ ID NO: 109, SEQ ID NO: 113, SEQ ID NO: 120, SEQ ID NO: 127, SEQ ID NO: 132, SEQ ID NO: 142, SEQ ID NO: 148, SEQ ID NO: 154, SEQ ID NO: 158, SEQ ID NO: 164, SEQ ID NO: 173, SEQ ID NO: 181, SEQ ID NO: 191, SEQ ID NO: 198, or SEQ ID NO: 206.

A11. The foregoing antibody-TCO conjugate of any one of A1-A10, wherein the anti-DLL3 antibody or antigen-binding fragment thereof comprises a light chain variable region comprising the amino acid sequence set forth in SEQ ID NO: 8, SEQ ID NO: 18, SEQ ID NO: 25, SEQ ID NO: 35, SEQ ID NO: 43, SEQ ID NO: 53, SEQ ID NO: 61, SEQ ID NO: 67, SEQ ID NO: 77, SEQ ID NO: 84, SEQ ID NO: 93, SEQ ID NO: 103, SEQ ID NO: 109, SEQ ID NO: 113, SEQ ID NO: 120, SEQ ID NO: 127, SEQ ID NO: 132, SEQ ID NO: 142, SEQ ID NO: 148, SEQ ID NO: 154, SEQ ID NO: 158, SEQ ID NO: 164, SEQ ID NO: 173, SEQ ID NO: 181, SEQ ID NO: 191, SEQ ID NO: 198, or SEQ ID NO: 206.

A12. The foregoing antibody-TCO conjugate of any one of A1-A11, wherein the anti-DLL3 antibody or antigen-binding fragment thereof comprises:
a) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 7, SEQ ID NO: 17, SEQ ID NO: 24, SEQ ID NO: 34, SEQ ID NO: 42, SEQ ID NO: 52, SEQ ID NO: 60, SEQ ID NO: 66, SEQ ID NO: 76, SEQ ID NO: 83, SEQ ID NO: 92, SEQ ID NO: 102, SEQ ID NO: 108, SEQ ID NO: 119, SEQ ID NO: 126, SEQ ID NO: 131, SEQ ID NO: 141, SEQ ID NO: 147, SEQ ID NO: 153, SEQ ID NO: 157, SEQ ID NO: 163, SEQ ID NO: 172, SEQ ID NO: 180, SEQ ID NO: 190, SEQ ID NO: 197, or SEQ ID NO: 205; and
b) a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 8, SEQ ID NO: 18, SEQ ID NO: 25, SEQ ID NO: 35, SEQ ID NO: 43, SEQ ID NO: 53, SEQ ID NO: 61, SEQ ID NO: 67, SEQ ID NO: 77, SEQ ID NO: 84, SEQ ID NO: 93, SEQ ID NO: 103, SEQ ID NO: 109, SEQ ID NO: 113, SEQ ID NO: 120, SEQ ID NO: 127, SEQ ID NO: 132, SEQ ID NO: 142, SEQ ID NO: 148, SEQ ID NO: 154, SEQ ID NO: 158, SEQ ID NO: 164, SEQ ID NO: 173, SEQ ID NO: 181, SEQ ID NO: 191, SEQ ID NO: 198, or SEQ ID NO: 206.

A13. The foregoing antibody-TCO conjugate of any one of A1-A12, wherein the anti-DLL3 antibody or antigen-binding fragment thereof comprises:
a) a heavy chain variable region comprising the amino acid sequence set forth in SEQ ID NO: 7, SEQ ID NO: 17, SEQ ID NO: 24, SEQ ID NO: 34, SEQ ID NO: 42, SEQ ID NO: 52, SEQ ID NO: 60, SEQ ID NO: 66, SEQ ID NO: 76, SEQ ID NO: 83, SEQ ID NO: 92, SEQ ID NO: 102, SEQ ID NO: 108, SEQ ID NO: 119, SEQ ID NO: 126, SEQ ID NO: 131, SEQ ID NO: 141, SEQ ID NO: 147, SEQ ID NO: 153, SEQ ID NO: 157, SEQ ID NO: 163, SEQ ID NO: 172, SEQ ID NO: 180, SEQ ID NO: 190, SEQ ID NO: 197, or SEQ ID NO: 205; and
b) a light chain variable region comprising the amino acid sequence set forth in SEQ ID NO: 8, SEQ ID NO: 18, SEQ ID NO: 25, SEQ ID NO: 35, SEQ ID NO: 43, SEQ ID NO: 53, SEQ ID NO: 61, SEQ ID NO: 67, SEQ ID NO: 77, SEQ ID NO: 84, SEQ ID NO: 93, SEQ ID NO: 103, SEQ ID NO: 109, SEQ ID NO: 113, SEQ ID NO: 120, SEQ ID NO: 127, SEQ ID NO: 132, SEQ ID NO: 142, SEQ ID NO: 148, SEQ ID NO: 154, SEQ ID NO: 158, SEQ ID NO: 164, SEQ ID NO: 173, SEQ ID NO: 181, SEQ ID NO: 191, SEQ ID NO: 198, or SEQ ID NO: 206.

A14. The foregoing antibody-TCO conjugate of any one of A1-A13, wherein the anti-DLL3 antibody or antigen-binding fragment thereof comprises:
a) a heavy chain variable region comprising the amino acid sequence set forth in SEQ ID NO: 7, and a light chain variable region comprising the amino acid sequence set forth in SEQ ID NO: 8;
b) a heavy chain variable region comprising the amino acid sequence set forth in SEQ ID NO: 17, and a light chain variable region comprising the amino acid sequence set forth in SEQ ID NO: 18;
c) a heavy chain variable region comprising the amino acid sequence set forth in SEQ ID NO: 24, and a light chain variable region comprising the amino acid sequence set forth in SEQ ID NO: 25;
d) a heavy chain variable region comprising the amino acid sequence set forth in SEQ ID NO: 34, and a light chain variable region comprising the amino acid sequence set forth in SEQ ID NO: 35;
e) a heavy chain variable region comprising the amino acid sequence set forth in SEQ ID NO: 42, and a light chain variable region comprising the amino acid sequence set forth in SEQ ID NO: 43;
f) a heavy chain variable region comprising the amino acid sequence set forth in SEQ ID NO: 52, and a light chain variable region comprising the amino acid sequence set forth in SEQ ID NO: 53;
g) a heavy chain variable region comprising the amino acid sequence set forth in SEQ ID NO: 60, and a light chain variable region comprising the amino acid sequence set forth in SEQ ID NO: 61;
h) a heavy chain variable region comprising the amino acid sequence set forth in SEQ ID NO: 66, and a light chain variable region comprising the amino acid sequence set forth in SEQ ID NO: 67;
i) a heavy chain variable region comprising the amino acid sequence set forth in SEQ ID NO: 76, and a light chain variable region comprising the amino acid sequence set forth in SEQ ID NO: 77;
j) a heavy chain variable region comprising the amino acid sequence set forth in SEQ ID NO: 83, and a light chain variable region comprising the amino acid sequence set forth in SEQ ID NO: 84;
k) a heavy chain variable region comprising the amino acid sequence set forth in SEQ ID NO: 92, and a light chain variable region comprising the amino acid sequence set forth in SEQ ID NO: 93;
l) a heavy chain variable region comprising the amino acid sequence set forth in SEQ ID NO: 102, and a light chain variable region comprising the amino acid sequence set forth in SEQ ID NO: 103;
m) a heavy chain variable region comprising the amino acid sequence set forth in SEQ ID NO: 108, and a light chain variable region comprising the amino acid sequence set forth in SEQ ID NO: 109;
n) a heavy chain variable region comprising the amino acid sequence set forth in SEQ ID NO: 108, and a light chain variable region comprising the amino acid sequence set forth in SEQ ID NO: 113;
o) a heavy chain variable region comprising the amino acid sequence set forth in SEQ ID NO: 119, and a light chain variable region comprising the amino acid sequence set forth in SEQ ID NO: 120;
p) a heavy chain variable region comprising the amino acid sequence set forth in SEQ ID NO: 126, and a light chain variable region comprising the amino acid sequence set forth in SEQ ID NO: 127;
q) a heavy chain variable region comprising the amino acid sequence set forth in SEQ ID NO: 131, and a light chain variable region comprising the amino acid sequence set forth in SEQ ID NO: 132;
r) a heavy chain variable region comprising the amino acid sequence set forth in SEQ ID NO: 141, and a light chain variable region comprising the amino acid sequence set forth in SEQ ID NO: 142;
s) a heavy chain variable region comprising the amino acid sequence set forth in SEQ ID NO: 147, and a light chain variable region comprising the amino acid sequence set forth in SEQ ID NO: 148;
t) a heavy chain variable region comprising the amino acid sequence set forth in SEQ ID NO: 153, and a light chain variable region comprising the amino acid sequence set forth in SEQ ID NO: 154;
u) a heavy chain variable region comprising the amino acid sequence set forth in SEQ ID NO: 157, and a light chain variable region comprising the amino acid sequence set forth in SEQ ID NO: 158;
v) a heavy chain variable region comprising the amino acid sequence set forth in SEQ ID NO: 163, and a light chain variable region comprising the amino acid sequence set forth in SEQ ID NO: 164;
w) a heavy chain variable region comprising the amino acid sequence set forth in SEQ ID NO: 172, and a light chain variable region comprising the amino acid sequence set forth in SEQ ID NO: 173;
x) a heavy chain variable region comprising the amino acid sequence set forth in SEQ ID NO: 180, and a light chain variable region comprising the amino acid sequence set forth in SEQ ID NO: 181;
y) a heavy chain variable region comprising the amino acid sequence set forth in SEQ ID NO: 190, and a light chain variable region comprising the amino acid sequence set forth in SEQ ID NO: 191;
z) a heavy chain variable region comprising the amino acid sequence set forth in SEQ ID NO: 197, and a light chain variable region comprising the amino acid sequence set forth in SEQ ID NO: 198; or aa) a heavy chain variable region comprising the amino acid sequence set forth in SEQ ID NO: 205, and a light chain variable region comprising the amino acid sequence set forth in SEQ ID NO: 206.

A15. The foregoing antibody-TCO conjugate of any one of A1-A14, comprising:
a) a heavy chain variable region comprising the amino acid sequence set forth in SEQ ID NO: 7, and a light chain variable region comprising the amino acid sequence set forth in SEQ ID NO: 8;
b) a heavy chain variable region comprising the amino acid sequence set forth in SEQ ID NO: 34, and a light chain variable region comprising the amino acid sequence set forth in SEQ ID NO: 35; or
c) a heavy chain variable region comprising the amino acid sequence set forth in SEQ ID NO: 42, and a light chain variable region comprising the amino acid sequence set forth in SEQ ID NO: 43.

A16. The foregoing antibody-TCO conjugate of any one of A1-A15, wherein the TCO has formula

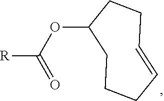

and wherein R is N, C, or an ester.

A17. The foregoing antibody-TCO conjugate of A16, wherein the TCO has formula

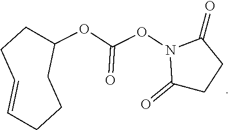 [SMILES: O=C(OC1CC/C=C/CCC1)ON1C(=O)CCC1=O]

B1. In certain non-limiting embodiments, the presently disclosed subject matter provides a composition comprising the antibody-TCO conjugate of any one of A1-A17.

B2. The foregoing composition of B1, which is pharmaceutical composition further comprising pharmaceutically acceptable carrier.

C1. In certain non-limiting embodiments, the presently disclosed subject matter provides a radioimmunoconjugate comprising the antibody-TCO conjugate of any one of A1-A17 and a radioligand comprising a tetrazine moiety (Tz) and a radiolabel.

C2. The foregoing radioimmunoconjugate of C1, wherein the radiolabel comprises a radioactive isotope selected from the group consisting of ^{99m}Tc, ¹¹¹In, ⁴⁴Sc, ⁴⁷Sc, ⁶⁴Cu, ⁶⁷Ga, ⁶⁸Ga, ⁷²As, ⁷⁷As, ⁵²Mn, ¹⁸⁶Re, ¹⁵³Sm, ¹⁷⁷Lu, ⁶⁷Cu, ¹²³I, ¹²⁴I, ¹²⁵I, ¹¹C, ¹³N, ¹⁵O, ¹⁸F, ¹⁶⁶Ho ¹⁴⁹Pm, ⁹⁰Y, ⁸⁶Y, ²¹³Bi, ¹⁰³Pd, ¹⁰⁹Pd ¹⁵⁹Gd, ¹⁴⁰La, ¹⁹⁸Au, ¹⁹⁹Au, ¹⁷⁵Yb, ¹⁶⁹Yb, ¹⁶⁵Dy, ¹⁶⁶Dy, ¹⁰⁵Ph, ¹¹¹Ag, ⁸⁹Zr, ¹⁹²Ir, ²²⁵Ac ²¹²Bi, ²¹³Bi ⁷⁶Br, ⁵⁵Co, ⁶⁰Cu, ⁶¹Cu, ⁶²Cu, ⁶⁶Ga, ⁹⁰Nb, ²¹²Pb, ¹⁵²Tb, ⁷⁷Br, ²¹¹At, ²²⁷Th, ²²⁴Ra, and ¹⁴⁹Tb.

C3. The foregoing radioimmunoconjugate of C1 or C2, wherein the radiolabel comprises a ⁶⁴Cu radioactive isotope.

C4. The foregoing radioimmunoconjugate of C1 or C2, wherein the radiolabel comprises a ²²⁵Ac radioactive isotope.

C5. The foregoing radioimmunoconjugate of any one of C1-C4, further comprising a chelator.

C6. The foregoing radioimmunoconjugate of C5, wherein the chelator is a sarcophagine chelator.

C7. The foregoing radioimmunoconjugate of C5, wherein the chelator is 1,4,7-triazonane-1,4,7-triyl-triacetic acid (NOTA) or 1,4,7-triazacyclononane-1,4-diacetate (NODA).

C8. The foregoing radioimmunoconjugate of any one of C1-C7, further comprising a linker attaching the tetrazine moiety (Tz) to the chelator.

C9. The foregoing radioimmunoconjugate of C8, wherein the linker comprises is polyethylene glycol (PEG) or (poly)-L-lysine.

C10. The foregoing radioimmunoconjugate of C9, wherein the polyethylene glycol (PEG) comprises from about 1 unit to about 100 units.

C11. The foregoing radioimmunoconjugate of C9, wherein the (poly)-L-lysine comprises from about 1 unit to about 200 units.

C12. The foregoing radioimmunoconjugate of any one of C1-C11, wherein the tetrazine moiety (Tz) and the chelator form a compound selected from the group consisting of:
a) 2,2′,2″-(3-(4-(3-(1-(4-(1,2,4,5-tetrazin-3-yl)phenyl)-1-oxo-5,8,11,14,17,20,23-heptaoxa-2-azapentacosan-25-yl)thioureido)benzyl)-1,4,7-triazonane-2,5,8-triyl)triacetic acid;
b) 2,2′,2″-(3-(4-(3-(1-(4-(1,2,4,5-tetrazin-3-yl)phenyl)-1-oxo-5,8,11,14,17,20,23,26,29,32,35-undecaoxa-2-azaheptatriacontan-37-yl)thioureido)benzyl)-1,4,7-triazonane-2,5,8-triyl)triacetic acid;
c) 2,2′-(7-(4-(3-(1-(4-(1,2,4,5-tetrazin-3-yl)phenyl)-1-oxo-5,8,11,14,17,20,23,26,29,32,35-undecaoxa-2-azaheptatriacontan-37-yl)thioureido)benzyl)-1,4,7-triazonane-1,4-diyl)diacetic acid;
d) 2,2′,2″-(3-(4-(3-(1-(4-(1,2,4,5-tetrazin-3-yl)phenyl)-3,7-dioxo-11,14,17,20,23,26,29-heptaoxa-2,8-diazahentriacontan-31-yl)thioureido)benzyl)-1,4,7-triazonane-2,5,8-triyl)triacetic acid;
e) 2,2′,2″-(3-(4-(3-(1-(4-(1,2,4,5-tetrazin-3-yl)phenyl)-3,7-dioxo-11,14,17,20,23,26,29,32,35,38,41-undecaoxa-2,8-diazatritetracontan-43-yl)thioureido)benzyl)-1,4,7-triazonane-2,5,8-triyl)triacetic acid;
f) 2,2′,2″-(3-(4-(3-(25,28-dioxo-28-((6-(6-(pyridin-2-yl)-1,2,4,5-tetrazin-3-yl)pyridin-3-yl)amino)-3,6,9,12,15,18,21-heptaoxa-24-azaoctacosyl)thioureido)benzyl)-1,4,7-triazonane-2,5,8-triyl)triacetic acid;
g) 2,2′,2″-(3-(4-(3-(37,40-dioxo-40-((6-(6-(pyridin-2-yl)-1,2,4,5-tetrazin-3-yl)pyridin-3-yl)amino)-3,6,9,12,15,18,21,24,27,30,33-undecaoxa-36-azatetracontyl)thioureido)benzyl)-1,4,7-triazonane-2,5,8-triyl)triacetic acid;
h) 2,2′,2″-(3-(4-(1-(4-(6-methyl-1,2,4,5-tetrazin-3-yl)phenyl)-3-oxo-6,9,12,15,18,21,24-heptaoxa-2-azaheptacosan-27-amido)benzyl)-1,4,7-triazonane-2,5,8-triyl)triacetic acid;
i) 2,2′,2″-(2-(4-(1-(4-(6-methyl-1,2,4,5-tetrazin-3-yl)phenoxy)-3,6,9,12,15,18,21,24,27,30,33-undecaoxahexatriacontan-36-amido)benzyl)-1,4,7-triazonane-1,4,7-triyl)triacetic acid;
j) 2,2′,2″-(3-(4-(3-(5-amino-6-((4-(6-methyl-1,2,4,5-tetrazin-3-yl)benzyl)amino)-6-oxohexyl)thioureido)benzyl)-1,4,7-triazonane-2,5,8-triyl)triacetic acid;
k) 2,2′-(7-(4-(3-(5-amino-6-((4-6-methyl-1,2,4,5-tetrazin-3-yl)benzyl)amino)-6-oxohexyl)thioureido)benzyl)-1,4,7-triazonane-1,4-diyl)diacetic acid;
l) 2,2′,2″-(3-(4-(3-(5-amino-6-((5-amino-6-((4-(6-methyl-1,2,4,5-tetrazin-3-yl)benzyl)amino)-6-oxohexyl)amino)-6-oxohexyl)thioureido)benzyl)-1,4,7-triazonane-2,5,8-triyl)triacetic acid; and
m) 2,2′,2″-(3-(4-(3-(5-amino-6-((5-amino-6-((5-amino-6-((4-(6-methyl-1,2,4,5-tetrazin-3-yl)benzyl)amino)-6-oxohexyl)amino)-6-oxohexyl)amino)-6-oxohexyl)thioureido)benzyl)-1,4,7-triazonane-2,5,8-triyl)triacetic acid.

C13. The foregoing radioimmunoconjugate of any one of C1-C12, wherein the antibody-TCO conjugate and the radioligand bind together to form the radioimmunoconjugate via an in vivo click reaction.

D1. In certain embodiments, the presently disclosed subject matter provides a method of in vivo diagnosis of a disease or disorder associated with DLL3 in a subject, comprising:
a) administering to the subject the antibody-TCO conjugate of any one of A1-A17 or the composition of B1 or B2, wherein a portion of the antibody-TCO conjugate localizes at cells of the disease or disorder and unbound antibody-TCO conjugate is cleared from blood, from renal system, and/or from the subject after an accumulation interval,
b) administering to the subject the a radioligand comprising a tetrazine moiety (Tz) and a radiolabel or a composition comprising the radioligand, wherein the antibody-TCO conjugate and the radioligand bind together to form a radioimmunoconjugate via an in vivo click reaction at the cells of the disease or disorder within a region of the subject; and
c) imaging the radioimmunoconjugate accumulated in the region of the subject.

D2. The foregoing method of D1, wherein the radioligand is delivered to the subject after the accumulation interval.

D3. The foregoing method of D1 or D2, wherein the imaging occurs via positron emission tomography (PET).

D4. The foregoing method of any one of D1-D3, wherein the imaging occurs within a time period up to about 7 days from the administering of the radioligand.

D5. The foregoing method of claim any one of D1-D4, wherein the radiolabel comprises a radioactive isotope selected from the group consisting of ^{99m}Tc, ¹¹¹In, ⁴⁴Sc, ⁴⁷Sc, ⁶⁴Cu, ⁶⁷Ga, ⁶⁸Ga, ⁷²As, ⁷⁷As, ⁵²Mn, ¹⁸⁶Re, ¹⁵³Sm, ¹⁷⁷Lu, ⁶⁷Cu, ¹²³I, ¹²⁴I, ¹²⁵I, ¹¹C, ¹³N, ¹⁵O, ¹⁸F, ¹⁶⁶Ho, ¹⁴⁹Pm, ⁹⁰Y, ⁸⁶Y, ²¹³Bi, ¹⁰³Pd, ¹⁰⁹Pd, ¹⁵⁹Gd, ¹⁴⁰La, ¹⁹⁸Au, ¹⁹⁹Au, ¹⁷⁵Yb, ¹⁶⁹Yb, ¹⁶⁵Dy, ¹⁶⁶Dy, ¹⁰⁵Ph, ¹¹¹Ag, ⁸⁹Zr, ¹⁹²Ir, ²²⁵Ac, ²¹²Bi, ²¹³Bi, ⁷⁶Br, ⁵⁵Co, ⁶⁰Cu, ⁶¹Cu, ⁶²Cu, ⁶⁶Ga, ⁹⁰Nb, ²¹²Pb, ¹⁵²Tb, ⁷⁷Br, ²¹¹At, ²²⁷Th, ²²⁴Ra, and ¹⁴⁹Tb.

D6. The foregoing method of any one of D1-D5, wherein the radiolabel comprises a ⁶⁴Cu radioactive isotope.

D7. The foregoing method of any one of D1-D5, wherein the radiolabel comprises a 18F radioactive isotope.

D8. The foregoing method of any one of D1-D7, wherein the radioligand further comprises a chelator.

D9. The foregoing method of D8, wherein the chelator is a sarcophagine chelator.

D10. The foregoing method of D9, wherein the chelator is 1,4,7-triazonane-1,4,7-triyl-triacetic acid (NOTA) or 1,4,7-triazacyclononane-1,4-diacetate (NODA).

D11. The foregoing method of any one of D1-D10, wherein the radioligand further comprises a linker attaching the tetrazine moiety (Tz) to the chelator.

D12. The foregoing method of D11, wherein the linker comprises is polyethylene glycol (PEG) or (poly)-L-lysine.

D13. The foregoing method of D12, wherein the polyethylene glycol (PEG) comprises from about 1 unit to about 100 units.

D14. The foregoing method of D12, wherein the (poly)-L-lysine comprises from about 1 unit to about 200 units.

D15. The foregoing method of D1-D14, wherein the tetrazine moiety (Tz) and the chelator form a compound selected from the group consisting of:
a) 2,2′,2″-(3-(4-(3-(1-(4-(1,2,4,5-tetrazin-3-yl)phenyl)-1-oxo-5,8,11,14,17,20,23-heptaoxa-2-azapentacosan-25-yl)thioureido)benzyl)-1,4,7-triazonane-2,5,8-triyl)triacetic acid;
b) 2,2′,2″-(3-(4-(3-(1-(4-(1,2,4,5-tetrazin-3-yl)phenyl)-1-oxo-5,8,11,14,17,20,23,26,29,32,35-undecaoxa-2-azaheptatriacontan-37-yl)thioureido)benzyl)-1,4,7-triazonane-2,5,8-triyl)triacetic acid;
c) 2,2′-(7-(4-(3-(1-(4-(1,2,4,5-tetrazin-3-yl)phenyl)-1-oxo-5,8,11,14,17,20,23,26,29,32,35-undecaoxa-2-azaheptatriacontan-37-yl)thioureido)benzyl)-1,4,7-triazonane-1,4-diyl)diacetic acid;
d) 2,2′,2″-(3-(4-(3-(1-(4-(1,2,4,5-tetrazin-3-yl)phenyl)-3,7-dioxo-11,14,17,20,23,26,29-heptaoxa-2,8-diazahentriacontan-31-yl)thioureido)benzyl)-1,4,7-triazonane-2,5,8-triyl)triacetic acid;
e) 2,2′,2″-(3-(4-(3-(1-(4-(1,2,4,5-tetrazin-3-yl)phenyl)-3,7-dioxo-11,14,17,20,23,26,29,32,35,38,41-undecaoxa-2,8-diazatritetracontan-43-yl)thioureido)benzyl)-1,4,7-triazonane-2,5,8-triyl)triacetic acid;
f) 2,2′,2″-(3-(4-(3-(25,28-dioxo-28-((6-(6-(pyridin-2-yl)-1,2,4,5-tetrazin-3-yl)pyridin-3-yl)amino)-3,6,9,12,15,18,21-heptaoxa-24-azaoctacosyl)thioureido)benzyl)-1,4,7-triazonane-2,5,8-triyl)triacetic acid;
g) 2,2′,2″-(3-(4-(3-(37,40-dioxo-40-((6-(6-(pyridin-2-yl)-1,2,4,5-tetrazin-3-yl)pyridin-3-yl)amino)-3,6,9,12,15,18,21,24,27,30,33-undecaoxa-36-azatetracontyl)thioureido)benzyl)-1,4,7-triazonane-2,5,8-triyl)triacetic acid;
h) 2,2′,2″-(3-(4-(1-(4-(6-methyl-1,2,4,5-tetrazin-3-yl)phenyl)-3-oxo-6,9,12,15,18,21,24-heptaoxa-2-azaheptacosan-27-amido)benzyl)-1,4,7-triazonane-2,5,8-triyl)triacetic acid;
i) 2,2′,2″-(2-(4-(1-(4-(6-methyl-1,2,4,5-tetrazin-3-yl)phenoxy)-3,6,9,12,15,18,21,24,27,30,33-undecaoxahexatriacontan-36-amido)benzyl)-1,4,7-triazonane-1,4,7-triyl)triacetic acid;
j) 2,2′,2″-(3-(4-(3-(5-amino-6-((4-(6-methyl-1,2,4,5-tetrazin-3-yl)benzyl)amino)-6-oxohexyl)thioureido)benzyl)-1,4,7-triazonane-2,5,8-triyl)triacetic acid;
k) 2,2′-(7-(4-(3-(5-amino-6-((4-6-methyl-1,2,4,5-tetrazin-3-yl)benzyl)amino)-6-oxohexyl)thioureido)benzyl)-1,4,7-triazonane-1,4-diyl)diacetic acid;
l) 2,2′,2″-(3-(4-(3-(5-amino-6-((5-amino-6-((4-(6-methyl-1,2,4,5-tetrazin-3-yl)benzyl)amino)-6-oxohexyl)amino)-6-oxohexyl)thioureido)benzyl)-1,4,7-triazonane-2,5,8-triyl)triacetic acid; and
m) 2,2′,2″-(3-(4-(3-(5-amino-6-((5-amino-6-((5-amino-6-((4-(6-methyl-1,2,4,5-tetrazin-3-yl)benzyl)amino)-6-oxohexyl)amino)-6-oxohexyl)amino)-6-oxohexyl)thioureido)benzyl)-1,4,7-triazonane-2,5,8-triyl)triacetic acid.

D16. The foregoing method of any one of D1-D15, wherein the imaging comprises detecting the radioactive levels emitted by the radioimmunoconjugate.

D17. The foregoing method of D16, wherein the radioactive levels emitted by the radioimmunoconjugate are expressed as the percentage injected dose per gram tissue (% ID/g).

E1. In certain non-limiting embodiments, the presently disclosed subject matter provides a method for treating a disease or disorder associated with DLL3 in a subject, comprising:
a) administering to the subject the antibody-TCO conjugate of any one of A1-A17 or the composition of B1 or B2, wherein a portion of the antibody-TCO conjugate localizes at cells of the disease or disorder and unbound antibody-TCO conjugate is cleared from blood, from renal system, and/or from the subject after an accumulation interval; and
b) administering to the subject the a radioligand comprising a tetrazine moiety (Tz) and a radiolabel or a composition comprising the radioligand, wherein the antibody-TCO conjugate and the radioligand bind together to form a radioimmunoconjugate via an in vivo click reaction at the cells of the disease or disorder within a region of the subject, and wherein the radiolabel comprises a radioactive isotope selected from the group consisting of ^{99m}Tc, ¹¹¹In, ⁴⁴Sc, ⁴⁷Sc, ⁶⁴Cu, ⁶⁷Ga, ⁶⁸Ga, ⁷²As, ⁷⁷As, ⁵²Mn, ¹⁸⁶Re, ¹⁵³Sm, ¹⁷⁷Lu, ⁶⁷Cu, ¹²³I, ¹²⁴I, ¹²⁵I, ¹¹C, ¹³N, ¹⁵O, ¹⁸F, ¹⁶⁶Ho, ¹⁴⁹Pm, ⁹⁰Y, ⁸⁶Y, ²¹³Bi, ¹⁰³Pd, ¹⁰⁹Pd, ¹⁵⁹Gd, ¹⁴⁰La, ¹⁹⁸Au, ¹⁹⁹Au, ¹⁷⁵Yb, ¹⁶⁹Yb, ¹⁶⁵Dy, ¹⁶⁶Dy, ¹⁰⁵Ph, ¹¹¹Ag, ⁸⁹Zr, ¹⁹²Ir, ²²⁵Ac, ²¹²Bi, ²¹³Bi, ⁷⁶Br, ⁵⁵Co, ⁶⁰Cu, ⁶¹Cu, ⁶²Cu, ⁶⁶Ga, ⁹⁰Nb, ²¹²Pb, ¹⁵²Tb, ⁷⁷Br, ²¹¹At, ²²⁷Th, ²²⁴Ra, and ¹⁴⁹Tb.

E2. The foregoing method of E1, wherein the radioligand is delivered to the subject after the accumulation interval.

E2. The foregoing method of E1 or E2, wherein the radioligand further comprises a chelator.

E3. The foregoing method of E2, wherein the chelator is a sarcophagine chelator.

E4. The foregoing method of E2, wherein the chelator is 1,4,7-triazonane-1,4,7-triyl-triacetic acid (NOTA) or 1,4,7-triazacyclononane-1,4-diacetate (NODA).

E5. The foregoing method of any one of E1-E4, wherein the radioligand further comprises a linker attaching the tetrazine moiety (Tz) to the chelator.

E6. The foregoing method of E5, wherein the linker comprises is polyethylene glycol (PEG) or (poly)-L-lysine.

E7. The foregoing method of E6, wherein the polyethylene glycol (PEG) comprises from about 1 unit to about 100 units.

E8. The foregoing method of E6, wherein the (poly)-L-lysine comprises from about 1 unit to about 200 units.

E9. The foregoing method of any one of E1-E8, wherein the tetrazine moiety (Tz) and the chelator form a compound selected from the group consisting of:
a) 2,2′,2″-(3-(4-(3-(1-(4-(1,2,4,5-tetrazin-3-yl)phenyl)-1-oxo-5,8,11,14,17,20,23-heptaoxa-2-azapentacosan-25-yl)thioureido)benzyl)-1,4,7-triazonane-2,5,8-triyl)triacetic acid;
b) 2,2′,2″-(3-(4-(3-(1-(4-(1,2,4,5-tetrazin-3-yl)phenyl)-1-oxo-5,8,11,14,17,20,23,26,29,32,35-undecaoxa-2-azaheptatriacontan-37-yl)thioureido)benzyl)-1,4,7-triazonane-2,5,8-triyl)triacetic acid;
c) 2,2′-(7-(4-(3-(1-(4-(1,2,4,5-tetrazin-3-yl)phenyl)-1-oxo-5,8,11,14,17,20,23,26,29,32,35-undecaoxa-2-azaheptatriacontan-37-yl)thioureido)benzyl)-1,4,7-triazonane-1,4-diyl)diacetic acid;
d) 2,2′,2″-(3-(4-(3-(1-(4-(1,2,4,5-tetrazin-3-yl)phenyl)-3,7-dioxo-11,14,17,20,23,26,29-heptaoxa-2,8-diazahentriacontan-31-yl)thioureido)benzyl)-1,4,7-triazonane-2,5,8-triyl)triacetic acid;
e) 2,2′,2″-(3-(4-(3-(1-(4-(1,2,4,5-tetrazin-3-yl)phenyl)-3,7-dioxo-11,14,17,20,23,26,29,32,35,38,41-undecaoxa-2,8-diazatritetracontan-43-yl)thioureido)benzyl)-1,4,7-triazonane-2,5,8-triyl)triacetic acid;
f) 2,2′,2″-(3-(4-(3-(25,28-dioxo-28-((6-(6-(pyridin-2-yl)-1,2,4,5-tetrazin-3-yl)pyridin-3-yl)amino)-3,6,9,12,15,18,21-heptaoxa-24-azaoctacosyl)thioureido)benzyl)-1,4,7-triazonane-2,5,8-triyl)triacetic acid;
g) 2,2′,2″-(3-(4-(3-(37,40-dioxo-40-((6-(6-(pyridin-2-yl)-1,2,4,5-tetrazin-3-yl)pyridin-3-yl)amino)-3,6,9,12,15,18,21,24,27,30,33-undecaoxa-36-azatetracontyl)thioureido)benzyl)-1,4,7-triazonane-2,5,8-triyl)triacetic acid;
h) 2,2′,2″-(3-(4-(1-(4-(6-methyl-1,2,4,5-tetrazin-3-yl)phenyl)-3-oxo-6,9,12,15,18,21,24-heptaoxa-2-azaheptacosan-27-amido)benzyl)-1,4,7-triazonane-2,5,8-triyl)triacetic acid;
i) 2,2′,2″-(2-(4-(1-(4-(6-methyl-1,2,4,5-tetrazin-3-yl)phenoxy)-3,6,9,12,15,18,21,24,27,30,33-undecaoxahexatriacontan-36-amido)benzyl)-1,4,7-triazonane-1,4,7-triyl)triacetic acid;
j) 2,2′,2″-(3-(4-(3-(5-amino-6-((4-(6-methyl-1,2,4,5-tetrazin-3-yl)benzyl)amino)-6-oxohexyl)thioureido)benzyl)-1,4,7-triazonane-2,5,8-triyl)triacetic acid;
k) 2,2′-(7-(4-(3-(5-amino-6-((4-6-methyl-1,2,4,5-tetrazin-3-yl)benzyl)amino)-6-oxohexyl)thioureido)benzyl)-1,4,7-triazonane-1,4-diyl)diacetic acid;
l) 2,2′,2″-(3-(4-(3-(5-amino-6-((5-amino-6-((4-(6-methyl-1,2,4,5-tetrazin-3-yl)benzyl)amino)-6-oxohexyl)amino)-6-oxohexyl)thioureido)benzyl)-1,4,7-triazonane-2,5,8-triyl)triacetic acid; and
m) 2,2′,2″-(3-(4-(3-(5-amino-6-((5-amino-6-((5-amino-6-((4-(6-methyl-1,2,4,5-tetrazin-3-yl)benzyl)amino)-6-oxohexyl)amino)-6-oxohexyl)amino)-6-oxohexyl)thioureido)benzyl)-1,4,7-triazonane-2,5,8-triyl)triacetic acid.

E10. The foregoing method of any one of E1-E9, wherein the disease or disorder associated with DLL3 is a tumor.

E11. The foregoing method of E10, wherein the tumor is a cancer.

E12. The foregoing method of any one of E1-E11, wherein the disease or disorder associated with DLL3 is selected from neuroendocrine tumors of the lung, extrapulmonary neuroendocrine carcinomas, melanoma, neuroendocrine prostate cancer, and breast cancer.

E13. The method of E12, wherein the neuroendocrine tumors of the lung are selected from the group consisting of pulmonary neuroendocrine cancer, large cell neuroendocrine carcinoma, and small-cell lung cancer.

E14. The method of any one of E1-E13, wherein the subject is a human.

F1. In certain non-limiting embodiments, the presently disclosed subject matter provides a kit for in vivo diagnosis and/or treatment of a disease or disorder associated with DLL3 in a subject, comprising the antibody-TCO conjugate of any one of A1-A17 or the composition of B1 or B2, and a radioligand comprising a tetrazine moiety (Tz) and a radiolabel or a composition thereof.

F2. The foregoing kit of F1, wherein the kit further comprises written instructions for using the antibody-TCO conjugate and the radioligand for in vivo diagnosis and/or treatment of a disease or disorder associated with DLL3 in a subject.

### 6. EXAMPLES

The following examples are put forth so as to provide those of ordinary skill in the art with a complete disclosure and description of how to make and use the antibodies, multi-specific antibodies, compositions comprising thereof, screening, and therapeutic methods of the presently disclosed subject matter, and are not intended to limit the scope of what the inventors regard as their presently disclosed subject matter. It is understood that various other embodiments may be practiced, given the general description provided above.

### Example 1—Generation of the Presently Disclosed Anti-DLL3 Antibodies and scFvs

The extracellular domain (ECD) of DLL3 (GenBank accession number Q9NY J7-1) corresponding to amino acids Ala27-Ala479 with a C-terminal 6×His tag produced in HEK293T cells stably expressing full length DLL3 were used as immunogens. Ablexis AlivaMAb Kappa Mice (Ablexis, San Diego, CA) harboring a human immunoglobulin repertoire were immunized either with soluble DLL3-ECD following standard immunization techniques over a period of 3 weeks. Splenocytes and draining lymph node cells from mice with high serum titers specific for DLL3 were harvested and fused with mouse myeloma cells to generate hybridomas using electrofusion. These hybridomas were then screened to identify the presence of antibodies that bound specifically to soluble DLL3-ECD by ELISA and full-length DLL3 protein on stably expressing 293 cells by flow cytometry versus parental 293 cells. In addition, these hybridomas were screened for reactivity with HEK293 cells stably expressing cynomolgus DLL3 or mouse DLL3. Hybridomas were selected for further investigation by ranking in flow cytometry for staining intensity on 293 DLL3 transfectants along with 4° C./37° C. staining to evaluate the internalization of the antibodies.

### Example 2—Epitope Binning of Panel of the Presently Disclosed Anti-DLL3 Monoclonal Antibodies

The panel of purified anti-DLL3 monoclonal antibodies and the reference monoclonal antibody were subjected to pairwise epitope binning on a Carterra® array surface plasmon resonance (SPR) assay platform (Carterra*Inc., Salt Lake City UT) where each monoclonal antibody was tested for the capture of Histidine-tagged DLL3 antigen (DLL3-His), and also for competing with every other antibody in the panel for the binding to DLL3-His. The antibodies were immobilized on a HC200M chip (ligand) through standard amine coupling techniques by the print array method. Then in each cycle antigen was injected across the entire array followed by a single antibody (analyte). At the end of each cycle the surface was regenerated to remove antigen and analyte before a new cycle started. As shown in the Table 28 below. These results demonstrate that the anti-DLL3-antibodies disclosed herein bind to distinct epitopes present in DLL3 protein. Accordingly, the anti-DLL3-antibodies disclosed herein can be used in combination with each other for delivering multiple therapeutic agents to tumor cells expressing DLL3

TABLE 28

| Monoclonal | |
|---|---|
| Antibodies | Epitope Bin |
| E23 | 7 |
| F9 | 4 |
| E9 | 4 |
| B22 | 2 |
| N12 | 8 |
| N10 | 7 |
| B16-v1 | 4 |
| B16-v2 | 4 |
| L22 | 8 |
| A18 | 6 |
| D8 | 2 |
| G16 | 4 |
| K19 | 5 |
| G3 | 1 |
| J23 | 2 |
| J8 | 4 |
| L12 | 4 |
| B2 | 4 |
| C22 | 9 |
| F21 | 5 |
| O24 | 4 |
| M11 | 2 |
| P4 | 5 |

### Example 3—Binding Affinities of the Presently Disclosed Anti-DLL3 Antibodies

Binding affinities of the anti-DDL3 antibodies disclosed herein were determined by biolayer interferometry (BLI) using the Octet HTX instrument at 25° C. using PBS 0.1% BSA 0.02% Tween 20 as the binding buffer and 10 mM Glycine pH 1.7 as the regeneration buffer. The 23 purified monoclonal antibodies (5 μg/mL each) were loaded onto anti-mouse Fc sensors. Loaded sensors were dipped into a serial dilutions of Recombinant Human DLL3 Protein, (amino acids Ala27-Ala479, cat #9749-DL, R&D Systems) at a 200 nM starting concentration, with 7 serial 1:3 dilutions. The binding affinities of all the monoclonal antibodies are shown in Table 29 below.

These results demonstrate that the anti-DLL3 antibodies disclosed herein specifically bind DLL3 with high affinity.

TABLE 29

| Monoclonal | |
|---|---|
| antibodies | Kd (nM) |
| E23 | 0.511 |
| F9 | 3.010 |
| E9 | 0.300 |
| B22 | 1.840 |
| N12 | 0.467 |
| N10 | 1.940 |
| L22 | 3.550 |
| A18 | 0.236 |
| D8 | 0.438 |
| G16 | 4.880 |
| K19 | 1.890 |
| G3 | 0.908 |
| J23 | 0.998 |
| J8 | 0.001 |
| L12 | 3.100 |
| B2 | 1.460 |
| C22 | 0.431 |
| F21 | 6.120 |
| O24 | 1.280 |
| M11 | 2.760 |
| P4 | 6.500 |

Next, affinity, binding domain, and specificity of antibodies including scFv designated as J8, B2, and L22 were analyzed. Affinity, binding domain and specificity of novel human anti-DLL3 antibodies are depicted in Table 30 below.

TABLE 30

| | Affinity | Binding | ELISA | ELISA | ELISA |
|---|---|---|---|---|---|
| Name | hDLL3 | domain | hDLL3 | hDLL1 | hDLL4 |
| B2 | 3.55 nM | EGF-like 5 | 1.205 | 0.4 | 0.043 |
| | | and EGF-like | | | |
| | | 6 | | | |
| J8 | <0.001 nM | EGF-like 5 | 1.377 | 0.095 | 0.085 |
| | | and EGF-like | | | |
| | | 6 | | | |
| L22 | 1.46 nM | EGF-like 3 | 1.639 | 0.105 | 0.056 |
| | | and EGF-like | | | |
| | | 4 | | | |

### Example 4—Screening of Novel Anti-DLL3 Antibodies for Pretargeted Imagining and Radioimmunotherapy

Small cell lung cancer (SCLC) is a particularly lethal form of lung cancer, accounting for roughly 15% of new lung cancer cases (Saunders et al., Sci Transl Med 2015; 7(302):302ra136). Delta-like ligand 3 (DLL3) has recently emerged as a therapeutic target in SCLC, in part because DLL3 cell surface expression is restricted almost exclusively to malignant tissue. Multiple DLL3-targeted therapies have been tested clinically, including the antibody-drug conjugate rovalpituzumab teserine (Rova-T); the bispecific T-cell engager AMG 757; and the CAR-T cell AMG 119 (Owen et al., J Hematol Oncol 2019; 12(1):61). As a companion diagnostic to evaluate DLL3 expression, an immunoPET strategy was developed by directly radiolabeling the DLL3-targeting SC16 antibody component of Rova-T with Zr-89 (Sharma et al., Cancer Res 2017; 77(14):3931-41). While the immunoPET tracer was able to clearly delineate subcutaneous, orthotopic, and metastatic SCLC xenografts in mice, optimal imaging was not achieved until 120 post-injection of the [⁸⁹Zr]Zr-DFO-SC16 because of the antibody's slow pharmacokinetics.

Pretargeting strategies that combine antibodies with small molecule radioligands have recently gained popularity for both in vivo imaging and radioimmunotherapy of cancer (Zeglis et al., Mol Pharm 2015; 12(10):3575-87). Pretargeting, as illustrated in FIG. 1, takes advantage of the excellent affinity and specificity of antibodies while simultaneously evading the limitations associated with their slow pharmacokinetics. This approach not only allows for faster imaging post-administration of the radiotracer, but also lowers systemic radiation dose to the patient. Using pretargeting to lower radiation dose becomes especially relevant in radioimmunotherapy, as the decrease in systemic dose widens the therapeutic index and reduces the risk of toxicity (Poty et al., Clin Cancer Res 2019; 25(2):868-80). Because pretargeting cannot be performed with the SC16 antibody (SC16 is rapidly internalized by SCLC cells upon its binding to DLL3), a panel of anti-DLL3 antibodies was generated in the pursuit of finding a non-internalizing antibody for pretargeted immunoPET imaging and radioimmunotherapy.

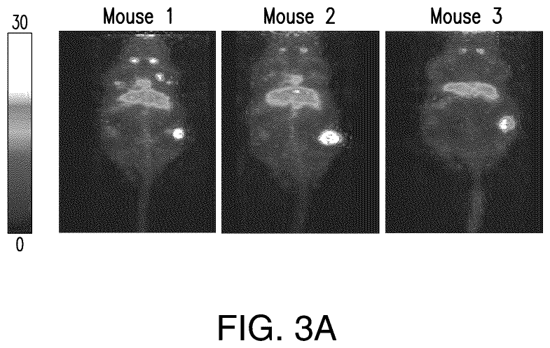

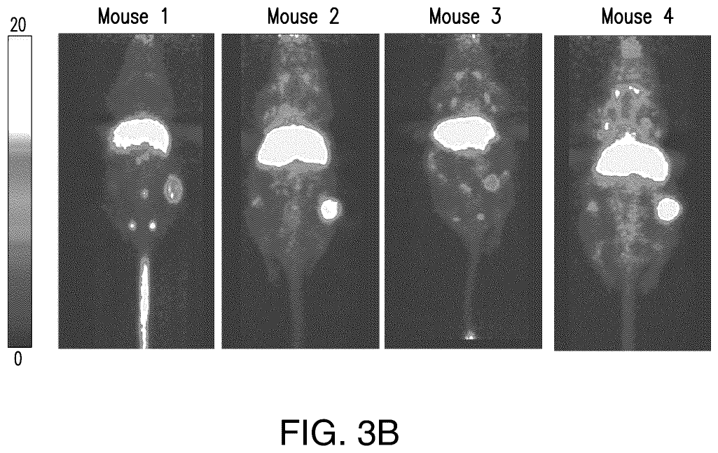

The presently disclosed subject matter reports the preclinical imaging of anti-DLL3 monoclonal antibodies to find the antibody clone best suited for pretargeted immunoPET imaging and radioimmunotherapy. After in vitro screening of 27 anti-DLL3 antibodies for low internalization rates, three monoclonal IgG4 antibodies were further evaluated in vivo: A18, E9, and J8. To ensure adequate binding to DLL3 in vivo, the three antibodies were first bioconjugated to p-SCN-Bn-DFO chelators and then radiolabeled with Zr-89 for injection via tail vein into athymic female mice bearing right flank subcutaneous H82 xenografts (250-300 Ci, 30 μg). ImmunoPET imaging was performed every 24 h until 120 h, when a terminal ex vivo biodistribution study was performed. The immunoPET images and ex vivo biodistribution profiles indicated strong tumor uptake of the antibodies in the xenografts, ranging from 20-50% ID/g, confirming that these antibodies have sufficient binding to DLL3 in vivo (see FIGS. 3A and 3B).

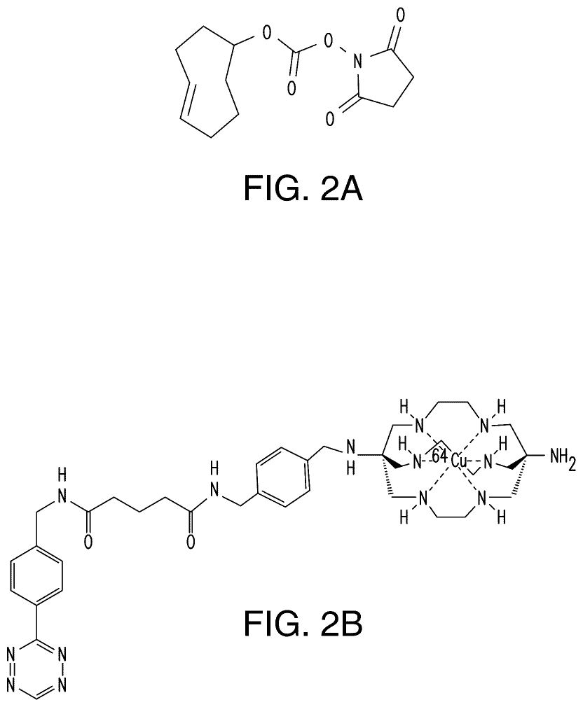

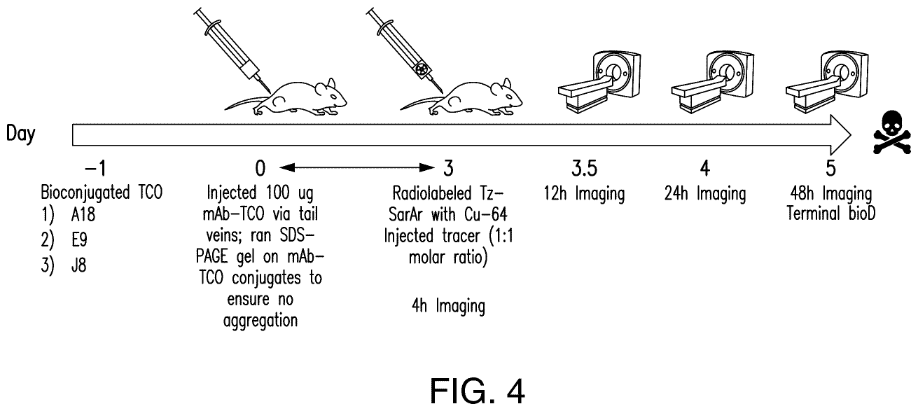

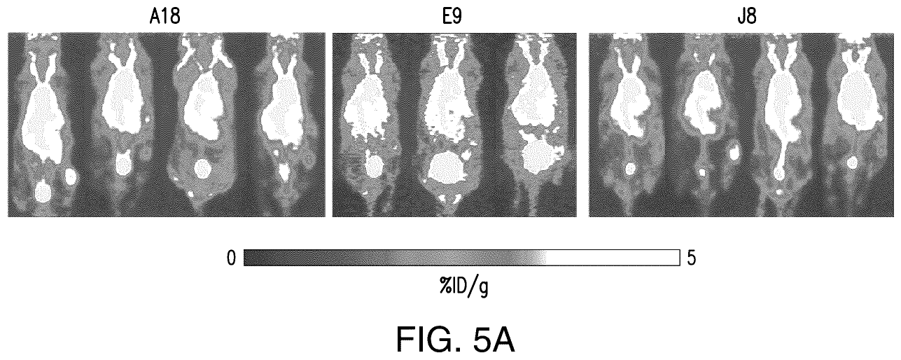

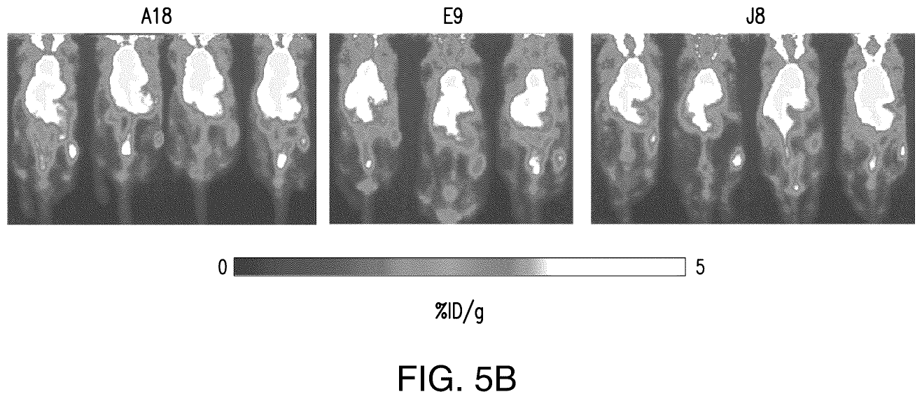

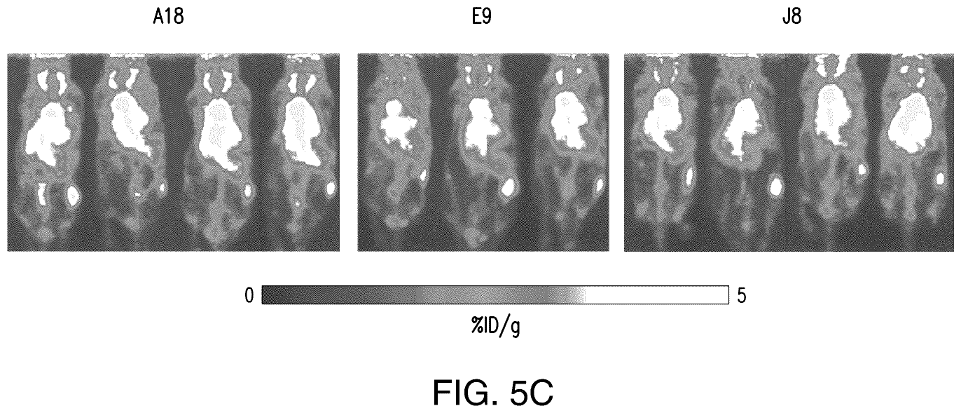

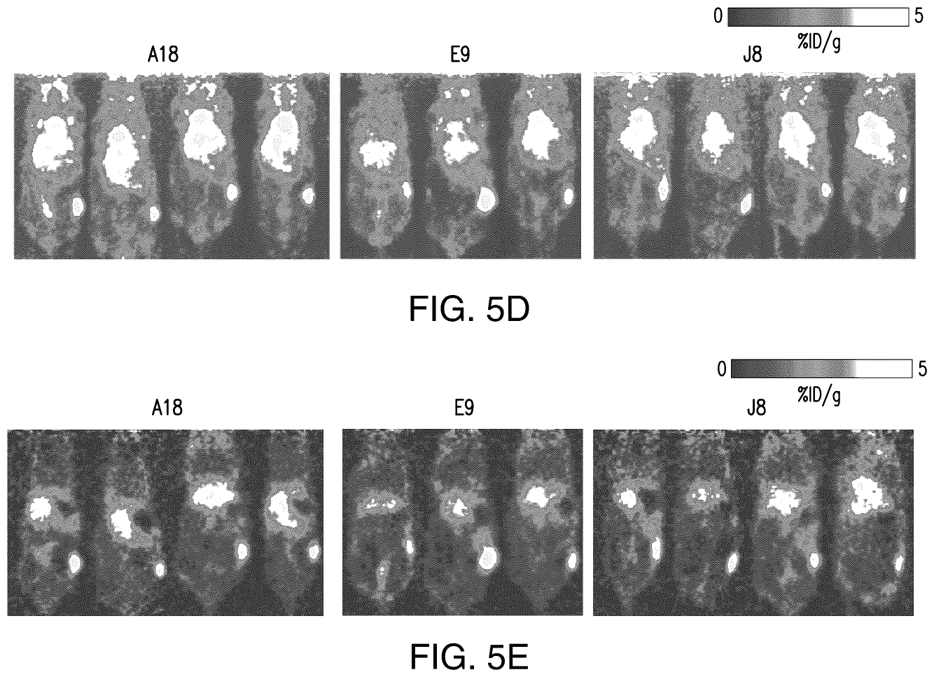

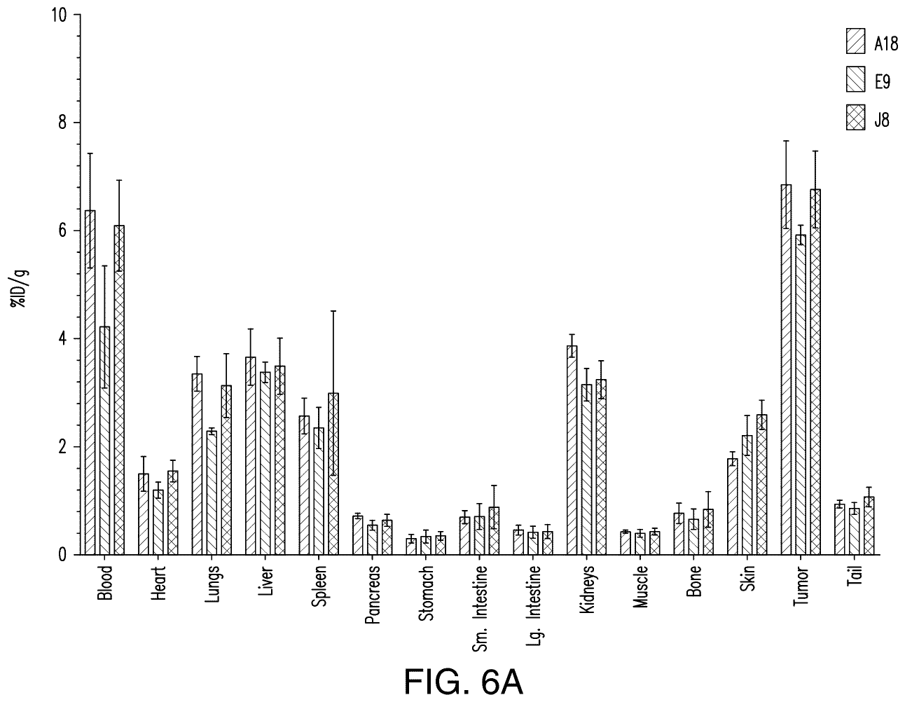

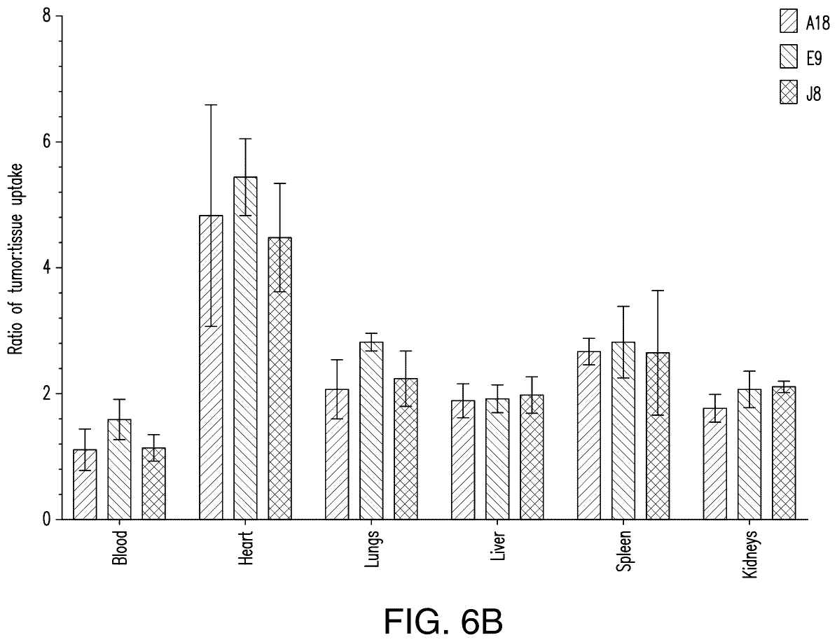

Pilot pretargeting experiments were then performed using the bioorthogonal IEDDA chemistry between trans-cyclooctene (TCO, see FIG. 2A) and tetrazine (Tz, see FIG. 2B). TCO-NHS ester was bioconjugated to the antibodies and purified anti-DLL3 antibody-TCO conjugates were injected via tail vein into the same xenograft model as previously described (100 μg, 0.67 nmol). After allowing the anti-DLL3 antibody-TCO conjugates to accumulate at the tumor site and clear from non-target tissue for 72 h, [⁶⁴Cu]Cu-SarAr-Tz was injected (490-540 μCi, 0.67 nmol) and immunoPET images were taken 4 h, 12 h, 24 h, and 48 h post-administration before a terminal ex vivo biodistribution was performed (FIG. 4). While absolute tumor uptake was significantly lower in the pretargeting experiments than the directly labeled Zr-89 experiments (5-8% ID/g compared to 20-50% ID/g) (see FIGS. 3A and 3B), the tumors were still able to be clearly delineated in the PET images (see FIGS. 5A-5E), and the ex vivo tumor-to-tissue ratios were quite high (see FIGS. 6A and 6B). All three antibodies performed very similarly, and there was no antibody that was clearly superior to the others.

For pretargeted radioimmunotherapy, after allowing the antibody-TCO conjugates to accumulate at the tumor site and clear from non-target tissue for 72 h, a [¹⁷⁷Lu]Lu-DOTA-Tz or a [²²⁵Ac]Ac-DOTA-Tz is injected.

### Embodiments of the Presently Disclosed Subject Matter

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. An antibody-transcyclooctene (TCO) conjugate comprising an anti-DLL3 antibody or an antigen-binding fragment thereof comprising a heavy chain variable region that comprises CDR1, CDR2, and CDR3 domains; and a light chain variable region that comprises CDR1, CDR2, and CDR3 domains, wherein the heavy chain variable region and light chain variable region CDR3 domains are selected from the group consisting of:
a) a heavy chain variable region CDR3 comprising the amino acid sequence set forth in SEQ ID NO: 3 and a conservative modification thereof; and a light chain variable region CDR3 comprising the amino acid sequence set forth in SEQ ID NO: 6 and a conservative modification thereof;
b) a heavy chain variable region CDR3 comprising the amino acid sequence set forth in SEQ ID NO: 13 and a conservative modification thereof; and a light chain variable region CDR3 comprising the amino acid sequence set forth in SEQ ID NO: 16 and a conservative modification thereof;
c) a heavy chain variable region CDR3 comprising the amino acid sequence set forth in SEQ ID NO: 22 and a conservative modification thereof; and a light chain variable region CDR3 comprising the amino acid sequence set forth in SEQ ID NO: 23 and a conservative modification thereof;
d) a heavy chain variable region CDR3 comprising the amino acid sequence set forth in SEQ ID NO: 30 and a conservative modification thereof; and a light chain variable region CDR3 comprising the amino acid sequence set forth in SEQ ID NO: 33 and a conservative modification thereof;
e) a heavy chain variable region CDR3 comprising the amino acid sequence set forth in SEQ ID NO: 39 and a conservative modification thereof; and a light chain variable region CDR3 comprising the amino acid sequence set forth in SEQ ID NO: 41 and a conservative modification thereof;
f) a heavy chain variable region CDR3 comprising the amino acid sequence set forth in SEQ ID NO: 48 and a conservative modification thereof; and a light chain variable region CDR3 comprising the amino acid sequence set forth in SEQ ID NO: 51 and a conservative modification thereof;
g) a heavy chain variable region CDR3 comprising the amino acid sequence set forth in SEQ ID NO: 56 and a conservative modification thereof; and a light chain variable region CDR3 comprising the amino acid sequence set forth in SEQ ID NO: 59 and a conservative modification thereof;
h) a heavy chain variable region CDR3 comprising the amino acid sequence set forth in SEQ ID NO: 64 and a conservative modification thereof; and a light chain variable region CDR3 comprising the amino acid sequence set forth in SEQ ID NO: 65 and a conservative modification thereof;
i) a heavy chain variable region CDR3 comprising the amino acid sequence set forth in SEQ ID NO: 72 and a conservative modification thereof; and a light chain variable region CDR3 comprising the amino acid sequence set forth in SEQ ID NO: 75 and a conservative modification thereof;
j) a heavy chain variable region CDR3 comprising the amino acid sequence set forth in SEQ ID NO: 81 and a conservative modification thereof; and a light chain variable region CDR3 comprising the amino acid sequence set forth in SEQ ID NO: 82 and a conservative modification thereof;
k) a heavy chain variable region CDR3 comprising the amino acid sequence set forth in SEQ ID NO: 89 and a conservative modification thereof; and a light chain variable region CDR3 comprising the amino acid sequence set forth in SEQ ID NO: 91 and a conservative modification thereof;
l) a heavy chain variable region CDR3 comprising the amino acid sequence set forth in SEQ ID NO: 98 and a conservative modification thereof; and a light chain variable region CDR3 comprising the amino acid sequence set forth in SEQ ID NO: 101 and a conservative modification thereof;
m) a heavy chain variable region CDR3 comprising the amino acid sequence set forth in SEQ ID NO: 107 and a conservative modification thereof; and a light chain variable region CDR3 comprising the amino acid sequence set forth in SEQ ID NO: 82 and a conservative modification thereof;
n) a heavy chain variable region CDR3 comprising the amino acid sequence set forth in SEQ ID NO: 107 and a conservative modification thereof; and a light chain variable region CDR3 comprising the amino acid sequence set forth in SEQ ID NO: 112 and a conservative modification thereof;
o) a heavy chain variable region CDR3 comprising the amino acid sequence set forth in SEQ ID NO: 116 and a conservative modification thereof; and a light chain variable region CDR3 comprising the amino acid sequence set forth in SEQ ID NO: 118 and a conservative modification thereof;
p) a heavy chain variable region CDR3 comprising the amino acid sequence set forth in SEQ ID NO: 123 and a conservative modification thereof; and a light chain variable region CDR3 comprising the amino acid sequence set forth in SEQ ID NO: 125 and a conservative modification thereof;
q) a heavy chain variable region CDR3 comprising the amino acid sequence set forth in SEQ ID NO: 56 and a conservative modification thereof; and a light chain variable region CDR3 comprising the amino acid sequence set forth in SEQ ID NO: 130 and a conservative modification thereof;
r) a heavy chain variable region CDR3 comprising the amino acid sequence set forth in SEQ ID NO: 137 and a conservative modification thereof; and a light chain variable region CDR3 comprising the amino acid sequence set forth in SEQ ID NO: 140 and a conservative modification thereof;
s) a heavy chain variable region CDR3 comprising the amino acid sequence set forth in SEQ ID NO: 145 and a conservative modification thereof; and a light chain variable region CDR3 comprising the amino acid sequence set forth in SEQ ID NO: 125 and a conservative modification thereof;
t) a heavy chain variable region CDR3 comprising the amino acid sequence set forth in SEQ ID NO: 152 and a conservative modification thereof; and a light chain variable region CDR3 comprising the amino acid sequence set forth in SEQ ID NO: 82 and a conservative modification thereof;
u) a heavy chain variable region CDR3 comprising the amino acid sequence set forth in SEQ ID NO: 123 and a conservative modification thereof; and a light chain variable region CDR3 comprising the amino acid sequence set forth in SEQ ID NO: 59 and a conservative modification thereof;
v) a heavy chain variable region CDR3 comprising the amino acid sequence set forth in SEQ ID NO: 161 and a conservative modification thereof; and a light chain variable region CDR3 comprising the amino acid sequence set forth in SEQ ID NO: 162 and a conservative modification thereof;
w) a heavy chain variable region CDR3 comprising the amino acid sequence set forth in SEQ ID NO: 168 and a conservative modification thereof; and a light chain variable region CDR3 comprising the amino acid sequence set forth in SEQ ID NO: 171 and a conservative modification thereof;
x) a heavy chain variable region CDR3 comprising the amino acid sequence set forth in SEQ ID NO: 177 and a conservative modification thereof; and a light chain variable region CDR3 comprising the amino acid sequence set forth in SEQ ID NO: 179 and a conservative modification thereof;
y) a heavy chain variable region CDR3 comprising the amino acid sequence set forth in SEQ ID NO: 186 and a conservative modification thereof; and a light chain variable region CDR3 comprising the amino acid sequence set forth in SEQ ID NO: 189 and a conservative modification thereof;
z) a heavy chain variable region CDR3 comprising the amino acid sequence set forth in SEQ ID NO: 195 and a conservative modification thereof; and a light chain variable region CDR3 comprising the amino acid sequence set forth in SEQ ID NO: 196 and a conservative modification thereof; and
aa) a heavy chain variable region CDR3 comprising the amino acid sequence set forth in SEQ ID NO: 203 and a conservative modification thereof; and a light chain variable region CDR3 comprising the amino acid sequence set forth in SEQ ID NO: 204 and a conservative modification thereof.

2. The antibody-TCO conjugate of claim 1, wherein the heavy chain variable region and light chain variable region CDR2 domains are selected from the group consisting of:
a) a heavy chain variable region CDR2 comprising the amino acid sequence set forth in SEQ ID NO: 2 and a conservative modification thereof; and a light chain variable region CDR2 comprising the amino acid sequence set forth in SEQ ID NO: 5 and a conservative modification thereof;
b) a heavy chain variable region CDR2 comprising the amino acid sequence set forth in SEQ ID NO: 12 and a conservative modification thereof; and a light chain variable region CDR2 comprising the amino acid sequence set forth in SEQ ID NO: 15 and a conservative modification thereof;
c) a heavy chain variable region CDR2 comprising the amino acid sequence set forth in SEQ ID NO: 29 and a conservative modification thereof; and a light chain variable region CDR2 comprising the amino acid sequence set forth in SEQ ID NO: 32 and a conservative modification thereof;
d) a heavy chain variable region CDR2 comprising the amino acid sequence set forth in SEQ ID NO: 38 and a conservative modification thereof; and a light chain variable region CDR2 comprising the amino acid sequence set forth in SEQ ID NO: 5 and a conservative modification thereof;
e) a heavy chain variable region CDR2 comprising the amino acid sequence set forth in SEQ ID NO: 47 and a conservative modification thereof; and a light chain variable region CDR2 comprising the amino acid sequence set forth in SEQ ID NO: 50 and a conservative modification thereof;
f) a heavy chain variable region CDR2 comprising the amino acid sequence set forth in SEQ ID NO: 2 and a conservative modification thereof; and a light chain variable region CDR2 comprising the amino acid sequence set forth in SEQ ID NO: 58 and a conservative modification thereof;
g) a heavy chain variable region CDR2 comprising the amino acid sequence set forth in SEQ ID NO: 71 and a conservative modification thereof; and a light chain variable region CDR2 comprising the amino acid sequence set forth in SEQ ID NO: 74 and a conservative modification thereof;
h) a heavy chain variable region CDR2 comprising the amino acid sequence set forth in SEQ ID NO: 80 and a conservative modification thereof; and a light chain variable region CDR2 comprising the amino acid sequence set forth in SEQ ID NO: 58 and a conservative modification thereof;
i) a heavy chain variable region CDR2 comprising the amino acid sequence set forth in SEQ ID NO: 88 and a conservative modification thereof; and a light chain variable region CDR2 comprising the amino acid sequence set forth in SEQ ID NO: 210 and a conservative modification thereof;
j) a heavy chain variable region CDR2 comprising the amino acid sequence set forth in SEQ ID NO: 97 and a conservative modification thereof; and a light chain variable region CDR2 comprising the amino acid sequence set forth in SEQ ID NO: 100 and a conservative modification thereof;
k) a heavy chain variable region CDR2 comprising the amino acid sequence set forth in SEQ ID NO: 106 and a conservative modification thereof; and a light chain variable region CDR2 comprising the amino acid sequence set forth in SEQ ID NO: 58 and a conservative modification thereof;
l) a heavy chain variable region CDR2 comprising the amino acid sequence set forth in SEQ ID NO: 106 and a conservative modification thereof; and a light chain variable region CDR2 comprising the amino acid sequence set forth in SEQ ID NO: 5 and a conservative modification thereof;
m) a heavy chain variable region CDR2 comprising the amino acid sequence set forth in SEQ ID NO: 115 and a conservative modification thereof; and a light chain variable region CDR2 comprising the amino acid sequence set forth in SEQ ID NO: 100 and a conservative modification thereof;
n) a heavy chain variable region CDR2 comprising the amino acid sequence set forth in SEQ ID NO: 2 and a conservative modification thereof; and a light chain variable region CDR2 comprising the amino acid sequence set forth in SEQ ID NO: 58 and a conservative modification thereof;
o) a heavy chain variable region CDR2 comprising the amino acid sequence set forth in SEQ ID NO: 136 and a conservative modification thereof; and a light chain variable region CDR2 comprising the amino acid sequence set forth in SEQ ID NO: 139 and a conservative modification thereof;
p) a heavy chain variable region CDR2 comprising the amino acid sequence set forth in SEQ ID NO: 2 and a conservative modification thereof; and a light chain variable region CDR2 comprising the amino acid sequence set forth in SEQ ID NO: 146 and a conservative modification thereof;
q) a heavy chain variable region CDR2 comprising the amino acid sequence set forth in SEQ ID NO: 136 and a conservative modification thereof; and a light chain variable region CDR2 comprising the amino acid sequence set forth in SEQ ID NO: 74 and a conservative modification thereof; and
r) a heavy chain variable region CDR2 comprising the amino acid sequence set forth in SEQ ID NO: 167 and a conservative modification thereof; and a light chain variable region CDR2 comprising the amino acid sequence set forth in SEQ ID NO: 170 and a conservative modification thereof;
s) a heavy chain variable region CDR2 comprising the amino acid sequence set forth in SEQ ID NO: 176 and a conservative modification thereof; and a light chain variable region CDR2 comprising the amino acid sequence set forth in SEQ ID NO: 50 and a conservative modification thereof;
t) a heavy chain variable region CDR2 comprising the amino acid sequence set forth in SEQ ID NO: 185 and a conservative modification thereof; and a light chain variable region CDR2 comprising the amino acid sequence set forth in SEQ ID NO: 188 and a conservative modification thereof;
u) a heavy chain variable region CDR2 comprising the amino acid sequence set forth in SEQ ID NO: 194 and a conservative modification thereof; and a light chain variable region CDR2 comprising the amino acid sequence set forth in SEQ ID NO: 5 and a conservative modification thereof; and
v) a heavy chain variable region CDR2 comprising the amino acid sequence set forth in SEQ ID NO: 202 and a conservative modification thereof; and a light chain variable region CDR2 comprising the amino acid sequence set forth in SEQ ID NO: 5 and a conservative modification thereof.

3. The antibody-TCO conjugate of claim 1 or claim 2, wherein the heavy chain variable region and light chain variable region CDR1 domains are selected from the group consisting of:
a) a heavy chain variable region CDR1 comprising the amino acid sequence set forth in SEQ ID NO: 1 and a conservative modification thereof; and a light chain variable region CDR1 comprising the amino acid sequence set forth in SEQ ID NO: 4 and a conservative modification thereof;
b) a heavy chain variable region CDR1 comprising the amino acid sequence set forth in SEQ ID NO: 11 and a conservative modification thereof; and a light chain variable region CDR1 comprising the amino acid sequence set forth in SEQ ID NO: 14 and a conservative modification thereof;
c) a heavy chain variable region CDR1 comprising the amino acid sequence set forth in SEQ ID NO: 21 and a conservative modification thereof; and a light chain variable region CDR1 comprising the amino acid sequence set forth in SEQ ID NO: 4 and a conservative modification thereof;
d) a heavy chain variable region CDR1 comprising the amino acid sequence set forth in SEQ ID NO: 28 and a conservative modification thereof; and a light chain variable region CDR1 comprising the amino acid sequence set forth in SEQ ID NO: 31 and a conservative modification thereof;
e) a heavy chain variable region CDR1 comprising the amino acid sequence set forth in SEQ ID NO: 21 and a conservative modification thereof; and a light chain variable region CDR1 comprising the amino acid sequence set forth in SEQ ID NO: 40 and a conservative modification thereof;
f) a heavy chain variable region CDR1 comprising the amino acid sequence set forth in SEQ ID NO: 46 and a conservative modification thereof; and a light chain variable region CDR1 comprising the amino acid sequence set forth in SEQ ID NO: 49 and a conservative modification thereof;
g) a heavy chain variable region CDR1 comprising the amino acid sequence set forth in SEQ ID NO: 21 and a conservative modification thereof; and a light chain variable region CDR1 comprising the amino acid sequence set forth in SEQ ID NO: 57 and a conservative modification thereof;
h) a heavy chain variable region CDR1 comprising the amino acid sequence set forth in SEQ ID NO: 70 and a conservative modification thereof; and a light chain variable region CDR1 comprising the amino acid sequence set forth in SEQ ID NO: 73 and a conservative modification thereof;
i) a heavy chain variable region CDR1 comprising the amino acid sequence set forth in SEQ ID NO: 87 and a conservative modification thereof; and a light chain variable region CDR1 comprising the amino acid sequence set forth in SEQ ID NO: 90 and a conservative modification thereof;
j) a heavy chain variable region CDR1 comprising the amino acid sequence set forth in SEQ ID NO: 96 and a conservative modification thereof; and a light chain variable region CDR1 comprising the amino acid sequence set forth in SEQ ID NO: 99 and a conservative modification thereof;
k) a heavy chain variable region CDR1 comprising the amino acid sequence set forth in SEQ ID NO: 96 and a conservative modification thereof; and a light chain variable region CDR1 comprising the amino acid sequence set forth in SEQ ID NO: 117 and a conservative modification thereof;
l) a heavy chain variable region CDR1 comprising the amino acid sequence set forth in SEQ ID NO: 21 and a conservative modification thereof; and a light chain variable region CDR1 comprising the amino acid sequence set forth in SEQ ID NO: 124 and a conservative modification thereof;
m) a heavy chain variable region CDR1 comprising the amino acid sequence set forth in SEQ ID NO: 135 and a conservative modification thereof; and a light chain variable region CDR1 comprising the amino acid sequence set forth in SEQ ID NO: 138 and a conservative modification thereof;
n) a heavy chain variable region CDR1 comprising the amino acid sequence set forth in SEQ ID NO: 151 and a conservative modification thereof; and a light chain variable region CDR1 comprising the amino acid sequence set forth in SEQ ID NO: 57 and a conservative modification thereof;
o) a heavy chain variable region CDR1 comprising the amino acid sequence set forth in SEQ ID NO: 11 and a conservative modification thereof; and a light chain variable region CDR1 comprising the amino acid sequence set forth in SEQ ID NO: 73 and a conservative modification thereof;
p) a heavy chain variable region CDR1 comprising the amino acid sequence set forth in SEQ ID NO: 96 and a conservative modification thereof; and a light chain variable region CDR1 comprising the amino acid sequence set forth in SEQ ID NO: 169 and a conservative modification thereof;
q) a heavy chain variable region CDR1 comprising the amino acid sequence set forth in SEQ ID NO: 21 and a conservative modification thereof; and a light chain variable region CDR1 comprising the amino acid sequence set forth in SEQ ID NO: 178 and a conservative modification thereof;
r) a heavy chain variable region CDR1 comprising the amino acid sequence set forth in SEQ ID NO: 184 and a conservative modification thereof; and a light chain variable region CDR1 comprising the amino acid sequence set forth in SEQ ID NO: 187 and a conservative modification thereof;
s) a heavy chain variable region CDR1 comprising the amino acid sequence set forth in SEQ ID NO: 11 and a conservative modification thereof; and a light chain variable region CDR1 comprising the amino acid sequence set forth in SEQ ID NO: 4 and a conservative modification thereof; and
t) a heavy chain variable region CDR1 comprising the amino acid sequence set forth in SEQ ID NO: 201 and a conservative modification thereof; and a light chain variable region CDR1 comprising the amino acid sequence set forth in SEQ ID NO: 57 and a conservative modification thereof.

4. The antibody-TCO conjugate of any one of claims 1-3, wherein the anti-DLL3 antibody or antigen-binding fragment thereof comprises:
a) a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3;
b) a heavy chain variable region comprising a CDR1 comprising the amino acid sequence set forth in SEQ ID NO: 11, a CDR2 comprising the amino acid sequence set forth in SEQ ID NO: 12, and a CDR3 comprising the amino acid sequence set forth in SEQ ID NO: 13;
c) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 22;
d) a heavy chain variable region comprising a CDR1 comprising the amino acid sequence set forth in SEQ ID NO: 28, a CDR2 comprising the amino acid sequence set forth in SEQ ID NO: 29, and a CDR3 comprising the amino acid sequence set forth in SEQ ID NO: 30;
e) a heavy chain variable region comprising a CDR1 comprising the amino acid sequence set forth in SEQ ID NO: 21, a CDR2 comprising the amino acid sequence set forth in SEQ ID NO: 38, and a CDR3 comprising the amino acid sequence set forth in SEQ ID NO: 39;
f) a heavy chain variable region comprising a CDR1 comprising the amino acid sequence set forth in SEQ ID NO: 46, a CDR2 comprising the amino acid sequence set forth in SEQ ID NO: 47, and a CDR3 comprising the amino acid sequence set forth in SEQ ID NO: 48;
g) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 56;
h) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 64;
i) a heavy chain variable region comprising a CDR1 comprising the amino acid sequence set forth in SEQ ID NO: 70, a CDR2 comprising the amino acid sequence set forth in SEQ ID NO: 71 and a CDR3 comprising the amino acid sequence set forth in SEQ ID NO: 72;
j) a heavy chain variable region comprising a CDR1 comprising the amino acid sequence set forth in SEQ ID NO: 21, a CDR2 comprising the amino acid sequence set forth in SEQ ID NO: 80, and a CDR3 comprising the amino acid sequence set forth in SEQ ID NO: 81;
k) a heavy chain variable region comprising a CDR1 comprising the amino acid sequence set forth in SEQ ID NO: 87, a CDR2 comprising the amino acid sequence set forth in SEQ ID NO: 88, and a CDR3 comprising the amino acid sequence set forth in SEQ ID NO: 89;
l) a heavy chain variable region comprising a CDR1 comprising the amino acid sequence set forth in SEQ ID NO: 96, a CDR2 comprising the amino acid sequence set forth in SEQ ID NO: 97, and a CDR3 comprising the amino acid sequence set forth in SEQ ID NO: 98;
m) a heavy chain variable region comprising a CDR1 comprising the amino acid sequence set forth in SEQ ID NO: 21, a CDR2 comprising the amino acid sequence set forth in SEQ ID NO: 106, and a CDR3 comprising the amino acid sequence set forth in SEQ ID NO: 107;
n) a heavy chain variable region comprising a CDR1 comprising the amino acid sequence set forth in SEQ ID NO: 96, a CDR2 comprising the amino acid sequence set forth in SEQ ID NO: 115, and a CDR3 comprising the amino acid sequence set forth in SEQ ID NO: 116;
o) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 123;
p) a heavy chain variable region comprising a CDR1 comprising the amino acid sequence set forth in SEQ ID NO: 135, a CDR2 comprising the amino acid sequence set forth in SEQ ID NO: 136, and a CDR3 comprising the amino acid sequence set forth in SEQ ID NO: 137;
q) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 145;
r) a heavy chain variable region comprising a CDR1 comprising the amino acid sequence set forth in SEQ ID NO: 151, a CDR2 comprising the amino acid sequence set forth in SEQ ID NO: 2, and a CDR3 comprising the amino acid sequence set forth in SEQ ID NO: 152;
s) a heavy chain variable region comprising a CDR1 comprising the amino acid sequence set forth in SEQ ID NO: 11, a CDR2 comprising the amino acid sequence set forth in SEQ ID NO: 136, and a CDR3 comprising the amino acid sequence set forth in SEQ ID NO: 161;
t) a heavy chain variable region comprising a CDR1 comprising the amino acid sequence set forth in SEQ ID NO: 96, a CDR2 comprising the amino acid sequence set forth in SEQ ID NO: 167, and CDR3 comprising the amino acid sequence set forth in SEQ ID NO: 168;
u) a heavy chain variable region comprising a CDR1 comprising the amino acid sequence set forth in SEQ ID NO: 21, a CDR2 comprising the amino acid sequence set forth in SEQ ID NO: 176, and a CDR3 comprising the amino acid sequence set forth in SEQ ID NO: 177;
v) a heavy chain variable region comprising a CDR1 comprising the amino acid sequence set forth in SEQ ID NO: 184, a CDR2 comprising the amino acid sequence set forth in SEQ ID NO: 185, and a CDR3 comprising the amino acid sequence set forth in SEQ ID NO: 186;
w) a heavy chain variable region comprising a CDR1 comprising the amino acid sequence set forth in SEQ ID NO: 11, a CDR2 comprising the amino acid sequence set forth in SEQ ID NO: 194, and a CDR3 comprising the amino acid sequence set forth in SEQ ID NO: 195; or
x) a heavy chain variable region comprising a CDR1 comprising the amino acid sequence set forth in SEQ ID NO: 201, a CDR2 comprising the amino acid sequence set forth in SEQ ID NO: 202, and a CDR3 comprising the amino acid sequence set forth in SEQ ID NO: 203.

5. The antibody-TCO conjugate of any one of claims 1-4, wherein the anti-DLL3 antibody or antigen-binding fragment thereof comprises:
a) a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6;
b) a light chain variable region comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16;
c) a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 23;
d) a light chain variable region comprising a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33;
e) a light chain variable region comprising a CDR1 comprising the amino acid sequence set forth in SEQ ID NO: 40, a CDR2 comprising the amino acid sequence set forth in SEQ ID NO: 5, and a CDR3 comprising the amino acid sequence set forth in SEQ ID NO: 41;
f) a light chain variable region comprising a CDR1 comprising the amino acid sequence set forth in SEQ ID NO: 49, a CDR2 comprising the amino acid sequence set forth in SEQ ID NO: 50, and a CDR3 comprising the amino acid sequence set forth in SEQ ID NO: 51;
g) a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 59;
h) a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 65;
i) a light chain variable region comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 75;
j) a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82;
k) a light chain variable region comprising a CDR1 comprising the amino acid sequence set forth in SEQ ID NO: 90, a CDR2 comprising the amino acid sequence set forth in SEQ ID NO: 210; and a CDR3 comprising the amino acid sequence set forth in SEQ ID NO: 91;
l) a light chain variable region comprising a CDR1 comprising the amino acid sequence set forth in SEQ ID NO: 99, a CDR2 comprising the amino acid sequence set forth in SEQ ID NO: 100, and a CDR3 comprising the amino acid sequence set forth in SEQ ID NO: 101;
m) a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 112;
n) a light chain variable region comprising a CDR1 comprising the amino acid sequence set forth in SEQ ID NO: 117, a CDR2 comprising the amino acid sequence set forth in SEQ ID NO: 100, and a CDR3 comprising the amino acid sequence set forth in SEQ ID NO: 118;
o) a light chain variable region comprising a CDR1 comprising the amino acid sequence set forth in SEQ ID NO: 124, a CDR2 comprising the amino acid sequence set forth in SEQ ID NO: 58, and a CDR3 comprising the amino acid sequence set forth in SEQ ID NO: 125;
p) a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 130;
q) a light chain variable region comprising a CDR1 comprising the amino acid sequence set forth in SEQ ID NO: 138, a CDR2 comprising the amino acid sequence set forth in SEQ ID NO: 139, and a CDR3 comprising the amino acid sequence set forth in SEQ ID NO: 140;
r) a light chain variable region comprising a CDR1 comprising the amino acid sequence set forth in SEQ ID NO: 57, a CDR2 comprising the amino acid sequence set forth in SEQ ID NO: 146, and a CDR3 comprising the amino acid sequence set forth in SEQ ID NO: 125;
s) a light chain variable region comprising a CDR1 comprising the amino acid sequence set forth in SEQ ID NO: 124, a CDR2 comprising the amino acid sequence set forth in SEQ ID NO: 58, and CDR3 comprising the amino acid sequence set forth in SEQ ID NO: 59;
t) a light chain variable region comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 162;
u) a light chain variable region comprising a CDR1 comprising the amino acid sequence set forth in SEQ ID NO: 169, a light chain variable region CDR2 comprising the amino acid sequence set forth in SEQ ID NO: 170, and a CDR3 comprising the amino acid sequence set forth in SEQ ID NO: 171;
v) a light chain variable region comprising a CDR1 comprising the amino acid sequence set forth in SEQ ID NO: 178, a CDR2 comprising the amino acid sequence set forth in SEQ ID NO: 50, and a CDR3 comprising the amino acid sequence set forth in SEQ ID NO: 179;
w) a light chain variable region comprising a CDR1 comprising the amino acid sequence set forth in SEQ ID NO: 187, a CDR2 comprising the amino acid sequence set forth in SEQ ID NO: 188, and a CDR3 comprising the amino acid sequence set forth in SEQ ID NO: 189;
x) a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 196; or
y) a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 204.

6. The antibody-TCO conjugate of any one of claims 1-5, wherein the anti-DLL3 antibody or antigen-binding fragment thereof comprises:
a) a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a region CDR3 comprising the amino acid sequence set forth in SEQ ID NO: 6;
b) a heavy chain variable region comprising a CDR1 comprising the amino acid sequence set forth in SEQ ID NO: 11, a CDR2 comprising an amino acid sequence set forth in SEQ ID NO: 12, and a CDR3 comprising the amino acid sequence set forth in SEQ ID NO: 13; and a light chain variable region comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16;
c) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 22; and a light chain variable region comprising a CDR1 comprising the amino acid sequence set forth in SEQ ID NO: 4, a CDR2 comprising the amino acid sequence set forth in SEQ ID NO: 5, and a region CDR3 comprising the amino acid sequence set forth in SEQ ID NO: 23;
d) a heavy chain variable region comprising a CDR1 comprising the amino acid sequence set forth in SEQ ID NO: 28, a CDR2 comprising the amino acid sequence set forth in SEQ ID NO: 29, and a CDR3 comprising the amino acid sequence set forth in SEQ ID NO: 30; and a light chain variable region comprising a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33;
e) a heavy chain variable region comprising a CDR1 comprising the amino acid sequence set forth in SEQ ID NO: 21, a CDR2 comprising the amino acid sequence set forth in SEQ ID NO: 38, and a CDR3 comprising the amino acid sequence set forth in SEQ ID NO: 39; and a light chain variable region comprising a CDR1 comprising the amino acid sequence set forth in SEQ ID NO: 40, a CDR2 comprising the amino acid sequence set forth in SEQ ID NO: 5, and a CDR3 comprising the amino acid sequence set forth in SEQ ID NO: 41;
f) a heavy chain variable region comprising a CDR1 comprising the amino acid sequence set forth in SEQ ID NO: 46, a CDR2 comprising the amino acid sequence set forth in SEQ ID NO: 47, and a CDR3 comprising the amino acid sequence set forth in SEQ ID NO: 48; and a light chain variable region comprising a CDR1 comprising the amino acid sequence set forth in SEQ ID NO: 49, a CDR2 comprising the amino acid sequence set forth in SEQ ID NO: 50, and a CDR3 comprising the amino acid sequence set forth in SEQ ID NO: 51;
g) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 56; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 59;
h) a heavy chain variable region comprising a CDR1 comprising the amino acid sequence set forth in SEQ ID NO: 21, CDR2 comprising the amino acid sequence set forth in SEQ ID NO: 2 and a CDR3 comprising the amino acid sequence set forth in SEQ ID NO: 64; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 65;
i) a heavy chain variable region comprising a CDR1 comprising the amino acid sequence set forth in SEQ ID NO: 70, a CDR2 comprising the amino acid sequence set forth in SEQ ID NO: 71, and a CDR3 comprising the amino acid sequence set forth in SEQ ID NO: 72; and a light chain variable region comprising a CDR1 comprising the amino acid sequence set forth in SEQ ID NO: 73, a CDR2 comprising the amino acid sequence set forth in SEQ ID NO: 74, and CDR3 comprising the amino acid sequence set forth in SEQ ID NO: 75;
j) a heavy chain variable region comprising a CDR1 comprising the amino acid sequence set forth in SEQ ID NO: 21, a CDR2 comprising the amino acid sequence set forth in SEQ ID NO: 80, and a CDR3 comprising the amino acid sequence set forth in SEQ ID NO: 81; and a light chain variable region comprising a CDR1 comprising the amino acid sequence set forth in SEQ ID NO: 57, a CDR2 comprising the amino acid sequence set forth in SEQ ID NO: 58, and CDR3 comprising the amino acid sequence set forth in SEQ ID NO: 82;
k) a heavy chain variable region comprising a CDR1 comprising the amino acid sequence set forth in SEQ ID NO: 87, a CDR2 comprising the amino acid sequence set forth in SEQ ID NO: 88, and a CDR3 comprising the amino acid sequence set forth in SEQ ID NO: 89; and a light chain variable region comprising a CDR1 comprising the amino acid sequence set forth in SEQ ID NO: 90, a CDR2 comprising the amino acid sequence set forth in SEQ ID NO: 210, and a CDR3 comprising the amino acid sequence set forth in SEQ ID NO: 91;
l) a heavy chain variable region comprising a CDR1 comprising the amino acid sequence set forth in SEQ ID NO: 96, a CDR2 comprising the amino acid sequence set forth in SEQ ID NO: 97, and a CDR3 comprising the amino acid sequence set forth in SEQ ID NO: 98; and a light chain variable region comprising a CDR1 comprising the amino acid sequence set forth in SEQ ID NO: 99, a CDR2 comprising the amino acid sequence set forth in SEQ ID NO: 100, and a CDR3 comprising the amino acid sequence set forth in SEQ ID NO: 101;
m) a heavy chain variable region comprising a CDR1 comprising the amino acid sequence set forth in SEQ ID NO: 21, a CDR2 comprising the amino acid sequence set forth in SEQ ID NO: 106, and a CDR3 comprising the amino acid sequence set forth in SEQ ID NO: 107; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82;
n) a heavy chain variable region comprising a CDR1 comprising the amino acid sequence set forth in SEQ ID NO: 21, CDR2 comprising the amino acid sequence set forth in SEQ ID NO: 106, and a CDR3 comprising the amino acid sequence set forth in SEQ ID NO: 107; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 112;
o) a heavy chain variable region comprising a CDR1 comprising the amino acid sequence set forth in SEQ ID NO: 96, a CDR2 comprising the amino acid sequence set forth in SEQ ID NO: 115, and a CDR3 comprising the amino acid sequence set forth in SEQ ID NO: 116; and a light chain variable region comprising a CDR1 comprising the amino acid sequence set forth in SEQ ID NO: 117, a CDR2 comprising the amino acid sequence set forth in SEQ ID NO: 100, and a CDR3 comprising the amino acid sequence set forth in SEQ ID NO: 118;
p) a heavy chain variable region comprising a CDR1 comprising the amino acid sequence set forth in SEQ ID NO: 21, CDR2 comprising the amino acid sequence set forth in SEQ ID NO: 2, and a CDR3 comprising the amino acid sequence set forth in SEQ ID NO: 123; and a light chain variable region comprising a CDR1 comprising the amino acid sequence set forth in SEQ ID NO: 124, a CDR2 comprising the amino acid sequence set forth in SEQ ID NO: 58, and a CDR3 comprising the amino acid sequence set forth in SEQ ID NO: 125;
q) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 56; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 130;
r) a heavy chain variable region comprising a CDR1 comprising the amino acid sequence set forth in SEQ ID NO: 135, a CDR2 comprising the amino acid sequence set forth in SEQ ID NO: 136, and a CDR3 comprising the amino acid sequence set forth in SEQ ID NO: 137; and a light chain variable region comprising a CDR1 comprising the amino acid sequence set forth in SEQ ID NO: 138, a CDR2 comprising the amino acid sequence set forth in SEQ ID NO: 139, and a CDR3 comprising the amino acid sequence set forth in SEQ ID NO: 140;
s) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 145; and a light chain variable region comprising a CDR1 comprising the amino acid sequence set forth in SEQ ID NO: 57, a CDR2 comprising the amino acid sequence set forth in SEQ ID NO: 146, and a CDR3 comprising the amino acid sequence set forth in SEQ ID NO: 125;
t) a heavy chain variable region comprising a CDR1 comprising the amino acid sequence set forth in SEQ ID NO: 151, a CDR2 comprising the amino acid sequence set forth in SEQ ID NO: 2, and a CDR3 comprising the amino acid sequence set forth in SEQ ID NO: 152; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82;
u) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 123; and a light chain variable region comprising a CDR1 comprising the amino acid sequence set forth in SEQ ID NO: 124, a CDR2 comprising the amino acid sequence set forth in SEQ ID NO: 58, and a CDR3 comprising the amino acid sequence set forth in SEQ ID NO: 59;
v) a heavy chain variable region comprising a CDR1 comprising the amino acid sequence set forth in SEQ ID NO: 11, a CDR2 comprising the amino acid sequence set forth in SEQ ID NO: 136, and a CDR3 comprising the amino acid sequence set forth in SEQ ID NO: 161; and a light chain variable region comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 162;
w) a heavy chain variable region comprising a CDR1 comprising the amino acid sequence set forth in SEQ ID NO: 96, a CDR2 comprising the amino acid sequence set forth in SEQ ID NO: 167, and a CDR3 comprising the amino acid sequence set forth in SEQ ID NO: 168; and a light chain variable region comprising a CDR1 comprising the amino acid sequence set forth in SEQ ID NO: 169, a CDR2 comprising the amino acid sequence set forth in SEQ ID NO: 170, and a CDR3 comprising the amino acid sequence set forth in SEQ ID NO: 171;
x) a heavy chain variable region comprising a CDR1 comprising the amino acid sequence set forth in SEQ ID NO: 21, a CDR2 comprising the amino acid sequence set forth in SEQ ID NO: 176, and a CDR3 comprising the amino acid sequence set forth in SEQ ID NO: 177; and a light chain variable region comprising a CDR1 comprising the amino acid sequence set forth in SEQ ID NO: 178, a CDR2 comprising the amino acid sequence set forth in SEQ ID NO: 50, and a CDR3 comprising the amino acid sequence set forth in SEQ ID NO: 179;
y) a heavy chain variable region comprising a CDR1 comprising the amino acid sequence set forth in SEQ ID NO: 184, a CDR2 comprising the amino acid sequence set forth in SEQ ID NO: 185, and a CDR3 comprising the amino acid sequence set forth in SEQ ID NO: 186; and a light chain variable region comprising a CDR1 comprising the amino acid sequence set forth in SEQ ID NO: 187, a CDR2 comprising the amino acid sequence set forth in SEQ ID NO: 188, and a CDR3 comprising the amino acid sequence set forth in SEQ ID NO: 188;
z) a heavy chain variable region comprising a CDR1 comprising the amino acid sequence set forth in SEQ ID NO: 11, a CDR2 comprising the amino acid sequence set forth in SEQ ID NO: 194, and a CDR3 comprising the amino acid sequence set forth in SEQ ID NO: 195; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 196; or
aa) a heavy chain variable region comprising a CDR1 comprising the amino acid sequence set forth in SEQ ID NO: 201, a CDR2 comprising the amino acid sequence set forth in SEQ ID NO: 202, and a CDR3 comprising the amino acid sequence set forth in SEQ ID NO: 203; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 204.

7. The antibody-TCO conjugate of any one of claims 1-6, wherein the anti-DLL3 antibody or antigen-binding fragment thereof comprises:
a) a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6;
b) a heavy chain variable region comprising a CDR1 comprising the amino acid sequence set forth in SEQ ID NO: 28, a CDR2 comprising the amino acid sequence set forth in SEQ ID NO: 29, and a CDR3 comprising the amino acid sequence set forth in SEQ ID NO: 30; and a light chain variable region comprising a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33; or
c) a heavy chain variable region comprising a CDR1 comprising the amino acid sequence set forth in SEQ ID NO: 21, a CDR2 comprising the amino acid sequence set forth in SEQ ID NO: 38, and a CDR3 comprising the amino acid sequence set forth in SEQ ID NO: 39; and a light chain variable region comprising a CDR1 comprising the amino acid sequence set forth in SEQ ID NO: 40, a CDR2 comprising the amino acid sequence set forth in SEQ ID NO: 5, and a CDR3 comprising the amino acid sequence set forth in SEQ ID NO: 41.

8. The antibody-TCO conjugate of any one of claims 1-7, wherein the anti-DLL3 antibody or antigen-binding fragment thereof comprises a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 7, SEQ ID NO: 17, SEQ ID NO: 24, SEQ ID NO: 34, SEQ ID NO: 42, SEQ ID NO: 52, SEQ ID NO: 60, SEQ ID NO: 66, SEQ ID NO: 76, SEQ ID NO: 83, SEQ ID NO: 92, SEQ ID NO: 102, SEQ ID NO: 108, SEQ ID NO: 119, SEQ ID NO: 126, SEQ ID NO: 131, SEQ ID NO: 141, SEQ ID NO: 147, SEQ ID NO: 153, SEQ ID NO: 157, SEQ ID NO: 163, SEQ ID NO: 172, SEQ ID NO: 180, SEQ ID NO: 190, SEQ ID NO: 197, or SEQ ID NO: 205.

9. The antibody-TCO conjugate of any one of claims 1-8, wherein the anti-DLL3 antibody or antigen-binding fragment thereof comprises a heavy chain variable region comprising the amino acid sequence set forth in SEQ ID NO: 7, SEQ ID NO: 17, SEQ ID NO: 24, SEQ ID NO: 34, SEQ ID NO: 42, SEQ ID NO: 52, SEQ ID NO: 60, SEQ ID NO: 66, SEQ ID NO: 76, SEQ ID NO: 83, SEQ ID NO: 92, SEQ ID NO: 102, SEQ ID NO: 108, SEQ ID NO: 119, SEQ ID NO: 126, SEQ ID NO: 131, SEQ ID NO: 141, SEQ ID NO: 147, SEQ ID NO: 153, SEQ ID NO: 157, SEQ ID NO: 163, SEQ ID NO: 172, SEQ ID NO: 180, SEQ ID NO: 190, SEQ ID NO: 197, or SEQ ID NO: 205.

10. The antibody-TCO conjugate of any one of claims 1-9, wherein the anti-DLL3 antibody or antigen-binding fragment thereof comprises a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 8, SEQ ID NO: 18, SEQ ID NO: 25, SEQ ID NO: 35, SEQ ID NO: 43, SEQ ID NO: 53, SEQ ID NO: 61, SEQ ID NO: 67, SEQ ID NO: 77, SEQ ID NO: 84, SEQ ID NO: 93, SEQ ID NO: 103, SEQ ID NO: 109, SEQ ID NO: 113, SEQ ID NO: 120, SEQ ID NO: 127, SEQ ID NO: 132, SEQ ID NO: 142, SEQ ID NO: 148, SEQ ID NO: 154, SEQ ID NO: 158, SEQ ID NO: 164, SEQ ID NO: 173, SEQ ID NO: 181, SEQ ID NO: 191, SEQ ID NO: 198, or SEQ ID NO: 206.

11. The antibody-TCO conjugate of any one of claims 1-10, wherein the anti-DLL3 antibody or antigen-binding fragment thereof comprises a light chain variable region comprising the amino acid sequence set forth in SEQ ID NO: 8, SEQ ID NO: 18, SEQ ID NO: 25, SEQ ID NO: 35, SEQ ID NO: 43, SEQ ID NO: 53, SEQ ID NO: 61, SEQ ID NO: 67, SEQ ID NO: 77, SEQ ID NO: 84, SEQ ID NO: 93, SEQ ID NO: 103, SEQ ID NO: 109, SEQ ID NO: 113, SEQ ID NO: 120, SEQ ID NO: 127, SEQ ID NO: 132, SEQ ID NO: 142, SEQ ID NO: 148, SEQ ID NO: 154, SEQ ID NO: 158, SEQ ID NO: 164, SEQ ID NO: 173, SEQ ID NO: 181, SEQ ID NO: 191, SEQ ID NO: 198, or SEQ ID NO: 206.

12. The antibody-TCO conjugate of any one of claims 1-11, wherein the anti-DLL3 antibody or antigen-binding fragment thereof comprises:
a) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 7, SEQ ID NO: 17, SEQ ID NO: 24, SEQ ID NO: 34, SEQ ID NO: 42, SEQ ID NO: 52, SEQ ID NO: 60, SEQ ID NO: 66, SEQ ID NO: 76, SEQ ID NO: 83, SEQ ID NO: 92, SEQ ID NO: 102, SEQ ID NO: 108, SEQ ID NO: 119, SEQ ID NO: 126, SEQ ID NO: 131, SEQ ID NO: 141, SEQ ID NO: 147, SEQ ID NO: 153, SEQ ID NO: 157, SEQ ID NO: 163, SEQ ID NO: 172, SEQ ID NO: 180, SEQ ID NO: 190, SEQ ID NO: 197, or SEQ ID NO: 205; and
b) a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 8, SEQ ID NO: 18, SEQ ID NO: 25, SEQ ID NO: 35, SEQ ID NO: 43, SEQ ID NO: 53, SEQ ID NO: 61, SEQ ID NO: 67, SEQ ID NO: 77, SEQ ID NO: 84, SEQ ID NO: 93, SEQ ID NO: 103, SEQ ID NO: 109, SEQ ID NO: 113, SEQ ID NO: 120, SEQ ID NO: 127, SEQ ID NO: 132, SEQ ID NO: 142, SEQ ID NO: 148, SEQ ID NO: 154, SEQ ID NO: 158, SEQ ID NO: 164, SEQ ID NO: 173, SEQ ID NO: 181, SEQ ID NO: 191, SEQ ID NO: 198, or SEQ ID NO: 206.

13. The antibody-TCO conjugate of any one of claims 1-12, wherein the anti-DLL3 antibody or antigen-binding fragment thereof comprises:
a) a heavy chain variable region comprising the amino acid sequence set forth in SEQ ID NO: 7, SEQ ID NO: 17, SEQ ID NO: 24, SEQ ID NO: 34, SEQ ID NO: 42, SEQ ID NO: 52, SEQ ID NO: 60, SEQ ID NO: 66, SEQ ID NO: 76, SEQ ID NO: 83, SEQ ID NO: 92, SEQ ID NO: 102, SEQ ID NO: 108, SEQ ID NO: 119, SEQ ID NO: 126, SEQ ID NO: 131, SEQ ID NO: 141, SEQ ID NO: 147, SEQ ID NO: 153, SEQ ID NO: 157, SEQ ID NO: 163, SEQ ID NO: 172, SEQ ID NO: 180, SEQ ID NO: 190, SEQ ID NO: 197, or SEQ ID NO: 205; and
b) a light chain variable region comprising the amino acid sequence set forth in SEQ ID NO: 8, SEQ ID NO: 18, SEQ ID NO: 25, SEQ ID NO: 35, SEQ ID NO: 43, SEQ ID NO: 53, SEQ ID NO: 61, SEQ ID NO: 67, SEQ ID NO: 77, SEQ ID NO: 84, SEQ ID NO: 93, SEQ ID NO: 103, SEQ ID NO: 109, SEQ ID NO: 113, SEQ ID NO: 120, SEQ ID NO: 127, SEQ ID NO: 132, SEQ ID NO: 142, SEQ ID NO: 148, SEQ ID NO: 154, SEQ ID NO: 158, SEQ ID NO: 164, SEQ ID NO: 173, SEQ ID NO: 181, SEQ ID NO: 191, SEQ ID NO: 198, or SEQ ID NO: 206.

14. The antibody-TCO conjugate of any one of claims 1-13, wherein the anti-DLL3 antibody or antigen-binding fragment thereof comprises:
a) a heavy chain variable region comprising the amino acid sequence set forth in SEQ ID NO: 7, and a light chain variable region comprising the amino acid sequence set forth in SEQ ID NO: 8;
b) a heavy chain variable region comprising the amino acid sequence set forth in SEQ ID NO: 17, and a light chain variable region comprising the amino acid sequence set forth in SEQ ID NO: 18;
c) a heavy chain variable region comprising the amino acid sequence set forth in SEQ ID NO: 24, and a light chain variable region comprising the amino acid sequence set forth in SEQ ID NO: 25;
d) a heavy chain variable region comprising the amino acid sequence set forth in SEQ ID NO: 34, and a light chain variable region comprising the amino acid sequence set forth in SEQ ID NO: 35;
e) a heavy chain variable region comprising the amino acid sequence set forth in SEQ ID NO: 42, and a light chain variable region comprising the amino acid sequence set forth in SEQ ID NO: 43;
f) a heavy chain variable region comprising the amino acid sequence set forth in SEQ ID NO: 52, and a light chain variable region comprising the amino acid sequence set forth in SEQ ID NO: 53;
g) a heavy chain variable region comprising the amino acid sequence set forth in SEQ ID NO: 60, and a light chain variable region comprising the amino acid sequence set forth in SEQ ID NO: 61;
h) a heavy chain variable region comprising the amino acid sequence set forth in SEQ ID NO: 66, and a light chain variable region comprising the amino acid sequence set forth in SEQ ID NO: 67;
i) a heavy chain variable region comprising the amino acid sequence set forth in SEQ ID NO: 76, and a light chain variable region comprising the amino acid sequence set forth in SEQ ID NO: 77;
j) a heavy chain variable region comprising the amino acid sequence set forth in SEQ ID NO: 83, and a light chain variable region comprising the amino acid sequence set forth in SEQ ID NO: 84;
k) a heavy chain variable region comprising the amino acid sequence set forth in SEQ ID NO: 92, and a light chain variable region comprising the amino acid sequence set forth in SEQ ID NO: 93;
l) a heavy chain variable region comprising the amino acid sequence set forth in SEQ ID NO: 102, and a light chain variable region comprising the amino acid sequence set forth in SEQ ID NO: 103;
m) a heavy chain variable region comprising the amino acid sequence set forth in SEQ ID NO: 108, and a light chain variable region comprising the amino acid sequence set forth in SEQ ID NO: 109;
n) a heavy chain variable region comprising the amino acid sequence set forth in SEQ ID NO: 108, and a light chain variable region comprising the amino acid sequence set forth in SEQ ID NO: 113;
o) a heavy chain variable region comprising the amino acid sequence set forth in SEQ ID NO: 119, and a light chain variable region comprising the amino acid sequence set forth in SEQ ID NO: 120;
p) a heavy chain variable region comprising the amino acid sequence set forth in SEQ ID NO: 126, and a light chain variable region comprising the amino acid sequence set forth in SEQ ID NO: 127;
q) a heavy chain variable region comprising the amino acid sequence set forth in SEQ ID NO: 131, and a light chain variable region comprising the amino acid sequence set forth in SEQ ID NO: 132;
r) a heavy chain variable region comprising the amino acid sequence set forth in SEQ ID NO: 141, and a light chain variable region comprising the amino acid sequence set forth in SEQ ID NO: 142;
s) a heavy chain variable region comprising the amino acid sequence set forth in SEQ ID NO: 147, and a light chain variable region comprising the amino acid sequence set forth in SEQ ID NO: 148;
t) a heavy chain variable region comprising the amino acid sequence set forth in SEQ ID NO: 153, and a light chain variable region comprising the amino acid sequence set forth in SEQ ID NO: 154;
u) a heavy chain variable region comprising the amino acid sequence set forth in SEQ ID NO: 157, and a light chain variable region comprising the amino acid sequence set forth in SEQ ID NO: 158;
v) a heavy chain variable region comprising the amino acid sequence set forth in SEQ ID NO: 163, and a light chain variable region comprising the amino acid sequence set forth in SEQ ID NO: 164;
w) a heavy chain variable region comprising the amino acid sequence set forth in SEQ ID NO: 172, and a light chain variable region comprising the amino acid sequence set forth in SEQ ID NO: 173;
x) a heavy chain variable region comprising the amino acid sequence set forth in SEQ ID NO: 180, and a light chain variable region comprising the amino acid sequence set forth in SEQ ID NO: 181;
y) a heavy chain variable region comprising the amino acid sequence set forth in SEQ ID NO: 190, and a light chain variable region comprising the amino acid sequence set forth in SEQ ID NO: 191;
z) a heavy chain variable region comprising the amino acid sequence set forth in SEQ ID NO: 197, and a light chain variable region comprising the amino acid sequence set forth in SEQ ID NO: 198; or
aa) a heavy chain variable region comprising the amino acid sequence set forth in SEQ ID NO: 205, and a light chain variable region comprising the amino acid sequence set forth in SEQ ID NO: 206.

15. The antibody-TCO conjugate of any one of claims 1-14, comprising:
a) a heavy chain variable region comprising the amino acid sequence set forth in SEQ ID NO: 7, and a light chain variable region comprising the amino acid sequence set forth in SEQ ID NO: 8;
b) a heavy chain variable region comprising the amino acid sequence set forth in SEQ ID NO: 34, and a light chain variable region comprising the amino acid sequence set forth in SEQ ID NO: 35; or
c) a heavy chain variable region comprising the amino acid sequence set forth in SEQ ID NO: 42, and a light chain variable region comprising the amino acid sequence set forth in SEQ ID NO: 43.

16. The antibody-TCO conjugate of any one of claims 1-15, wherein the TCO has formula 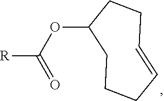
and wherein R is N, C, or an ester.

17. The antibody-TCO conjugate of claim 16, wherein the TCO has formula

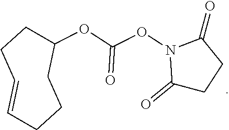 [SMILES: O=C(OC1CC/C=C/CCC1)ON1C(=O)CCC1=O]

18. A composition comprising the antibody-TCO conjugate of any one of claims 1-17.

19. The composition of claim 18, which is a pharmaceutical composition further comprising pharmaceutically acceptable carrier.

20. A radioimmunoconjugate comprising the antibody-TCO conjugate of any one of claims 1-17 and a radioligand comprising a tetrazine moiety (Tz) and a radiolabel.

21. The radioimmunoconjugate of claim 20, wherein the radiolabel comprises a radioactive isotope selected from the group consisting of ^{99m}Tc, ¹¹¹In, ⁴⁴Sc, ⁴⁷Sc, ⁶⁴Cu, ⁶⁷Ga, ⁶⁸Ga, ⁷²As, ⁷⁷As, ⁵²Mn, ¹⁸⁶Re, ¹⁵³Sm, ¹⁷⁷Lu, ⁶⁷Cu, ¹²³I, ¹²⁴I, ¹²⁵I, ¹¹C, ¹³N, ¹⁵O, ¹⁸F, ¹⁶⁶Ho, ¹⁴⁹Pm, ⁹⁰Y, ⁸⁶Y, ²¹³Bi, ¹⁰³Pd, ¹⁰⁹Pd, ¹⁵⁹Gd, ¹⁴⁰La, ¹⁹⁸Au, ¹⁹⁹Au, ¹⁷⁵Yb, ¹⁶⁹Yb, ¹⁶⁵Dy, ¹⁶⁶Dy, ¹⁰⁵Rh, ¹¹¹Ag, ⁸⁹Zr, ¹⁹²Ir, ²²⁵Ac, ²¹²Bi, ²¹³Bi, ⁷⁶Br, ⁵⁵Co, ⁶⁰Cu, ⁶¹Cu, ⁶²Cu, ⁶⁶Ga, ⁹⁰Nb, ²¹²Pb, ¹⁵²Tb, ⁷⁷Br, ²¹¹At, ²²⁷Th, ²²⁴Ra, and ¹⁴⁹Tb.

22. The radioimmunoconjugate of claim 20 or 21, wherein the radiolabel comprises a ⁶⁴Cu radioactive isotope.

23. The radioimmunoconjugate of claim 20 or 21, wherein the radiolabel comprises a ²²⁵Ac radioactive isotope.

24. The radioimmunoconjugate of any one of claims 20-23, further comprising a chelator.

25. The radioimmunoconjugate of claim 24, wherein the chelator is a sarcophagine chelator.

26. The radioimmunoconjugate of claim 24, wherein the chelator is 1,4,7-triazonane-1,4,7-triyl-triacetic acid (NOTA) or 1,4,7-triazacyclononane-1,4-diacetate (NODA).

27. The radioimmunoconjugate of any one of claims 20-26, further comprising a linker attaching the tetrazine moiety (Tz) to the chelator.

28. The radioimmunoconjugate of claim 27, wherein the linker comprises is polyethylene glycol (PEG) or (poly)-L-lysine.

29. The radioimmunoconjugate of claim 28, wherein the polyethylene glycol (PEG) comprises from about 1 unit to about 100 units.

30. The radioimmunoconjugate of claim 28, wherein the (poly)-L-lysine comprises from about 1 unit to about 200 units.

31. The radioimmunoconjugate of any one of claims 24-30, wherein the tetrazine moiety (Tz) and the chelator form a compound selected from the group consisting of:
a) 2,2′,2″-(3-(4-(3-(1-(4-(1,2,4,5-tetrazin-3-yl)phenyl)-1-oxo-5,8,11,14,17,20,23-heptaoxa-2-azapentacosan-25-yl)thioureido)benzyl)-1,4,7-triazonane-2,5,8-triyl)triacetic acid;
b) 2,2′,2″-(3-(4-(3-(1-(4-(1,2,4,5-tetrazin-3-yl)phenyl)-1-oxo-5,8,11,14,17,20,23,26,29,32,35-undecaoxa-2-azaheptatriacontan-37-yl)thioureido)benzyl)-1,4,7-triazonane-2,5,8-triyl)triacetic acid;
c) 2,2′-(7-(4-(3-(1-(4-(1,2,4,5-tetrazin-3-yl)phenyl)-1-oxo-5,8,11,14,17,20,23,26,29,32,35-undecaoxa-2-azaheptatriacontan-37-yl)thioureido)benzyl)-1,4,7-triazonane-1,4-diyl)diacetic acid;
d) 2,2′,2″-(3-(4-(3-(1-(4-(1,2,4,5-tetrazin-3-yl)phenyl)-3,7-dioxo-11,14,17,20,23,26,29-heptaoxa-2,8-diazahentriacontan-31-yl)thioureido)benzyl)-1,4,7-triazonane-2,5,8-triyl)triacetic acid;
e) 2,2′,2″-(3-(4-(3-(1-(4-(1,2,4,5-tetrazin-3-yl)phenyl)-3,7-dioxo-11,14,17,20,23,26,29,32,35,38,41-undecaoxa-2,8-diazatritetracontan-43-yl)thioureido)benzyl)-1,4,7-triazonane-2,5,8-triyl)triacetic acid;
f) 2,2′,2″-(3-(4-(3-(25,28-dioxo-28-((6-(6-(pyridin-2-yl)-1,2,4,5-tetrazin-3-yl)pyridin-3-yl)amino)-3,6,9,12,15,18,21-heptaoxa-24-azaoctacosyl)thioureido)benzyl)-1,4,7-triazonane-2,5,8-triyl)triacetic acid;
g) 2,2′,2″-(3-(4-(3-(37,40-dioxo-40-((6-(6-(pyridin-2-yl)-1,2,4,5-tetrazin-3-yl)pyridin-3-yl)amino)-3,6,9,12,15,18,21,24,27,30,33-undecaoxa-36-azatetracontyl)thioureido)benzyl)-1,4,7-triazonane-2,5,8-triyl)triacetic acid;
h) 2,2′,2″-(3-(4-(1-(4-(6-methyl-1,2,4,5-tetrazin-3-yl)phenyl)-3-oxo-6,9,12,15,18,21,24-heptaoxa-2-azaheptacosan-27-amido)benzyl)-1,4,7-triazonane-2,5,8-triyl)triacetic acid;
i) 2,2′,2″-(2-(4-(1-(4-(6-methyl-1,2,4,5-tetrazin-3-yl)phenoxy)-3,6,9,12,15,18,21,24,27,30,33-undecaoxahexatriacontan-36-amido)benzyl)-1,4,7-triazonane-1,4,7-triyl)triacetic acid;
j) 2,2′,2″-(3-(4-(3-(5-amino-6-((4-(6-methyl-1,2,4,5-tetrazin-3-yl)benzyl)amino)-6-oxohexyl)thioureido)benzyl)-1,4,7-triazonane-2,5,8-triyl)triacetic acid;
k) 2,2′-(7-(4-(3-(5-amino-6-((4-6-methyl-1,2,4,5-tetrazin-3-yl)benzyl)amino)-6-oxohexyl)thioureido)benzyl)-1,4,7-triazonane-1,4-diyl)diacetic acid;
l) 2,2′,2″-(3-(4-(3-(5-amino-6-((5-amino-6-((4-(6-methyl-1,2,4,5-tetrazin-3-yl)benzyl)amino)-6-oxohexyl)amino)-6-oxohexyl)thioureido)benzyl)-1,4,7-triazonane-2,5,8-triyl)triacetic acid; and
m) 2,2′,2″-(3-(4-(3-(5-amino-6-((5-amino-6-((5-amino-6-((4-(6-methyl-1,2,4,5-tetrazin-3-yl)benzyl)amino)-6-oxohexyl)amino)-6-oxohexyl)amino)-6-oxohexyl)thioureido)benzyl)-1,4,7-triazonane-2,5,8-triyl)triacetic acid.

32. The radioimmunoconjugate of any one of claims 20-31, wherein the antibody-TCO conjugate and the radioligand bind together to form the radioimmunoconjugate via an in vivo click reaction.

33. A method of in vivo diagnosis of a disease or disorder associated with DLL3 in a subject, comprising:
a) administering to the subject the antibody-TCO conjugate of any one of claims 1-17 or the composition of claim 18 or 19, wherein a portion of the antibody-TCO conjugate localizes at cells of the disease or disorder and unbound antibody-TCO conjugate is cleared from blood, from renal system, and/or from the subject after an accumulation interval,
b) administering to the subject the a radioligand comprising a tetrazine moiety (Tz) and a radiolabel or a composition comprising the radioligand, wherein the antibody-TCO conjugate and the radioligand bind together to form a radioimmunoconjugate via an in vivo click reaction at the cells of the disease or disorder within a region of the subject; and
c) imaging the radioimmunoconjugate accumulated in the region of the subject.

34. The method of claim 33, wherein the radioligand is delivered to the subject after the accumulation interval.

35. The method of claim 33 or 34, wherein the imaging occurs via positron emission tomography (PET).

36. The method of any one of claims 33-35, wherein the imaging occurs within a time period up to about 7 days from the administering of the radioligand.

37. The method of claim any one of claims 33-36, wherein the radiolabel comprises a radioactive isotope selected from the group consisting of ^{99m}Tc, ¹¹¹In, ⁴⁴Sc, ⁴⁷Sc, ⁶⁴Cu, ⁶⁷Ga, ⁶⁸Ga, ⁷²As, ⁷⁷As, ⁵²Mn, ¹⁸⁶Re, ¹⁵³Sm, ¹⁷⁷Lu, ⁶⁷Cu, ¹²³I, ¹²⁴I, ¹²⁵I, ¹¹C, ¹³N, ¹⁵O, ¹⁸F, ¹⁶⁶Ho, ¹⁴⁹Pm, ⁹⁰Y, ⁸⁶Y, ²¹³Bi, ¹⁰³Pd, ¹⁰⁹Pd ¹⁵⁹Gd, ¹⁴⁰La, ¹⁹⁸Au, ¹⁹⁹Au, ¹⁷⁵Yb, ¹⁶⁹Yb, ¹⁶⁵Dy, ¹⁶⁶Dy, ¹⁰⁵R ¹¹¹Ag, ⁸⁹Zr, ¹⁹²Ir, ²²⁵Ac, ²¹²Bi, ²¹³Bi, ⁷⁶Br, ⁵⁵Co, ⁶⁰Cu, ⁶¹Cu, ⁶²Cu, ⁶⁶Ga, ⁹⁰Nb, ²¹²Pb, ¹⁵²Tb, ⁷⁷Br, ²¹¹At, ²²⁷Th, ²²⁴Ra and ¹⁴⁹Tb.

38. The method of claim any one of claims 33-37, wherein the radiolabel comprises a ⁶⁴Cu radioactive isotope.

39. The method of claim any one of claims 33-38, wherein the radiolabel comprises a ¹⁸F radioactive isotope.

40. The method of any one of claims 33-39, wherein the radioligand further comprises a chelator.

41. The method of claim 40, wherein the chelator is a sarcophagine chelator.

42. The method of claim 40, wherein the chelator is 1,4,7-triazonane-1,4,7-triyl-triacetic acid (NOTA) or 1,4,7-triazacyclononane-1,4-diacetate (NODA).

43. The method of any one of claims 33-42, wherein the radioligand further comprises a linker attaching the tetrazine moiety (Tz) to the chelator.

44. The method of claim 43, wherein the linker comprises is polyethylene glycol (PEG) or (poly)-L-lysine.

45. The method of claim 44, wherein the polyethylene glycol (PEG) comprises from about 1 unit to about 100 units.

46. The method of claim 44, wherein the (poly)-L-lysine comprises from about 1 unit to about 200 units.

47. The method of any one of claims 33-46, wherein the tetrazine moiety (Tz) and the chelator form a compound selected from the group consisting of:
a) 2,2′,2″-(3-(4-(3-(1-(4-(1,2,4,5-tetrazin-3-yl)phenyl)-1-oxo-5,8,11,14,17,20,23-heptaoxa-2-azapentacosan-25-yl)thioureido)benzyl)-1,4,7-triazonane-2,5,8-triyl)triacetic acid;
b) 2,2′,2″-(3-(4-(3-(1-(4-(1,2,4,5-tetrazin-3-yl)phenyl)-1-oxo-5,8,11,14,17,20,23,26,29,32,35-undecaoxa-2-azaheptatriacontan-37-yl)thioureido)benzyl)-1,4,7-triazonane-2,5,8-triyl)triacetic acid;
c) 2,2′-(7-(4-(3-(1-(4-(1,2,4,5-tetrazin-3-yl)phenyl)-1-oxo-5,8,11,14,17,20,23,26,29,32,35-undecaoxa-2-azaheptatriacontan-37-yl)thioureido)benzyl)-1,4,7-triazonane-1,4-diyl)diacetic acid;
d) 2,2′,2″-(3-(4-(3-(1-(4-(1,2,4,5-tetrazin-3-yl)phenyl)-3,7-dioxo-11,14,17,20,23,26,29-heptaoxa-2,8-diazahentriacontan-31-yl)thioureido)benzyl)-1,4,7-triazonane-2,5,8-triyl)triacetic acid;
e) 2,2′,2″-(3-(4-(3-(1-(4-(1,2,4,5-tetrazin-3-yl)phenyl)-3,7-dioxo-11,14,17,20,23,26,29,32,35,38,41-undecaoxa-2,8-diazatritetracontan-43-yl)thioureido)benzyl)-1,4,7-triazonane-2,5,8-triyl)triacetic acid;
f) 2,2′,2″-(3-(4-(3-(25,28-dioxo-28-((6-(6-(pyridin-2-yl)-1,2,4,5-tetrazin-3-yl)pyridin-3-yl)amino)-3,6,9,12,15,18,21-heptaoxa-24-azaoctacosyl)thioureido)benzyl)-1,4,7-triazonane-2,5,8-triyl)triacetic acid;
g) 2,2′,2″-(3-(4-(3-(37,40-dioxo-40-((6-(6-(pyridin-2-yl)-1,2,4,5-tetrazin-3-yl)pyridin-3-yl)amino)-3,6,9,12,15,18,21,24,27,30,33-undecaoxa-36-azatetracontyl)thioureido)benzyl)-1,4,7-triazonane-2,5,8-triyl)triacetic acid;
h) 2,2′,2″-(3-(4-(1-(4-(6-methyl-1,2,4,5-tetrazin-3-yl)phenyl)-3-oxo-6,9,12,15,18,21,24-heptaoxa-2-azaheptacosan-27-amido)benzyl)-1,4,7-triazonane-2,5,8-triyl)triacetic acid;
i) 2,2′,2″-(2-(4-(1-(4-(6-methyl-1,2,4,5-tetrazin-3-yl)phenoxy)-3,6,9,12,15,18,21,24,27,30,33-undecaoxahexatriacontan-36-amido)benzyl)-1,4,7-triazonane-1,4,7-triyl)triacetic acid;
j) 2,2′,2″-(3-(4-(3-(5-amino-6-((4-(6-methyl-1,2,4,5-tetrazin-3-yl)benzyl)amino)-6-oxohexyl)thioureido)benzyl)-1,4,7-triazonane-2,5,8-triyl)triacetic acid;
k) 2,2′-(7-(4-(3-(5-amino-6-((4-6-methyl-1,2,4,5-tetrazin-3-yl)benzyl)amino)-6-oxohexyl)thioureido)benzyl)-1,4,7-triazonane-1,4-diyl)diacetic acid;
l) 2,2′,2″-(3-(4-(3-(5-amino-6-((5-amino-6-((4-(6-methyl-1,2,4,5-tetrazin-3-yl)benzyl)amino)-6-oxohexyl)amino)-6-oxohexyl)thioureido)benzyl)-1,4,7-triazonane-2,5,8-triyl)triacetic acid; and
m) 2,2′,2″-(3-(4-(3-(5-amino-6-((5-amino-6-((5-amino-6-((4-(6-methyl-1,2,4,5-tetrazin-3-yl)benzyl)amino)-6-oxohexyl)amino)-6-oxohexyl)amino)-6-oxohexyl)thioureido)benzyl)-1,4,7-triazonane-2,5,8-triyl)triacetic acid.

48. The method of any one of claims 33-47, wherein the imaging comprises detecting the radioactive levels emitted by the radioimmunoconjugate.

49. The method of claim 48, wherein the radioactive levels emitted by the radioimmunoconjugate are expressed as the percentage injected dose per gram tissue (% ID/g).

50. A method for treating a disease or disorder associated with DLL3 in a subject, comprising:
a) administering to the subject the antibody-TCO conjugate of any one of claims 1-17 or the composition of claim 18 or 19, wherein a portion of the antibody-TCO conjugate localizes at cells of the disease or disorder and unbound antibody-TCO conjugate is cleared from blood, from renal system, and/or from the subject after an accumulation interval; and
b) administering to the subject the a radioligand comprising a tetrazine moiety (Tz) and a radiolabel or a composition comprising the radioligand, wherein the antibody-TCO conjugate and the radioligand bind together to form a radioimmunoconjugate via an in vivo click reaction at the cells of the disease or disorder within a region of the subject, and wherein the radiolabel comprises a radioactive isotope selected from the group consisting of ^{99m}Tc, ¹¹¹In, ⁴⁴Sc, ⁴⁷Sc, ⁶⁴Cu, ⁶⁷Ga, ⁶⁸Ga, ⁷²As, ⁷⁷As, ⁵²Mn, ¹⁸⁶Re, ¹⁵³Sm, ¹⁷⁷Lu, ⁶⁷Cu, ¹²³I, ¹²⁴I, ¹²⁵I, ¹¹C, ¹³N, ¹⁵O, ¹⁸F, ¹⁶⁶Ho, ¹⁴⁹Pm, 90Y, ⁸⁶Y, ²¹³Bi, ¹⁰³Pd, ¹⁰⁹Pd, ¹⁵⁹Gd, ¹⁴⁰La, ¹⁹⁸Au, ¹⁹⁹Au, ¹⁷⁵Yb, ¹⁶⁹Yb, ¹⁶⁵Dy, ¹⁶⁶Dy, ¹⁰⁵Rh, ¹¹¹Ag, ⁸⁹Zr, ¹⁹²Ir, ²²⁵Ac, ²¹²Bi, ²¹³Bi, ⁷⁶Br, ⁵⁵Co, ⁶⁰Cu, ⁶¹Cu, ⁶²Cu, ⁶⁶Ga, ⁹⁰Nb, ²¹²Pb, ¹⁵²Tb, ⁷⁷Br, ²¹¹At, ²²⁷Th, ²²⁴Ra and ¹⁴⁹Tb.

51. The method of claim 50, wherein the radioligand is delivered to the subject after the accumulation interval.

52. The method of claim 50 or 51, wherein the radioligand further comprises a chelator.

53. The method of claim 52, wherein the chelator is a sarcophagine chelator.

54. The method of claim 52, wherein the chelator is 1,4,7-triazonane-1,4,7-triyl-triacetic acid (NOTA) or 1,4,7-triazacyclononane-1,4-diacetate (NODA).

55. The method of any one of claims 50-54, wherein the radioligand further comprises a linker attaching the tetrazine moiety (Tz) to the chelator.

56. The method of claim 55, wherein the linker comprises is polyethylene glycol (PEG) or (poly)-L-lysine.

57. The method of claim 56, wherein the polyethylene glycol (PEG) comprises from about 1 unit to about 100 units.

58. The method of claim 56, wherein the (poly)-L-lysine comprises from about 1 unit to about 200 units.

59. The method of any one of claims 50-58, wherein the tetrazine moiety (Tz) and the chelator form a compound selected from the group consisting of:
a) 2,2′,2″-(3-(4-(3-(1-(4-(1,2,4,5-tetrazin-3-yl)phenyl)-1-oxo-5,8,11,14,17,20,23-heptaoxa-2-azapentacosan-25-yl)thioureido)benzyl)-1,4,7-triazonane-2,5,8-triyl)triacetic acid;
b) 2,2′,2″-(3-(4-(3-(1-(4-(1,2,4,5-tetrazin-3-yl)phenyl)-1-oxo-5,8,11,14,17,20,23,26,29,32,35-undecaoxa-2-azaheptatriacontan-37-yl)thioureido)benzyl)-1,4,7-triazonane-2,5,8-triyl)triacetic acid;
c) 2,2′-(7-(4-(3-(1-(4-(1,2,4,5-tetrazin-3-yl)phenyl)-1-oxo-5,8,11,14,17,20,23,26,29,32,35-undecaoxa-2-azaheptatriacontan-37-yl)thioureido)benzyl)-1,4,7-triazonane-1,4-diyl)diacetic acid;
d) 2,2′,2″-(3-(4-(3-(1-(4-(1,2,4,5-tetrazin-3-yl)phenyl)-3,7-dioxo-11,14,17,20,23,26,29-heptaoxa-2,8-diazahentriacontan-31-yl)thioureido)benzyl)-1,4,7-triazonane-2,5,8-triyl)triacetic acid;
e) 2,2′,2″-(3-(4-(3-(1-(4-(1,2,4,5-tetrazin-3-yl)phenyl)-3,7-dioxo-11,14,17,20,23,26,29,32,35,38,41-undecaoxa-2,8-diazatritetracontan-43-yl)thioureido)benzyl)-1,4,7-triazonane-2,5,8-triyl)triacetic acid;
f) 2,2′,2″-(3-(4-(3-(25,28-dioxo-28-((6-(6-(pyridin-2-yl)-1,2,4,5-tetrazin-3-yl)pyridin-3-yl)amino)-3,6,9,12,15,18,21-heptaoxa-24-azaoctacosyl)thioureido)benzyl)-1,4,7-triazonane-2,5,8-triyl)triacetic acid;
g) 2,2′,2″-(3-(4-(3-(37,40-dioxo-40-((6-(6-(pyridin-2-yl)-1,2,4,5-tetrazin-3-yl)pyridin-3-yl)amino)-3,6,9,12,15,18,21,24,27,30,33-undecaoxa-36-azatetracontyl)thioureido)benzyl)-1,4,7-triazonane-2,5,8-triyl)triacetic acid;
h) 2,2′,2″-(3-(4-(1-(4-(6-methyl-1,2,4,5-tetrazin-3-yl)phenyl)-3-oxo-6,9,12,15,18,21,24-heptaoxa-2-azaheptacosan-27-amido)benzyl)-1,4,7-triazonane-2,5,8-triyl)triacetic acid;
i) 2,2′,2″-(2-(4-(1-(4-(6-methyl-1,2,4,5-tetrazin-3-yl)phenoxy)-3,6,9,12,15,18,21,24,27,30,33-undecaoxahexatriacontan-36-amido)benzyl)-1,4,7-triazonane-1,4,7-triyl)triacetic acid;
j) 2,2′,2″-(3-(4-(3-(5-amino-6-((4-(6-methyl-1,2,4,5-tetrazin-3-yl)benzyl)amino)-6-oxohexyl)thioureido)benzyl)-1,4,7-triazonane-2,5,8-triyl)triacetic acid;
k) 2,2′-(7-(4-(3-(5-amino-6-((4-6-methyl-1,2,4,5-tetrazin-3-yl)benzyl)amino)-6-oxohexyl)thioureido)benzyl)-1,4,7-triazonane-1,4-diyl)diacetic acid;
l) 2,2′,2″-(3-(4-(3-(5-amino-6-((5-amino-6-((4-(6-methyl-1,2,4,5-tetrazin-3-yl)benzyl)amino)-6-oxohexyl)amino)-6-oxohexyl)thioureido)benzyl)-1,4,7-triazonane-2,5,8-triyl)triacetic acid; and
m) 2,2′,2″-(3-(4-(3-(5-amino-6-((5-amino-6-((5-amino-6-((4-(6-methyl-1,2,4,5-tetrazin-3-yl)benzyl)amino)-6-oxohexyl)amino)-6-oxohexyl)amino)-6-oxohexyl)thioureido)benzyl)-1,4,7-triazonane-2,5,8-triyl)triacetic acid.

60. The method of any one of claims 33-59, wherein the disease or disorder associated with DLL3 is a tumor.

61. The method of claim 60, wherein the tumor is a cancer.

62. The method of any one of claims 33-61, wherein the disease or disorder associated with DLL3 is selected from neuroendocrine tumors of the lung, extrapulmonary neuroendocrine carcinomas, melanoma, neuroendocrine prostate cancer, and breast cancer.

63. The method of claim 62, wherein the neuroendocrine tumors of the lung are selected from the group consisting of pulmonary neuroendocrine cancer, large cell neuroendocrine carcinoma, and small-cell lung cancer.

64. The method of any one of claims 33-63, wherein the subject is a human.

65. A kit for in vivo diagnosis and/or treatment of a disease or disorder associated with DLL3 in a subject, comprising the antibody-TCO conjugate of any one of claims 1-17 or the composition of claim 18 or 19, and a radioligand comprising a tetrazine moiety (Tz) and a radiolabel or a composition thereof.

66. The kit of claim 65, wherein the kit further comprises written instructions for using the antibody-TCO conjugate and the radioligand for in vivo diagnosis and/or treatment of a disease or disorder associated with DLL3 in a subject.
